(19) United States
(12) Patent Application Publication
(10) Pub. No.: US 2026/0034119 A1
(43) Pub. Date: Feb. 5, 2026

(54) 5-METHOXY-N,N-DIMETHYLTRYPTAMINE ANALOGS, THEIR SYNTHESIS, AND METHODS FOR TREATMENT OF NEUROLOGICAL, PSYCHIATRIC, AND SUBSTANCE USE DISORDERS

(71) Applicants: The Trustees of Columbia University in the City of New York, New York, NY (US); ICAHN SCHOOL OF MEDICINE AT MOUNT SINAI, New York, NY (US)

(72) Inventors: David LANKRI, Brooklyn, NY (US); Dalibor SAMES, New York (NY); Inis SERRANO, Woodhaven, NY (US); Michael CUNINGHAM, Asheville, NC (US); Audrey WARREN, New York, NY (US); Daniel WACKER, New York, NY (US); Lyonna PARISE, New York, NY (US); Scott RUSSO, New York, NY (US)

(73) Assignees: The Trustees of Columbia University in the City of New York, New York, NY (US); ICAHN SCHOOL OF MEDICINE AT MOUNT SINAI, New York, NY (US)

(21) Appl. No.: 19/295,282

(22) Filed: Aug. 8, 2025

Related U.S. Application Data
(63) Continuation of application No. PCT/US2024/014902, filed on Feb. 7, 2024.
(60) Provisional application No. 63/444,171, filed on Feb. 8, 2023.
(60) Provisional application No. 63/613,234, filed on Dec. 21, 2023.

(51) Int. Cl. A61K 31/4545; A61K 31/343; A61K 31/4025; A61K 31/4045; A61K 31/407; A61K 31/4178; A61K 31/437; A61K 31/439; A61K 31/443; A61K 31/4439; A61K 31/4525; A61K 31/454; A61P 25/16; A61P 25/22; A61P 25/24; C07D 209/12; C07D 209/16; C07D 307/81; C07D 401/06; C07D 403/06; C07D 405/06; C07D 471/04; C07D 471/08; C07D 491/056; C07D 519/00

(52) U.S. Cl. CPC A61K 31/4545; A61K 31/343; A61K 31/4025; A61K 31/4045; A61K 31/407; A61K 31/4178; A61K 31/437; A61K 31/439; A61K 31/443; A61K 31/4439; A61K 31/4525; A61K 31/454; A61P 25/16; A61P 25/22; A61P 25/24; C07D 209/12; C07D 209/16; C07D 307/81; C07D 401/06; C07D 403/06; C07D 405/06; C07D 471/04; C07D 471/08; C07D 491/056; C07D 519/00

(57) ABSTRACT

Disclosed are compounds having the structure:

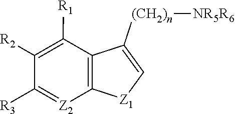

wherein
   Z₁ is NR₄, O, or S;
   Z₂ is CR₇ or N;
   n is 1 to 5;
   R₁ is —H, halogen, —O-alkyl, or together with R₂ forms —O—CH₂—O—;
   R₂ is —H, —OH, —O-alkyl, —O-alkyl-aryl, —S-alkyl, —S-alkyl-aryl, or together with R₁ or R₃ forms —O—CH₂—O—;
   R₃ is —H, —O-alkyl or together with R₂ forms —O—CH₂—O—;
   R₄ is H or alkyl;
   R₅ and R₆ are each independently H, alkyl, alkenyl, alkynyl or NR₅R₆ together form a substituted or unsubstituted heterocycle, wherein the heterocycle may be monocyclic or bicyclic,
   R₇ is H or alkyl;
      wherein R₁ is F, when R₂ is —O-(n-propyl) or when at least one of R₅ or R₆ is H, and
      wherein the compound is other than a compound where
         Z₁ is NR₄ and R₁, R₂, R₃, and R₈ are H;
         n is 2, R₁ is halogen, R₂ is —OCH₃, R₃ is —H, R₄ is —H, and R₅ and R₆ are both methyl;
         n is 2, R₁ is —F, R₂ is —OCH₃, R₃ is —H, R₄ is —H, and NR₅R₆ together are —N-pyrrolidine;
         n is 2, R₁ and R₂ together forms —O—CH₂—O—, R₃ is —H, R₄ is —H, and R₅ and R₆ are both methyl; and
         n is 2, R₁ and R₂ together forms —O—CH₂—O—, R₃ is —H,
         R₄ is —H; and R₅ and R₆ are both isopropyl,
   or a pharmaceutically acceptable salt thereof.

Also disclosed are processes for synthesizing the compounds, and methods for activating or selectively activating the 5HT1A receptor, or of simultaneously activating the 5HT1A and 5HT2A receptors, and of treating a subject afflicted with a neurological disease, psychiatric disorder, or substance use disorder, using the compounds.

This application is a continuation of PCT International Application No. PCT/US2024/014902, filed Feb. 7, 2024, claiming the benefit of U.S. Provisional Application No. 63/613,234 filed Dec. 21, 2023, and U.S. Provisional Application No. 63/444,171 filed Feb. 8, 2023, the contents of each of which are hereby incorporated by reference into the subject application.

This invention was made with government support under GM133504, MH132317, GM062754, DA053558, MH127820, MH104559, GM129539, GM103310, and TR004419 awarded by the National Institutes of Health and KP160711 by the DOE. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this disclosure pertains.

### BACKGROUND OF THE INVENTION

Recent scientific inquiry has demonstrated both rapid and long-lasting anxiolytic and antidepressant effects of classic serotoninergic psychedelics such as psilocybin and LSD. Although the mind-altering, psychedelic effects have been ascribed to actions at 5-HT2A serotonin (5-hydroxytryptamine, 5-HT) receptors, studies indicate a modulatory role of other 5-HT receptors. The complex behavioral effects of the lesser studied hallucinogen 5-methoxy-N,N-dimethyltryptamine (5-MeO-DMT), found in the poison of the Colorado River toad (Incilius alvarius) (Weil et al. 1994, Schwelm, 2021), are particularly reliant on the drug's actions at the 5-HT1A receptor. For instance, the discriminatory stimulus of 5-MeO-DMT as well as its effects on exploratory behaviors and sedation appear largely driven by 5-HT1A agonist activity in vivo (Winter et al. 2000, Krebs-Thomson et al. 2006). Epidemiological surveys, capturing non-traditional medical use of 5-MeO-DMT, indicate that 5-MeO-DMT generates a rapid and sustained reduction in depression and anxiety symptoms, as well as induction of meaningful and spiritually significant experiences (Eramkova et al. 2022, Davis et al. 2019, Uthaug et al 2019). 5-MeO-DMT is also used in combination with the hallucinogen ibogaine in clinics outside the U.S. A recent survey of U.S. Special Operation Forces veterans highlights the therapeutic promise in the treatment of PTSD (suicidal ideation, cognitive impairment), depression, and anxiety (Davis et al. 2020, Armstrong et al. 2023). In fact, 5-MeO-DMT is currently in development as a therapeutic for a range of indications including depression, substance use disorders, and neurological disorders (Reckweg et al. 2012). Although based largely on open-label trials and naturalistic surveys, the existing evidence suggests 5-MeO-DMT produces rapid and profound effects across neuropsychiatric diagnostic indications. Given that 5-HT1A is the primary target of several approved anxiolytic and antidepressant medications, such as buspirone (Buspar®) (Loane et al. 2012) and vilazodone (Viibryd®) (Chauhan et al. 2022), and in vivo studies demonstrate a key role for 5-HT1A in 5-MeO-DMT effects, this receptor may contribute to the reported therapeutic effects of 5-MeO-DMT.

While we and others have recently uncovered much about the molecular mechanisms of LSD and other psychedelics at 5-HT2A (Kim et al. 2020, Wacker et al. 2017) little is known about how 5-MeO-DMT, related tryptamines, and classic psychedelics bind to and signal via 5-HT1A. Much of psychedelic research and development of novel probes has focused on the 5-HT2A receptor (Cameron et al. 2021, Cao et al. 2022, Kaplan et al. 2022), while notably less effort has been dedicated to investigating the role of other 5-HT receptors in the complex polypharmacology of psychedelics. This is despite 5-HT1A's and 5-HT2A's proposed complementary roles in moderating anxiety and stress (Carhart-Harris et al. 2017), which are some of the major areas for potential psychedelic-based therapies. We herein disclose a detailed structural and functional exploration of the molecular and atomic level mechanisms by which classic psychedelics, 5-methoxytryptamines, and prescription drugs bind to and activate 5-HT1A. We also disclose the therapeutic action of 5-HT1A-selective tryptamines in a rodent model of depression, thereby highlighting the application of our novel tryptamine-based 5-HT1A compounds.

The present invention focuses on 5-HT1A-selective analogs of 5-MeO-DMT and their method of action.

### SUMMARY OF THE INVENTION

The present invention provides a compound having the structure:

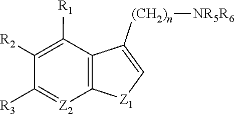

wherein
   Z₁ is NR₄, O, or S;
   Z₂ is CR₇ or N;
   n is 1 to 5;
   R₁ is —H, halogen, —O-alkyl, or together with R₂ forms —O—CH₂—O—;
   R₂ is —H, —OH, —O-alkyl, —O-alkyl-aryl, —S-alkyl, —S-alkyl-aryl, or together with R₁ or R₃ forms —O—CH₂—O—;
   R₃ is —H, —O-alkyl or together with R₂ forms —O—CH₂—O—;
   R₄ is H or alkyl;
   R₅ and R₆ are each independently H, alkyl, alkenyl, alkynyl or NR₅R₆ together form a substituted or unsubstituted heterocycle,
   wherein the heterocycle may be monocyclic or bicyclic,
   R₇ is H or alkyl;
      wherein R₁ is F, when R₂ is —O-(n-propyl) or when at least one of R₅ or R₆ is H, and
   wherein the compound is other than a compound where
      Z₁ is NR₄ and R₁, R₂, R₃, and R₈ are H;
      n is 2, R₁ is halogen, R₂ is —OCH₃, R₃ is —H, R₄ is —H, and R₅ and R₆ are both methyl;
      n is 2, R₁ is —F, R₂ is —OCH₃, R₃ is —H, R₄ is —H, and NR₅R₆ together are —N-pyrrolidine;
      n is 2, R₁ and R₂ together forms —O—CH₂—O—, R₃ is —H, R₄ is —H, and R₅ and R₆ are both methyl; and
      n is 2, R₁ and R₂ together forms —O—CH₂—O—, R₃ is —H, R₄ is —H; and R₅ and R₆ are both isopropyl,
   or a pharmaceutically acceptable salt thereof.

The present invention also provides a method of activating or selectively
activating the 5HT1A receptor, or of simultaneously activating the 5HT1A
and 5HT2A receptors, in a subject comprising administering to the subject
an amount of the compound of the present invention effective to activate or selectively activate the 5HT1A receptor, or simultaneously activate the 5HT1A and 5HT2A receptors.

The present invention further provides a method of treating a subject afflicted with a neurological disease, psychiatric disorder, or substance use disorder, comprising administering to the subject an amount of the compound or the pharmaceutical composition of the present invention effective to treat the subject.

The present invention still further provides a process for the preparation of the compound of the present invention comprising a step of dissolving a compound having the structure:

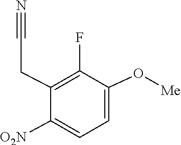 [SMILES: COc1ccc([N+](=O)[O-])c(CC#N)c1F]

in a suitable solvent followed by treatment with a suitable acid in the presence of a palladium catalyst at elevated pressure in an atmosphere of hydrogen.

The present invention yet further provides a process for the preparation of the compound of the present invention comprising a step of dissolving a compound having the structure:

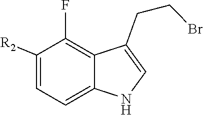

in a suitable solvent followed by treatment with a suitable amine.

### BRIEF DESCRIPTION OF THE DRAWINGS

FIG. 1 Pharmacokinetics (PK) of 4-F, 5-MeO-PyrT at 1 mg/kg SC administration in mice.

FIGS. 2A-B PK of 5-MeO-MET at 1 (A) and 10 mg/kg (B) in mice.

FIG. 3 PK of 4-F, 5-MeO-MET at 10 mg/kg SC administration in mice.

FIG. 4 PK of 4-F, 5-MeO-DPT at 10 mg/kg SC administration in mice.

FIGS. 5A-B Human (A) and mouse (B) microsomal stability characterizations of 4-F, 5-MeO-DMT.

FIGS. 6A-B Human (A) and mouse (B) microsomal stability characterizations of 4-F, 5-MeO-MET, showing greater stability in human microsomes versus the DMT analogs.

FIGS. 7A-B Human (A) and mouse (B) microsomal stability characterizations of 4-F, 5-MeO-MPT, showing greater stability in human microsomes versus the DMT analogs.

FIGS. 8A-B Human (A) and mouse (B) microsomal stability characterizations of 4-F, 5-MeO-MALT, showing greater stability in human microsomes versus the DMT analogs.

FIGS. 9A-B Human (A) and mouse (B) microsomal stability characterizations of 4-F, 5-MeO-EPT, showing greater stability in human microsomes versus the DMT analogs.

FIG. 10 Human and mouse microsomal stability characterizations of 4-F, 5-MeO-DPT, showing greater stability in human microsomes versus the DMT analogs.

FIGS. 11A-B MAO-A (A) and MAO-B (B) activity comparison of reference compound Kynuramine.

FIGS. 12A-B MAO A-(A) and MAO-B (B) activity comparison of 4-F, 5-MeO-DMT.

FIGS. 13A-B MAO A and MAO B activity comparison of 4-F, 5-MeO-MPT, showing markedly slower MAO A metabolism versus the DMT analogs.

FIGS. 14A-B MAO-A (A) and MAO-B (B) activity comparison of 4-F, 5-MeO MALT, showing markedly slower MAO A metabolism versus the DMT analogs.

FIGS. 15A-B MAO A and MAO B activity comparison of 4-F, 5-MeO-DPT, showing markedly slower MAO A metabolism versus the DMT analogs.

FIGS. 16A-B Dose-response curves in 5-HT1A Gi1 and 5-HT2A Gq (BRET) for selected tryptamines, showing greater 5-HT1A selectivity for 4-fluorinated compounds with different aliphatic amine substitution pattern.

FIG. 17 Dose-response curves in 5-HT1A Gi1 (BRET) for selected Tryptamines.

FIG. 18 Dose-response curves in 5-HT2A (IP1 assay) for selected Tryptamines. 4-fluorination has a smaller effect on signaling potency at 5-HT2A compared to 5-HT1A.

FIG. 19 Dose-response curves in 5-HT2B (IP1 assay) for selected tryptamines, showing that 4-fluoro analogs exhibit decreased signaling efficacy at this receptor in comparison to the non-fluorinated compounds, revealing an unexpected effect of 4-fluorination across this tryptamine class, and thus increasing safety margins for these compounds.

FIG. 20 Dose-response curves in 5-HT2A (IP1 assay) and 5-HT1A (BRET) for 4-F, 5-MeO-PyrT, demonstrating that the high 5-HT1A vs 5-HT2A selectivity is due to both an increase in signaling potency at 5-HT1A and decrease in signaling potency at 5-HT2A.

FIGS. 21A-C Social interaction (A), corner time (B), and locomotor activity (C) of 4-F, 5-MeO-PyrT in mice. The social interaction deficit induced by social defeat chronic stress is rescued by one administration of 4-F, 5-MeO-PyrT (1 mg/kg, s.c.), a preclinical measure of antidepressant effect. Increased corner time is also rescued, a measure of anxiolytic effects of the compound, while there are no effects of the pharmacological intervention on general locomotion.

FIGS. 22A-C Social interaction (A), corner time (B), and locomotor activity (C) in response to 4-F, 5-MeO-MET (3 and 10 mg/kg, s.c.). The social interaction deficit induced by social defeat chronic stress is rescued by one administration of 4-F, 5-MeO-MET, a preclinical measure of antidepressant effect. Increased corner time is also rescued, a measure of anxiolytic effects of the compound, while there are no effects on general locomotion.

FIG. 23 Head-Twitch-Response (HTR) as a measure of 5-HT2A-mediated hallucinogenic activity in mice. 4-F, 5-MeO-PyrT shows no preclinical signs of hallucinogenic effects in either the presence or absence of 5-HT1A antagonist (WAY100). 5-MeO-MET was used as a control compound, which shows robust HTR in the presence of WAY100. This demonstrates no or much attenuated psychedelic potential of the 4-fluorinated compounds.

FIG. 24 The sucrose preference test is a paradigm that has been used extensively to assess the effects of stress-induced anhedonia. Stressed mice treated with 4-F, 5-MeO-PyrT had significantly increased sucrose preference relative to vehicle-treated animals and were essentially indistinguishable from non-stressed mice receiving either vehicle or drug.

FIGS. 25A-D (A) A cataleptic dose response curve for tetrabenazine (n=5-7/group, s.c., assessed 60 min post inj.) Tetrabenazine (10.0 & 20.0 mg/kg) significantly induces catalepsy compared to vehicle control, **p≤0.01 and ***p≤0.01. (B) Effect of 4F, 5-MeO-PyrT (1.0 mg/kg, s.c.) on TBZ-induced catalepsy, n=5/group. (C) Effect of 8-OH-DPAT (0.3, 1.0 mg/kg, s.c.) on TBZ-induced catalepsy, n=5/group. (D) Effect of 4F, 5-MeO-PyrT (1.0 mg/kg, s.c.) on haloperidol-induced catalepsy, n=5/group. Analysis was done in GraphPad Prism using one-way ANOVA.

FIGS. 26A-C Cryo-EM Structure Determination of drug-bound 5-HT1A-Gαi1/Gβ1/Gγ2 Complexes. (A) Representative structure determination of 5-MeO-DMT-bound 5-HT1A signaling complex. Top Left, Analytical size exclusion chromatography and SDS-PAGE show monodisperse and pure protein of intact complex and its components. Right, representative Cryo-EM micrograph and data processing schematic exemplified by 5-MeO-DMT-bound 5-HT1A-Gi1 structure: After particle picking, 2D classification and multiple rounds of 3D classification, the final particle stack was refined using non-uniform refinement. A final map was obtained and resolutions were estimated applying the 0.143 cutoff in GS-FSC. Initial models were built in COOT, and then further refined in PHENIX for the generation of final coordinates shown in this manuscript. (B) Local resolution map of a 5-MeO-DMT-bound 5-HT1A-Gi1 complex (left) and FSC curves (right) calculated the final reconstruction in Cryosparc. (C) 5-MeO-DMT in the orthosteric binding pocket from the side (left) and rotated 45° towards the top of the receptor (right) with the map of ligand and surround residue densities shown at 50.

FIGS. 27A-E Comparison of different 5-HT1A structures and differences in binding of LSD to 5-HT1A and 5-HT2A. (A) Superposition of herein reported 5-MeO-DMT-bound 5-HT1A-Gi complex with the previously reported 5-HT-bound 5-HT1A-Gi structure (PDB ID: 7e2y) shows similar conformations. Additional residues in 5-HT1A's EL2 and G proteins not observed in previous structures are highlighted in red. (B) Lipids and cholesterol are bound to similar sites as observed before. (C) Local resolution map of a LSD-bound 5-HT1A-Gi1 complex (left) and FSC curves (right) calculated the final reconstruction in Cryosparc. (D) LSD in the orthosteric binding pocket from the side (top) and rotated 45° towards the top of the receptor (bottom) with the map of ligand and surround residue densities shown at 5a. (E) LSD shows distinct binding modes bound to 5-HT1A-Gi signaling complex and 5-HT2A (PDB ID: 6wgt). Left, 5-HT1A-bound LSD sits deeper in the binding pocket compared to 5-HT2A-bound LSD (orange). Zoom in of LSD in 5-HT1A-Gi structure (middle) and 5-HT2A structure (right) highlights differential stereochemistry and receptor-specific interactions of diethylamide moiety. Hydrogen bonds are indicated as grey dashed lines.

FIGS. 28A-B Global structure-activity landscape and synthesis of tryptamine psychedelics at 5-HT1A and 5-HT2A receptors. (A) Overview of the cryo-EM structure of the 5HT1A receptor-Gi signaling complex bound to 5-MeO-DMT (center). Classic psychedelics such as the prototypical compounds DMT and LSD are agonists of both 5-HT1A and 5-HT2A receptors (left semi-circle). 5-MeO-DMT (top of the circle), a major psychoactive compound found in bufotoxin, shows comparable potency and efficacy at both 5-HT1A and 5-HT2A receptors. Systematic structural mapping via elaboration of the core 5-MeO-DMT structure identifies a class of 5-MeO-tryptamines with increasing 5-HT1A selectivity (right hemi-circle). 5-MeO-DMT can be viewed as a deconstruction of Ibogaine, an atypical psychedelic with complex polycyclic tryptamine structure (bottom of the circle). Iboga compounds show no activity at 5-HT1A and 5-HT2A receptors, but activity re-emerges by deconstruction of the isoquinuclidine core to simple mono-cyclic tryptamines such as 5-MeO-PipT (5-methoxypiperidinyl-tryptamine) and 4-F, 5-MeO-PyrT (4-Fluoro, 5-methoxypyrrolidinyl-tryptamine, right hemi-circle). (B) General synthesis methodology of tryptamines: a. oxalyl chloride, b. MeOH, LAH, c. PPh3, CBr4, d. Amine, TEA, e. Amine, f. LAH

FIGS. 29A-D Global structure-activity landscape and synthesis of tryptamine psychedelics at 5-HT1A and 5-HT2A receptors. (A) Local resolution map of a 4-F, 5-MeO-PyrT-bound 5-HT1A-Gi1 complex (left) and FSC curves (right) calculated from the final reconstruction in Cryosparc. (B) 4-F, 5-MeO-PyrT in the orthosteric binding pocket from the side (left) and rotated 45° towards the top of the receptor (right) with the map of ligand and surround residue densities shown at 5. (C) structural side-by-side comparison of 5-HT1A orthosteric site bound to 5-MeO-DMT and 4-F, 5-MeO-PyrT. (D) cAMP accumulation assays using wildtype and mutant 5-HT1A, and different drugs. Concentration-response experiment reveal different sensitivities of distinct drugs to F361L mutation. All signaling studies were performed in triplicates and are averaged from two to three independent experiments. Data have been normalized against 5-HT and errors bars denote s.e.m.

FIGS. 30A-H Comparison of 4-F, 5-MeO-PyrT binding pose to that of different clinical 5-HT1A drugs. (A) and (E) Local resolution maps of vilazodone-bound (A) and buspirone-bound (E) 5-HT1A-Gi1 complexes and corresponding FSC curves calculated from the final reconstructions in Cryosparc. (B) and (F), Vilazodone (B) and buspirone (F) in the orthosteric binding pocket from the side (left) and rotated 45° towards the top of the receptor (right) with the density map of ligand and surrounding residue shown at 5. (C-H), Extracellular view of 4-F, 5-MeO-PyrT (C), Vilazodone (D), Aripiprazole (G, PDB ID: 7e2z), and Buspirone (H) binding poses in 5-HT1A's orthosteric site.

FIGS. 31A-C Selectivity of different 5-MeO-DMT analogs and off-target activity of 4-F, 5-MeO-PyrT. (A) 5-HT1A-Gi and 5-HT2A-Gq BRET of 5-HT, 5-MeO-DMT, 5-MeO-MET, and 4-F, 5-MeO-PyrT with respective potencies and 5-HT1A>5-HT2A selectivities. (B) Off-target inhibition of transporters SERT, PMAT, Oct1, and Oct2 by 4-F, 5-MeO-PyrT and known inhibitors. (C) Arrestin-recruitment of 5-HT and 4-F, 5-MeO-PyrT at all human 5-HT receptor subtypes. All functional studies were performed in triplicates and are averaged from two to three independent experiments. Data have been normalized against 5-HT, Citalopram, and Decynium-22, and errors bars denote s.e.m.

FIGS. 32A-H Effects of 5-MeO-DMT derivatives on rodent behavior. (A) Evaluation of 4-F, 5-MeO-PyrT in open field for two hours (n=3-4/group). (B) Exemplary traces of the ambulatory distance traveled in open field demonstrating how the pretreatment of 5-HT1A receptor antagonist WAY-100635 (1 mg/kg, s.c., 15 min prior) negates the sedative effects of 4-F, 5-MeO-PyrT (1 mg/kg, s.c.). (C) Effect of WAY-100635 (1 mg/kg, s.c., 15 min prior) on 4-F, 5-MeO-PyrT's and 5-MeO-MET's effect on total locomotion (n=7-8/group, 30 min). (D) To determine the optimal dose of the 5-HT1A receptor antagonist WAY100635, we assessed administration of 1 mg/kg and 2 mg/kg WAY100635 on 4-F, 5-MeO-PyrT's effects on total locomotion (n=7-8/group) and induced HTR (n=6/group). Analysis was done using one-way ANOVA with multiple comparisons with Tukey's post hoc test. All values represented as mean±s.e.m. E-H, Effects of saline or 4-F, 5-MeO-PyrT administration on control (CON) or chronically defeated mice (Stress). Determination of (E) social interaction as a measure of anxiety- and depression-related phenotype, (F), distance moved as a measure of locomotor activity, (G) corner time as a measure of anxiety-like behavior. Number of mice for each group is indicated below the data for each respective cohort. Significant differences were determined by two-way ANOVA and exact p values have been denoted in the Figure.

FIGS. 33A-B Activity of psychedelics at 5-HT1A and 5-HT2A, and Cryo-EM structures of 5-MeO-DMT and LSD bound to the 5-HT1A signaling complex. (A) 5-HT1A-mediated Gi1 activation and 5-HT2A-mediated Gq activation by psychedelics determined by BRET. Concentration response experiments were performed in triplicates and are averaged from two (Mescaline) or three independent experiments. Data have been normalized against 5-HT and errors bars denote s.e.m. (B) CryoEM structure of 5-HT1A receptor-Gαi1/Gβ1/Gy2 signaling complexes. Zoom in shows 5-HT1A orthosteric site bound to 5-MeO-DMT and LSD, with ionic interactions and hydrogen bonds indicated by dashed lines. Bottom panels show superposition of 5-HT1A orthosteric binding sites comparing binding poses of 5-MeO-DMT and LSD, as well as 5-MeO-DMT and 5-HT (PDB ID: 7e2y).

FIGS. 34A-D Differential pharmacological effects of amine and indole modifications to the 5-MeO-DMT scaffold at 5-HT1A and 5-HT2A. 5-HT1A-Gi and 5-HT2A-Gq BRET mediated by (A) “designer” tryptamines and (B) cyclized tryptamines. Effects of indole modifications at the (C) 5th position and (D) 4-fluorination on 5-HT1A-Gi and 5-HT2A-Gq BRET. All signaling experiments were performed in triplicates and are averaged from two (5-MeO-MiPT, 5-MeO-3-PyrrolineT, 5-MeO-PipT) or three independent experiments. Data have been normalized against 5-HT and errors bars denote s.e.m.

FIGS. 35A-B Elucidation of potency and selectivity determinants of tryptamines at 5-HT1A and 5-HT2A. (A) 5-HT1A-Gi and 5-HT2A-Gq signaling mediated by tryptamine compounds at mutant and wildtype receptors according to BRET assays. Middle insert shows residue differences in orthosteric binding pockets of 5-HT1A (pink) and 5-HT2A (green, PDB: 6WGT). (B) Heatmap showing the effect of tryptamine modifications on the signaling potency (pEC50) at mutant and wild type 5-HT1A and 5-HT2A receptors. Grey boxes indicate compound not tested. All signaling experiments were performed in triplicates and are averaged from two (5-HT1A N386V, 5-HT2A V366N, 5-HT1A A365N: 4-F, 5-Meo-PyrT, 5-HT2A N343A: 4-F, 5-Meo-PyrT) or three independent experiments. Data have been normalized against 5-HT and errors bars denote s.e.m.

FIG. 36 (A) Structural and functional comparison of 4-F, 5-MeO-PyrT and clinical 5-HT1A medications at 5-HT1A. 2D structures of 4-F, 5-MeO-PyrT, vilazodone (Viibryd), aripiprazole (Abilify), and buspirone (BuSpar). Side-by-side structural comparison of drug binding poses of 4-F, 5-MeO-PyrT, vilazodone, and buspirone determined in this study, as well as previous structure of aripiprazole (PDB ID: 7e2z) similar binding modes. Buspirone assumes a kinked conformation binding to previously not described extended pocket (EBP2), while vilazodone and aripiprazole stretch towards the extracellular space forming interactions in a distinct extended binding pocket (EBP1). All compounds assume similar overall poses in 5-HT1A's orthosteric binding pocket (OBP). (B) 5-HT1A-Gi BRET of 4-F, 5-MeO-PyrT, vilazodone, aripiprazole, and buspirone. All signaling experiments were performed in triplicates and are averaged from two (aripiprazole) or three independent experiments. Data have been normalized against 5-HT and errors bars denote s.e.m.

FIGS. 37A-E Effects of 5-MeO-DMT derivatives on rodent behavior. (A) Pharmacokinetic (PK) profile of 4-F, 5-MeO-PyrT following s.c. administration of 1 mg/kg. (B) Head-Twitch-Response (HTR) as a measure of 5-HT2A-mediated hallucinogenic activity in mice in the presence and absence of 5-HT1A selective antagonist WAY-100635 (1 mg/kg). (C) Schematic of the chronic social defeat stress (SD) paradigm. (D-E) Effects of saline, 4-F, 5-MeO-PyrT, and WAY-100635 administration on control or chronically defeated (stressed) mice. Determination of social interaction (D) and preference for 1% sucrose solution over water in a two-bottle choice test (E) as a measure of a depressive-like phenotype. Compounds are dosed at 1 mg/kg unless otherwise indicated. Data has been averaged from three (D) and two (E) independent experiments, and number of mice for each group is indicated below the data for each respective cohort. Differences were determined by two-way ANOVA with multiple comparisons using Fisher's LSD post hoc test, and exact p values have been denoted in the Figure.

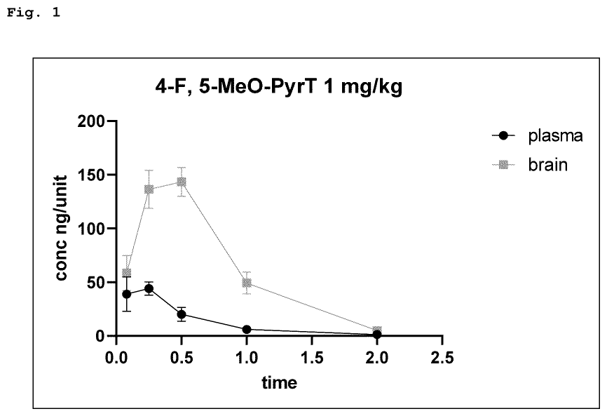

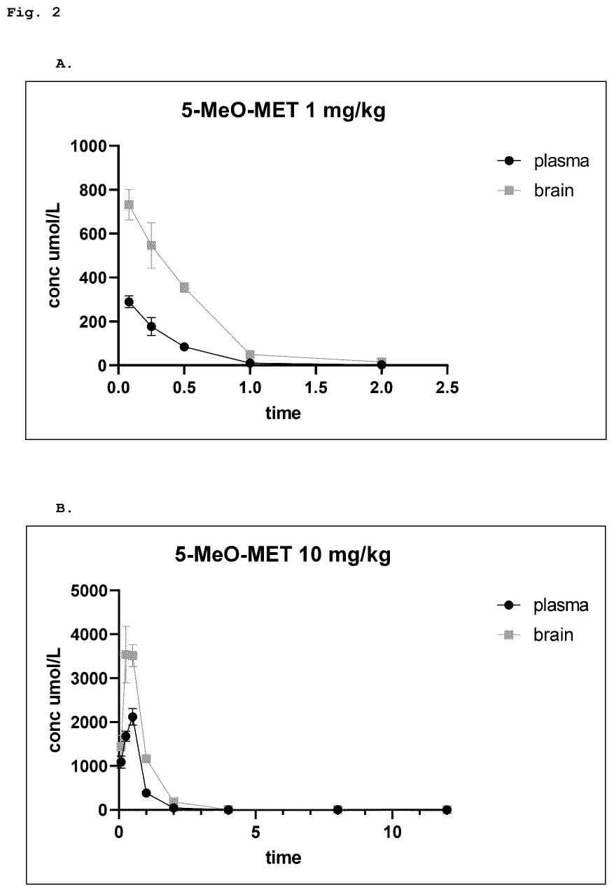

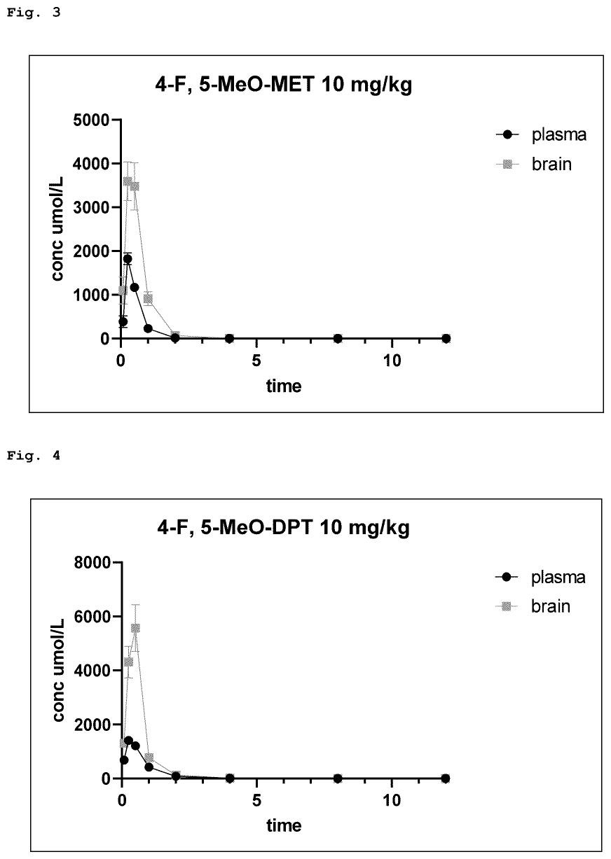

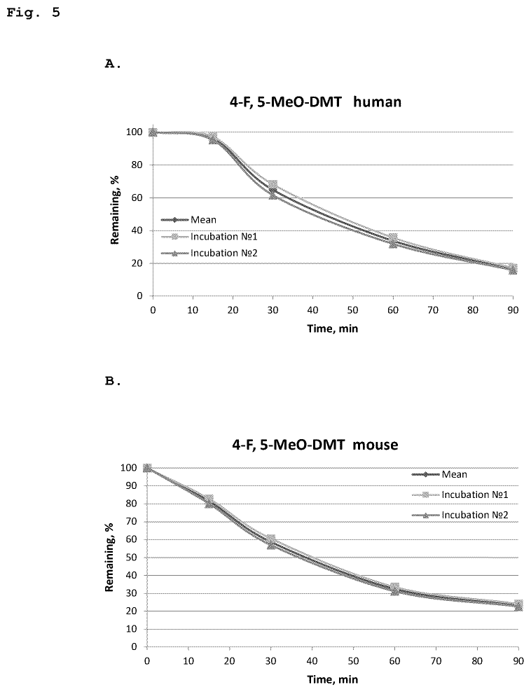

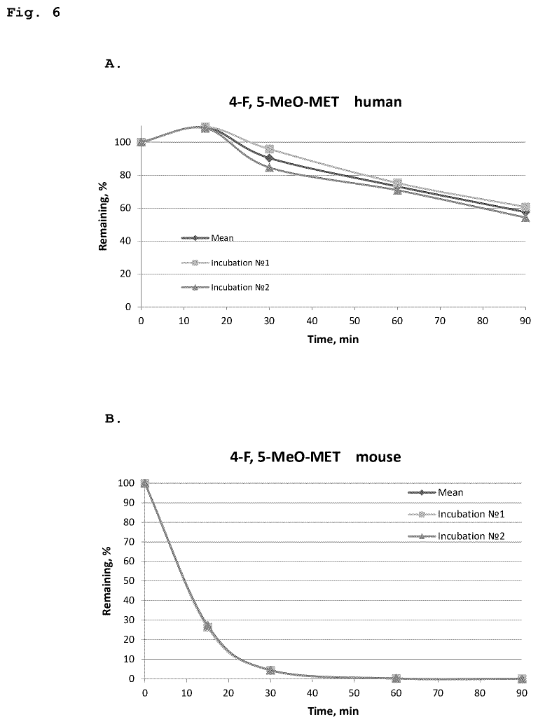

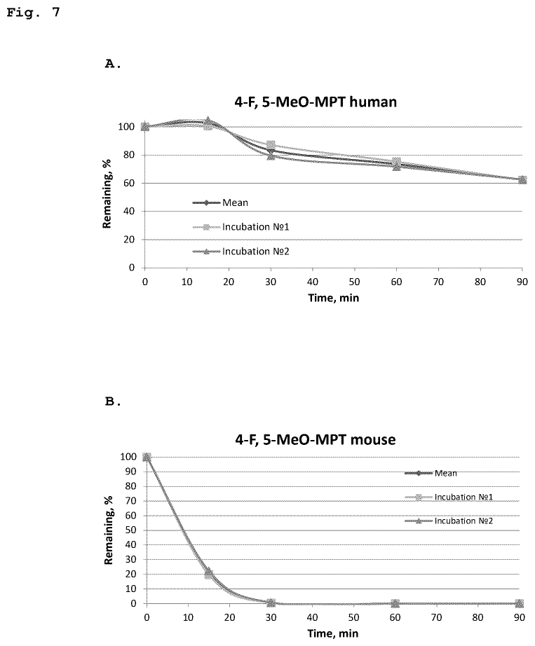

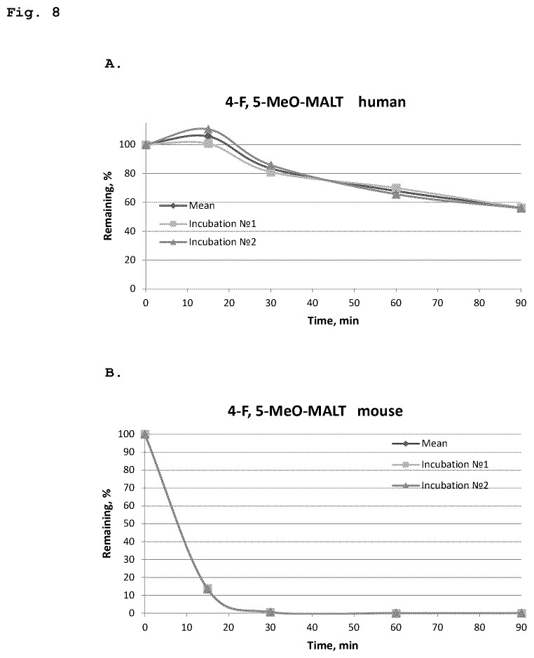

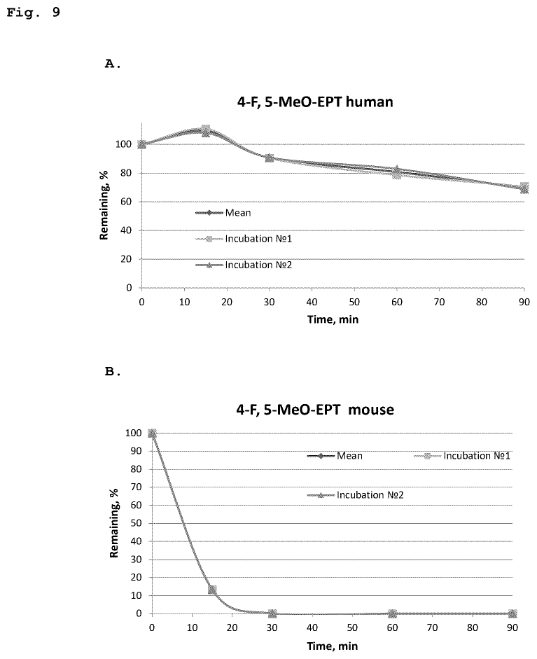

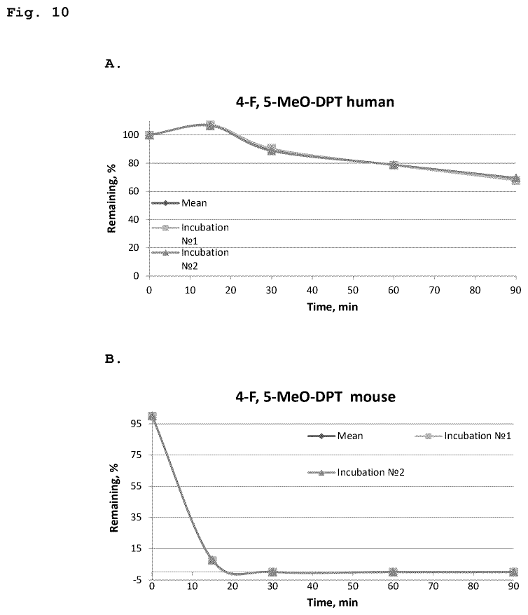

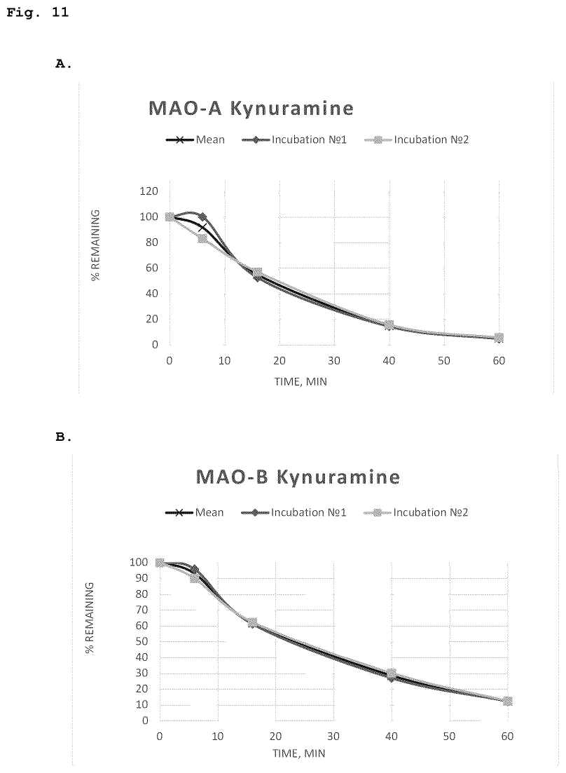

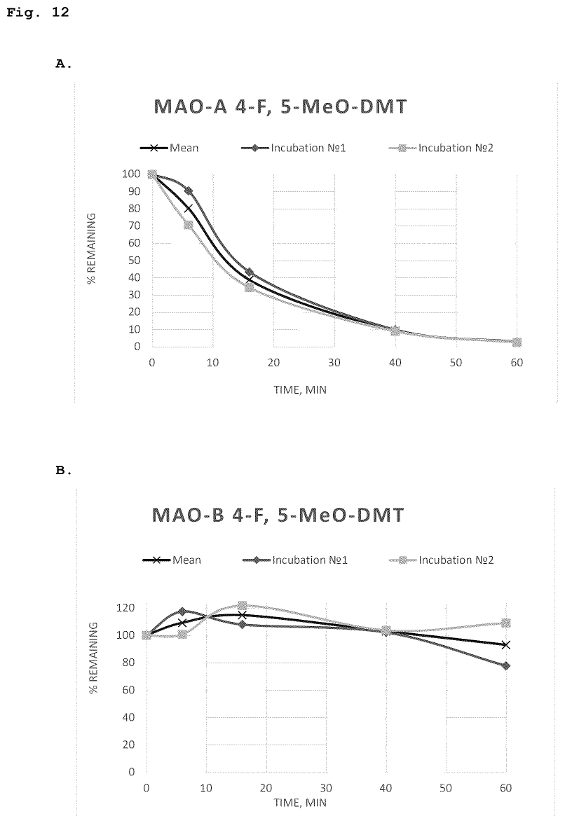

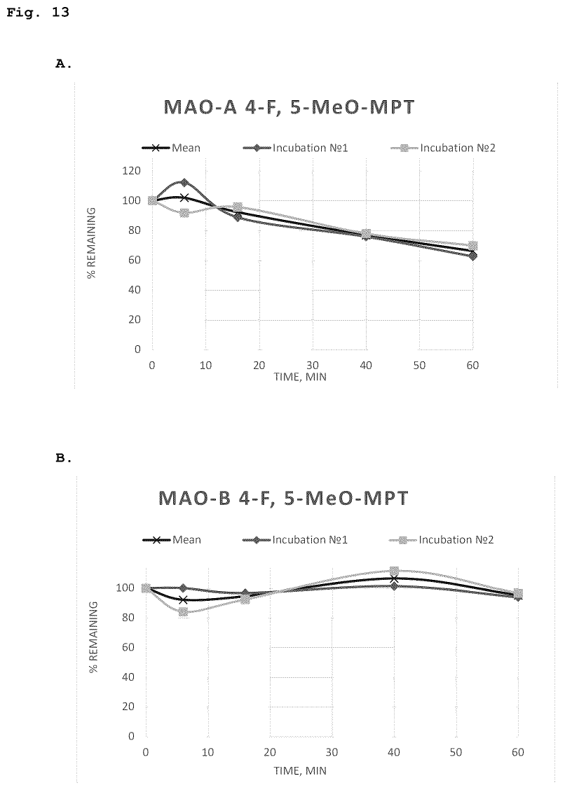

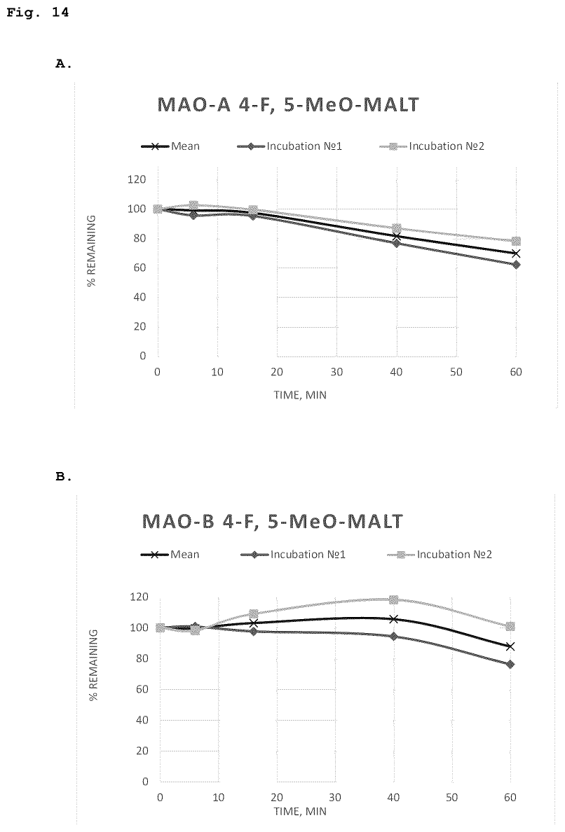

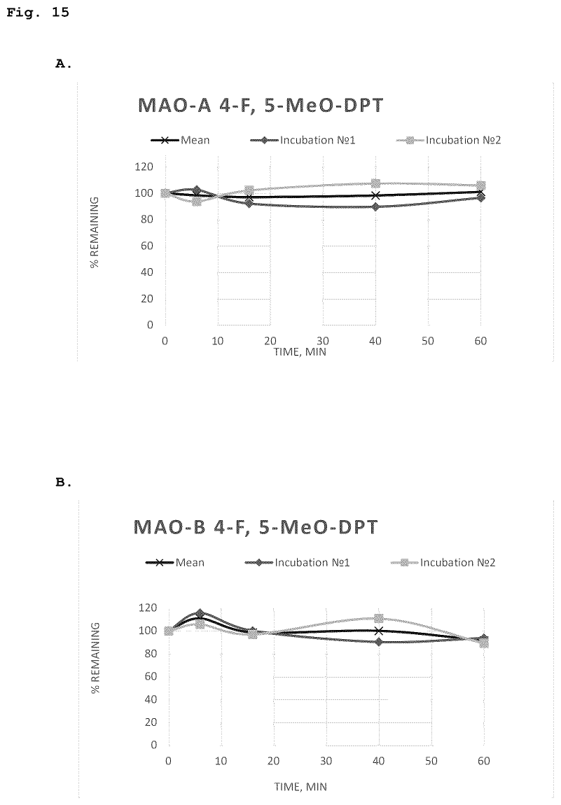

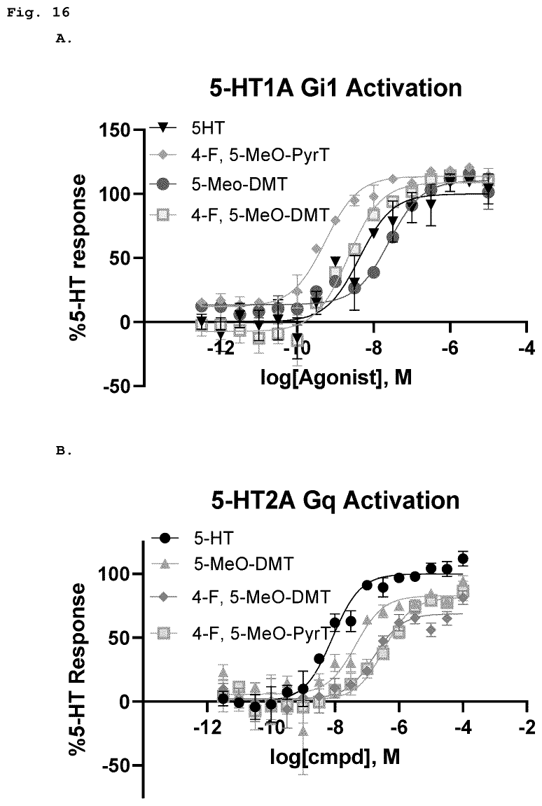

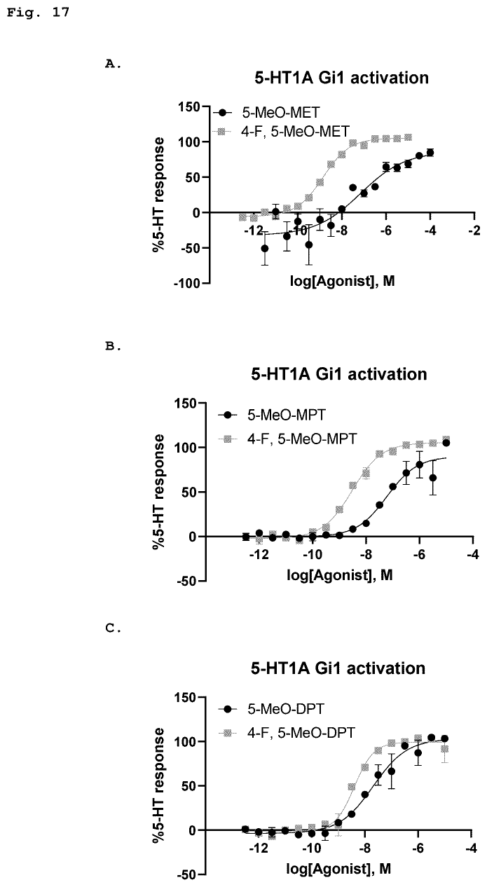

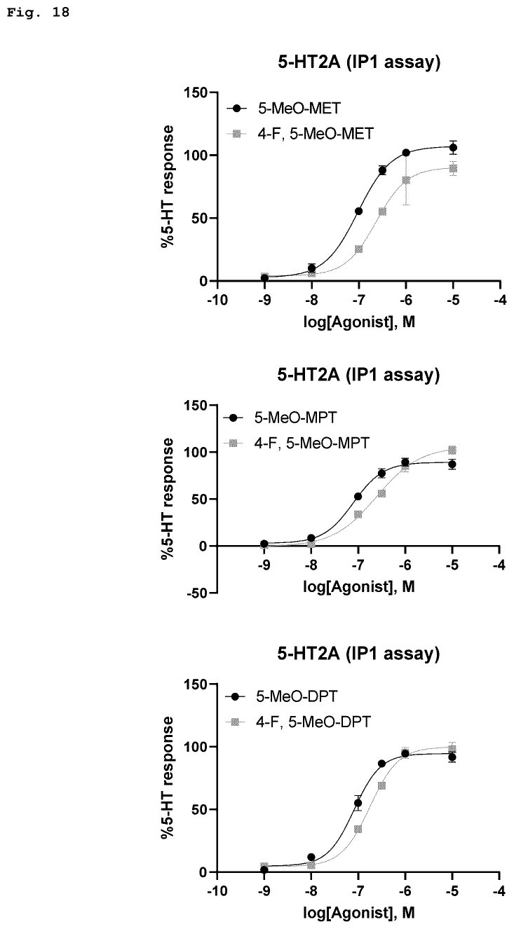

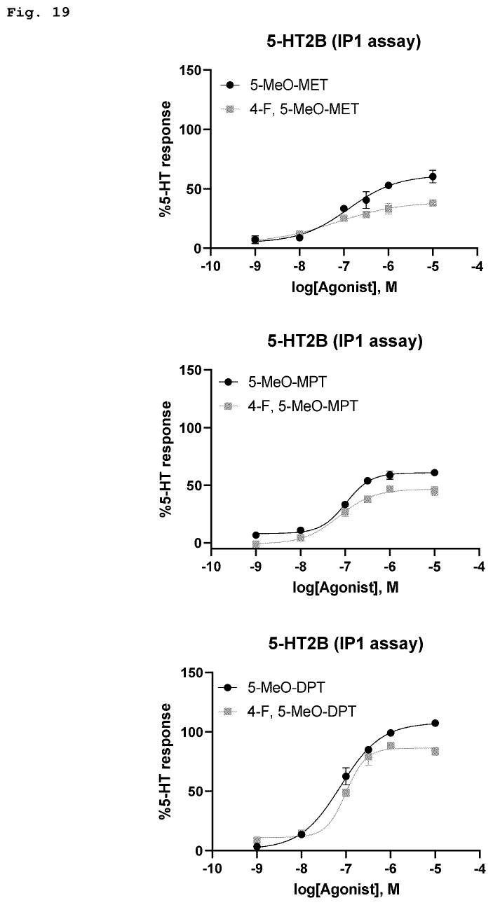

### DETAILED DESCRIPTION OF THE INVENTION

The present invention provides a compound having the structure:

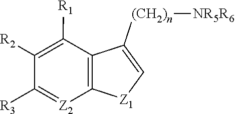

wherein
   Z₁ is NR₄, O, or S;
   Z₂ is CR₇ or N;
   n is 1 to 5;
   R₁ is —H, halogen, —O-alkyl, or together with R₂ forms —O—CH₂—O—
   R₂ is —H, —OH, —O-alkyl, —O-alkyl-aryl, —S-alkyl, —S-alkyl-aryl, or together with R₁ or R₃ forms —O—CH₂—O—;
   R₃ is —H, —O-alkyl or together with R₂ forms —O—CH₂—O—;
   R₄ is H or alkyl;
   R₅ and R₆ are each independently H, alkyl, alkenyl, alkynyl or NR₅R₆ together form a substituted or unsubstituted heterocycle, wherein the heterocycle may be monocyclic or bicyclic,
   R₇ is H or alkyl;
      wherein R₁ is F, when R₂ is —O-(n-propyl) or when at least one of R₅ or R₆ is H, and
      wherein the compound is other than a compound where
         Z₁ is NR₄ and R₁, R₂, R₃, and R₈ are H;
         n is 2, R₁ is halogen, R₂ is —OCH₃, R₃ is —H, R₄ is —H, and R₅ and R₆ are both methyl;
         n is 2, R₁ is —F, R₂ is —OCH₃, R₃ is —H, R₄ is —H, and NR₅R₆ together are —N-pyrrolidine;
         n is 2, R₁ and R₂ together forms —O—CH₂—O—, R₃ is —H, R₄ is —H, and R₅ and R₆ are both methyl; and
         n is 2, R₁ and R₂ together forms —O—CH₂—O—, R₃ is —H, R₄ is —H; and R₅ and R₆ are both isopropyl,
   or a pharmaceutically acceptable salt thereof.

The present invention also provides a compound having the structure:

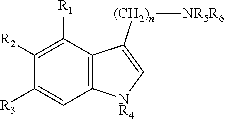

wherein
   n is 1 to 5;
   R₁ is a halogen or together with R₂ forms —O—CH₂—O—;
   R₂ is —OH, —O-alkyl, —O-alkyl-aryl, —S-alkyl, —S-alkyl-aryl, or together with R₁ or R₃ forms —O—CH₂—O—;
   R₃ is H or together with R₂ forms —O—CH₂—O—;
   R₄ is H or alkyl; and
   R₅ and R₆ are each independently H, alkyl, alkenyl, alkynyl or NR₅R₆ together form a substituted or unsubstituted heterocycle,
   wherein the heterocycle may be monocyclic or bicyclic,
      wherein R₁ is F, when R₂ is —O-(n-propyl) or when at least one of R₅ or R₆ is H, and
      wherein the compound is other than a compound where
         n is 2, R₁ is halogen, R₂ is —OCH₃, R₃ is —H, R₄ is —H, and R₅ and R₆ are both methyl;
         n is 2, R₁ is —F, R₂ is —OCH₃, R₃ is —H, R₄ is —H, and NR₅R₆ together are —N-pyrrolidine;
         n is 2, R₁ and R₂ together forms —O—CH₂—O—, R₃ is —H, R₄ is —H, and R₅ and R₆ are both methyl; and
         n is 2, R₁ and R₂ together forms —O—CH₂—O—, R₃ is —H, R₄ is —H; and R₅ and R₆ are both isopropyl,
   or a pharmaceutically acceptable salt thereof.

In some embodiments, R₁ is —F, —Cl or —Br.

In other embodiments, R₁ is —F or —Cl.

In yet other embodiments, R₁ is —F or —Br.

In further embodiments, R₁ is —F.

The present invention also provides a compound having the structure:

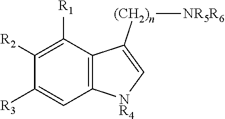

wherein n is 1 to 5;
   R₁ is —F or together with R₂ forms —O—CH₂—O—;
   R₂ is —OH, —O-alkyl, —O-alkyl-aryl, —S-alkyl, —S-alkyl-aryl, or together with R₁ or R₃ forms —O—CH₂—O—;
   R₃ is H or together with R₂ forms —O—CH₂—O—;
   R₄ is H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic, and
wherein the compound is other than a compound where
   n is 2, R₁ is —F, R₂ is —OCH₃, R₃ is —H, R₄ is —H, and R₅ and R₆ are both methyl;
   n is 2, R₁ is —F, R₂ is —OCH₃, R₃ is —H, R₄ is —H, and NR₅R₆ together are —N-pyrrolidine;
   n is 2, R₁ and R₂ together forms —O—CH₂—O—, R₃ is —H, R₄ is —H, and R₅ and R₆ are both methyl; and
   n is 2, R₁ and R₂ together forms —O—CH₂—O—, R₃ is —H, R₄ is —H; and R₅ and R₆ are both isopropyl,
or a pharmaceutically acceptable salt thereof.

In some embodiments, n is 2. In other embodiments, R₃ is —H. In other embodiments, the compound has the structure wherein R₄ is H or C₁-C₂ alkyl.

In some embodiments, the compound has the structure wherein R₂ is —OH, —O-alkyl, —O-alkyl-aryl, —S-alkyl or —S-alkyl-aryl. In other embodiments, the compound has the structure wherein R₂ is —OH, —O—CH₃, —O—CH₂-Ph, —S—CH₃, or —S—CH₂-Ph.

In some embodiments, the compound has the structure wherein R₅ and R₆ are each independently methyl, ethyl, propyl, isopropyl, butyl, tert-butyl, or allyl; or wherein NR₅R₆ together form —N-azetidine, —N-pyrrolidine, —N-(3-methyl-pyrrolidine), —N-piperidine, —N-(3-pyrolline), —N-(1,2,3,6-tetrahydropyridine), —N2 azabicyclo[2.2.2]oct-5-ene, or —N-7-ethyl-2-azabicyclo[2.2.2]oct-5-ene.

In other embodiments, there compound has the structure wherein R₅ and R₆ are each independently methyl, ethyl, propyl, isopropyl, butyl, tert-butyl, or allyl; or wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure wherein —NR₅R₆ is:

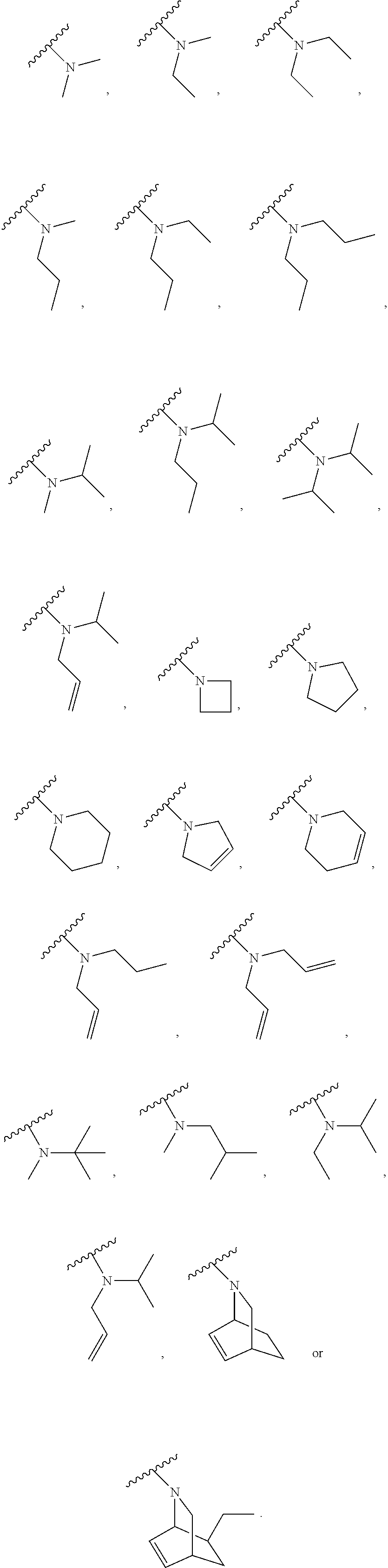

In other embodiments, the compound has the structure wherein —NR₅R₆ is:

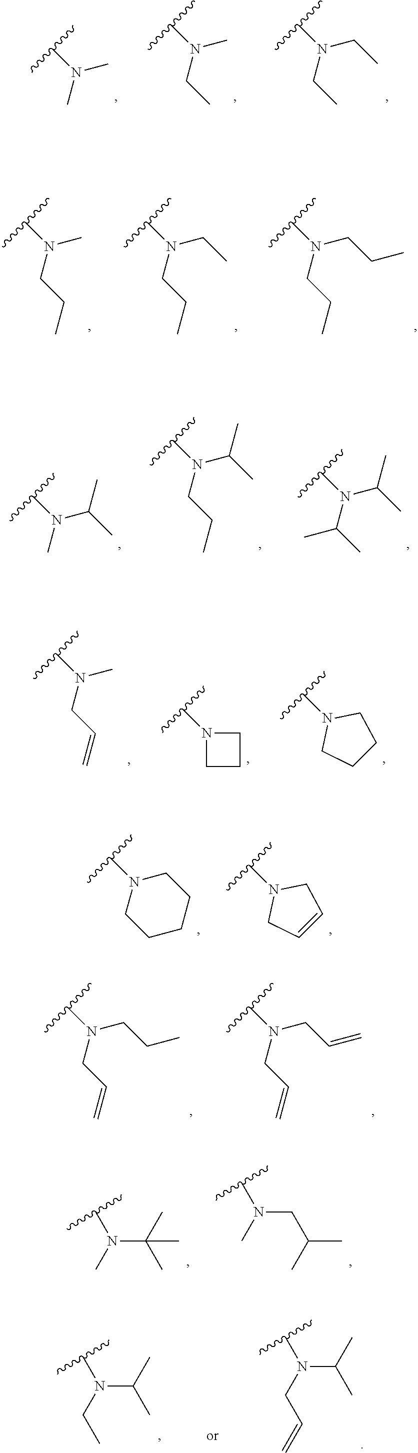

In some embodiments, the compound has the structure:

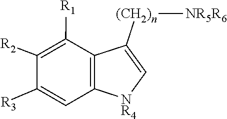

wherein n is 2;
   R₂ is —OH, —O—CH₃, —S—CH₃, —O—CH₂-Ph, or —S—CH₂-Ph;
   R₃ is —H; R₄ is H or —CH₃, and
   R₅ and R₆ are each independently methyl, ethyl, propyl, isopropyl, tert-butyl, or allyl; or —NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

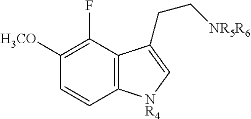

wherein R₄ is —H or —CH₃; and
R₅ and R₆ are each independently methyl, ethyl, propyl, isopropyl, tert-butyl, or allyl; or wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

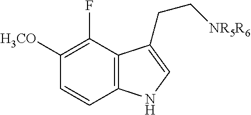

wherein R₅ and R₆ are each independently methyl, ethyl, propyl, isopropyl, tert-butyl, or allyl; or wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

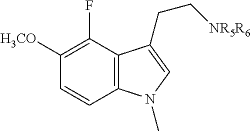

wherein R₅ and R₅ are each independently methyl, ethyl, propyl, isopropyl, tert-butyl, or allyl; or NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

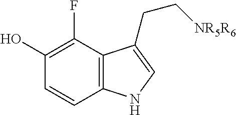

wherein R₅ and R₆ are each independently methyl, ethyl, propyl, isopropyl, tert-butyl, or allyl; or NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

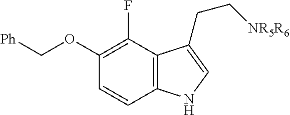

wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

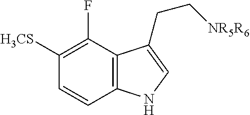

wherein R₅ and R₆ are each independently methyl, ethyl, propyl, isopropyl, tert-butyl, or allyl; or NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

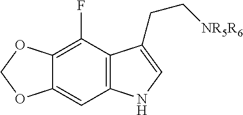

wherein R₄ and R₅ are each independently methyl, ethyl, propyl, isopropyl, tert-butyl, or allyl; or NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

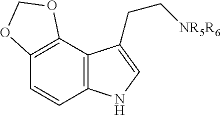

wherein R₄ and R₅ are each independently methyl, ethyl, propyl, isopropyl, tert-butyl, or allyl; or NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

In some embodiments, the compound has the structure:

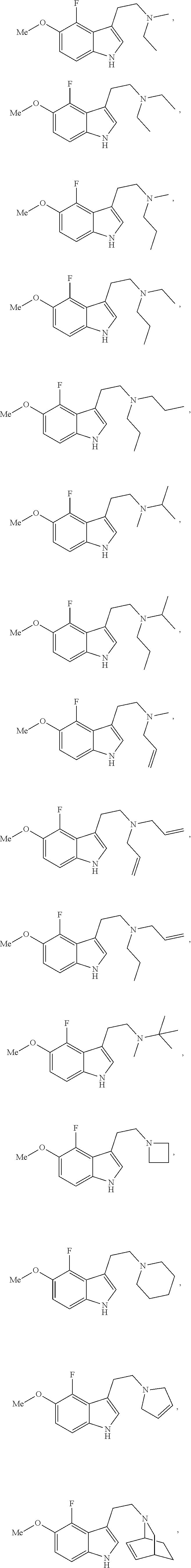

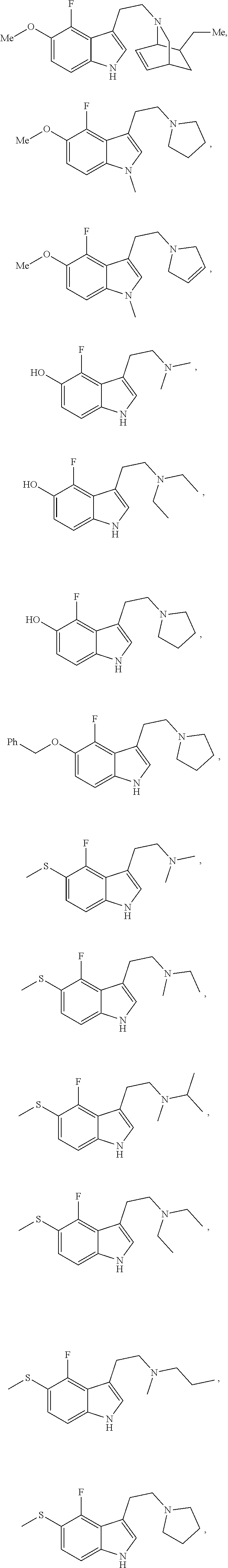

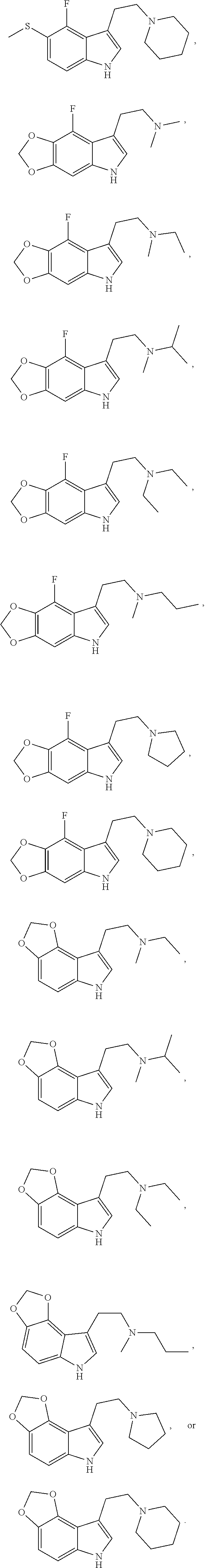

In some embodiments, the compound has the structure:

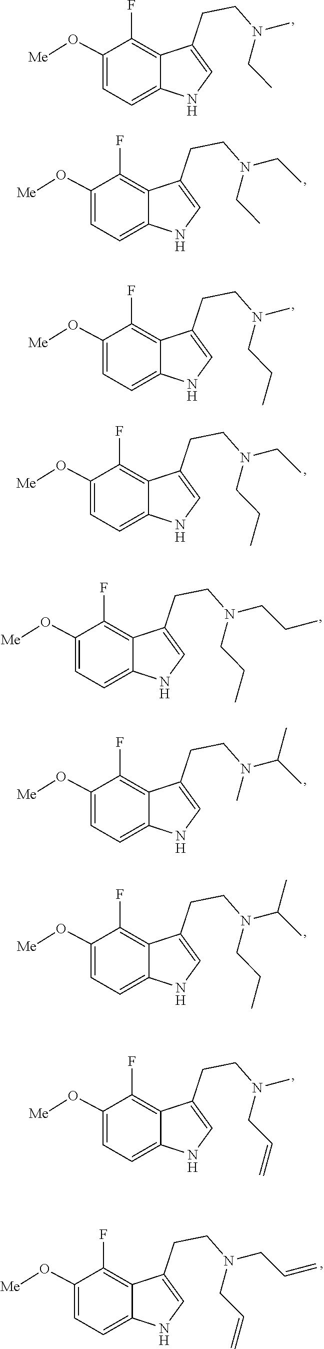

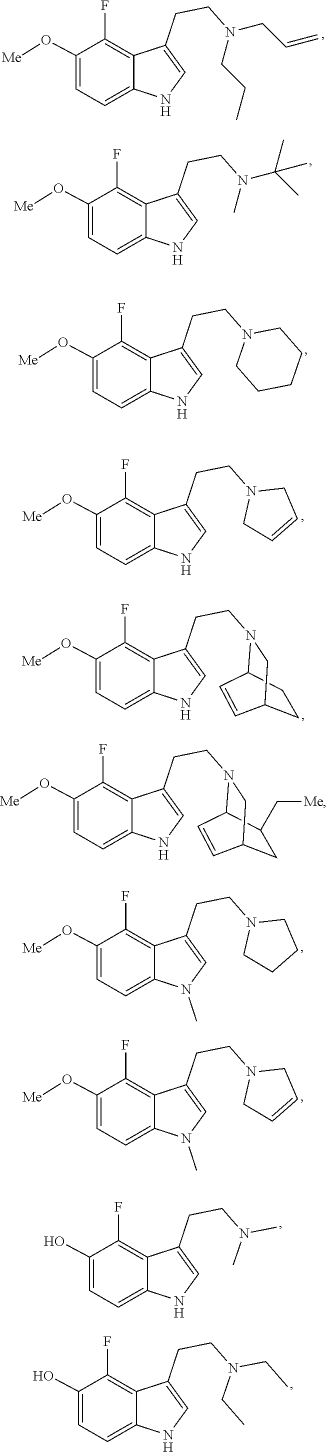

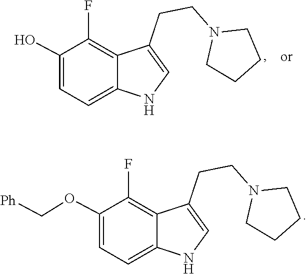 [SMILES: Oc1ccc2[nH]cc(CCN3CCCC3)c2c1F ; Fc1c(OCc2ccccc2)ccc2[nH]cc(CCN3CCCC3)c12]

In other embodiments, the compound has the structure:

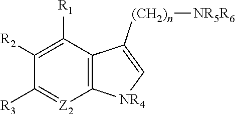

wherein
Z₂ is CR₇ or N;
n is 1 to 5;
R₁ is —H, halogen, —O-alkyl, or together with R₂ forms —O—CH₂—O—
R₂ is —H, —OH, —O-alkyl, —O-alkyl-aryl, —S-alkyl, —S-alkyl-aryl, or together with R₁ or R₃ forms —O—CH₂—O—;
   R₃ is —H, —O-alkyl or together with R₂ forms —O—CH₂—O—;
   R₄ is —H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; and
   R₇ is H or alkyl.

In still other embodiments, the compound has the structure:

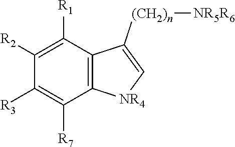

wherein n is 1 to 5;
   R₁ is —H, a halogen, —O-alkyl, or together with R₂ forms —O—CH₂—O—;
   R₂ is —H, —OH, —O-alkyl, —O-alkyl-aryl, —S-alkyl, —S— alkyl-aryl, or together with R₁ or R₃ forms —O—CH₂—O—;
   R₃ is —H, —O-alkyl or together with R₂ forms —O—CH₂—O—;
   R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl.

In et further embodiments, the compound has the structure:

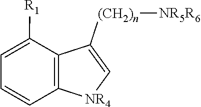

wherein n is 2;
   R₁ is a halogen or O-alkyl;
   R₄ is H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; or

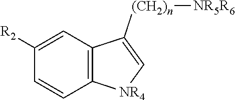

wherein n is 1 to 5;
   R₂ is —OH, —O-alkyl, —O-alkyl-aryl, —S-alkyl, or —S— alkyl-aryl;
   R₄ is H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; or

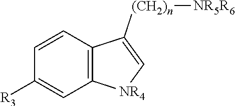

wherein n is 1 to 5;
   R₃ is —H or —O-alkyl;
   R₄ is H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; or

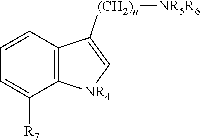

wherein n is 2;
   R₄ is H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; and
   R₇ is alkyl.

In some embodiments, the compound has the structure:

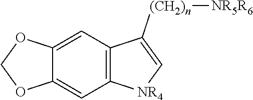

wherein n is 1 to 5:
   R₄ is —H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic; and
   R₇ is H or alkyl.

In other embodiments, the compound has the structure:

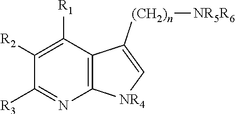

wherein n is 1 to 5;
   R₁ is —H, halogen, —O-alkyl, or together with R₂ forms —O—CH₂—O—;
   R₂ is —H, —OH, —O-alkyl, —O-alkyl-aryl, —S-alkyl, —S— alkyl-aryl, or together with R₁ or R₃ forms —O—CH₂—O—;
   R₃ is —H, —O-alkyl or together with R₂ forms —O—CH₂—O—;
   R₄ is —H or alkyl; and
   R₅ and R₆ are each independently H, alkyl, alkenyl, alkynyl or NR₅R₆ together form a substituted or unsubstituted heterocycle, wherein the heterocycle may be monocyclic or bicyclic.

In yet other embodiments the compound has the structure:

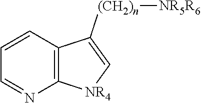

wherein n is 1 to 5;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic.

In some embodiments, the compound has the structure:

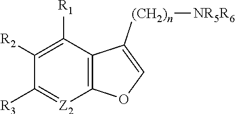

wherein
   Z₂ is CR₇ or N;
   n is 1 to 5;
   R₁ is a halogen or together with R₂ forms —O—CH₂—O—;
   R₂ is —OH, —O-alkyl, —O-alkyl-aryl, —S-alkyl, —S-alkyl-aryl, or together with R₁ or R₃ forms —O—CH₂—O—;
   R₃ is H or together with R₂ forms —O—CH₂—O—; and
   R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl.

In some embodiments, the compound has structure:

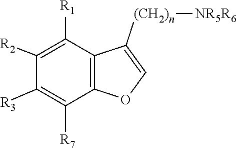

wherein n is 1 to 5;
   R₁ is —F or together with R₂ forms —O—CH₂—O—;
   R₂ is —OH, —O-alkyl, —O-alkyl-aryl, —S-alkyl, —S— alkyl-aryl, or together with R₁ or R₃ forms —O—CH₂—O—
   R₃ is H or together with R₂ forms —O—CH₂—O—; and
   R₅ and R₆ are each independently H, alkyl, alkenyl, alkynyl or NR₅R₆ together form a substituted or unsubstituted heterocycle, wherein the heterocycle may be monocyclic or bicyclic.

In other embodiments, the compound has the structure:

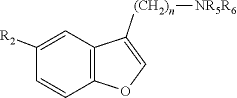

wherein n is 1 to 5;
R₂ is —OH, —O-alkyl, —O-alkyl-aryl, —S-alkyl, —S-alkyl-aryl, or together with R₁ or R₃ forms —O—CH₂—O—;
R₅ and R₆ are each independently H, alkyl, alkenyl, alkynyl or NR₅R₆ together form a substituted or unsubstituted heterocycle, wherein the heterocycle may be monocyclic or bicyclic.

In other embodiments, the compound has the structure:

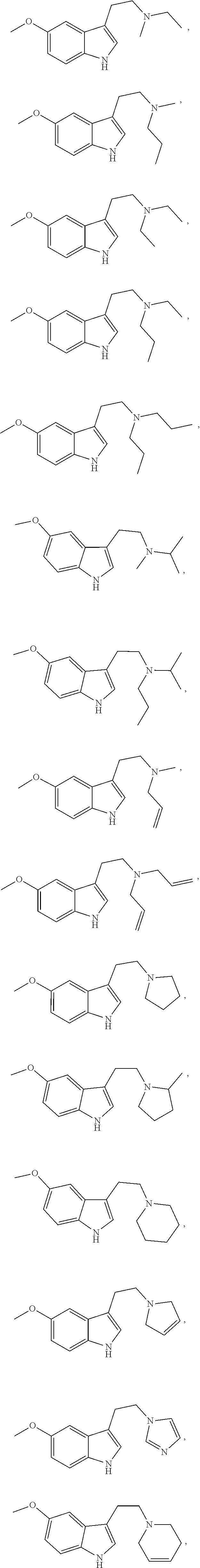

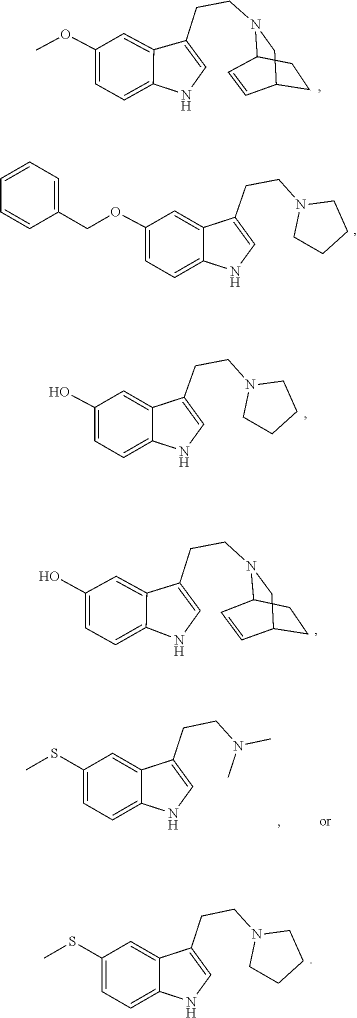

In other embodiments, the compound has the structure:

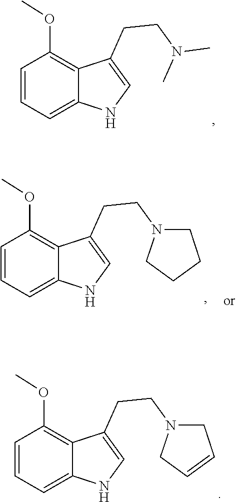 [SMILES: COc1cccc2[nH]cc(CCN(C)C)c12 ; COc1cccc2[nH]cc(CCN3CCCC3)c12 ; COc1cccc2[nH]cc(CCN3CC=CC3)c12]

In other embodiments, the compound has the structure:

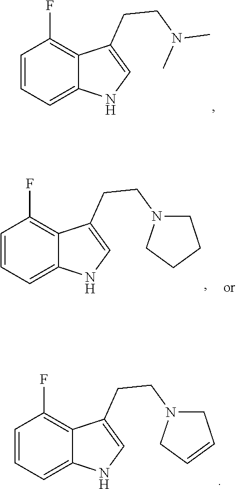 [SMILES: CN(C)CCc1c[nH]c2cccc(F)c12 ; Fc1cccc2[nH]cc(CCN3CCCC3)c12 ; Fc1cccc2[nH]cc(CCN3CC=CC3)c12]

In other embodiments, the compound has the structure:

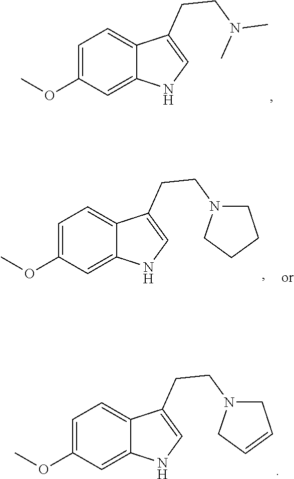

In other embodiments, the compound has the structure:

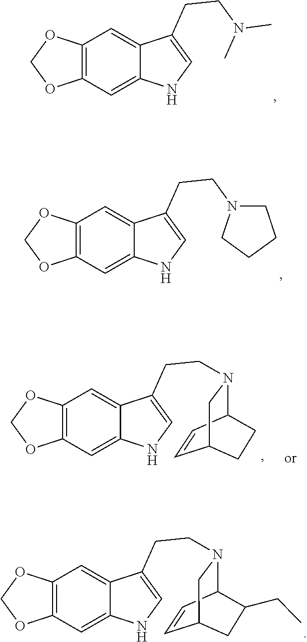 [SMILES: CN(C)CCc1c[nH]c2cc3c(cc12)OCO3 ; c1[nH]c2cc3c(cc2c1CCN1CCCC1)OCO3 ; C1=CC2CCC1CN2CCc1c[nH]c2cc3c(cc12)OCO3 ; CCC1CC2C=CC1N(CCc1c[nH]c3cc4c(cc13)OCO4)C2]

In an embodiment, the compound has the structure:

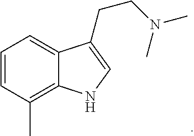

In other embodiments, the compound has the structure:

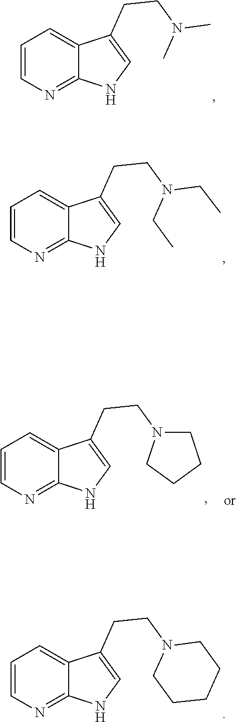 [SMILES: CN(C)CCc1c[nH]c2ncccc12 ; CCN(CC)CCc1c[nH]c2ncccc12 ; c1cnc2[nH]cc(CCN3CCCC3)c2c1 ; c1cnc2[nH]cc(CCN3CCCCC3)c2c1]

In other embodiments, the compound has the structure:

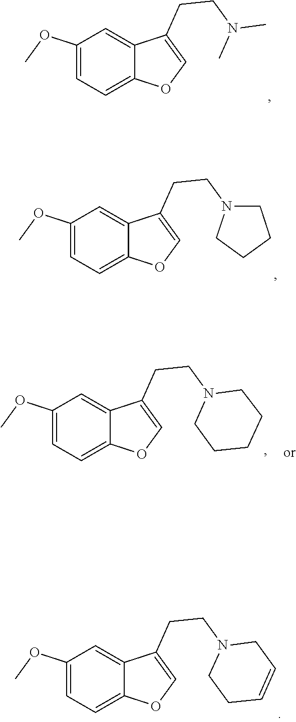

In yet other embodiments, the compound has the structure:

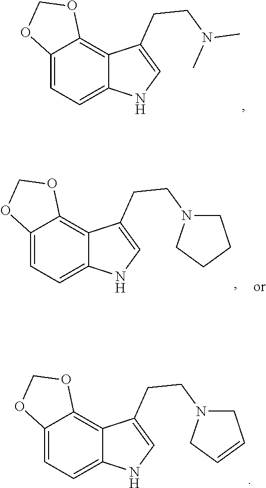 [SMILES: CN(C)CCc1c[nH]c2ccc3c(c12)OCO3 ; c1cc2[nH]cc(CCN3CCCC3)c2c2c1OCO2 ; C1=CCN(CCc2c[nH]c3ccc4c(c23)OCO4)C1]

The present invention also provides a composition comprising the compound of the present invention and a carrier. In other embodiments, the carrier is a pharmaceutically acceptable carrier. In yet other embodiments, the composition is a pharmaceutically acceptable composition. In still further embodiments, the pharmaceutical composition comprises the compound of the invention and a pharmaceutically acceptable carrier.

In some embodiments, the subject is a mammal. In other embodiments, the mammal is a human.

In some embodiments, the effective amount of the compound of the present invention is from 25 mg to 500 mg of the compound. In other embodiments, the effective amount of the compound of the present invention is from 100 mg to 300 mg of the compound. In yet further embodiments, the effective amount of the compound of the present invention is 0.1-20 mg of compound per kilogram of body weight.

The present invention also provides a method for the preparation of the compounds described herein.

The present invention also provides a process for the preparation of the compound of the present invention comprising a step of dissolving a compound having the structure:

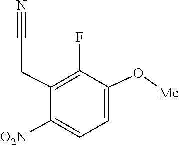 [SMILES: COc1ccc([N+](=O)[O-])c(CC#N)c1F]

in a suitable solvent followed by treatment with a suitable acid in the presence of a palladium catalyst at elevated pressure in an atmosphere of hydrogen.

In one embodiment, the solvent is ethanol. In another embodiment, the acid is acetic acid. In yet another embodiment, the pressure is 60 psi. In some embodiments, the solvent is ethanol, the acid is acetic acid and/or the pressure is 60 psi. The present invention also provides a process for the preparation of the compound of the present invention comprising a step of dissolving a compound having the structure:

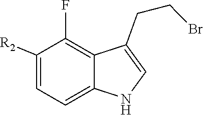

in a suitable solvent followed by treatment with a suitable amine.

In one embodiment, the solvent is methanol. In another embodiment, the amine is a symmetric or unsymmetric alkyl amine; or the amine is a substituted or unsubstituted heterocyclic amine. In some embodiments, the solvent is methanol and/or the amine is a substituted or unsubstituted heterocyclic amine.

The present invention also provides a method of treating a subject afflicted with a substance use disorder comprising administering an effective amount of the compound of the present invention or the composition of the present invention to the subject so as to treat the substance use disorder.

In some embodiments of the above methods, the substance use disorder is opioid use disorder, alcohol use disorder or stimulant use disorder including nicotine use disorder. In some embodiments of the above methods, the substance is an opioid.

The present invention further provides a method of treating a subject afflicted with of a neurological disease, psychiatric disorder, or substance abuse disorder comprising administering to the subject a compound of the present invention in combination with a compound having the structure:

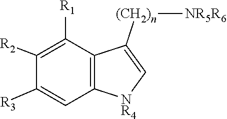

wherein n is 1 to 5;
R₁ is a halogen or together with R₂ forms —O—CH₂—O—;
R₂ is OH, O-alkyl, O-alkyl-aryl, S-alkyl, S-alkyl-aryl, or together with R₁ or R₃ forms —O—CH₂—O—;
R₃ is H or together with R₂ forms —O—CH₂—O—;
R₄ is H or alkyl; and
R₅ and R₆ are each independently H, alkyl, alkenyl, alkynyl or NR₅R₆ together form a substituted or unsubstituted heterocycle, and wherein the heterocycle may be mono or bicyclic;
together in an amount effective to treat the subject.

The present invention further provides a method of activating or selectively activating the 5HT1A receptor, or of simultaneously activating the 5HT1A and 5HT2A receptors, in a subject comprising administering to the subject an amount of a compound having the structure:

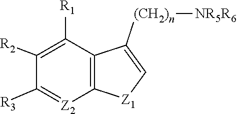

wherein
   Z₁ is NR₄, O, or S;
   Z₂ is CR₇ or N;
   n is 1 to 5;
   R₁ is —H, halogen, —O-alkyl, or together with R₂ forms —O—CH₂—O—
   R₂ is —H, —OH, —O-alkyl, —O-alkyl-aryl, —S-alkyl, —S-alkyl-aryl, or together with R₁ or R₃ forms —O—CH₂—O—;
   R₃ is —H, —O-alkyl or together with R₂ forms —O—CH₂—O—;
   R₄ is H or alkyl;
   R₅ and R₆ are each independently H, alkyl, alkenyl, alkynyl or NR₅R₆ together form a substituted or unsubstituted heterocycle, wherein the heterocycle may be monocyclic or bicyclic; and
   R₇ is H or alkyl;
   or pharmaceutically acceptable salt thereof,
      so as to thereby to activate or selectively activate the 5HT1A receptor, or simultaneously activate the 5HT1A and 5HT2A receptors in the subject.

The present invention further comprises the compound and a pharmaceutically acceptable carrier.

The present invention also provides a method of treating a subject afflicted with a substance use disorder comprising administering an effective amount of the compound of the present invention or the composition of the present invention to the subject so as to treat the substance use disorder.

In some embodiments of the above methods, the substance use disorder is opioid use disorder, alcohol use disorder or stimulant use disorder including nicotine use disorder. In some embodiments of the above methods, the substance is an opioid.

In some embodiments, the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine. In other embodiments, the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil.

In some embodiments of any of the above methods, the stimulant is cocaine, amphetamine, methamphetamine or cathinone and its derivatives. In some embodiments of any of the above methods, the stimulant is nicotine.

The present invention also provides a method of treating a subject afflicted with opioid withdrawal symptoms comprising administering an effective amount of the compound of the present invention or the composition of the present invention to the subject so as to treat the opioid withdrawal symptoms.

In some embodiments of the above methods, a symptom of substance use disorder is opioid withdrawal or mitigation of relapse to opioid use or SUD. In some embodiments, the risk of relapse to the use of opioids, alcohol or stimulants is reduced. In other embodiments, self-administration of an opioid, alcohol or stimulant is reduced.

The present invention also provides a method comprising administering to the subject a compound in a pharmaceutical composition comprising the compound and a pharmaceutically acceptable carrier. In some embodiments, a pharmaceutically acceptable salt of the compound of the present invention is used in any of the methods, uses, packages or compositions of the present invention. Any of the compound of the present invention may be used in any of the disclosed methods, uses, packages or pharmaceutical compositions.

The terms “movement disorder,” “neurological movement disorder” or “neurological condition,” as used herein, are well within the understanding of one of ordinary skill in the art and are used broadly and to refer to any brain disease, anomaly, or condition causing a subject to have abnormal voluntary and/or involuntary movements, or slow, reduced movements. Examples of movement disorders include, but are not limited to, Parkinson's disease, dystonia, Huntington's disease, essential tremor, anxiety, ataxia, chorea, myoclonus, ballismus, dysmetria, postural disorders, spasticity (e.g. focal spasticity from stroke, upper limb spasticity), blepharospasm, multiple sclerosis, cerebral palsy, mood disorders, sleep disorders, obesity, anorexia, and chronic pain disorders.

Opioid use disorder (OUD) involves, but is not limited to, misuse of opioid medications or use of illicitly obtained opioids. The Diagnostic and Statistical Manual of Mental Disorders, 5th Edition (American Psychiatric Association: Diagnostic and Statistical Manual of Mental Disorders: Diagnostic and Statistical Manual of Mental Disorders, Fifth Edition. Arlington, VA: American Psychiatric Association, 2013), which is hereby incorporated by reference, describes opioid use disorder as a problematic pattern of opioid use leading to problems or distress, with at least two of the following occurring within a 12-month period:
Taking larger amounts or taking drugs over a longer period than intended.
Persistent desire or unsuccessful efforts to cut down or control opioid use.
Spending a great deal of time obtaining or using the opioid or recovering from its effects.
Craving, or a strong desire or urge to use opioids.
Problems fulfilling obligations at work, school, or home.
Continued opioid use despite having recurring social or interpersonal problems.
Giving up or reducing activities because of opioid use.
Using opioids in physically hazardous situations.
Continued opioid use despite ongoing physical or psychological problem likely to have been caused or worsened by opioids.
Tolerance (i.e., need for increased amounts or diminished effect with continued use of the same amount).
Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5th Edition describes alcohol use disorder as a problematic pattern of alcohol use leading to problems or distress, with at least two of the following occurring within a 12-month period:
Being unable to limit the amount of alcohol you drink.
Wanting to cut down on how much you drink or making unsuccessful attempts to do so.
Spending a lot of time drinking, getting alcohol, or recovering from alcohol use.
Feeling a strong craving or urge to drink alcohol.
Failing to fulfill major obligations at work, school or home due to repeated alcohol use.
Continuing to drink alcohol even though you know it is causing physical, social, or interpersonal problems.
Giving up or reducing social and work activities and hobbies.
Using alcohol in situations where it is not safe, such as when driving or swimming.
Developing a tolerance to alcohol so you need more to feel its effect, or you have a reduced effect from the same amount.
Experiencing withdrawal symptoms—such as nausea, sweating and shaking—when you do not drink, or drinking to avoid these symptoms.

Stimulant use disorder involves, but is not limited to, a pattern of problematic use of amphetamine, methamphetamine, cocaine, or other stimulants except caffeine or nicotine, leading to at least two of the following problems within a 12-month period:
Taking more stimulants than intended.
Unsuccessful in trying to cut down or control use of stimulants, despite wanting to do so.
Spending excessive amounts of time to activities surrounding stimulant use.
Urges and cravings for stimulants.
Failing in the obligations of home, school, or work.
Carrying on taking stimulants, even though it has led to relationship or social problems.
Giving up or reducing important recreational, social, or work-related activities because of using stimulants.
Using stimulants in a physically hazardous way.
Continuing to use stimulants even while knowing that it is causing or worsening a physical or psychological problem.
Tolerance to stimulants.
Withdrawal from stimulants if you do not take them.

Polydrug use disorder or polysubstance use disorder involves, but is not limited to, dependence on multiple drugs or substances.

The term “5HTA” refers to the serotonin 1A receptor.

The term “5HT1B” refers to the serotonin 1B receptor.

Any of the compounds used in the disclosed methods, uses, packages or pharmaceutical compositions may be replaced with any other compound disclosed in the present invention.

Any of the above generic compounds may be used in any of the disclosed methods, uses, packages or compositions. Except where otherwise specified, the structure of a compound of this invention includes an asymmetric carbon atom, it is understood that the compound occurs as a racemate, racemic mixture, a scalemic mixture and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H (D), or ³H (T). Furthermore, any compounds containing ²H or ³H may specifically have the structure of any of the compounds disclosed herein. Isotopically-labeled compounds can generally be prepared by conventional techniques known to those skilled in the art using appropriate isotopically-labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes 1H (hydrogen or protium), D (2H or deuterium), and T (3H or tritium). The natural abundance of deuterium is 0.0156%. Thus, a compound with a level of deuterium at any site of hydrogen atom in the compound that has been enriched to be greater than its natural abundance of 0.0156%, is novel over its non-enriched counterpart.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

The term “alkylcycloalkyl” refers to alkyl groups as described above wherein one or more bonds to hydrogen contained therein are replaced by a bond to a cycloalkyl group as described above. It is understood that an “alkylcycloalkyl” group is connected to a core molecule through a bond from the alkyl group and that the cycloalkyl group acts as a substituent on the alkyl group.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R—O—CO—R′ group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition. As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### General Outline and Procedures for the Syntheses of 5-Methoxytryptamine Analogs

### General Considerations

Reagents and solvents (including anhydrous solvents) were obtained from commercial sources and were used without further purification unless otherwise stated. Compounds not synthesized in-house were purchased from the following sources: 5-HT (Sigma), 5-MeO-DMT (Cerilliant), DMT (Cerilliant), Psilocin (Cerilliant), 4-OH-MET (Cayman Chemical), LSD (Cerilliant), Mescaline (Cerilliant), Vilazodone (Sigma), Buspirone (Alfa Aesar), Bufotenine (Cayman Chemical), Aripiprazole (Sigma), Citalopram (TCI), FFN246 (Aobious), IDT307 (Sigma), ASP+ (Invitrogen), WAY 100635 (Biovision), 8-OH-DPAT (MedChemExpress), SEP-363856 (MedChemExpress), N-(1-Napthyl)piperazine (Alfa Aesar), Tandospirone (Toronto Research Chemicals), Gepirone (Toronto Research Chemicals).

Reactions were monitored by TLC using solvent mixtures appropriate to each reaction. All column chromatography was performed on silica gel (40-63 μm). Preparative TLC was performed on 20×20 cm plates with a 1 mm silica layer. For compounds containing a basic nitrogen, triethylamine (Et₃N) was used in the mobile phase to provide better resolution when using silica gel chromatography. Nuclear magnetic resonance spectra were recorded on Bruker 300, 400 or 500 MHz instruments, as indicated. Chemical shifts are reported as δ values in ppm referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); dq (doublet of quartets); ddd (doublet of doublet of doublets); ddt (doublet of doublet of triplets); m (multiplet); br (broad). When certain carbon peaks overlap and thus represent two carbons they were indicated by (2C) designation. Low-resolution mass spectra were recorded on an Advion ExpressIon® CMS-L with automated TLC plate reader instrument (ionization mode: APCI+ or ESI+) or on GC/MS (ionization mode: EI) analysis was performed with ‘Agilent 7890B’ gas chromatograph equipped with ‘Agilent 5977B’ quadrupole mass selective detector (ionization mode: EI). High-resolution mass spectra (HRMS) were acquired on a high-resolution Waters XEVO G2-XS QToF mass spectrometer equipped with a UPC2 SFC inlet, on-board fluidics and an ESI probe.

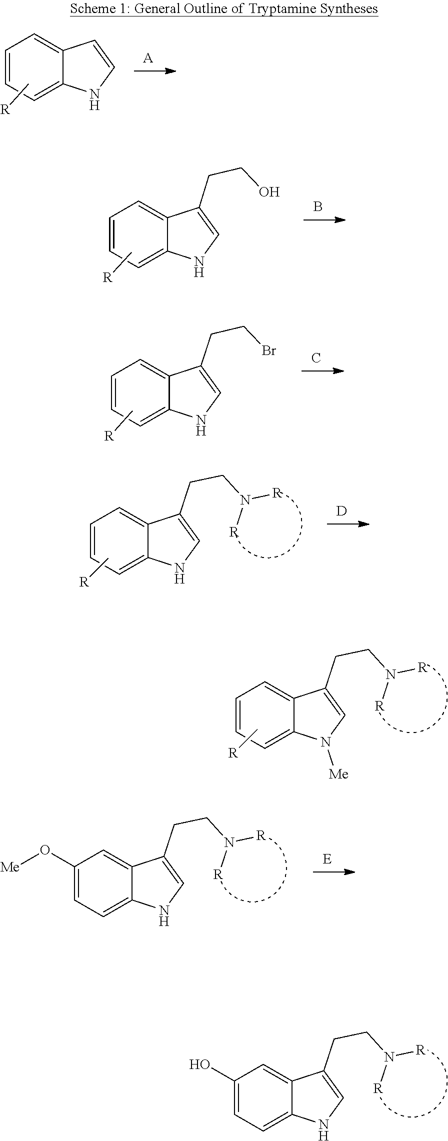

### Speeter-Anthony Synthesis of Tryptamines

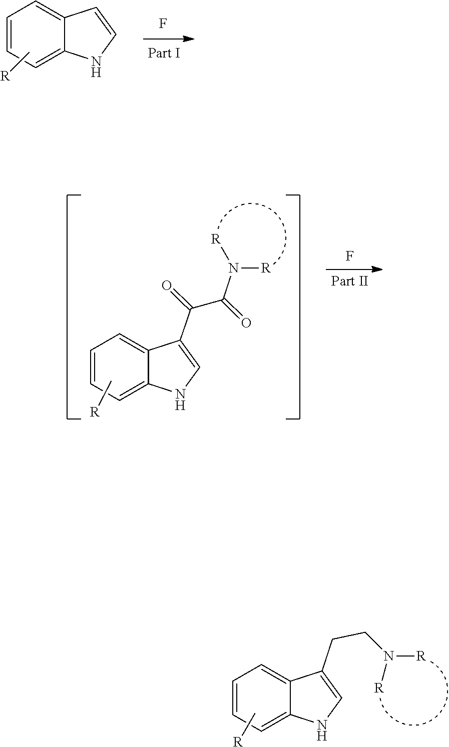

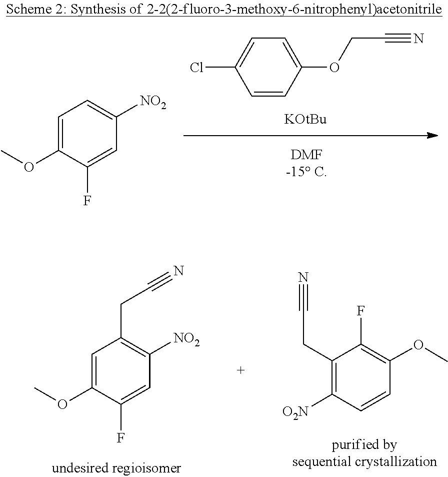

The above-mentioned 2-(2-fluoro-3-methoxy-6-nitrophenyl)acetonitrile was synthesized according to publication WO2003/064413 with slight modifications. The regioisomeric intermediates (4-fluoro-5-methoxy-2-nitro-phenyl)-acetonitrile and (2-fluoro-3-m ethoxy-6-nitro-phenyl)-acetonitrile are separated by a sequence of crystallization of (4-fluoro-5-methoxy-2-nitro-phenyl)-acetonitrile from 2-propanol followed by crystallization of (2-fluoro-3-methoxy-6-nitro-phenyl)-acetonitrile (m/z (MH+)=166.1) from toluene using the mother liquid of the previous crystallization (as described in publication WO 2007/096393).

### Synthesis of 4-fluoro-5-MeO-indole

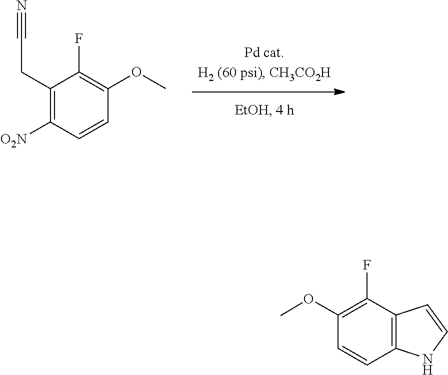

The above-mentioned 4-fluoro-5-methoxy-indole was synthesized according to publication WO2003/064413. In a high pressure tube 2-(2-fluoro-3-methoxy-6-nitrophenyl)acetonitrile was dissolved in ethanol (1 M) and acetic acid (7.5 M). 10% Pd on charcoal was added (10 mol %) and mixture was hydrogenated under 60 psi pressure for 2 h. The mixture was filtered, the volatiles were removed, and the crude residue was purified over silica gel.

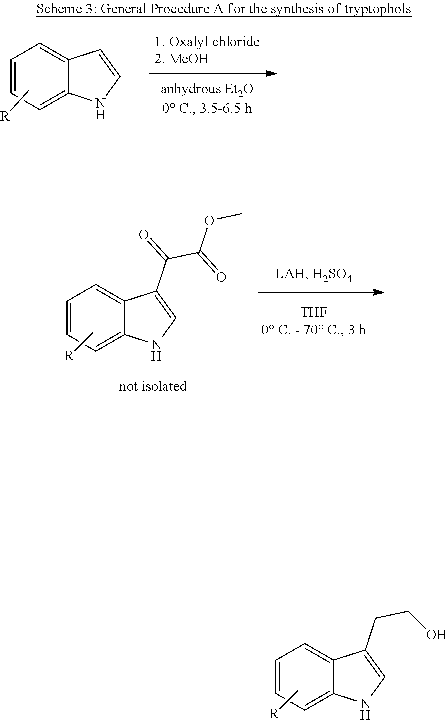

In a reaction flask under argon, 1 equiv. of indole was dissolved in dry diethyl ether (0.2 M), and oxalyl chloride (1.5 equiv.) was added dropwise to the reaction solution at 0° C. The reaction was continued for 3-6 h at room temperature to form a suspension. The reaction mixture was further cooled to 0° C., 20 equiv. of methanol was added dropwise to the reaction, and the mixture was stirred for 30 min. The resulting mixture was filtered and washed with cold diethyl ether to finally obtain the glyoxyester as a fine powder. The powder was used for the next step without further purification.

Reduction Method I: A suspension of LiAlH₄ (LAH) (5 equiv.) in THF (1 M) was cooled to 0° C. and H₂SO₄ (2.5 equiv.) diluted in THF was added dropwise at a slow rate. To the light-gray suspension was added the glyoxyester suspended in THF (0.5 M) at a slow rate keeping the temperature at 0-5° C. After the addition the mixture stirred at r.t. for 30 min and then heated to 70° C. for 4 h. The mixture was carefully quenched with water (1:1 w/v in respect to grams of LAH), aqueous NaOH solution (1:1 w/v, 15% NaOH), and finally, more water (3:1 w/v) filter through a plug of celite, washed with ethyl acetate and the combined organic were reduced in vacuum. The tryptophol product was purified over silica gel.

Reduction Method II: A suspension of LAH (5 equiv.) in THF (1 M) was cooled to 0° C. and a solution of H₂SO₄ (2.5 equiv.) in THF was added dropwise at a slow rate. To the light-gray suspension was added the glyoxyester suspended in THF (0.5 M) at a slow rate keeping the temperature at 0-5° C. After the addition the mixture was stirred at r.t. for 30 min and then heated to 70° C. for 4 h. The mixture was carefully quenched with water and 15% NaOH solution, filtered through celite, washed with ethyl acetate and the combined organic were reduced in vacuum. The tryptophol product was purified over silica gel.

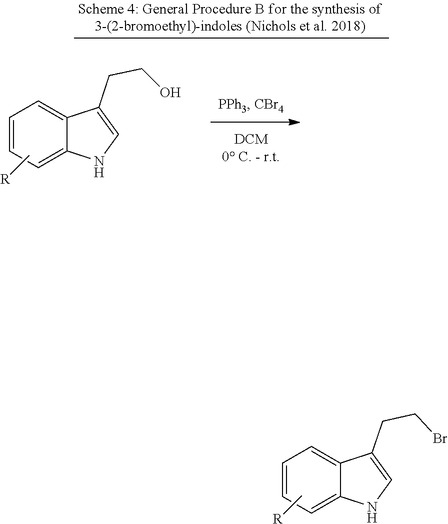

The desired tryptophol and CBr₄ were dissolved in dry DCM (0.8 M), cooled to 0° C., and PPh₃ dissolved in dry DCM (2.5 M) was added dropwise over 10 min. The reaction mixture was stirred at r.t for 1-3 h (followed by TLC). The DCM was evaporated, and the crude mixture was purified by a silica gel column.

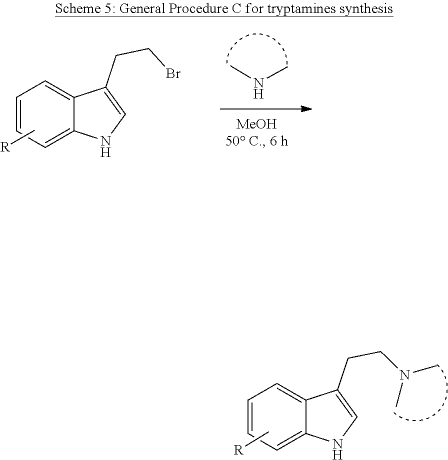

Method I: The desired 3-(2-bromoethyl)-1H-indole derivative was dissolved in MeOH and the appropriate alkyl amine (10 equiv.) was added in one portion. The resulting mixture was stirred at 50° C. for 4-6 h (followed by TLC). At completion, the solvent reduced in vacuum and the crude mixture was loaded on silica gel column or preparative TLC plate.

Method II (Alper et al. 1999): The desired 3-(2-bromoethyl)-1H-indole derivative was dissolved in MeCN (0.2 M) and the appropriate alkyl amine (1.5 equiv.) was added together with 4 equiv. of NaHCO₃. The resulting mixture was stirred at 50-80° C. for 4-6 h (followed by TLC). At completion, the reaction mixture was filtered over celite, the solvent was reduced in vacuum and the crude mixture was loaded on silica gel column or preparative TLC plate.

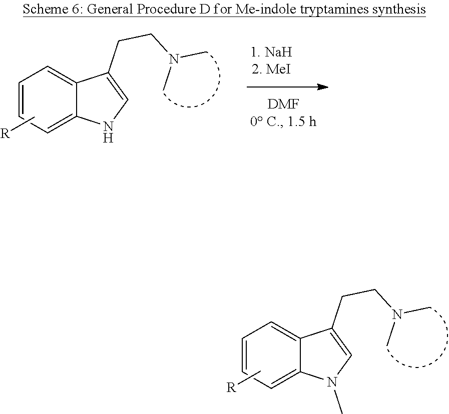

The desired tryptamine derivative was dissolved in DMF (0.1 M) and the mixture was cooled to 0° C. NaH (60% dispersion in mineral oil, 4 equiv.) was carefully added portion wise and the resulting mixture was stirred at r.t. for 1 h. MeI (1 equiv.) was added in one portion and the mixture was further stirred for 30 min. At completion, the reaction was quenched with water and extracted ether (×3). The combined organic layers were dried over Na₂SO₄ and concentrated to provide the crude product. The crude mixture was purified by a silica gel column or preparative TLC plate.

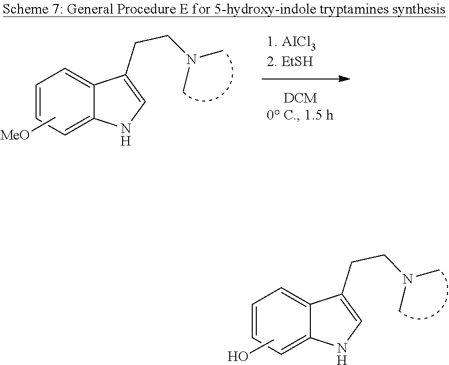

To a solution of the desired methoxy tryptamine (1 equiv.) in dry dichloromethane (0.125 M) at 0° C. was added aluminum chloride (6 equiv.) followed by ethanethiol (18 equiv.), and the resulting mixture was allowed to warm to room temperature and stirred until TLC indicated the complete consumption of starting material (typically <1.5 h). The reaction was then quenched with saturated aqueous NaHCO₃ (100 mL per mmol of starting material) and extracted with DCM:iPrOH 9:1 (4×-6×, until no further extraction was visible by TLC). The combined organic layers were dried over Na₂SO₄ and concentrated to provide the crude product. The crude mixture was purified by a silica gel column or TLC plate.

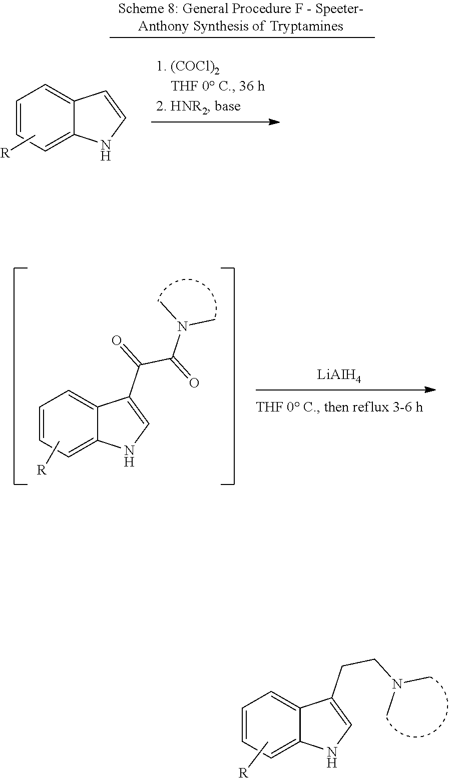

Part I: In a reaction flask flushed with argon, solution of indole (1 equiv.) in dry diethyl ether (0.2 M) was treated dropwise with oxalyl chloride (1.5 equiv.) at 0° C. The reaction was further continued for 3-6 h at room temperature to form a suspension. The solvent and excess oxalyl chloride were removed under reduced pressure and the residue dissolved in 1.0 M of dry THF (in some cases the suspension was filtered and the solid was used for the next steps without further purification. After cooling to 0° C., a solution of the suitable N-substituted-amine* (3 or more equiv.) in dry THF (0.3 M) was added, and the resulting mixture was stirred at room temperature for 1.5 h. The solvent was removed under reduced pressure, the residue partitioned between EtOAc and water, and the aqueous phase extracted with EtOAc tree times. The combined organic extracts were washed with brine, dried (Na₂SO₄) and concentrated under reduced pressure, to give a crude residue of the glyoxyamide. * In some cases, TEA (1.1 equiv) was used instead of an excess of the desired amine.

Part II: The crude glyoxyl amide material was suspended in dry THF (0.5 M) and slowly added to a suspension of LiAlH₄ in dry THF (1 M) at 0° C. under argon**. The mixture was further refluxed for 3 h and after cooling to RT it was quenched with 1 part of water, 1 part of 15% NaOH and 3 parts of water (w/w with respect to the LAH). After the salts turned white and loose, solids were filtered and washed successively with EtOAc. The washes were combined and concentrated, and the crude residue was purified by a silica gel column chromatography. ** In some cases, the AlH₃ (Alane) was produced by the addition of H₂SO₄ to the LAH solution as described in general procedure A, Reduction method II.

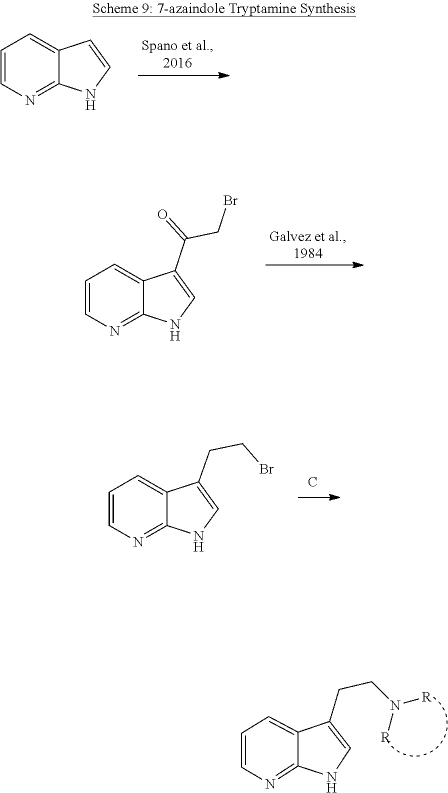

### N-ethyl-2-(1H-indol-3-yl)-N-methylethan-1-amine (2)

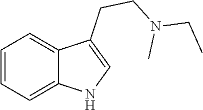

Synthesized according to the general procedure C Method I. 3-(2-bromoethyl)-1H-indole (400 mg, 1.78 mmol, 1 equiv.) was dissolved in 9 mL of MeOH (0.2 M). N-methyl-N-ethylamine (14.28 mmol, 1.2 mL, 8 equiv.) was added. The product was isolated using column chromatography (3:1 Hexanes:EtOAc with 2% TEA) to yield 350 mg of the product, 97%. ¹H NMR (400 MHz, MeOD) δ 7.53 (dt, J=7.9, 1.0 Hz, 1H), 7.33 (dt, J=8.2, 0.9 Hz, 1H), 7.13-7.05 (m, 1H), 7.00 (ddd, J=8.0, 7.0, 1.1 Hz, 1H), 3.01-2.94 (m, 2H), 2.84-2.78 (m, 2H), 2.67 (q, J=7.2 Hz, 2H), 2.43 (s, 3H), 1.16 (t, J=7.2 Hz, 3H). ¹³C NMR (101 MHz, MeOD) δ 136.81, 127.19, 121.78, 120.95, 118.19, 117.69, 111.83, 110.88, 57.33, 50.78, 40.06, 21.98, 10.23. HRMS (ESI+): calcd. for C₁₃H₁₉N₂ [M+H]⁺=203.1548. found [M+H]⁺=203.1547.

### N-(2-(1H-indol-3-yl)ethyl)-N-methylpropan-2-amine (3)

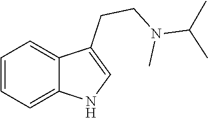 [SMILES: CC(C)N(C)CCc1c[nH]c2ccccc12]

Synthesized according to the general procedure C Method I. 3-(2-bromoethyl)-1H-indole (45 mg, 0.2 mmol, 1 equiv.) was dissolved in 1 mL of MeOH (0.2 M). N-methyl-N-isopropylamine (1.6 mmol, 0.2 mL, 8 equiv.) was added. The product was isolated using column chromatography (3:1 Hexanes:EtOAc with 2% TEA) to yield 34 mg of the product, 78%. ¹H NMR (500 MHz, CDCl₃) δ 8.03 (br s, 1H), 7.63 (d, J=0.8 Hz, 1H), 7.36 (d, J=0.9 Hz, 1H), 7.19 (ddd, J=8.2, 7.0, 1.2 Hz, 1H), 7.12 (ddd, J=8.0, 7.1, 1.1 Hz, 1H), 7.03 (d, J=1.0 Hz, 1H), 3.03-2.87 (m, 3H), 2.79-2.69 (m, 2H), 2.37 (s, 3H), 1.06 (d, J=6.5 Hz, 6H). ¹³C NMR (126 MHz, CDCl₃) δ 136.27, 127.60, 121.94, 121.41, 119.21, 118.90, 114.87, 111.10, 54.24, 53.57, 37.23, 24.20, 18.07. HRS (ESI+): calcd. for C₁₄H₂₁N₂[M+H]⁺=217.1705. found [M+H]⁺=217.1703.

### N-(2-(1H-indol-3-yl)ethyl)-N-isopropylpropan-2-amine (4)

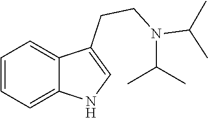 [SMILES: CC(C)N(CCc1c[nH]c2ccccc12)C(C)C]

Synthesized according to the general procedure C Method I. 3-(2-bromoethyl)-1H-indole (60 mg, 0.27 mmol, 1 equiv.) was dissolved in 1.3 mL of MeOH (0.2 M), and diisopropylamine (2.68 mmol, 0.38 mL, 10 equiv.) was added. The product was isolated using column chromatography (4:1 Hexanes:EtOAc with 2% TEA) to yield 40 mg of the product, 61%. ¹H NMR (400 MHz, CDCl₃) δ 8.56 (s, 1H), 7.54 (dt, J=7.8, 0.9 Hz, 1H), 7.43 (dt, J=8.1, 0.9 Hz, 1H), 7.20 (ddd, J=8.2, 7.0, 1.2 Hz, 1H), 7.16-7.09 (m, 2H), 3.72 (hept, J=6.7 Hz, 2H), 3.53-3.42 (m, 2H), 3.24-3.13 (m, 2H), 1.53 (d, J=6.5 Hz, 12H). ¹³C NMR (126 MHz, CDCl₃) δ 136.74, 127.39, 121.50, 120.83, 118.10, 117.88, 113.05, 110.87, 49.46, 27.52, 19.20. HRMS (ESI+): calcd. for C₁₆H₂₅N₂[M+H]⁺=245.2018. found [M+H]⁺=245.2021.

### 3-(2-(pyrrolidin-1-yl)ethyl)-1H-indole (5)

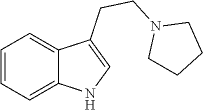 [SMILES: c1ccc2c(CCN3CCCC3)c[nH]c2c1]

Synthesized according to the general procedure C Method I. 3-(2-bromoethyl)-1H-indole (100 mg, 0.45 mmol, 1 equiv.) was dissolved in 2 mL of MeOH (0.2 M). Pyrrolidine (4.03 mmol, 0.34 mL, 4.7 equiv.) was added. The product was isolated using column chromatography (3:1 Hexanes:EtOAc with 2% TEA) to yield 13.6 mg of the product, 14%. ¹H NMR (500 MHz, CDCl₃) δ 8.65 (s, 1H), 7.57 (d, J=7.8 Hz, 1H), 7.41 (d, J=8.1 Hz, 1H), 7.19 (t, J=7.6 Hz, 1H), 7.14-7.06 (m, 2H), 3.47-2.95 (m, 8H), 2.13-1.99 (m, 4H). ¹³C NMR (126 MHz, CDCl₃) δ 136.20, 126.53, 122.68, 122.16, 119.52, 118.14, 111.51, 110.33, 55.92, 53.80, 23.27, 22.33. HRMS (ESI+): calcd. for C₁₄H₁₉N₂[M+H]⁺=215.1548. found [M+H]⁺=215.1550.

### 3-(2-(piperidin-1-yl)ethyl)-1H-indole (6)

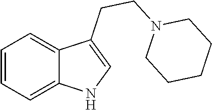 [SMILES: c1ccc2c(CCN3CCCCC3)c[nH]c2c1]

Synthesized according to general procedure C Method I. 3-(2-bromoethyl)-1H-indole (500 mg, 2.23 mmol, 1 equiv.) was dissolved in 8.5 mL of MeOH (0.2 M). Piperidine (22.27 mmol, 2.2 mL, 5.2 equiv.) was added. The product was isolated using column chromatography (EtOAc with 2% TEA) to yield 51.4 mg of the product, 10%. ¹H NMR (500 MHz, CDCl₃) δ 8.44 (s, 1H), 7.62 (d, J=7.9 Hz, 1H), 7.34 (d, J=8.1 Hz, 1H), 7.18 (t, J=7.6 Hz, 1H), 7.11 (t, J=7.5 Hz, 1H), 6.99 (s, 1H), 3.07-2.98 (m, 2H), 2.79-2.68 (m, 2H), 2.68-2.47 (m, 4H), 1.77-1.63 (m, 4H), 1.62-1.41 (m, 2H). ¹³C NMR (126 MHz, CDCl₃) δ 136.36, 127.53, 121.94, 121.71, 119.21, 118.89, 114.17, 111.27, 60.02, 54.59, 25.80, 24.36, 22.69. HRMS (ESI+): calcd. for C₁₅H₂₁N₂[M+H]⁺=229.1705. found [M+H]⁺=229.1704.

### (1S,4R)-2-(2-(1H-indol-3-yl)ethyl)-2-azabicyclo[2.2.2]oct-5-ene (7)

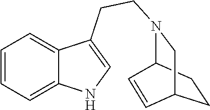 [SMILES: C1=CC2CCC1CN2CCc1c[nH]c2ccccc12]

Synthesized according to general procedure C Method II. 3-(2-bromoethyl)-4-fluoro-5-methoxy-1H-indole (448 mg, 2.0 mmol, 1 equiv.) was dissolved in 10 mL of MeCN (0.2 M). 2-azabicyclo[2.2.2]oct-5-ene hydroiodide (474 mg, 2.0 mmol, 1 equiv.) and NaHCO₃ (672 mg, 8.0 mmol, 4 equiv.) were added. The resulting mixture was heated to 80° C. for 48 h. The crude material was purified by silica gel column chromatography (gradient of 1:2, 1:1 to 3:1 EtOAc in hexanes+2% TEA), followed by preparative TLC (3:1 EtOAc:hexanes and 2% TEA) to yield 377 mg of the product as beige solid, 75%. ¹H NMR (500 MHz, CDCl₃) δ 8.10 (s, 1H), 7.61 (d, J=7.9 Hz, 1H), 7.34 (d, J=8.1 Hz, 1H), 7.18 (t, J=7.5 Hz, 1H), 7.11 (t, J=7.4 Hz, 1H), 6.99 (s, 1H), 6.40 (t, J=7.4 Hz, 1H), 6.33-6.24 (m, 1H), 3.54-3.47 (m, 1H), 3.12 (dd, J=9.6, 2.1 Hz, 1H), 3.02-2.79 (m, 3H), 2.62-2.50 (m, 2H), 2.23-2.12 (m, 1H), 2.11-1.99 (m, 1H), 1.65-1.55 (m, 1H), 1.34 (tt, J=12.2, 3.1 Hz, 1H), 1.31-1.21 (m, 1H). ¹³C NMR (126 MHz, CDCl₃) δ 136.3, 133.3, 132.0, 127.7, 122.0, 121.5, 119.2, 119.0, 114.8, 111.2, 59.4, 55.7, 52.8, 30.9, 26.7, 24.5, 22.2. HRMS (ESI+): calcd. for C₁₇H₂₁N₂[M+H]⁺=253.1705. found [M+H]⁺=253.1707.

### N-ethyl-2-(5-methoxy-1H-indol-3-yl)-N-methylethan-1-amine (9)

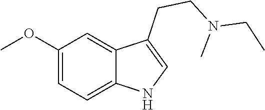 [SMILES: CCN(C)CCc1c[nH]c2ccc(OC)cc12]

Synthesized according to general procedure C Method I. 3-(2-bromoethyl)-5-methoxy-1H-indole (700 mg, 2.75 mmol, 1 equiv.) was dissolved in 14 mL of MeOH (0.2 M). N-methyl-N-ethylamine (27.55 mmol, 2.36 mL, 10 equiv.) were added. The product was isolated on column chromatography (9:1 EtOAc:MeOH and 2% TEA) to yield 256 mg of the product, 40%. ¹H NMR (400 MHz, CDCl₃) δ 8.59 (s, 1H), 7.21 (d, J=8.8 Hz, 1H), 7.07 (d, J=2.5 Hz, 1H), 6.96 (d, J=2.4 Hz, 1H), 6.86 (dd, J=8.8, 2.5 Hz, 1H), 3.87 (s, 3H), 3.03-2.90 (m, 2H), 2.80-2.69 (m, 2H), 2.59 (q, J=7.2 Hz, 2H), 2.40 (s, 3H), 1.15 (t, J=7.2 Hz, 3H). ¹³C NMR (101 MHz, CDCl₃) δ 153.83, 131.64, 127.90, 122.57, 113.84, 111.97, 100.80, 57.77, 56.01, 51.35, 41.55, 23.24, 12.16. HRRS (ESI+): calcd. for C₁₄H₂₁N₂O [M+H]⁺=233.1654. found [M+H]⁺=233.1664.

### N-(2-(5-methoxy-1H-indol-3-yl)ethyl)-N-methylpropan-1-amine (10)

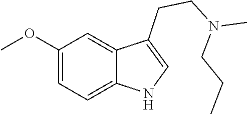

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and N-methyl-N-propylamine (1.97 mmol, 0.2 mL, 10 equiv.). The product was isolated on preparative TLC (9:1 EtOAc:MeOH and 2% TEA) to yield 20.0 mg of the product, 41%. ¹H NMR (500 MHz, CDCl₃) δ 8.17 (br s, 1H), 7.19 (d, J=8.7 Hz, 1H), 7.02 (d, J=2.5 Hz, 1H), 6.95 (d, J=2.3 Hz, 1H), 6.82 (dd, J=8.8, 2.5 Hz, 1H), 3.83 (s, 3H), 2.95-2.83 (m, 2H), 2.74-2.65 (m, 2H), 2.45-2.39 (m, 2H), 2.34 (s, 3H), 1.58-1.48 (m, 2H), 0.90 (t, J=7.4 Hz, 3H). ¹³C NMR (126 MHz, CDCl₃) δ 153.50, 131.16, 127.59, 122.05, 113.85, 111.68, 111.53, 100.43, 59.50, 57.91, 55.64, 41.96, 22.90, 20.14, 11.71. HRRS (ESI+): calcd. for C₁₅H₂₃N₂O [M+H]⁺=247.1810. found [M+H]⁺=247.1812.

### N,N-diethyl-2-(5-methoxy-1H-indol-3-yl)ethan-1-amine (11)

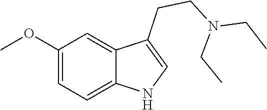 [SMILES: CCN(CC)CCc1c[nH]c2ccc(OC)cc12]

Synthesized according to general procedure C Method I. 3-(2-bromoethyl)-5-methoxy-1H-indole (100 mg, 0.39 mmol, 1 equiv.) was dissolved in 2 mL of MeOH (0.2 M), and diethylamine (3.94 mmol, 0.4 mL, 10 equiv.). The product was isolated on preparative TLC (9:1 EtOAc:MeOH and 2% TEA) to yield 60.0 mg of the product, 58%. ¹H NMR (500 MHz, MeOD) δ 7.22 (d, J=8.7 Hz, 1H), 7.05-6.97 (m, 2H), 6.76 (dd, J=8.7, 2.4 Hz, 1H), 3.81 (s, 3H), 2.94-2.85 (m, 2H), 2.84-2.79 (m, 2H), 2.72 (q, J=7.2 Hz, 4H), 1.12 (t, J=7.2 Hz, 6H. ¹³C NMR (126 MHz, MeOD) δ 153.56, 132.05, 127.55, 122.51, 111.98, 111.58, 111.11, 99.84, 54.91, 52.88, 46.36, 21.48, 9.79. HRMS (ESI+): calcd. for C₁₅H₂₃N₂O [M+H]⁺=247.1810. found [M+H]⁺=247.1816.

### N-ethyl-N-(2-(5-methoxy-1H-indol-3-yl)ethyl)propan-1-amine (12)

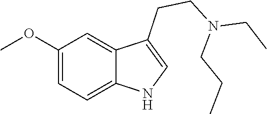 [SMILES: CCCN(CC)CCc1c[nH]c2ccc(OC)cc12]

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and N-ethyl-N-propylamine (1.97 mmol, 0.19 mL, 10 equiv.). The product was isolated on preparative TLC (9:1 EtOAc:MeOH and 2% TEA) to yield 37.0 mg of the product, 77%. ¹H NMR (500 MHz, CDCl₃) δ 8.09 (br s, 1H), 7.28-7.17 (m, 1H), 7.06 (d, J=2.5 Hz, 1H), 6.99 (d, J=2.3 Hz, 1H), 6.86 (dd, J=8.8, 2.5 Hz, 1H), 3.87 (s, 3H), 2.96-2.88 (m, 2H), 2.85-2.77 (m, 2H), 2.69 (q, J=7.2 Hz, 2H), 2.61-2.49 (m, 2H), 1.63-1.49 (m, 2H), 1.11 (t, J=7.2 Hz, 3H), 0.93 (t, J=7.4 Hz, 3H). ¹³C NMR (126 MHz, CDCl₃) δ 153.81, 131.42, 127.92, 122.24, 114.37, 111.97, 111.78, 100.74, 55.89, 55.59, 54.02, 47.53, 22.88, 20.21, 12.03, 11.72. HRMS (ESI+): calcd. for C₁₆H₂₅N₂O [M+H]⁺=261.1967. found [M+H]⁺=261.1966.

### N-(2-(5-methoxy-1H-indol-3-yl)ethyl)-N-propylpropan-1-amine (13)

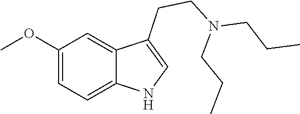 [SMILES: CCCN(CCC)CCc1c[nH]c2ccc(OC)cc12]

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and N-methylprop-2-en-1-amine (1.97 mmol, 0.27 mL, 10 equiv.). The product was isolated on preparative TLC (9:1 EtOAc:MeOH and 2% TEA) to yield 30.0 mg of the product, 56%. ¹H NMR (500 MHz, CDCl₃) δ 8.00 (br s, 1H), 7.32-7.23 (m, 1H), 7.08 (d, J=2.5 Hz, 1H), 7.02 (d, J=2.4 Hz, 1H), 6.88 (dd, J=8.8, 2.5 Hz, 1H), 3.89 (s, 3H), 2.96-2.88 (m, 2H), 2.87-2.79 (m, 2H), 2.63-2.49 (m, 4H), 1.64-1.49 (m, 4H), 0.94 (t, J=7.3 Hz, 6H). ¹³C NMR (126 MHz, CDCl₃) δ 153.88, 131.46, 127.99, 122.27, 114.50, 112.04, 111.81, 100.82, 56.25, 55.95, 54.67, 22.91, 20.27, 12.04. HRRS (ESI+): calcd. for C₁₇H₂₇N₂O [M+H]⁺=275.2123. found [M+H]⁺=275.2119.

### N-(2-(5-methoxy-1H-indol-3-yl)ethyl)-N-methylpropan-2-amine (14)

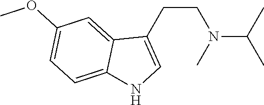 [SMILES: COc1ccc2[nH]cc(CCN(C)C(C)C)c2c1]

Synthesized according to general procedure F. Part I: 5-methoxy-1H-indole (192 mg, 1.31 mmol, 1 equiv.) was dissolved in 4.5 mL of THF (0.3 M), and oxalylchloride (1.96 mmol, 0.17 mL, 1.5 equiv.) was added. After filtration of the glyoxychloride intermediate, the material was resuspended in THF and N-methylisopropylamine (0.41 mL, 3.93 mmol, 3 equiv.) was added. Part II: The glyoxyamide intermediate was suspended in THF and added dropwise to 1 M solution of LAH (249 mg, 6.56 mmol, 5 equiv.) in THF (6.5 mL) at 0° C. The product was isolated on silica column (9:1 EtOAc:MeOH and 2% TEA) to yield 134 mg of the product, 41% over 2 steps. ¹H NMR (500 MHz, MeOD) δ 7.21 (d, J=8.8 Hz, 1H), 7.04-6.97 (m, 2H), 6.75 (dd, J=8.8, 2.4 Hz, 1H), 3.79 (s, 3H), 2.93 (p, J=6.5 Hz, 1H), 2.89-2.84 (m, 2H), 2.75-2.68 (m, 2H), 2.30 (s, 3H), 1.04 (d, J=6.6 Hz, 6H). ¹³C NMR (126 MHz, MeOD) δ 154.88, 133.36, 128.92, 123.91, 113.30, 112.96, 112.44, 101.24, 56.25, 55.13, 54.71, 37.08, 24.04, 17.82. HRIS (ESI+): calcd. for C₁₅H₂₃N₂O [M+H]⁺=247.1810. found [M+H]⁺=247.1815.

### N-isopropyl-N-(2-(5-methoxy-1H-indol-3-yl)ethyl)propan-1-amine (15)

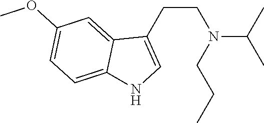 [SMILES: CCCN(CCc1c[nH]c2ccc(OC)cc12)C(C)C]

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and N-methylprop-2-eN-1-amine (1.97 mmol, 0.27 mL, 10 equiv.). The product was isolated on preparative TLC (9:1 EtOAc:MeOH and 2% TEA) to yield 29.0 mg of the product, 54%. ¹H NMR (500 MHz, CDCl₃) δ 8.04 (br s, 1H), 7.24 (d, J=8.8 Hz, 1H), 7.07 (d, J=2.4 Hz, 1H), 7.00 (d, J=2.4 Hz, 1H), 6.86 (dd, J=8.8, 2.5 Hz, 1H), 3.87 (s, 3H), 3.08 (p, J=6.6 Hz, 1H), 2.91-2.84 (m, 2H), 2.79-2.70 (m, 2H), 2.52-2.46 (m, 2H), 1.59-1.50 (m, 2H), 1.06 (d, J=6.6 Hz, 6H), 0.93 (t, J=7.4 Hz, 3H). ¹³C NMR (126 MHz, CDCl₃) δ 153.84, 131.44, 128.03, 122.31, 114.71, 111.99, 111.83, 100.85, 55.93, 52.60, 51.14, 50.89, 25.39, 22.29, 18.44, 12.08. HRRS (ESI+): calcd. for C₁₇H₂₇N₂O [M+H]⁺=275.2123. found [M+H]⁺=275.2125.

### N-(2-(5-methoxy-1H-indol-3-yl)ethyl)-N-methylprop-2-on-1-amine (16)

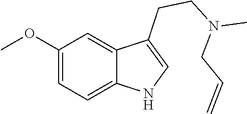

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and N-methylprop-2-en-1-amine (1.97 mmol, 0.19 mL, 10 equiv.). The product was isolated on preparative TLC (9:1 EtOAc:MeOH and 2% TEA) to yield 32.0 mg of the product, 67%. ¹H NMR (500 MHz, CDCl₃) δ 8.17 (s, 1H), 7.22 (d, J=8.8 Hz, 1H), 7.05 (d, J=2.4 Hz, 1H), 6.98 (d, J=2.4 Hz, 1H), 6.86 (dd, J=8.7, 2.5 Hz, 1H), 6.01-5.87 (m, 1H), 5.28-5.13 (m, 2H), 3.87 (s, 3H), 3.18-3.10 (m, 2H), 2.99-2.90 (m, 2H), 2.79-2.70 (m, 2H), 2.38 (s, 3H). ¹³C NMR (126 MHz, CDCl₃) δ 153.86, 135.53, 131.49, 127.89, 122.42, 117.83, 114.01, 112.07, 111.88, 100.75, 60.92, 57.66, 55.97, 42.07, 23.31. HRMS (ESI+): calcd. for C₁₅H₂₁N₂O [M+H]⁺=245.1654. found [M+H]⁺=245.1650.

### N-allyl-N-(2-(5-methoxy-1H-indol-3-yl)ethyl)prop-2-on-1-amine (17)

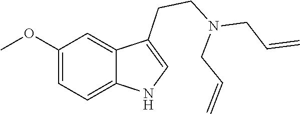 [SMILES: C=CCN(CC=C)CCc1c[nH]c2ccc(OC)cc12]

Synthesized according to general procedure C Method I. 3-(2-bromoethyl)-5-methoxy-1H-indole (50 mg, 0.2 mmol, 1 equiv.) was dissolved in 1 mL of MeOH (0.2 M), and diallylamine (1.97 mmol, 0.27 mL, 10 equiv.). The product was isolated on preparative TLC with 98:2 EtOAc:TEA as the modifier to yield 16 mg of the product, 30%. ¹H NMR (500 MHz, MeOD) δ 7.20 (dd, J=8.7, 0.6 Hz, 1H), 6.98 (d, J=2.8 Hz, 2H), 6.74 (dd, J=8.7, 2.5 Hz, 1H), 6.00-5.88 (m, 2H), 5.33-5.20 (m, 4H), 3.80 (s, 3H), 3.27 (dt, J=6.8, 1.2 Hz, 4H), 2.91-2.87 (m, 2H), 2.83-2.77 (m, 2H). ¹³C NMR (126 MHz, MeOD) δ 154.87, 135.41, 133.38, 128.93, 123.89, 119.50, 113.39, 112.92, 112.52, 101.25, 57.64, 56.27, 54.73, 22.97. HRMS (ESI+): calcd. for C₁₇H₂₃N₂O [M+H]⁺=271.1810. found [M+H]⁺=271.1808.

### 5-methoxy-3-(2-(pyrrolidin-1-yl)ethyl)-1H-indole (18)

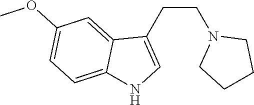 [SMILES: COc1ccc2[nH]cc(CCN3CCCC3)c2c1]

Synthesized according to general procedure F with slight modifications. Part I: 5-methoxy-1H-indole (1.47 g, 9.99 mmol, 1 equiv.) was dissolved in 30 mL of THF (0.3 M), and oxalylchloride (14.98 mmol, 1.28 mL, 1.5 equiv.) was added. After filtration of the glyoxychloride intermediate, the material was resuspended in THF and pyrrolidine (2.5 mL, 29.96 mmol, 3 equiv.) were added. Part II: The glyoxyamide intermediate was suspended in THF (30 mL) and LAH was added portion wise (1.9 g, 49.95 mmol, 5 equiv., based on the indole starting material). The product was isolated on silica column with 1:1 DCM:ether and 2% TEA as the modifier to yield 1.2 g of the product, 50% over 3 steps. ¹H NMR (500 MHz, MeOD) δ 7.21 (d, J=8.7 Hz, 1H), 7.04-6.96 (m, 2H), 6.75 (dd, J=8.8, 2.5 Hz, 1H), 3.80 (s, 3H), 2.96-2.87 (m, 2H), 2.77-2.70 (m, 2H), 2.64-2.56 (m, 4H), 1.87-1.76 (m, 4H). ¹³C NMR (126 MHz, MeOD) δ 154.89, 133.39, 128.96, 123.78, 113.65, 112.89, 112.44, 101.30, 58.29, 56.29, 54.95, 25.66, 24.17. HRMS (ESI+): calcd. for C₁₅H₂₁N₂O [M+H]⁺=245.1654. found [M+H]⁺=245.1665.

### 5-methoxy-3-(2-(2-methylpyrrolidin-1-yl)ethyl)-1H-indole (19)

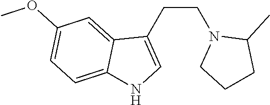

Synthesized according to general procedure C Method I. 3-(2-bromoethyl)-5-methoxy-1H-indole (100 mg, 0.39 mmol, 1 equiv.) was dissolved in 1.5 mL of MeOH (0.2 M). 2-methylpyrrolidine (3.9 mmol, 0.4 mL, 10 equiv.) was added. The product was isolated on column chromatography with 9:1 EtOAc:Hexanes with 2% TEA as the modifier to yield 88 mg of the product, 87%. ¹H NMR (500 MHz, DMSO) δ 7.29-7.22 (m, 2H), 7.09 (d, J=2.5 Hz, 1H), 6.75 (dd, J=8.7, 2.5 Hz, 1H), 3.79 (s, 3H), 3.74-3.65 (m, 1H), 3.58-3.45 (m, 2H), 3.28-3.19 (m, 2H), 3.12-3.01 (m, 2H), 2.26-2.17 (m, 1H), 2.08-1.88 (m, 2H), 1.68-1.56 (m, 1H), 1.39 (d, J=6.5 Hz, 3H). ¹³C NMR (126 MHz, MeOD) δ 153.94, 132.05, 127.05, 123.52, 111.88, 111.57, 108.61, 99.68, 64.18, 55.03, 53.18, 30.81, 23.03, 21.74, 20.99, 16.08, 14.81. HRIS (ESI+): calcd. for C₁₆H₂₃N₂O [M+H]⁺=259.1810. found [M+H]⁺=259.1814.

### 5-methoxy-3-(2-(piperidin-1-yl)ethyl)-1H-indole (20)

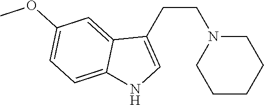 [SMILES: COc1ccc2[nH]cc(CCN3CCCCC3)c2c1]

Synthesized according to general procedure C Method I. 3-(2-bromoethyl)-5-methoxy-1H-indole (500 mg, 1.97 mmol, 1 equiv.) was dissolved in 8.5 mL of MeOH (0.2 M). Piperidine (22.27 mmol, 2.2 mL, 6.6 equiv.) was added. The product was isolated on column chromatography with 1:1 EtOAc:Hexanes with 2% TEA as the modifier to yield 330 mg of the product, 65%. ¹H NMR (500 MHz, CDCl₃) δ 8.28 (s, 1H), 7.23 (d, J=8.8 Hz, 1H), 7.06 (d, J=2.4 Hz, 1H), 6.98 (d, J=2.5 Hz, 1H), 6.84 (dd, J=8.8, 2.4 Hz, 1H), 3.85 (s, 3H), 3.11-2.91 (m, 2H), 2.82-2.63 (m, 2H), 2.67-2.49 (m, 4H), 1.81-1.65 (m, 4H), 1.59-1.42 (m, 2H). ¹³C NMR (126 MHz, CDCl₃) δ 153.97, 131.53, 127.89, 122.55, 113.74, 112.19, 112.00, 100.79, 59.81, 56.09, 54.56, 25.67, 24.27, 22.67. HRRS (ESI+): calcd. for C₁₆H₂₃N₂O [M+H]⁺=259.1810. found [M+H]⁺=259.1822.

### 3-(2-(2,5-dihydro-1H-pyrrol-1-yl)ethyl)-5-methoxy-1H-indole (21)

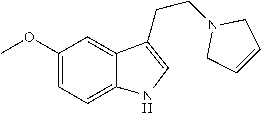 [SMILES: COc1ccc2[nH]cc(CCN3CC=CC3)c2c1]

Synthesized according to general procedure C Method I.3-(2-bromoethyl)-5-methoxy-1H-indole (109 mg, 0.43 mmol, 1 equiv.) was dissolved in 2 mL of MeOH and 3-pyrroline (30.43 mmol, 0.26 mL, 8 equiv.). The product was isolated on preparative TLC (9:1 EtOAc:MeOH and 2% TEA) to yield 63 mg of the product, 61%. ¹H NMR (300 MHz, DMSO) δ 10.58 (s, 1H), 7.21 (d, J=8.8 Hz, 1H), 7.10 (d, J=2.3 Hz, 1H), 6.98 (d, J=2.4 Hz, 1H), 6.70 (dd, J=8.7, 2.4 Hz, 1H), 5.81 (s, 2H), 3.76 (s, 3H), 3.57-3.42 (m, 4H), 2.89-2.69 (m, 4H). ¹³C NMR (126 MHz, MeOD) δ 155.13, 133.57, 129.08, 128.28, 124.14, 113.24, 113.10, 112.70, 101.43, 60.80, 58.21, 56.47, 25.70. HRMS (ESI+): calcd. for C₁₅H₁₉N₂O [M+H]⁺=243.1497. found [M+H]⁺=243.1495.

### 3-(2-(1H-imidazol-1-yl)ethyl)-5-methoxy-1H-indole (22)

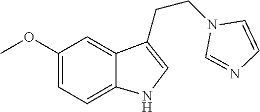 [SMILES: COc1ccc2[nH]cc(CCn3ccnc3)c2c1]

Synthesized according to general procedure C Method I. 3-(2-bromoethyl)-5-methoxy-1H-indole (81 mg, 0.32 mmol, 1 equiv.) was dissolved in 1.5 mL of MeOH. 1H-imidazole (0.32 mmol, 22 mg, 1 equiv.) and NaHCO₃ (87 mg, 1.27 mmol, 4 equiv.) were added. The product was isolated on preparative TLC (9:1 EtOAc:MeOH and 2% TEA) to yield 26 mg of the product, 34%. ¹H NMR (500 MHz, MeOD) δ 7.47 (s, 1H), 7.23 (d, J=8.7 Hz, 1H), 7.16-7.07 (m, 1H), 7.01-6.93 (m, 1H), 6.90-6.86 (m, 2H), 6.76 (dd, J=8.7, 2.4 Hz, 1H), 4.32 (t, J=6.7 Hz, 2H), 3.82 (s, 3H), 3.20 (t, J=6.7 Hz, 2H). ¹³C NMR (126 MHz, MeOD) δ 153.73, 137.08, 131.87, 127.35, 127.31, 119.30, 111.57, 111.39, 110.48, 99.41, 54.87, 47.73, 27.07. HRMS (ESI+): calcd. for C₁₄H₁₆N₃O [M+H]⁺=242.1293. found [M+H]⁺=242.1296.

### 3-(2-(3,6-dihydropyridin-1(2H)-yl)ethyl)-5-methoxy-1H-indole (23)

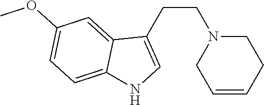 [SMILES: COc1ccc2[nH]cc(CCN3CC=CCC3)c2c1]

Synthesized according to general procedure F. 5-methoxy-1H-indole (2.0 g, 12.1 mmol, 1 equiv.) was dissolved in 50 mL of dry diethyl ether (0.2 M), and oxalyl chloride (20.8 mmol, 1.75 mL, 1.5 equiv.) was added. The product was filtered and used directly in the next steps. The glyoxychloride (1 g, 4.2 mmol, 1 equiv.) was suspended in 1.0 M of THF and a mixture of TEA (0.87 mL, 6.3 mmol, 1.5 equiv.) and 1,2,3,6-tetrahydropyridine (0.46 mL, 5.0 mmol, 1.2 equiv.) in 20 mL THF (0.2 M) were added. The Crude glyoxyamide was suspended in 8 mL of dry THF (0.5 M) and added dropwise to a cold suspension of LAH (5 equiv.) in dry THF (1.0 M). The crude material was purified on silica gel column chromatography (9:1 EtOAc:MeOH and 2% TEA) to yield 1.01 g of the product, 91%. ¹H NMR (500 MHz, CDCl₃) δ 8.20 (s, 1H), 7.23 (dd, J=8.8, 0.6 Hz, 1H), 7.07 (d, J=2.5 Hz, 1H), 6.99 (d, J=2.4 Hz, 1H), 6.85 (dd, J=8.8, 2.4 Hz, 1H), 5.85-5.78 (m, 1H), 5.75-5.67 (m, 1H), 3.86 (s, 3H), 3.16-3.10 (m, 2H), 3.05-2.97 (m, 2H), 2.81-2.76 (m, 2H), 2.71 (t, J=5.7 Hz, 2H), 2.32-2.22 (m, 2H). ¹³C NMR (126 MHz, CDCl₃) δ 153.88, 131.46, 127.87, 125.35, 125.22, 122.44, 114.00, 112.08, 111.90, 100.73, 59.22, 55.99, 52.72, 50.16, 26.18, 23.18. HRIS (ESI+): calcd. for C₁₆H₂₁N₂O [M+H]⁺=257.1654. found [M+H]⁺=257.1661.

### (1S,4R)-2-(2-(5-methoxy-1H-indol-3-yl)ethyl)-2-azabicyclo[2.2.2]oct-5-one (24)

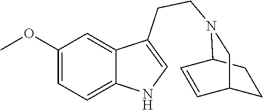 [SMILES: COc1ccc2[nH]cc(CCN3CC4C=CC3CC4)c2c1]

Synthesized according to general procedure F with some modifications. 5-methoxy-1H-indole (200 mg, 1.36 mmol, 1 equiv.) was dissolved in 4 mL of dry diethyl ether (0.2 M), and oxalyl chloride (134 μL, 1.56 mmol, 1.15 equiv.) was added. The product was filtered and used directly in the next steps. The glyoxychloride was suspended in 0.2 M of MeCN (2 mL) and a mixture of TEA (753 μL, 5.4 mmol, 4 equiv. based on starting indole) and 2-azabicyclo[2.2.2]oct-5-ene hydroiodide (161 mg, 0.68 mmol, 0.5 equiv. based on starting indole) were added. The Crude glyoxyamide was suspended in 7 mL of dry THF and added dropwise to a cold suspension of LAH (5 equiv.) in dry THF (1.0 M). The crude material was purified on silica gel column chromatography (first column: gradient of 1:2, 1:1 to 2:1 AcOEt in hexanes+2% TEA and second column: diethyl ether to diethyl ether+2% TEA) to yield 131 mg of the product as beige solid, 68% over two steps. ¹H NMR (400 MHz, CDCl₃) δ 7.81 (s, 1H), 7.23 (d, J=8.8 Hz, 1H), 7.05 (d, J=2.4 Hz, 1H), 6.98 (d, J=2.3 Hz, 1H), 6.84 (dd, J=8.8, 2.5 Hz, 1H), 6.39 (t, J=7.4 Hz, 1H), 6.31-6.25 (m, 1H), 3.86 (s, 3H), 3.54-3.46 (m, 1H), 3.11 (dd, J=9.6, 2.1 Hz, 1H), 2.96-2.77 (m, 3H), 2.59-2.47 (m, 2H), 2.14 (dt, J=9.6, 2.7 Hz, 1H), 2.10-1.97 (m, 1H), 1.65-1.54 (m, 1H), 1.38-1.29 (m, 1H), 1.29-1.20 (m, 1H). ¹³C NMR (126 MHz, CDCl₃) δ 153.9, 133.4, 131.9, 131.5, 128.0, 122.4, 114.4, 112.1, 111.9, 100.9, 59.2, 56.1, 55.6, 52.8, 30.9, 26.6, 24.4, 22.2. HRRS (ESI+): calcd. for C₁₈H₂₃N₂O [M+H]⁺=283.1810. found [M+H]1=283.1822.

### 2-(4-fluoro-5-methoxy-1H-indol-3-yl)-N,N-dimethylethane-1-amine (25)

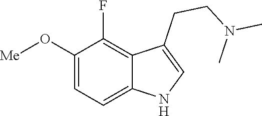 [SMILES: COc1ccc2[nH]cc(CCN(C)C)c2c1F]

Synthesized according to general procedure C, method I. 3-(2-bromoethyl)-4-fluoro-5-methoxy-1H-indole (100 mg, 0.37 mmol, 1 equiv.) was dissolved in 1.8 mL of 2M dimethylamine solution (3.67 mmol, 10 equiv.). The product was isolated on preparative TLC with 9:1 EtOAc:MeOH and 2% TEA as the modifier to yield 60 mg of the product, 69%. ¹H NMR (500 MHz, MeOD) δ 7.07-6.99 (m, 2H), 6.95-6.88 (m, 1H), 3.87 (s, 3H), 3.04-2.96 (m, 2H), 2.72-2.63 (m, 2H), 2.36 (s, 6H). ¹³C NMR (126 MHz, MeOD) δ 147.58, 145.65, 139.28, 139.20, 134.92, 134.83, 123.55, 116.87, 116.74, 111.87, 110.49, 110.47, 106.37, 106.34, 60.88, 60.86, 58.18, 43.88, 23.79. ¹⁹F MR (471 MHz, MeOD) δ −148.51. HRRS (ESI+): calcd. for C₁₃H₁₇FN₂O [M+H]⁺=237.1403. found [M+H]⁺=237.1409.

### N-ethyl-2-(4-fluoro-5-methoxy-1H-indol-3-yl)-N-methylethan-1-amine (26)

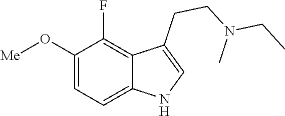 [SMILES: CCN(C)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N-ethyl-N-methylamine (1.84 mmol, 0.16 mL, 10 equiv.). The product was isolated on preparative TLC (9:1 EtOAc:MeOH and 2% TEA) to yield 23.0 mg of the product, 50%. ¹H NMR (500 MHz, MeOD) δ 7.06-6.97 (m, 2H), 6.90 (t, J=8.3 Hz, 1H), 3.86 (s, 3H), 3.03-2.92 (m, 2H), 2.75-2.65 (m, 2H), 2.55 (q, J=7.7 Hz, 2H), 2.33 (s, 3H), 1.11 (t, J=7.2 Hz, 3H). ¹³C NMR (500 MHz, MeOD) δ 147.58, 145.65, 139.27, 139.19, 134.91, 134.83, 123.52, 116.91, 116.77, 111.82, 110.73, 110.71, 106.39, 106.36, 58.39, 58.37, 58.16, 50.67, 40.28, 23.18, 10.48. ¹⁹F NMR (471 MHz, MeOD) δ −148.39. HRRS (ESI+): calcd. for C₁₄H₂₀FN₂O [M+H]⁺=251.1560. found [M+H]⁺=251.1568.

### N,N-diethyl-2-(4-fluoro-5-methoxy-1H-indol-3-yl)ethan-1-amine (27)

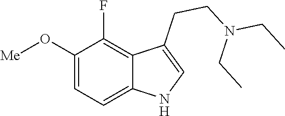 [SMILES: CCN(CC)CCc1c[nH]c2ccc(OC)c(F)c12]

Synthesized according to general procedure C, method I. 3-(2-bromoethyl)-4-fluoro-5-methoxy-1H-indole (100 mg, 0.37 mmol, 1 equiv.) was dissolved in 2 mL of MeOH and diethylamine (3.67 mmol, 0.3 mL, 10 equiv.). The product was isolated on preparative TLC with 9:1 EtOAc:MeOH and 2% TEA as the modifier to yield 92 mg product, 95%. ¹H NMR (500 MHz, CDCl₃) δ 8.90 (s, 1H), 6.98 (d, J=8.8 Hz, 1H), 6.94-6.85 (m, 2H), 3.05-2.94 (m, 2H), 2.86-2.78 (m, 2H), 2.70 (q, J=7.2 Hz, 4H), 1.11 (t, J=7.2 Hz, 6H). ¹³C NMR (500 MHz, CDCl₃) δ 147.80, 145.85, 139.75, 139.67, 134.39, 134.30, 123.41, 117.51, 117.37, 112.75, 112.73, 112.04, 106.46, 106.42, 59.03, 54.42, 54.41, 46.93, 23.64, 11.68. ¹⁹F NMR (471 MHz, MeOD) δ −148.45. HRMS (ESI+): cal. C₁₅H₂₂FN₂O [M+H]⁺=265.1716. found [M+H]⁺=265.1728.

### N-(2-(4-fluoro-5-methoxy-1H-indol-3-yl)ethyl)-N-methylpropan-1-amine (28)

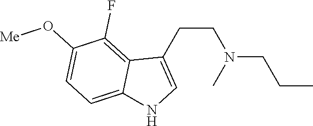 [SMILES: CCCN(C)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N-methyl-N-propylamine (1.84 mmol, 0.18 mL, 10 equiv.). The product was isolated on preparative TLC with 9:1 EtOAc:MeOH and 2% TEA as the modifier to yield 40.0 mg of the product, 82%. ¹H NMR (500 MHz, MeOD) δ 7.07-6.96 (m, 2H), 6.91 (t, J=8.3 Hz, 1H), 3.87 (s, 3H), 3.03-2.93 (m, 2H), 2.76-2.64 (m, 2H), 2.47-2.38 (m, 2H), 2.33 (s, 3H), 1.65-1.48 (m, 2H), 0.92 (t, J=7.4 Hz, 3H). ¹³C NMR (500 MHz, MeOD) δ 148.98, 147.05, 140.65, 140.57, 136.28, 136.20, 124.87, 118.32, 118.19, 113.19, 112.23, 112.21, 107.76, 107.73, 60.56, 60.24, 59.54, 42.34, 24.53, 20.84, 12.21. ¹⁹F NMR (471 MHz, MeOD) δ −147.17. HRRS (ESI+): calcd. for C₁₅H₂₂FN₂O [M+H]⁺=265.1716. found [M+H]⁺=265.1726.

### N-ethyl-(2-(4-fluoro-5-methoxy-1H-indol-3-yl)ethyl)propan-1-amine (29)

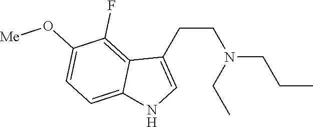 [SMILES: CCCN(CC)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and i-ethyl-N-propylamine (1.84 mmol, 0.23 mL, 10 equiv.). The product was isolated on preparative TLC with 9:1 EtOAc:MeOH and 2% TEA as the modifier to yield 44.0 mg of the product, 86%. ¹H NMR (500 MHz, MeOD) δ 7.06-6.98 (m, 2H), 6.94-6.84 (m, 1H), 3.87 (s, 3H), 3.02-2.92 (m, 2H), 2.86-2.76 (m, 2H), 2.70 (q, J=7.2 Hz, 2H), 2.60-2.50 (m, 2H), 1.62-1.50 (m, 2H), 1.12 (t, J=7.2 Hz, 3H), 0.92 (d, J=7.4 Hz, 3H). ¹³C NMR (500 MHz, MeOD) δ 148.97, 147.04, 140.63, 140.55, 136.29, 136.20, 124.87, 118.31, 118.17, 113.12, 112.34, 112.32, 107.76, 107.73, 59.52, 56.50, 56.05, 56.03, 48.40, 23.98, 20.47, 12.29, 11.46. ¹⁹F NMR (471 MHz, MeOD) δ −148.32. HRMS (ESI+): calcd. for C₁₆H₂₄FN₂O [M+H]⁺=279.1873. found [M+H]⁺=279.1872.

### N-(2-(4-fluoro-5-methoxy-1H-indol-3-yl)ethyl)-N-propylpropan-1-amine (30)

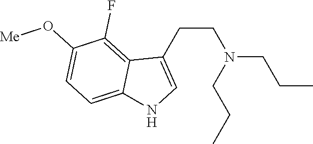 [SMILES: CCCN(CCC)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and dipropylamine (1.84 mmol, 0.25 mL, 10 equiv.). The product was isolated on preparative TLC with 9:1 EtOAc:MeOH and 2% TEA as the modifier to yield 45.0 mg of the product, 84%. ¹H NMR (500 MHz, MeOD) δ 7.08-6.95 (m, 2H), 6.89 (t, J=8.3 Hz, 1H), 3.85 (s, 3H), 2.97-2.88 (m, 2H), 2.82-2.72 (m, 2H), 2.57-2.45 (m, 4H), 1.61-1.46 (m, 4H), 0.90 (t, J=7.4 Hz, 6H). ¹³C NMR (500 MHz, MeOD) δ 147.59, 145.66, 139.24, 139.17, 134.90, 134.82, 123.52, 116.93, 116.80, 111.73, 110.94, 110.92, 106.39, 106.35, 58.13, 55.74, 55.31, 55.29, 22.66, 19.16, 10.88. ¹⁹F NMR (471 MHz, MeOD) δ −148.35. HRRS (ESI+): calcd. for C₁₇H₂₆FN₂O [M+H]⁺=293.2029. found [M+H]⁺=293.2039.

### N-(2-(4-fluoro-5-methoxy-1H-indol-3-yl)ethyl)-N-methylpropan-2-amine (31)

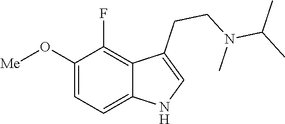 [SMILES: COc1ccc2[nH]cc(CCN(C)C(C)C)c2c1F]

Synthesized according to general procedure C Method I. 3-(2-bromoethyl)-4-fluoro-5-methoxy-1H-indole (100 mg, 0.37 mmol, 1 equiv.) was dissolved in 2 mL of MeOH and methyl isopropylamine (3.67 mmol, 0.4 mL, 10 equiv.). The product was isolated on preparative TLC with 9:1 EtOAc:MeOH and 2% TEA as the modifier to yield 97 mg of the product, 99%. ¹H NMR (500 MHz, CDCl₃) δ 8.90 (s, 1H), 6.99 (d, J=8.7 Hz, 1H), 6.95-6.86 (m, 2H), 3.92 (s, 3H), 3.05-2.89 (m, 3H), 2.82-2.72 (m, 2H), 2.38 (s, 3H), 1.06 (d, J=6.6 Hz, 6H). ¹³C NMR (500 MHz, CDCl₃) δ 147.72, 145.77, 139.71, 139.63, 134.34, 134.26, 123.44, 117.46, 117.32, 112.62, 111.98, 106.43, 106.40, 58.96, 55.02, 55.00, 53.64, 37.27, 24.97, 18.01. ¹⁹F NMR (471 MHz, MeOD) δ −148.45. HRRS (ESI+): calcd. for C₁₅H₂₂FN₂O [M+H]⁺=265.1716. found [M+H]⁺=265.1724.

### N-(2-(4-fluoro-5-methoxy-1H-indol-3-yl)ethyl)-N-isopropylpropan-1-amine (32)

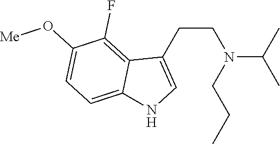 [SMILES: CCCN(CCc1c[nH]c2ccc(OC)c(F)c12)C(C)C]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N-propyl-N-isopropylamine (1.84 mmol, 0.25 mL, 10 equiv.). The product was isolated on preparative TLC with 9:1 EtOAc:MeOH and 2% TEA as the modifier to yield 40.0 mg of the product, 74%. ¹H NMR (500 MHz, MeOD) δ 7.04-6.97 (m, 2H), 6.89 (t, J=8.3 Hz, 1H), 3.86 (s, 3H), 3.04 (p, J=6.6 Hz, 1H), 2.95-2.88 (m, 2H), 2.76-2.69 (m, 2H), 2.53-2.46 (m, 2H), 1.58-1.48 (m, 2H), 1.05 (d, J=6.6 Hz, 6H), 0.91 (t, J=7.4 Hz, 3H). ¹³C NMR (500 MHz, MeOD) δ 148.98, 147.05, 140.61, 140.53, 136.30, 136.21, 124.87, 118.33, 118.19, 113.10, 112.50, 112.48, 107.74, 107.71, 59.54, 53.71, 53.47, 53.46, 52.24, 26.51, 22.27, 18.48, 12.29. ¹⁹F NMR (471 MHz, MeOD) δ −148.30. HRMS (ESI+): calcd. for C₁₇H₂₆FN₂O [M+H]⁺=293.2029. found [M+H]⁺=293.2039.

### N-(2-(4-fluoro-5-methoxy-1H-indol-3-yl)ethyl)-N-isopropylpropan-2-amine (33)

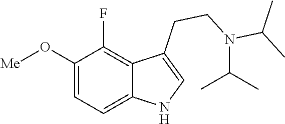 [SMILES: COc1ccc2[nH]cc(CCN(C(C)C)C(C)C)c2c1F]

Synthesized according to general procedure C, method I. 3-(2-bromoethyl)-4-fluoro-5-methoxy-1H-indole (100 mg, 0.37 mmol, 1 equiv.) was dissolved in 2 mL of MeOH and piperidine (3.67 mmol, 0.4 mL, 10 equiv.). The product was isolated on preparative TLC with 9:1 EtOAc:MeOH and 2% TEA as the modifier to yield 85 mg of the product, 79%. ¹H NMR (500 MHz, MeOD) δ 7.06-6.96 (m, 2H), 6.89 (t, J=8.3 Hz, 1H), 3.86 (s, 3H), 3.08 (hept, J=6.5 Hz, 2H), 2.93-2.83 (m, 2H), 2.76-2.65 (m, 2H), 1.09 (d, J=6.6 Hz, 12H). ¹³C NMR (500 MHz, MeOD) δ 148.97, 147.03, 140.57, 140.49, 136.29, 136.21, 124.75, 118.33, 118.20, 113.06, 112.69, 112.67, 107.70, 107.67, 59.56, 50.78, 49.59, 29.98, 20.53. ¹⁹F NMR (471 MHz, MeOD) δ −148.12. HRMS (ESI+): calcd. for C₁₇H₂₆FN₂O [M+H]⁺=293.2029. found [M+H]⁺=293.2039.

### N-(2-(4-fluoro-5-methoxy-1H-indol-3-yl)ethyl)-N-methylprop-2-en-1-amine (34)

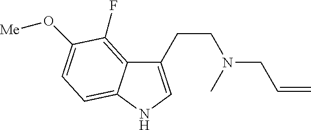 [SMILES: C=CCN(C)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N-propyl-N-isopropylamine (1.84 mmol, 0.25 mL, 10 equiv.). The product was isolated on preparative TLC with 9:1 EtOAc:MeOH and 2% TEA as the modifier to yield 40.0 mg of the product, 74%. ¹H NMR (500 MHz, MeOD) δ 7.05-6.97 (m, 2H), 6.90 (t, J=8.3 Hz, 1H), 5.90 (ddt, J=17.0, 10.2, 6.8 Hz, 1H), 5.28-5.15 (m, 2H), 3.86 (s, 3H), 3.16-3.09 (m, 2H), 3.00-2.95 (m, 2H), 2.75-2.65 (m, 2H), 2.33 (s, 3H). ¹³C NMR (500 MHz, MeOD) δ 147.59, 145.66, 139.26, 139.19, 134.90, 134.81, 134.08, 123.52, 117.91, 116.91, 116.77, 111.83, 110.67, 110.65, 106.38, 106.34, 60.18, 58.47, 58.46, 58.17, 40.64, 23.39. ¹⁹F NMR (471 MHz, MeOD) δ −148.35. HRRS (ESI+): calcd. for C₁₅H₂₀FN₂O [M+H]⁺=263.1560. found [M+H]⁺=263.1568.

### N-(2-(4-fluoro-5-methoxy-1H-indol-3-yl)ethyl)-N-propylprop-2-en-1-amine

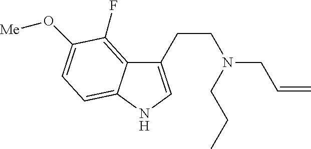 [SMILES: C=CCN(CCC)CCc1c[nH]c2ccc(OC)c(F)c12]

Synthesized according to general procedure C Method I with a slight modification. 3-(2-bromoethyl)-4-fluoro-5-methoxy-1H-indole (50 mg, 0.18 mmol, 1 equiv.) was dissolved in 1 mL of MeOH. N-propyl-N-allylamine hydro chloride (0.55 mmol, 75 mg, 3 equiv.) and NaHCO₃ (0.73 mmol, 62 mg, 4 equiv.) were added. The product was isolated on preparative TLC with 98% EtOAc and 2% TEA as the modifier to yield 35.0 mg of the product, 66%. ¹H NMR (500 MHz, MeOD) δ 7.03-6.94 (m, 2H), 6.93-6.84 (m, 1H), 5.98-5.82 (m, 1H), 5.28-5.11 (m, 2H), 3.85 (s, 3H), 3.23 (d, J=1.3 Hz, 2H), 3.00-2.90 (m, 2H), 2.84-2.73 (m, 2H), 2.58-2.47 (m, 2H), 1.65-1.48 (m, 2H), 0.90 (t, J=7.4 Hz, 3H). ¹³C NMR (500 MHz, MeOD) δ 147.59, 145.66, 139.25, 139.17, 134.89, 134.80, 134.45, 134.24, 123.53, 117.66, 116.92, 116.78, 111.77, 110.80, 110.78, 106.38, 106.35, 58.15, 56.77, 55.22, 54.98, 54.96, 22.77, 19.04, 10.79. ¹⁹F NMR (471 MHz, MeOD) δ −148.30. LRMS (APCI+) calcd. For C₁₇H₂₄FN₂O [M+H]⁺=291.2. found 291.6.

### N-allyl-N-(2-(4-fluoro-5-methoxy-1H-indol-3-yl)ethyl)prop-2-en-1-amine

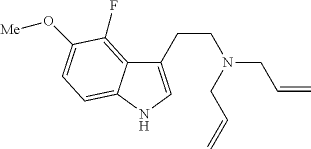 [SMILES: C=CCN(CC=C)CCc1c[nH]c2ccc(OC)c(F)c12]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N,N-diallylamine (1.84 mmol, 0.24 mL, 10 equiv.). The product was isolated on preparative TLC with 98% EtOAc and 2% TEA as the modifier to yield 36.0 mg of the product, 67%. ¹H NMR (500 MHz, MeOD) δ 7.02 (dd, J=8.7, 0.7 Hz, 1H), 6.97 (s, 1H), 6.89 (t, J=8.3 Hz, 1H), 5.95-5.83 (m, 2H), 5.25-5.15 (m, 4H), 3.85 (s, 3H), 3.21 (d, J=6.8 Hz, 4H), 3.01-2.92 (m, 2H), 2.83-2.72 (m, 2H). ¹³C NMR (500 MHz, MeOD) δ 147.61, 145.67, 139.24, 139.16, 134.87, 134.79, 134.38, 134.10, 123.50, 117.87, 117.79, 116.93, 116.80, 111.80, 110.78, 110.76, 106.37, 106.33, 58.17, 56.30, 56.25, 54.67, 54.65, 22.98. ¹⁹F NMR (471 MHz, MeOD) δ −148.17. LRMS (APCI+) calcd. For C₁₇H₂₂FN₂O [M+H]⁺=298.2. found 289.4.

### N-allyl-N-(2-(4-fluoro-5-methoxy-1H-indol-3-yl)ethyl)prop-2-en-1-amine

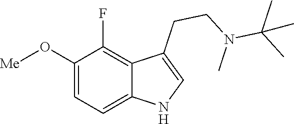 [SMILES: COc1ccc2[nH]cc(CCN(C)C(C)(C)C)c2c1F]

Synthesized according to general procedure C, method I. 3-(2-bromoethyl)-4-fluoro-5-methoxy-1H-indole (50 mg, 0.18 mmol, 1 equiv.) was dissolved in 1 mL of MeOH and N,N-diallylamine (1.84 mmol, 0.24 mL, 10 equiv.). The product was isolated on preparative TLC with 98% EtOAc and 2% TEA as the modifier to yield 36.0 mg of the product, 67%. ¹H NMR (500 MHz, MeOD) δ 7.08-6.97 (m, 2H), 6.90 (t, J=8.3 Hz, 1H), 3.85 (s, 3H), 3.01-2.92 (m, 2H), 2.74-2.67 (m, 2H), 2.37 (s, 3H), 1.11 (s, 9H). ¹³C NMR (500 MHz, MeOD) δ 148.94, 147.01, 140.65, 140.57, 136.31, 136.23, 124.96, 118.31, 118.18, 113.15, 112.20, 112.18, 107.79, 107.76, 59.51, 56.44, 54.60, 54.58, 35.51, 27.05, 25.92. ¹⁹F NMR (471 MHz, MeOD) δ −148.41. LRMS (APCI+) calcd. For C₁₆H₂₄FN₂O [M+H]⁺=279.2. found 279.4.

### 4-fluoro-5-methoxy-3-(2-(pyrrolidin-1-yl)ethyl)-1H-indole (35)

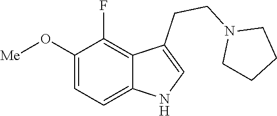 [SMILES: COc1ccc2[nH]cc(CCN3CCCC3)c2c1F]

Synthesized according to general procedure C, method I. 3-(2-bromoethyl)-4-fluoro-5-methoxy-1H-indole (100 mg, 0.37 mmol, 1 equiv.) was dissolved in 2 mL of MeOH and pyrrolidine (3.67 mmol, 0.3 mL, 10 equiv.). The product was isolated on preparative TLC with 9:1 EtOAc:MeOH and 2% TEA as the modifier to yield 92 mg of the product, 95%. ¹H NMR (500 MHz, MeOD) δ 7.08-6.98 (m, 1H), 6.96-6.86 (m, 2H), 3.87 (s, 3H), 3.08-2.97 (m, 2H), 2.83-2.74 (m, 2H), 2.69-2.59 (m, 4H), 1.88-1.78 (m, 4H). ¹³C NMR (500 MHz, MeOD) δ 148.95, 147.02, 140.66, 124.84, 113.17, 112.24, 107.75, 107.72, 59.52, 59.35, 54.96, 26.76, 24.15. ¹⁹F NMR (471 MHz, MeOD) δ −148.52. HRMS (ESI+): calcd. for C₁₅H₂₀FN₂O [M+H]⁺=263.1560. found [M+H]⁺=263.1569.

### 4-fluoro-5-methoxy-3-(2-(piperidin-1-yl)ethyl)-1H-indole (36)

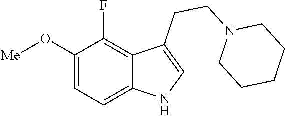 [SMILES: COc1ccc2[nH]cc(CCN3CCCCC3)c2c1F]

Synthesized according to general procedure C, method I. 3-(2-bromoethyl)-4-fluoro-5-methoxy-1H-indole (100 mg, 0.37 mmol, 1 equiv.) was dissolved in 2 mL of MeOH and piperidine (3.67 mmol, 0.42 mL, 10 equiv.). The product was isolated on preparative TLC with 9:1 EtOAc:MeOH and 2% TEA as the modifier to yield 92 mg of the product, 95%. ¹H NMR (500 MHz, CDCl₃) δ 8.92 (s, 1H), 6.93 (d, J=8.7 Hz, 1H), 6.91-6.81 (m, 2H), 3.91 (s, 3H), 3.08-2.97 (m, 2H), 2.78-2.65 (m, 2H), 2.63-2.49 (m, 4H), 1.74-1.59 (m, 4H), 1.54-1.39 (m, 2H). ¹³C NMR (500 MHz, CDCl₃) δ 147.72, 145.78, 139.66, 139.58, 134.31, 134.23, 123.33, 117.38, 117.24, 112.63, 112.03, 106.33, 106.29, 60.92, 60.90, 59.01, 54.45, 25.83, 24.40, 23.57. ¹⁹F NMR (471 MHz, MeOD) δ −148.37. HRRS (ESI+): calcd. for C₁₆H₂₂FN₂O [M+H]⁺=277.1716. found [M+H]⁺=277.1725.

### 3-(2-(2,5-dihydro-1H-pyrrol-1-yl)ethyl)-4-fluoro-5-methoxy-1H-indole (37)

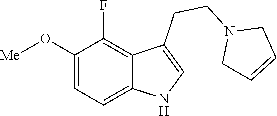 [SMILES: COc1ccc2[nH]cc(CCN3CC=CC3)c2c1F]

Synthesized according to general procedure C, method II. 3-(2-bromoethyl)-4-fluoro-5-methoxy-1H-indole (55 mg, 0.2 mmol, 1 equiv.) was dissolved in 1.5 mL of MeCN. 3-pyrroline (2.2 mmol, 17.0 mg, 1.2 equiv.) and NaHCO₃ (68 mg, 0.8 mmol, 4 equiv.) were added. The product was isolated on preparative TLC with 9:1 EtOAc:MeOH and 2% TEA as the modifier to yield 45 mg of the product, 86%. ¹H NMR (400 MHz, MeOD) δ 7.09-6.98 (m, 2H), 6.91 (t, J=8.3 Hz, 1H), 5.85-5.77 (m, 2H), 3.86 (s, 3H), 3.61-3.52 (m, 4H), 3.10-2.89 (m, 4H). ¹³C NMR (400 MHz, MeOD) δ 147.89, 145.47, 139.32, 134.94, 126.82, 123.57, 116.95, 111.96, 110.66, 106.40, 59.22, 58.25, 57.85, 25.41. ¹⁹F NMR (471 MHz, MeOD) δ −148.33. HRMS (ESI+): calcd. for C₁₅H₁₈FN₂O [M+H]⁺=261.1403. found [M+H]⁺=261.1412.

### (1S,4R)-2-(2-(4-fluoro-5-methoxy-1H-indol-3-yl)ethyl)-2-azabicyclo[2.2.2]oct-5-ene (38)

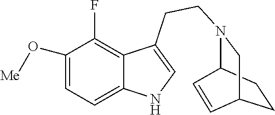 [SMILES: COc1ccc2[nH]cc(CCN3CC4C=CC3CC4)c2c1F]

Synthesized according to procedure C, method II. 3-(2-bromoethyl)-4-fluoro-5-methoxy-1H-indole (500 mg, 1.84 mmol, 1 equiv.) dissolved in 15 mL of MeCN. 2-azabicyclo[2.2.2]oct-5-ene hydroiodide (2.2 mmol, 523 mg, 1.2 equiv.) and NaHCO₃ (772 mg, 9.19 mmol, 5 equiv.) added. Product isolated on TLC with 9:1 EtOAc:MeOH and 2% TEA as the modifier to yield 455 mg product, 82%. ¹H NMR (500 MHz, MeOD) δ 6.83 (d, J=8.7 Hz, 1H), 6.78 (s, 1H), 6.70 (t, J=8.3 Hz, 1H), 6.25-6.17 (m, 1H), 6.11-6.04 (m, 1H), 3.67 (s, 3H), 3.16-3.09 (m, 1H), 2.83-2.76 (m, 2H), 2.71 (ddd, J=13.6, 11.6, 4.9 Hz, 1H), 2.58 (td, J=11.6, 5.2 Hz, 1H), 2.38-2.28 (m, 2H), 1.98 (dt, J=10.1, 2.7 Hz, 1H), 1.85 (ddd, J=13.2, 8.3, 3.5 Hz, 1H), 1.36 (tt, J=11.5, 2.9 Hz, 1H), 1.16-1.01 (m, 2H). ¹³C NMR (126 MHz, MeOD) δ 598.01, 596.08, 589.66, 589.58, 585.28, 585.20, 583.70, 581.77, 573.84, 567.34, 567.20, 562.18, 561.37, 561.35, 556.75, 556.72, 510.31, 510.30, 508.56, 504.85, 502.57, 480.77, 475.23, 474.74, 471.76. ¹⁹F NMR (471 MHz, MeOD) δ −148.40. HRMS (ESI+): calcd. C₁₈H₂₂FN₂O [M+H]⁺=301.1716. found [M+H]⁺=301.1724.

### (1S,4R,7S)-7-ethyl-2-(2-(4-fluoro-5-methoxy-1H-indol-3-yl)ethyl)-2-aza bicyclo[2.2.2]oct-5-ene (39)

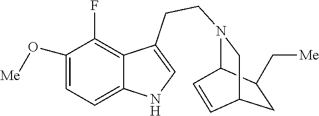 [SMILES: CCC1CC2C=CC1N(CCc1c[nH]c3ccc(OC)c(F)c13)C2]

Synthesized according to general procedure C, Method II. 3-(2-bromoethyl)-4-fluoro-5-methoxy-1H-indole (500 mg, 1.84 mmol, 1 equiv.) was dissolved in 15 mL of MeCN (0.125 M). 2-azabicyclo[2.2.2]oct-5-ene hydroiodide (2.2 mmol, 584 mg, 1.2 equiv.) and NaHCO₃ (772 mg, 9.19 mmol, 5 equiv.) were added. The product was isolated on preparative TLC with 9:1 EtOAc:MeOH and 2% TEA as the modifier to yield 455 mg of the product, 82%. ¹H NMR (500 MHz, MeOD) δ 7.05-6.93 (m, 2H), 6.87 (t, J=8.3 Hz, 1H), 6.35-6.24 (m, 2H), 3.85 (s, 3H), 3.04 (dd, J=9.3, 2.3 Hz, 1H), 2.94 (ddd, J=13.7, 10.2, 5.8 Hz, 1H), 2.83 (ddd, J=13.8, 10.2, 4.8 Hz, 1H), 2.75 (td, J=10.8, 5.8 Hz, 1H), 2.46 (td, J=10.9, 4.8 Hz, 1H), 2.42-2.37 (m, 1H), 1.98 (dt, J=9.3, 2.6 Hz, 1H), 1.62-1.43 (m, 3H), 1.34-1.22 (m, 1H), 0.93-0.84 (m, 4H). ¹³C NMR (500 MHz, MeOD) δ 149.15, 147.21, 140.51, 140.44, 136.24, 136.15, 133.98, 133.73, 124.71, 118.47, 118.33, 113.12, 113.10, 113.04, 107.58, 107.54, 61.29, 61.28, 59.64, 57.03, 56.80, 42.45, 32.84, 30.88, 28.09, 26.34, 12.88. ¹⁹F NMR (471 MHz, MeOD) δ −148.25. HRRS (ESI+): calcd. for C₂₀H₂₆FN₂O [M+H]⁺=329.2029. found [M+H]⁺=329.2036.

### 3-(2-(dimethylamino)ethyl)-4-fluoro-1H-indol-5-ol (40)

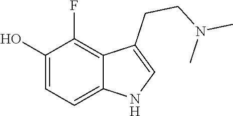 [SMILES: CN(C)CCc1c[nH]c2ccc(O)c(F)c12]

Synthesized according to general procedure E. 2-(4-fluoro-5-methoxy-1H-indol-3-yl)-N,N-dimethylethan-1-amine (20 mg, 0.09 mmol, 1 equiv.) was dissolved in 0.7 mL of dry DCM (0.125 M) and AlCl₃ (68 mg, 0.5 mmol, 6 equiv.) and ethanethiol (95 mg, 1.5 mmol, 18 equiv.) were added. After workup, the product was isolated on preparative TLC with 95:5 EtOAc:MeOH and 1% NH₄OH sat. sol. as the modifier to yield 10 mg of the product, 53%. ¹H NMR (500 MHz, MeOD) δ 7.00-6.89 (m, 2H), 6.74 (t, J=8.4 Hz, 1H), 3.04-2.96 (m, 2H), 2.73-2.65 (m, 2H), 2.37 (s, 6H). ¹³C NMR (500 MHz, MeOD) δ 146.86, 144.97, 137.04, 136.95, 134.95, 134.87, 125.85, 124.47, 118.15, 114.46, 113.16, 111.47, 111.28, 111.26, 107.86, 107.83, 62.23, 62.21, 45.20, 25.13, 14.46. ¹⁹F NMR (471 MHz, MeOD) δ −152.48. HRMS (ESI+): calcd. for C₁₂H₁₆FN₂O [M+H]⁺=223.1247. found [M+H]⁺=223.1251.

### 3-(2-(diethylamino)ethyl)-4-fluoro-1H-indol-5-ol (41)

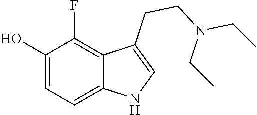 [SMILES: CCN(CC)CCc1c[nH]c2ccc(O)c(F)c12]

Synthesized according to general procedure E. N,N-diethyl-2-(4-fluoro-5-methoxy-1H-indol-3-yl)ethan-1-amine (20 mg, 0.08 mmol, 1 equiv.) was dissolved in 0.7 mL of dry DCM (0.125 M) and AlCl₃ (68 mg, 0.5 mmol, 6 equiv.) and ethanethiol (95 mg, 1.5 mmol, 18 equiv.) were added. After workup, the product was isolated on preparative TLC with 95:5 EtOAc:MeOH and 1% NH₄OH sat. sol. as the modifier to yield 10 mg of the product, 53%. ¹H NMR (500 MHz, MeOD) δ 7.02-6.90 (m, 2H), 6.75 (t, J=8.4 Hz, 1H), 3.04-2.97 (m, 2H), 2.95-2.88 (m, 2H), 2.81 (q, J=7.2 Hz, 4H), 1.17 (t, J=7.2 Hz, 6H). ¹³C NMR (500 MHz, MeOD) δ 146.83, 144.94, 137.09, 137.00, 134.88, 124.62, 117.95, 114.52, 111.00, 107.94, 107.91, 55.21, 47.98, 30.79, 23.70, 10.93. ¹⁹F NMR (471 MHz, MeOD) δ −152.52. HRRS (ESI+): calcd. for C₁₄H₂₀FN₂O [M+H]⁺=251.1560. found [M+H]⁺=251.1560.

### 4-fluoro-3-(2-(pyrrolidin-1-yl)ethyl)-1H-indol-5-ol (42)

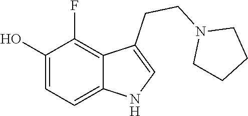 [SMILES: Oc1ccc2[nH]cc(CCN3CCCC3)c2c1F]

Synthesized according to general procedure E. 4-fluoro-5-methoxy-3-(2-(pyrrolidin-1-yl)ethyl)-1H-indole (20 mg, 0.08 mmol, 1 equiv.) was dissolved in 0.7 mL of dry DCM (0.125 M) and AlCl₃ (68 mg, 0.5 mmol, 6 equiv.) and ethanethiol (95 mg, 1.5 mmol, 18 equiv.) were added. After workup, the product was isolated on preparative TLC with 95:5 EtOAc:MeOH and 1% NH₄OH sat. sol. as the modifier to yield 12 mg of the product, 63%. ¹H NMR (500 MHz, MeOD) δ 7.00-6.89 (m, 2H), 6.74 (t, J=8.4 Hz, 1H), 3.07-2.97 (m, 2H), 2.87-2.80 (m, 2H), 2.72-2.64 (m, 4H), 1.89-1.81 (m, 4H). ¹³C NMR (126 MHz, MeOD) δ 146.87, 144.98, 137.05, 136.96, 134.93, 134.84, 124.38, 118.17, 118.03, 114.44, 111.62, 111.60, 107.83, 107.80, 59.36, 59.34, 55.01, 26.68, 24.16. ¹⁹F NMR (471 MHz, MeOD) δ −152.75. HRS (ESI+): calcd. for C₁₄H₁₈FN₂O [M+H]⁺=249.1403. found [M+H]⁺=249.1404.

### 5-methoxy-1-methyl-3-(2-(pyrrolidin-1-yl)ethyl)-1H-indole (43)

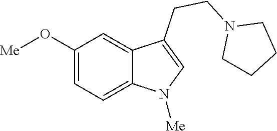 [SMILES: COc1ccc2c(c1)c(CCN1CCCC1)cn2C]

Synthesized according to general procedure D using 5-methoxy-3-(2-(pyrrolidin-1-yl)ethyl)-1H-indole (80 mg, 0.33 mmol, 1 equiv.), 2.5 mL of DMF, NaH (60% dispersion in mineral oil, 1.31 mmol, 52 mg, 4 equiv.) and MeI (0.33 mmol, 46 mg, 1 equiv.). The product was isolated on preparative TLC with 100% EtOAc and 2% TEA as the modifier to yield 70 mg of the product, 83%. ¹H NMR (500 MHz, CDCl₃) δ 7.17 (d, J=8.8 Hz, 1H), 7.06 (d, J=2.4 Hz, 1H), 6.91-6.85 (m, 2H), 3.86 (s, 3H), 3.71 (s, 3H), 2.77-2.65 (m, 4H), 1.93-1.83 (m, 4H). ¹³C NMR (126 MHz, CDCl₃) δ 153.71, 132.40, 128.02, 127.09, 111.74, 109.99, 100.87, 57.29, 56.07, 54.25, 32.78, 29.72, 23.48. HRIS (ESI+): calcd. for C₁₆H₂₃N₂O [M+H]⁺=259.1810. found [M+H]⁺=259.1811.

### 3-(2-(2,5-dihydro-1H-pyrrol-1-yl)ethyl)-5-methoxy-1-methyl-1H-indole (44), 3-(2-(1H-pyrrol-1-yl)ethyl)-5-methoxy-1-methyl-1H-indole (45)

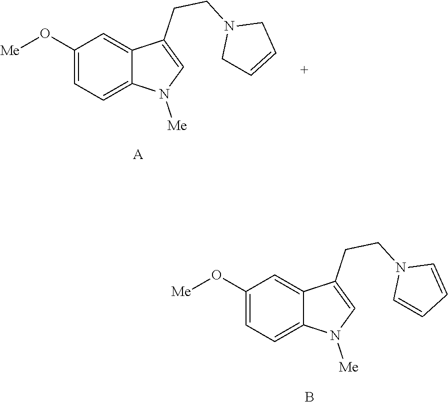

Synthesized according to general procedure D using 3-(2-(2,5-dihydro-1H-pyrrol-1-yl)ethyl)-5-methoxy-1H-indole (20 mg, 0.082 mmol, 1 equiv.), 0.8 mL of DMF, NaH (60% dispersion in mineral oil, 0.33 mmol, 13 mg, 4 equiv.) and MeI (0.082 mmol, 12 mg, 1 equiv.). The products were isolated on preparative TLC with 100% EtOAc and 2% TEA as the modifier to yield 3 mg of A, 14% and 8 mg of B, 38%. 44: ¹H NMR (500 MHz, MeOD) δ 7.18 (dd, J=8.8, 3.1 Hz, 1H), 7.02 (d, J=2.4 Hz, 1H), 6.92 (d, J=4.3 Hz, 1H), 6.80 (dd, J=8.8, 2.4 Hz, 1H), 5.82 (d, J=2.9 Hz, 2H), 3.81 (d, J=1.1 Hz, 3H), 3.67 (s, 3H), 3.58-3.49 (m, 4H), 2.98-2.83 (m, 4H). ¹³C NMR (126 MHz, MeOD) δ 155.06, 134.06, 129.45, 128.31, 128.28, 112.58, 112.49, 110.93, 101.63, 60.59, 58.16, 56.30, 32.74, 25.57. HRRS (ESI+): calcd. for C₁₆H₂₁N₂O [M+H]⁺=257.1654. found [M+H]⁺=257.1651. 45: ¹H NMR (500 MHz, MeOD) δ 7.17 (dd, J=8.9, 0.6 Hz, 1H), 6.84 (d, J=2.4 Hz, 1H), 6.79 (dd, J=8.8, 2.4 Hz, 1H), 6.73 (s, 1H), 6.61 (t, J=2.1 Hz, 2H), 5.99 (t, J=2.1 Hz, 2H), 4.11 (t, J=7.0 Hz, 2H), 3.79 (s, 3H), 3.66 (s, 3H), 3.10 (t, J=0.8 Hz, 2H). ¹³C NMR (126 MHz, MeOD) δ 155.10, 133.91, 129.45, 128.85, 121.50, 112.57, 112.07, 110.80, 108.54, 101.44, 56.30, 51.48, 32.73, 28.91. HRRS (ESI+): calcd. for C₁₆H₁₉N₂O [M+H]⁺=255.1497. found [M+H]⁺=255.1500.

### 4-fluoro-5-methoxy-1-methyl-3-(2-(pyrrolidin-1-yl)ethyl)-1H-indole (46)

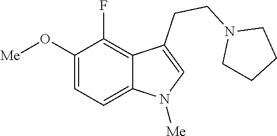 [SMILES: COc1ccc2c(c(CCN3CCCC3)cn2C)c1F]

Synthesized according to general procedure D using 4-fluoro-5-methoxy-3-(2-(pyrrolidin-1-yl)ethyl)-1H-indole (20 mg, 0.076 mmol, 1 equiv.), 0.7 mL of DMF, NaH (60% dispersion in mineral oil, 0.3 mmol, 12 mg, 4 equiv.) and MeI (0.076 mmol, 11 mg, 1 equiv.). The product was isolated on preparative TLC with 100% EtOAc and 2% TEA as the modifier to yield 5 mg of the product, 24%. ¹H NMR (500 MHz, MeOD) δ 7.07-6.92 (m, 3H), 3.88 (s, 3H), 3.70 (s, 3H), 3.05-2.96 (m, 2H), 2.89-2.80 (m, 2H), 2.78-2.67 (m, 4H), 1.95-1.78 (m, 4H). ¹³C NMR (126 MHz, CDCl₃) δ 149.98, 148.02, 141.90, 141.82, 137.29, 137.20, 129.83, 119.88, 119.74, 114.11, 113.73, 113.71, 106.53, 106.50, 61.29, 60.48, 56.43, 35.04, 28.23, 25.71. ¹⁹F NMR (471 MHz, CDCl₃) δ −145.86. HRMS (ESI+): calcd. for C₁₆H₂₂FN₂O [M+H]⁺=277.1716. found [M+H]⁺=277.1723.

### 3-(2-(2,5-dihydro-1H-pyrrol-1-yl)ethyl)-4-fluoro-5-methoxy-1-methyl-1H-indole (47)

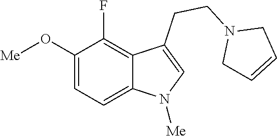 [SMILES: COc1ccc2c(c(CCN3CC=CC3)cn2C)c1F]

Synthesized according to general procedure D using 3-(2-(2,5-dihydro-1H-pyrrol-1-yl)ethyl)-4-fluoro-5-methoxy-1H-indole (20 mg, 0.076 mmol, 1 equiv.), 0.7 mL of DMF, NaH (60% dispersion in mineral oil, 0.3 mmol, 12 mg, 4 equiv.) and MeI (0.076 mmol, 11 mg, 1 equiv.). The product was isolated on preparative TLC with 100% EtOAc and 2% TEA as the modifier to yield 6 mg of the product, 28%. ¹H NMR (500 MHz, MeOD) δ 7.07-6.93 (m, 3H), 5.91-5.78 (m, 2H), 3.87 (s, 3H), 3.70 (s, 3H), 3.66-3.60 (m, 4H), 3.05-2.96 (m, 4H). ¹³C NMR (126 MHz, MeOD) δ 147.41, 139.58, 135.26, 128.13, 126.60, 117.02, 111.78, 109.34, 104.48, 104.45, 59.25, 58.03, 57.69, 57.67, 31.48, 24.98. ¹⁹F NMR (471 MHz, CDCl₃) δ −145.94. HRMS (ESI+): calcd. for C₁₆H₂₀FN₂O [M+H]⁺=275.1560. found [M+H]⁺=275.1567.

### 5-(benzyloxy)-3-(2-(pyrrolidin-1-yl)ethyl)-1H-indole (48)

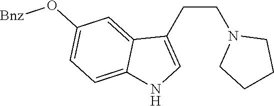

Synthesized according to general procedure F. 5-phenoxy-1H-indole (21.9 g, 104.6 mmol, 1 equiv.) was dissolved in 500 mL of dry diethyl ether (0.2 M), and oxalyl chloride (146.5 mmol, 12.57 mL, 1.4 equiv.) was added. The product was filtered and used directly in the next steps. The glyoxychloride (0.5 g, 1.67 mmol, 1 equiv.) was suspended in 1.0 M of THF and a mixture of TEA (0.35 mL, 2.5 mmol, 1.5 equiv.) and pyrrolidine (0.17 mL, 2.0 mmol, 1.2 equiv.) in 8 mL THF (0.2 M) were added. The Crude glyoxyamide was suspended in 4 mL of dry THF (0.5 M) and added dropwise to a cold suspension of LAH (5 equiv.) in dry THF (1.0 M). The crude material was purified on silica gel column chromatography (9:1 EtOAc:MeOH and 2% TEA) to yield 405 mg of the product, 76% over 2 steps. ¹H NMR (500 MHz, MeOD) δ 7.40-7.35 (m, 2H), 7.30-7.25 (m, 2H), 7.24-7.19 (m, 2H), 7.07 (d, J=2.4 Hz, 1H), 6.95 (s, 1H), 6.83 (dd, J=8.8, 2.4 Hz, 1H), 5.00 (s, 2H), 2.88-2.80 (m, 2H), 2.68-2.59 (m, 2H), 2.52-2.42 (m, 4H), 1.72 (p, J=3.2 Hz, 4H). ¹³C NMR (126 MHz, MeOD) δ 153.83, 139.35, 133.51, 129.34, 128.91, 128.57, 128.55, 123.88, 113.63, 113.21, 112.93, 103.33, 71.96, 58.14, 54.81, 25.61, 24.10. HRMS (ESI+): calcd. for C₂₁H₂₅N₂O [M+H]⁺=321.1967. found [M+H]⁺=321.1964.

### 3-(2-(pyrrolidine-1-yl)ethyl)-1H-indol-5-ol (50)

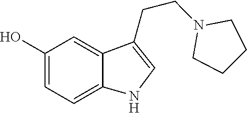 [SMILES: Oc1ccc2[nH]cc(CCN3CCCC3)c2c1]

5-methoxy-3-(2-(pyrrolidine-1-yl)ethyl)-1H-indole (25 mg, 0.10 mmol, 1 equiv.) was dissolved in 1 mL of DCM (0.100 M). BBr₃ (0.20 mmol, 205 μL, 2 equiv.) was added to the solution in a −70° C. dry ice and acetone bath. This yellow solution was stirred for 16 hours before quenching with distilled water (100 mL per mmol of starting material). The organic layer was extracted using 9:1 DCM:iPrOH 4×-6×, until no further visible by TLC), dried using Na₂SO₄, and concentrated to provide the crude product. This mixture was loaded on a preparative TLC plate and the product was isolated (9:1 DCM:iPrOH with 2% TEA) to yield 7.8 mg of the product, 33%. ¹H NMR (500 MHz, MeOD) δ 7.19 (d, J=8.6 Hz, 1H), 7.03 (s, 1H), 6.95 (d, J=2.3 Hz, 1H), 6.69 (dd, J=8.6, 2.3 Hz, 1H), 3.00-2.91 (m, 2H), 2.91-2.83 (m, 2H), 2.81-2.72 (m, 4H), 1.96-1.87 (m, 4H). ¹³C NMR (126 MHz, MeOD) δ 151.99, 133.98, 130.14, 124.81, 113.55, 113.22, 104.27, 59.09, 55.91, 26.36, 25.04. HRMS (ESI+): calcd. for C₁₄H₁₉N₂O [M+H]⁺=231.1497. found [M+H]⁺=231.1493.

### 3-(2-((1S,4R)-2-azabicyclo[2.2.2]oct-5-eN-2-yl)ethyl)-1H-indol-5-ol (51)

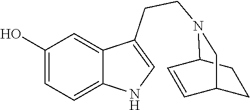 [SMILES: Oc1ccc2[nH]cc(CCN3CC4C=CC3CC4)c2c1]

Synthesized according to general procedure E. (1S,4R)-2-(2-(5-methoxy-1H-indol-3-yl)ethyl)-2-azabicyclo[2.2.2]oct-5-ene (67 mg, 0.24 mmol, 1 equiv.) was dissolved in 1.8 mL of dry DCM (0.125 M) and AlCl₃ (190 mg, 1.42 mmol, 6 equiv.) and ethanethiol (307 PL, 4.26 mmol, 18 equiv.) were added. After workup, the crude material was purified by silica gel column chromatography (gradient of 10 to 50% MeOH in acetone) followed by preparative TLC using 20% MeOH in DCM to yield 22 mg of the product, 35%. ¹H NMR (500 MHz, CDCl₃) δ 8.70 (s, 1H), 8.08 (s, 1H), 7.12 (d, J=8.6 Hz, 1H), 6.91-6.81 (m, 2H), 6.77 (dd, J=8.6, 2.3 Hz, 1H), 6.35 (t, J=7.4 Hz, 1H), 6.19 (dd, J=8.1, 5.4 Hz, 1H), 3.59-3.50 (m, 1H), 3.11 (dd, J=10.1, 2.1 Hz, 1H), 3.01-2.87 (m, 2H), 2.87-2.78 (m, 1H), 2.63-2.53 (m, 1H), 2.53-2.47 (m, 1H), 2.18-2.06 (m, 2H), 1.63-1.53 (m, 1H), 1.31-1.14 (m, 2H). ¹³C NMR (126 MHz, CDCl₃) δ 150.7, 133.8, 131.5, 131.3, 128.2, 122.7, 113.2, 113.0, 112.0, 103.7, 59.1, 54.8, 53.0, 30.6, 25.6, 23.8, 21.8. HRMS (ESI+): calcd. for C₁₇H₂₁FN₂O [M+H]⁺=269.1654. found [M+H]⁺=269.1650.

### 2-(4-methoxy-1H-indol-3-yl)-N,N-dimethylethan-1-amine (54)

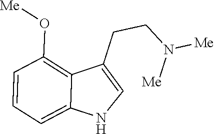 [SMILES: COc1cccc2[nH]cc(CCN(C)C)c12]

Synthesized according to general procedure C Method I. 3-(2-bromoethyl)-4-methoxy-1H-indole (100 mg, 0.39 mmol, 1 equiv.) was dissolved in 2 mL of 2 M dimethylamine solution in MeOH (3.9 mmol, 10 equiv.). The product was isolated on preparative TLC (9:1 EtOAc:MeOH and 2% TEA) to yield 44.0 mg of the product, 51%. ¹H NMR (500 MHz, MeOD) δ 6.99 (t, J=7.9 Hz, 1H), 6.95-6.89 (m, 2H), 6.45 (d, J=7.6 Hz, 1H), 3.89 (s, 3H), 3.13-3.02 (m, 2H), 2.87-2.77 (m, 2H), 2.47 (s, 6H). ¹³C NMR (126 MHz, MeOD) δ 155.66, 139.98, 123.31, 122.32, 118.31, 112.90, 105.88, 99.75, 62.42, 55.32, 44.94, 25.17. HRMS (ESI+): calcd. for C₁₃H₁₉N₂O [M+H]⁺=219.1497. found [M+H]⁺=219.1489.

### 4-methoxy-3-(2-(pyrrolidin-1-yl)ethyl)-1H-indole (55)

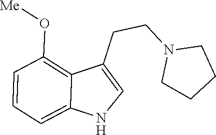 [SMILES: COc1cccc2[nH]cc(CCN3CCCC3)c12]

Synthesized according to general procedure C Method I. 3-(2-bromoethyl)-4-methoxy-1H-indole (100 mg, 0.39 mmol, 1 equiv.) was dissolved in 2 mL of MeOH (0.2 M), and pyrrolidine (0.32 mL, 3.9 mmol, 10 equiv.). The product was isolated on preparative TLC (9:1 EtOAc:MeOH and 2% TEA) to yield 45.0 mg of the product, 47%. ¹H NMR (500 MHz, MeOD) δ 6.97 (t, J=7.9 Hz, 1H), 6.92 (d, J=8.2 Hz, 1H), 6.88 (s, 1H), 6.42 (d, J=7.6 Hz, 1H), 3.86 (s, 3H), 3.11-3.03 (m, 2H), 2.84-2.75 (m, 2H), 2.71-2.61 (m, 4H), 1.87-1.76 (m, 4H). ¹³C NMR (126 MHz, MeOD) δ 155.74, 139.89, 123.18, 121.99, 118.41, 113.92, 105.80, 99.67, 59.80, 55.28, 54.96, 27.06, 24.15. HRMS (ESI+): calcd. for C₁₅H₂₁N₂O [M+H]⁺=245.1654. found [M+H]⁺=245.1663.

### 3-(2-(2,5-dihydro-1H-pyrrol-1-yl)ethyl)-4-methoxy-1H-indole (56)

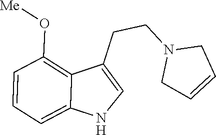 [SMILES: COc1cccc2[nH]cc(CCN3CC=CC3)c12]

Synthesized according to general procedure C Method II. 3-(2-bromoethyl)-4-methoxy-1H-indole (100 mg, 0.39 mmol, 1 equiv.) was dissolved in 4 mL of MeCN (0.1 M). 3-pyrroline (41.0 mg, 0.59 mmol, 1.5 equiv.) and NaHCO₃ (135 mg, 1.57 mmol, 4 equiv.) were added. The product was isolated on preparative TLC (9:1 EtOAc:MeOH and 2% TEA) to yield 27.0 mg of the product, 28%. ¹H NMR (500 MHz, MeOD) δ 6.97 (t, J=7.9 Hz, 1H), 6.91 (dd, J=8.2, 0.9 Hz, 1H), 6.87 (s, 1H), 6.42 (d, J=7.6 Hz, 1H), 5.80 (s, 2H), 3.86 (s, 3H), 3.57-3.50 (m, 4H), 3.05-2.99 (m, 2H), 2.94-2.88 (m, 2H). ¹³C NMR (126 MHz, MeOD) δ 155.74, 139.89, 128.25, 123.14, 122.01, 118.44, 113.95, 105.79, 99.65, 60.54, 59.86, 55.28, 27.26. HRMS (ESI+): calcd. for C₁₅H₁₉N₂O [M+H]⁺=243.1497. found [M+H]⁺=243.1508.

### 2-(4-fluoro-1H-indol-3-yl)-N,N-dimethylethan-1-amine (57)

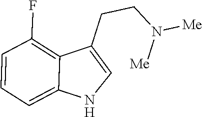 [SMILES: CN(C)CCc1c[nH]c2cccc(F)c12]

Synthesized according to general procedure C Method I. 3-(2-bromoethyl)-4-fluoro-1H-indole (200 mg, 0.39 mmol, 1 equiv.) was dissolved in 4 mL of dimethylamine solution in MeOH (2 M). The product was isolated on preparative TLC (9:1 EtOAc:MeOH and 2% TEA) to yield 110 mg of the product, 54%. ¹H NMR (500 MHz, MeOD) δ 7.16 (d, J=8.2 Hz, 1H), 7.09 (s, 1H), 7.06-7.00 (m, 1H), 6.68 (dd, J=11.5, 7.8 Hz, 1H), 3.13-3.04 (m, 2H), 2.92-2.83 (m, 2H), 2.50 (s, 6H). ¹³C NMR (126 MHz, MeOD) δ 159.31, 157.37, 141.23, 141.14, 124.09, 122.94, 122.88, 116.84, 111.05, 108.78, 108.75, 104.58, 104.42, 61.66, 61.64, 44.84, 24.67. ¹⁹F NMR (471 MHz, MeOD) δ −126.15. HRMS (ESI+): calcd. for C₁₂H₁₆FN₂ [M+H]⁺=207.1297. found [M+H]⁺=207.1303.

### 4-fluoro-3-(2-(pyrrolidin-1-yl)ethyl)-1H-indole (58)

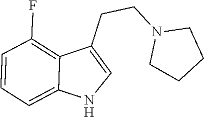 [SMILES: Fc1cccc2[nH]cc(CCN3CCCC3)c12]

Synthesized according to general procedure C Method I. 3-(2-bromoethyl)-4-fluoro-1H-indole (200 mg, 0.39 mmol, 1 equiv.) was dissolved in 4 mL of MeOH (0.2 M), and pyrrolidine (0.68 mL, 3.9 mmol, 10 equiv.). The product was isolated on preparative TLC (9:1 EtOAc:MeOH and 2% TEA) to yield 83 mg of the product, 37%. ¹H NMR (500 MHz, MeOD) δ 7.12 (d, J=8.1 Hz, 1H), 7.05-6.93 (m, 2H), 6.68-6.59 (m, 1H), 3.08-2.97 (m, 2H), 2.84-2.74 (m, 2H), 2.68-2.60 (m, 4H), 1.87-1.75 (m, 4H). ¹³C NMR (126 MHz, MeOD) δ 159.22, 157.29, 141.02, 140.92, 123.48, 122.59, 122.53, 116.99, 116.83, 112.21, 112.18, 108.46, 108.43, 104.28, 104.12, 59.12, 59.10, 54.79, 26.60, 23.98. ¹⁹F NMR (471 MHz, MeOD) δ −125.82. HRMS (ESI+): calcd. for C₁₄H₁₈FN₂ [M+H]⁺=233.1454. found [M+H]⁺=233.1459.

### 4-fluoro-3-(2-(pyrrolidin-1-yl)ethyl)-1H-indole (59)

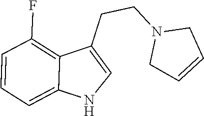 [SMILES: Fc1cccc2[nH]cc(CCN3CC=CC3)c12]

Synthesized according to general procedure C Method II. 3-(2-bromoethyl)-4-fluoro-1H-indole (25 mg, 0.1 mmol, 1 equiv.) was dissolved in 0.5 mL of MeCN (0.1 M). 3-pyrroline (8.0 mg, 0.11 mmol, 1.1 equiv.) and NaHCO₃ (35 mg, 0.4 mmol, 4 equiv.) were added. The product was isolated on preparative TLC (9:1 EtOAc:MeOH and 2% TEA) to yield 9.0 mg of the product, 32%. ¹H NMR (500 MHz, MeOD) δ 7.13 (d, J=8.1 Hz, 1H), 7.04 (s, 1H), 7.03-6.97 (m, 1H), 6.64 (dd, J=11.5, 7.7 Hz, 1H), 5.83 (d, J=1.1 Hz, 2H), 3.60 (d, J=1.2 Hz, 4H), 3.07-2.94 (m, 4H) ¹³C NMR (126 MHz, MeOD) δ 159.55, 157.62, 141.38, 128.31, 123.98, 122.97, 122.91, 112.16, 108.84, 108.81, 104.65, 104.49, 60.79, 59.35, 59.33, 26.94. ¹⁹F NMR (471 MHz, MeOD) δ −125.97. HRMS (ESI+): calcd. for C₁₄H₁₆FN₂ [M+H]⁺=231.1297. found [M+H]⁺=231.1295.

### 2-(6-methoxy-1H-indol-3-yl)-N,N-dimethylethan-1-amine (60)

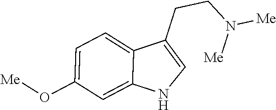 [SMILES: COc1ccc2c(CCN(C)C)c[nH]c2c1]

Synthesized according to general procedure C Method I. 3-(2-bromoethyl)-6-methoxy-1H-indole (50 mg, 0.2 mmol, 1 equiv.) was dissolved in 1 mL of 2 M dimethylamine solution in MeOH (1.97 mmol, 10 equiv.). The product was isolated on preparative TLC (9:1 EtOAc:MeOH and 2% TEA) to yield 37.0 mg of the product, 86%. ¹H NMR (500 MHz, MeOD) δ 7.46 (d, J=8.6 Hz, 1H), 7.06 (d, J=0.9 Hz, 1H), 6.92 (d, J=2.3 Hz, 1H), 6.72 (dd, J=8.6, 2.3 Hz, 1H), 3.79 (s, 3H), 3.28-3.21 (m, 2H), 3.14-3.07 (m, 2H), 2.79 (s, 6H). ¹³C NMR (126 MHz, MeOD) δ 157.83, 138.94, 122.84, 122.57, 119.69, 110.35, 110.26, 95.64, 59.51, 55.97, 43.86, 22.38. HRKS (ESI+): calcd. for C₁₃H₁₉N₂O [M+H]⁺=219.1497. found [M+H]⁺=219.1491.

### 6-methoxy-3-(2-(pyrrolidin-1-yl)ethyl)-1H-indole (61)

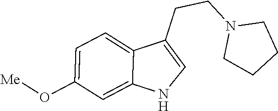 [SMILES: COc1ccc2c(CCN3CCCC3)c[nH]c2c1]

Synthesized according to general procedure C Method I. 3-(2-bromoethyl)-6-methoxy-1H-indole (50 mg, 0.2 mmol, 1 equiv.) was dissolved in 1 mL of MeOH (0.2 M), and pyrrolidine (0.16 mL, 3.9 mmol, 10 equiv.). The product was isolated on preparative TLC (9:1 EtOAc:MeOH and 2% TEA) to yield 37.0 mg of the product, 77%. ¹H NMR (500 MHz, MeOD) δ 7.38 (d, J=8.6 Hz, 1H), 6.90 (d, J=0.9 Hz, 1H), 6.86 (d, J=2.2 Hz, 1H), 6.68 (dd, J=8.6, 2.3 Hz, 1H), 3.77 (s, 3H), 2.95-2.88 (m, 2H), 2.79-2.73 (m, 2H), 2.65-2.57 (m, 4H), 1.85-1.75 (m, 4H). ¹³C NMR (126 MHz, MeOD) δ 156.19, 137.43, 121.79, 120.38, 118.42, 112.32, 108.42, 94.16, 56.98, 54.56, 53.57, 24.24, 22.78. HRRS (ESI+): calcd. for C₁₅H₂₁N₂O [M+H]⁺=245.1654. found [M+H]⁺=245.1656.

### 3-(2-(2,5-dihydro-1H-pyrrol-1-yl)ethyl)-6-methoxy-1H-indole (62)

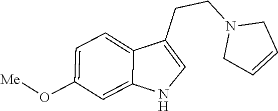 [SMILES: COc1ccc2c(CCN3CC=CC3)c[nH]c2c1]

Synthesized according to general procedure C Method II. 3-(2-bromoethyl)-6-methoxy-1H-indole (50 mg, 0.2 mmol, 1 equiv.) was dissolved in 2 mL of MeCN (0.1 M). 3-pyrroline (15.0 mg, 0.22 mmol, 1.1 equiv.) and NaHCO₃ (70 mg, 0.8 mmol, 4 equiv.) were added. The product was isolated on preparative TLC (9:1 EtOAc:MeOH and 2% TEA) to yield 34 mg of the product, 71%. ¹H NMR (500 MHz, MeOD) δ 7.38 (d, J=8.6 Hz, 1H), 6.91 (s, 1H), 6.86 (d, J=2.3 Hz, 1H), 6.68 (dd, J=8.6, 2.3 Hz, 1H), 5.79 (s, 2H), 3.78 (s, 3H), 3.55-3.45 (m, 4H), 2.95-2.83 (m, 4H). ¹³C NMR (126 MHz, MeOD) δ 157.55, 138.78, 128.22, 123.18, 121.80, 119.80, 113.56, 109.78, 95.53, 60.55, 58.24, 55.93, 25.77. HRMS (ESI+): calcd. for C₁₅H₁₉N₂O [M+H]⁺=243.1497. found [M+H]⁺=243.1491.

### 2-(5H-[1,3]dioxolo[4,5-f]indol-7-yl)-N,N-dimethylethan-1-amine (63)

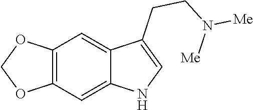 [SMILES: CN(C)CCc1c[nH]c2cc3c(cc12)OCO3]

Synthesized according to general procedure C Method I.7-(2-bromoethyl)-5H-[1,3]dioxolo[4,5-f]indole (100 mg, 0.37 mmol, 1 equiv.) was dissolved in 1.86 mL of 2 M dimethylamine solution in MeOH (3.7 mmol, 10 equiv.). The product was isolated on preparative TLC with 98% EtOAc and 2% TEA as the modifier to yield 53.0 mg of the product, 61%. ¹H NMR (400 MHz, MeOD) δ 6.94-6.84 (m, 2H), 6.79 (d, J=0.6 Hz, 1H), 5.83 (s, 2H), 2.88-2.76 (m, 2H), 2.64-2.54 (m, 2H), 2.31 (s, 6H). ¹³C NMR (101 MHz, MeOD) δ 145.89, 143.65, 132.98, 122.58, 121.71, 113.87, 101.49, 97.73, 92.96, 61.34, 45.35, 24.31. HRRS (ESI+): calcd. for C₁₃H₁₇N₂O₂[M+H]⁺=233.1290. found [M+H]⁺=233.1290.

### 7-(2-(pyrrolidin-1-yl)ethyl)-5H-[1,3]dioxolo[4,5-f]indole (64)

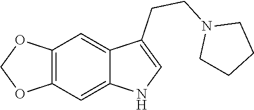 [SMILES: c1[nH]c2cc3c(cc2c1CCN1CCCC1)OCO3]

Synthesized according to general procedure C Method I. 7-(2-bromoethyl)-5H-[1,3]dioxolo[4,5-f]indole (120 mg, 0.45 mmol, 1 equiv.) was dissolved in 2 mL of MeOH (0.2 M), and pyrrolidine (0.37 mL, 4.5 mmol, 10 equiv.). The product was isolated on preparative TLC with 98% EtOAc and 2% TEA as the modifier to yield 102.0 mg of the product, 88%. ¹H NMR (400 MHz, MeOD) δ 6.92-6.85 (m, 2H), 6.79 (d, J=0.6 Hz, 1H), 5.83 (s, 2H), 2.90-2.81 (m, 2H), 2.74-2.68 (m, 2H), 2.61-2.52 (m, 4H), 1.85-1.75 (m, 4H). ¹³C MR (101 MHz, MeOD) δ 145.87, 143.62, 132.94, 122.59, 121.66, 114.16, 101.48, 97.80, 92.96, 58.35, 54.94, 25.72, 24.17. HRIS (ESI+): calcd. for C₁₅H₁₉N₂O₂ [M+H]⁺=259.1447. found [M+H]⁺=259.1450.

### 2-(5H-[1,3]dioxolo[4,5-f]indol-7-yl)-N,N-dimethylethan-1-amine (65)

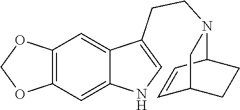 [SMILES: C1=CC2CCC1CN2CCc1c[nH]c2cc3c(cc12)OCO3]

Synthesized according to general procedure C Method II. 7-(2-bromoethyl)-5H-[1,3]dioxolo[4,5-f]indole (1.0 g, 3.73 mmol, 1 equiv.) was dissolved in 30 mL of MeCN (0.125 M). (1S,4R)-2-azabicyclo[2.2.2]oct-5-ene hydrochloride (1.06 g, 4.48 mmol, 1.2 equiv.) and NaHCO₃ (1.57 g, 18.65 mmol, 5 equiv.) were added. The product was isolated on column chromatography with 1:1 EtOAc:hexane and 2% TEA as the modifier to yield 844 mg of the product, 76%. ¹H NMR (400 MHz, CDCl₃) δ 8.28 (br s, 1H), 6.95 (s, 1H), 6.83 (t, J=1.4 Hz, 1H), 6.79-6.69 (m, 1H), 6.42-6.36 (m, 1H), 6.32-6.22 (m, 1H), 5.91 (s, 2H), 3.55-3.44 (m, 1H), 3.10 (dd, J=9.7, 2.1 Hz, 1H), 2.94-2.71 (m, 3H), 2.58-2.46 (m, 2H), 2.20-2.11 (m, 1H), 2.09-1.99 (m, 1H), 1.65-1.51 (m, 1H), 1.40-1.20 (m, 2H). ¹³C NMR (101 MHz, CDCl₃) δ 144.85, 142.63, 133.33, 132.02, 131.18, 121.54, 120.40, 114.79, 100.58, 97.60, 92.12, 59.30, 55.58, 52.78, 30.89, 26.57, 24.53, 22.20. HRRS (ESI+): calcd. for C₁₈H₂₁N₂O [M+H]⁺=297.1603. found [M+H]⁺=297.1609.

### 7-(2-((1S,4R)-7-ethyl-2-azabicyclo[2.2.2]oct-5-en-2-yl)ethyl)-5H-[1,3]dioxolo[4,5-f]indole (66)

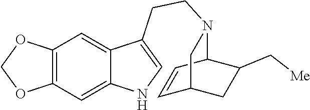 [SMILES: CCC1CC2C=CC1N(CCc1c[nH]c3cc4c(cc13)OCO4)C2]

Synthesized according to general procedure C Method II. 7-(2-bromoethyl)-5H-[1,3]dioxolo[4,5-f]indole (1.0 g, 3.73 mmol, 1 equiv.) was dissolved in 30 mL of MeCN (0.125 M). (1S,4R)-7-ethyl-2-azabicyclo[2.2.2]oct-5-ene hydrochloride (1.19 g, 4.48 mmol, 1.2 equiv.) and NaHCO₃ (1.57 g, 18.65 mmol, 5 equiv.) were added. The product was isolated on column chromatography with 1:1 EtOAc:hexane and 2% TEA as the modifier to yield 1.0 g of the product, 83%. ¹H NMR (400 MHz, CDCl₃) δ 7.84 (br s, 1H), 6.97 (s, 1H), 6.89 (d, J=2.3 Hz, 1H), 6.79 (s, 1H), 6.40-6.24 (m, 2H), 5.92 (s, 2H), 3.31-3.25 (m, 1H), 3.13 (dd, J=9.2, 2.3 Hz, 1H), 2.86-2.68 (m, 3H), 2.57-2.40 (m, 2H), 1.97 (dt, J=9.2, 2.6 Hz, 1H), 1.69-1.45 (m, 3H), 1.38-1.29 (m, 1H), 0.96 (dd, J=4.9, 2.2 Hz, 1H), 0.92 (t, J=7.4 Hz, 3H). ¹³C NMR (101 MHz, CDCl₃) δ 144.78, 142.57, 132.87, 132.72, 130.97, 121.65, 120.26, 115.28, 100.51, 97.60, 91.99, 59.00, 56.25, 55.96, 41.18, 31.63, 29.77, 27.21, 24.49, 12.55. HRMS (ESI+): calcd. for C₂₀H₂₅N₂O [M+H]⁺=325.1916. found [M+H]⁺=325.1927.

### N,N-dimethyl-2-(5-(methylthio)-1H-indol-3-yl)ethan-1-amine (67)

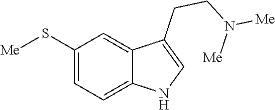 [SMILES: CSc1ccc2[nH]cc(CCN(C)C)c2c1]

Synthesized according to general procedure F. Part I: 5-(Methylthio)-1H-indole was used to prepare a stock of its corresponding glyoxychloride intermediate in the same manner described in the general procedure. The glyoxychloride (150 mg, 0.59 mmol, 1 equiv.) was resuspended in THF (2 mL, 0.3 M) and dimethylamine (1.8 mL of 2 M sol. in THF, 3.55 mmol, 6 equive.) was added. Part II: The glyoxyamide intermediate was suspended in THF and added dropwise to 1 M solution of LAH (131 mg, 3.45 mmol, 8 equiv.) and H₂SO₄ (963 uL, 1.72 mmol, 4 equiv.) in THF at 0° C. The product was isolated on silica column (9:1 EtOAc:MeOH and 2% TEA) to yield 30 mg of the product, 30% over 2 steps. ¹H NMR (500 MHz, CDCl₃) δ 8.47 (br s, 1H), 7.64-7.57 (m, 1H), 7.28-7.23 (m, 1H), 7.20 (dd, J=8.4, 1.7 Hz, 1H), 6.98 (d, J=2.3 Hz, 1H), 2.99-2.92 (m, 2H), 2.73-2.66 (m, 2H), 2.52 (s, 3H), 2.39 (s, 6H). ¹³C NMR (126 MHz, CDCl₃) δ 137.04, 130.22, 129.28, 126.11, 124.28, 121.86, 115.98, 113.67, 62.13, 47.38, 25.49, 21.03. HRMS (ESI+): calcd. for C₁₃H₁₈N₂S [M+H]⁺=235.1269. found [M+H]⁺=235.1267.

### N-ethyl-N-methyl-2-(5-(methylthio)-1H-indol-3-yl)ethan-1-amine (68)

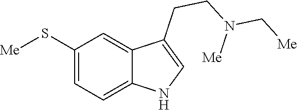 [SMILES: CCN(C)CCc1c[nH]c2ccc(SC)cc12]

Synthesized according to general procedure F. Part I: 5-(Methylthio)-1H-indole was used to prepare a stock of its corresponding glyoxychloride intermediate in the same manner described in the general procedure. The glyoxychloride (150 mg, 0.59 mmol, 1 equiv.) was resuspended in THF (2 mL, 0.3 M) and N-ethyl-N-methylamine (0.3 mL, 3.55 mmol, 6 equiv.) was added. Part II: The glyoxyamide intermediate was suspended in THF and added dropwise to 1 M solution of LAH (131 mg, 3.45 mmol, 8 equiv.) and H₂SO₄ (963 PL, 1.72 mmol, 4 equiv.) in THF at 0° C. The product was isolated on silica column (9:1 EtOAc:MeOH and 2% TEA) to yield 126 mg of the product, 77% over 2 steps. ¹H NMR (500 MHz, CDCl₃) δ 8.41 (br s, 1H), 7.61 (d, J=1.7 Hz, 1H), 7.27-7.18 (m, 2H), 6.98 (d, J=2.2 Hz, 1H), 3.01-2.86 (m, 2H), 2.78-2.67 (m, 2H), 2.57 (q, J=7.2 Hz, 2H), 2.52 (s, 3H), 2.37 (s, 3H), 1.12 (t, J=7.2 Hz, 3H). ¹³C NMR (126 MHz, CDCl₃) δ 137.28, 130.45, 129.37, 126.25, 124.58, 122.03, 116.17, 113.93, 59.95, 53.54, 43.70, 25.22, 21.24, 14.36. HIES (ESI+): calcd. for C₁₄H₂₀N₂S [M+H]⁺=249.1425. found [M+H]⁺=249.1420.

### 5-(methylthio)-3-(2-(pyrrolidin-1-yl)ethyl)-1H-indole (69)

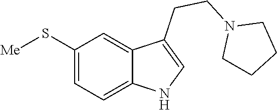 [SMILES: CSc1ccc2[nH]cc(CCN3CCCC3)c2c1]

Synthesized according to general procedure F. Part I: 5-(Methylthio)-1H-indole was used to prepare a stock of its corresponding glyoxychloride intermediate in the same manner described in the general procedure. The glyoxychloride (150 mg, 0.59 mmol, 1 equiv.) was resuspended in THF (2 mL, 0.3 M) and pyrrolidine (0.3 mL, 3.55 mmol, 6 equiv.) was added. Part II: The glyoxyamide intermediate was suspended in THF and added dropwise to 1 M solution of LAH (131 mg, 3.45 mmol, 8 equiv.) and H₂SO₄ (963 μL, 1.72 mmol, 4 equiv.) in THF at 0° C. The product was isolated on silica column (9:1 EtOAc:MeOH and 2% TEA) to yield 128 mg of the product, 75% over 2 steps. ¹H NMR (500 MHz, CDCl₃) δ 8.49 (br s, 1H), 7.62 (d, J=1.7 Hz, 1H), 7.24-7.15 (m, 2H), 6.98-6.95 (m, 1H), 3.01-2.94 (m, 2H), 2.85-2.79 (m, 2H), 2.69-2.63 (m, 4H), 2.51 (s, 3H), 1.88-1.80 (m, 4H). ¹³C NMR (126 MHz, CDCl₃) δ 136.79, 129.88, 128.80, 125.69, 124.12, 121.56, 115.69, 113.39, 58.79, 55.87, 26.57, 25.17, 20.73. HRMS (ESI+): calcd. for C₁₅H₂₁N₂S [M+H]⁺=261.1425. found [M+H]⁺=261.1435.

### N,N-dimethyl-2-(7-methyl-1H-indol-3-yl)ethan-1-amine (70)

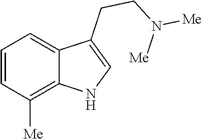 [SMILES: Cc1cccc2c(CCN(C)C)c[nH]c12]

Synthesized according to general procedure F. Part I: 7-methyl-1H-indole was used to prepare a stock of its corresponding glyoxychloride intermediate in the same manner described in the general procedure. The glyoxychloride (80 mg, 0.36 mmol, 1 equive.) was resuspended in THF (1.2 mL, 0.3 M) and dimethylamine (1.8 mL of 2 M sol. in THF, 3.55 mmol, 6 equive.) was added. Part II: The glyoxyamide intermediate was suspended in THF and added dropwise to 1 M solution of LAH (108 mg, 2.88 mmol, 8 equiv.) and H₂SO₄ (787 uL, 1.44 mmol, 4 equiv.) in THF at 0° C. The product was isolated on silica column (9:1 EtOAc:MeOH and 2% TEA) to yield 7 mg of the product, 10% over 2 steps. ¹H NMR (400 MHz, MeOD) δ 7.42-7.31 (m, 1H), 7.05 (d, J=0.9 Hz, 1H), 6.95-6.84 (m, 2H), 2.99-2.89 (m, 2H), 2.77-2.67 (m, 2H), 2.46 (s, 3H), 2.38 (s, 6H). ¹³C NMR (101 MHz, MeOD) δ 126.86, 121.58, 121.44, 120.33, 119.07, 118.46, 115.47, 112.28, 59.90, 43.83, 22.80, 15.45. HRRS (ESI+): calcd. for C₁₃H₁₉N₂ [M+H]⁺=203.1548. found [M+H]⁺=203.1542.

### N,N-dimethyl-2-(1H-pyrrolo[2,3-b]pyridin-3-yl)ethan-1-amine (72)

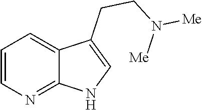 [SMILES: CN(C)CCc1c[nH]c2ncccc12]

Synthesized according to general procedure C, Method I. 3-(2-bromoethyl)-1H-pyrrolo[2,3-b]pyridine (prepared according to Spanò et al. 2016 and Gálvez et al. 1984) (30 mg, 0.13 mmol, 1 equiv.) was dissolved in 0.67 mL of 2 M dimethylamine solution in MeOH (1.33 mmol, 10 equiv.). The product was isolated on preparative TLC (9:1 EtOAc:MeOH and 2% TEA) to yield 14 mg of the product, 56%. ¹H NMR (500 MHz, CDCl₃) δ 8.15 (dd, J=4.8, 1.5 Hz, 1H), 8.00 (dd, J=7.9, 1.5 Hz, 1H), 7.21 (d, J=1.0 Hz, 1H), 7.08 (dd, J=7.9, 4.8 Hz, 1H), 3.00-2.86 (m, 2H), 2.71-2.59 (m, 2H), 2.33 (s, 6H). ¹³C NMR (126 MHz, CDCl₃) δ 148.06, 141.64, 127.22, 122.85, 120.41, 114.71, 111.68, 59.69, 43.97, 22.76. HRMS (ESI+): calcd. for C₁₁H₁₆N₃ [M+H]⁺=190.1344. found [M+H]⁺=190.1350.

### N,N-diethyl-2-(1H-pyrrolo[2,3-b]pyridin-3-yl)ethan-1-amine (73)

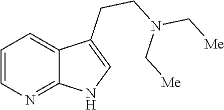 [SMILES: CCN(CC)CCc1c[nH]c2ncccc12]

Synthesized according to general procedure C, Method I. 3-(2-bromoethyl)-1H-pyrrolo[2,3-b]pyridine (prepared according to Spanò et al. 2016 and Gálvez et al. 1984) (30 mg, 0.13 mmol, 1 equiv.) was dissolved in 0.67 mL of MeOH (0.2 M). Diethylamine (1.33 mmol, 0.14 mL, 10 equiv.) was added. The product was isolated on column chromatography with 1:1 EtOAc:Hexanes with 2% TEA as the modifier to yield 14 mg of the product, 50%. ¹H NMR (500 MHz, CDCl₃) δ 8.16 (dd, J=4.8, 1.5 Hz, 1H), 7.99 (dd, J=7.9, 1.6 Hz, 1H), 7.22 (s, 1H), 7.08 (dd, J=7.8, 4.8 Hz, 1H), 2.97-2.85 (m, 2H), 2.85-2.76 (m, 2H), 2.69 (q, J=7.2 Hz, 4H), 1.11 (t, J=7.2 Hz, 6H). ¹³C NMR (126 MHz, CDCl₃) δ 149.45, 143.04, 128.57, 124.20, 121.83, 116.11, 113.35, 54.35, 47.70, 22.97, 11.34. HEMS (ESI+): calcd. for C₁₃H₁₈N₃ [M+H]⁺=218.1657. found [M+H]⁺=218.1664.

### 3-(2-(pyrrolidin-1-yl)ethyl)-1H-pyrrolo[2,3-b]pyridine (74)

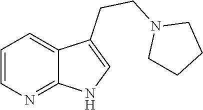 [SMILES: c1cnc2[nH]cc(CCN3CCCC3)c2c1]

Synthesized according to general procedure C, Method I. 3-(2-bromoethyl)-1H-pyrrolo[2,3-b]pyridine (prepared according to Spanò et al. 2016 and Gálvez et al. 1984) (30 mg, 0.13 mmol, 1 equiv.) was dissolved in 0.67 mL of MeOH (0.2 M). Pyrrolidine (1.33 mmol, 0.13 mL, 10 equiv.) was added. The product was isolated on column chromatography with 1:1 EtOAc:Hexanes with 2% TEA as the modifier to yield 19 mg of the product, 68%. ¹H NMR (500 MHz, CDCl₃) δ 8.15 (dd, J=4.8, 1.6 Hz, 1H), 8.00 (dd, J=7.9, 1.5 Hz, 1H), 7.21 (d, J=1.0 Hz, 1H), 7.07 (dd, J=7.8, 4.8 Hz, 1H), 3.01-2.91 (m, 2H), 2.83-2.75 (m, 2H), 2.68-2.55 (m, 4H), 1.88-1.77 (m, 4H). ¹³C NMR (126 MHz, CDCl₃) δ 149.61, 143.20, 128.82, 124.36, 121.97, 116.27, 113.45, 58.28, 55.16, 25.72, 24.37. HRRS (ESI+): calcd. for C₁₃H₁₈N₃[M+H]⁺=216.1501. found [M+H]⁺=216.1504.

### 3-(2-(piperidin-1-yl)ethyl)-1H-pyrrolo[2,3-b]pyridine (75)

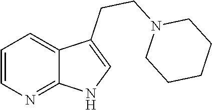 [SMILES: c1cnc2[nH]cc(CCN3CCCCC3)c2c1]

Synthesized according to general procedure C, Method I. 3-(2-bromoethyl)-1H-pyrrolo[2,3-b]pyridine (prepared according to Spanò et al. 2016 and Gálvez et al. 1984) (30 mg, 0.13 mmol, 1 equiv.) was dissolved in 0.67 mL of MeOH (0.2 M). Piperidine (1.33 mmol, 0.13 mL, 10 equiv.) was added. The product was isolated on column chromatography with 1:1 EtOAc:Hexanes with 2% TEA as the modifier to yield 21 mg of the product, 71%. ¹H NMR (400 MHz, MeOD) δ 8.15 (dd, J=4.8, 1.5 Hz, 1H), 7.99 (dd, J=7.9, 1.6 Hz, 1H), 7.19 (d, J=1.0 Hz, 1H), 7.06 (dd, J=7.8, 4.8 Hz, 1H), 2.99-2.85 (m, 2H), 2.67-2.59 (m, 2H), 2.58-2.46 (m, 4H), 1.69-1.58 (m, 4H), 1.52-1.42 (m, 2H). ¹³C NMR (126 MHz, MeOD) δ 148.05, 141.63, 127.25, 122.76, 120.43, 114.70, 111.95, 59.56, 54.00, 25.13, 23.80, 21.92.

HRMS (ESI+): calcd. for C₁₄H₂₀N₃[M+H]⁺=230.1657. found [M+H]⁺=230.1661.

### 2-(5-methoxybenzofuran-3-yl)-N,N-dimethylethan-1-amine (76)

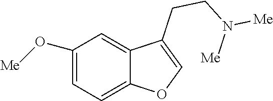 [SMILES: COc1ccc2occ(CCN(C)C)c2c1]

Synthesized according to general procedure C Method I. 3-(2-bromoethyl)-5-methoxybenzofuran (Sames et al. 2018) (50 mg, 0.20 mmol, 1 equiv.) was dissolved in 1 mL of 2 M dimethylamine solution in MeOH (2.0 mmol, 10 equiv.). The product was isolated on preparative TLC with 1:1 EtOAc:Hexane to yield 31 mg of the product, 73%. ¹H NMR (500 MHz, MeOD) δ 7.51 (d, J=1.1 Hz, 1H), 7.30 (d, J=8.9 Hz, 1H), 7.05 (d, J=2.6 Hz, 1H), 6.86 (dd, J=8.9, 2.6 Hz, 1H), 3.81 (s, 3H), 2.85-2.78 (m, 2H), 2.67-2.60 (m, 2H), 2.32 (s, 6H). ¹³C NMR (126 MHz, MeOD) δ 157.33, 151.69, 143.75, 129.78, 119.28, 113.93, 112.67, 102.98, 59.76, 56.30, 45.31, 22.48. HRMS (ESI+): calcd. for C₁₃H₁₈NO₂ [M+H]⁺=220.1338. found [M+H]⁺=220.1348.

### 1-(2-(5-methoxybenzofuran-3-yl)ethyl)pyrrolidine (77)

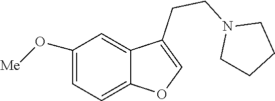 [SMILES: COc1ccc2occ(CCN3CCCC3)c2c1]

Synthesized according to general procedure C Method I. 3-(2-bromoethyl)-5-methoxybenzofuran (Sames et al. 2018) (50 mg, 0.20 mmol, 1 equiv.) was dissolved in 1 mL of MeOH (0.2 M), and pyrrolidine (0.19 mL, 2.0 mmol, 10 equiv.). The product was isolated on preparative TLC with 1:1 EtOAc:Hexane to yield 33 mg of the product, 67%. ¹H NMR (500 MHz, MeOD) δ 7.54 (d, J=1.1 Hz, 1H), 7.32 (d, J=8.9 Hz, 1H), 7.07 (d, J=2.6 Hz, 1H), 6.88 (dd, J=8.9, 2.6 Hz, 1H), 3.84 (s, 3H), 2.91-2.76 (m, 4H), 2.70-2.59 (m, 4H), 1.91-1.79 (m, 4H). ¹³C NMR (126 MHz, MeOD) δ 157.51, 151.87, 143.89, 129.97, 119.66, 114.10, 112.84, 103.19, 56.96, 56.48, 55.11, 24.37, 24.02. HRMS (ESI+): calcd. for C₁₅H₂₀NO₂ [M+H]⁺=246.1494. found [M+H]⁺=246.1502.

### 1-(2-(5-methoxybenzofuran-3-yl)ethyl)piperidine (78)

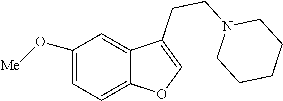 [SMILES: COc1ccc2occ(CCN3CCCCC3)c2c1]

Synthesized according to general procedure C Method I. 3-(2-bromoethyl)-5-methoxybenzofuran (Sames et al. 2018) (50 mg, 0.20 mmol, 1 equiv.) was dissolved in 1 mL of MeOH (0.2 M), and piperidine (0.19 mL, 2.0 mmol, 10 equiv.). The product was isolated on preparative TLC with 1:1 EtOAc:Hexane to yield 37 mg of the product, 73%. ¹H NMR (400 MHz, CDCl₃) δ 7.38 (s, 1H), 7.27 (d, J=8.6 Hz, 1H), 7.02 (d, J=2.6 Hz, 1H), 6.82 (dd, J=8.9, 2.6 Hz, 1H), 3.80 (s, 3H), 2.91 (t, J=8.2 Hz, 2H), 2.78-2.67 (m, 2H), 2.64-2.55 (m, 4H), 1.78-1.62 (m, 4H), 1.55-1.40 (m, 2H). ¹³C NMR (101 MHz, CDCl₃) δ 155.25, 149.60, 141.77, 127.88, 117.29, 112.44, 111.30, 101.56, 57.60, 55.49, 53.68, 24.61, 23.27, 20.28. HRMS (ESI+): calcd. for C₁₆H₂₂NO₂ [M+H]⁺=260.1650. found [M+H]⁺=260.1655.

### 1-(2-(5-methoxybenzofuran-3-yl)ethyl)-1,2,3,6-tetrahydropyridine (79)

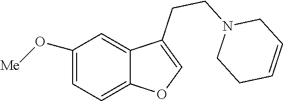 [SMILES: COc1ccc2occ(CCN3CC=CCC3)c2c1]

Synthesized according to general procedure C, Method I. 3-(2-bromoethyl)-5-methoxybenzofuran (Sames et al. 2018) (500 mg, 1.97 mmol, 1 equiv) was dissolved in 2 mL of MeOH (1 M), and 1,2,3,6-tetrahydropyridine (0.36 mL, 3.9 mmol, 2 equiv.). The product was isolated on silica gel column chromatography with 1:1 EtOAc:Hexane to yield 479 mg of the product, 94%. ¹H NMR (500 MHz, CDCl₃) δ 7.45 (d, J=1.1 Hz, 1H), 7.34 (d, J=8.9 Hz, 1H), 7.02 (d, J=2.6 Hz, 1H), 6.89 (dd, J=8.9, 2.6 Hz, 1H), 5.79 (dtt, J=9.4, 3.6, 2.1 Hz, 1H), 5.71 (dtt, J=10.1, 3.3, 1.9 Hz, 1H), 3.85 (s, 3H), 3.12-3.06 (m, 2H), 2.93-2.86 (m, 2H), 2.79-2.72 (m, 2H), 2.67 (t, J=5.7 Hz, 2H), 2.28-2.20 (m, 2H). ¹³C NMR (126 MHz, CDCl₃) δ 155.58, 150.06, 142.14, 128.57, 125.14, 124.96, 118.40, 112.62, 111.72, 102.02, 57.81, 55.83, 52.53, 49.96, 26.05, 21.60. HRRS (ESI+): calcd. for C₁₆H₂₀NO₂ [M+H]⁺=258.1494. found [M+H]⁺=258.1490.

### 2-(4-fluoro-5-methoxy-1H-indol-3-yl)ethan-1-ol (80)

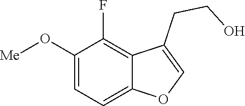 [SMILES: COc1ccc2occ(CCO)c2c1F]

Synthesized according to general procedure A method II using 4-fluoro-5-methoxy-1H-indole. ¹H NMR (500 MHz, CDCl₃) δ 8.09 (s, 1H), 7.00 (d, J=8.7 Hz, 1H), 6.91 (t, J=8.2 Hz, 1H), 4.06 (t, J=5.4 Hz, 2H), 3.95 (s, 3H), 3.00 (t, J=5.5 Hz, 2H). ¹³C NMR (126 MHz, CDCl₃) δ 147.78, 145.83, 139.52, 139.45, 134.61, 134.52, 124.47, 124.31, 117.21, 117.08, 112.16, 110.25, 110.23, 106.70, 106.67, 63.00, 59.05, 29.79.

### 3-(2-bromoethyl)-4-fluoro-5-methoxy-1H-indole (81)

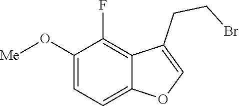 [SMILES: COc1ccc2occ(CCBr)c2c1F]

Synthesized according to general procedure B using 2-(4-fluoro-5-methoxy-1H-indol-3-yl)ethan-1-ol. ¹H NMR (500 MHz, CDCl₃) δ 8.40 (s, 1H), 7.05-6.98 (m, 2H), 6.94 (t, J=8.3 Hz, 1H), 4.04-3.85 (m, 5H), 3.09 (t, J=6.3 Hz, 3H). ¹³C NMR (126 MHz, CDCl₃) δ 147.42, 145.47, 139.76, 139.71, 139.69, 133.06, 132.97, 116.88, 116.73, 111.47, 105.80, 105.77, 104.28, 58.83, 27.45, 23.34. ¹⁹F NMR (376 MHz, MeOD) δ −149.76.

### 2-(5H-[1,3]dioxolo[4,5-]indol-7-yl)ethan-1-ol (82)

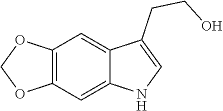 [SMILES: OCCc1c[nH]c2cc3c(cc12)OCO3]

Synthesized according to general procedure A method I using 5H-[1,3]dioxolo[4,5-f]indole. ¹H NMR (400 MHz, CDCl₃) δ 7.89 (br s, 1H), 7.00-6.91 (m, 2H), 6.82 (s, 1H), 5.93 (s, 2H), 3.60 (t, J=7.6 Hz, 2H), 3.30-3.15 (m, 2H).

### 3-(2-bromoethyl)-4-fluoro-5-methoxy-1H-indole (83)

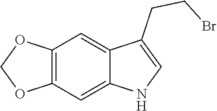 [SMILES: BrCCc1c[nH]c2cc3c(cc12)OCO3]

Synthesized according to general procedure B using 5H-[1,3]dioxolo[4,5-f]indole. ¹H NMR (400 MHz, CDCl₃) δ 7.89 (s, 1H), 6.99-6.90 (m, 2H), 6.82 (s, 1H), 5.94 (s, 2H), 3.60 (t, J=7.3 Hz, 2H), 3.25 (t, J=0.8 Hz, 2H). ¹³C NMR (126 MHz, CDCl₃) δ 144.60, 142.44, 131.46, 121.08, 120.86, 112.50, 100.17, 96.21, 91.63, 32.33, 29.27.

### 2-(6H-[1,3]dioxolo[4,5-e]indol-8-yl)ethan-1-ol

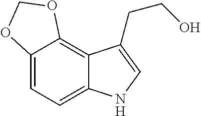 [SMILES: OCCc1c[nH]c2ccc3c(c12)OCO3]

Synthesized according to general procedure A method II using 6H-[1,3]dioxolo[4,5-e]indole. ¹H NMR (500 MHz, CDCl₃) δ 8.03 (bs, 1H), 7.01 (s, 1H), 6.84 (d, J=1.2 Hz, 2 kH), 6.01 (s, 2H), 3.95 (t, J=6.3 Hz, 2H), 3.06 (t, J=6.3, 0.8 Hz, 2H). ¹³C NMR (126 MHz, CDCl₃) δ 139.81, 138.60, 134.95, 124.02, 113.56, 109.69, 104.84, 102.98, 100.67, 63.15, 29.81.

### 8-(2-bromoethyl)-6H-[1,3]dioxolo[4,5-e]indole

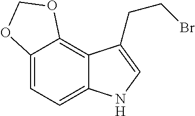 [SMILES: BrCCc1c[nH]c2ccc3c(c12)OCO3]

Synthesized according to general procedure B using 2-(6H-[1,3]dioxolo[4,5-e]indol-8-yl)ethan-1-ol. ¹H NMR (500 MHz, CDCl₃) δ 7.90 (s, 1H), 7.03 (d, J=2.1 Hz, 1H), 6.85 (d, J=1.2 Hz, 2H), 6.04 (s, 2H), 3.73 (t, J=7.5 Hz, 2H), 3.34 (t, J=0.8 Hz, 2H). ¹³C NMR (126 MHz, CDCl₃) δ 139.95, 138.43, 134.69, 123.77, 113.12, 110.86, 104.93, 102.98, 100.80, 33.60, 30.34.

### 2-(6H-[1,3]dioxolo[4,5-e]indol-8-yl)-N,N-dimethylethan-1-amine

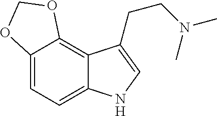 [SMILES: CN(C)CCc1c[nH]c2ccc3c(c12)OCO3]

Synthesized according to general procedure C Method I. 8-(2-bromoethyl)-6H-[1,3]dioxolo[4,5-e]indole (200 mg, 0.75 mmol, 1 equiv.) was dissolved in 4 mL of 2M dimethylamine solution (7.5 mmol, 10 equiv.). The product was isolated on preparative TLC with 9:1 EtOAc:MeOH and 2% TEA as the modifier to yield 122 mg of the product, 70%. ¹H NMR (500 MHz, MeOD) δ 6.97 (d, J=1.0 Hz, 1H), 6.82 (d, J=8.5 Hz, 1H), 6.73 (d, J=8.5 Hz, 1H), 5.91 (s, 2H), 2.95-2.87 (m, 2H), 2.69-2.62 (m, 2H), 2.28 (s, 6H). ¹³C NMR (126 MHz, MeOD) δ 139.08, 138.00, 135.39, 123.33, 113.39, 109.53, 103.66, 102.84, 100.06, 60.75, 43.93, 23.70.

### 8-(2-(pyrrolidin-1-yl)ethyl)-6H-[1,3]dioxolo[4,5-e]indole

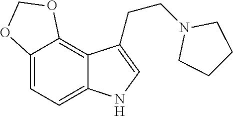 [SMILES: c1cc2[nH]cc(CCN3CCCC3)c2c2c1OCO2]

Synthesized according to general procedure C Method I. 8-(2-bromoethyl)-6H-[1,3]dioxolo[4,5-e]indole (50 mg, 0.18 mmol, 1 equiv.) was dissolved in 0.2 mL of MeOH and Pyrrolidine (1.8 mmol, 10 equiv.). The product was isolated on preparative TLC with 9:1 EtOAc:MeOH and 2% TEA as the modifier to yield 41 mg of the product, 85%. ¹H NMR (500 MHz, MeOD) δ 6.98 (d, J=1.1 Hz, 1H), 6.82 (d, J=8.5 Hz, 1H), 6.73 (d, J=8.5 Hz, 1H), 5.92 (s, 2H), 3.00-2.92 (m, 2H), 2.83-2.76 (m, 2H), 2.65-2.55 (m, 4H), 1.80 (h, J=3.2 Hz, 4H). ¹³C NMR (126 MHz, MeOD) δ 139.08, 138.00, 135.39, 123.33, 113.39, 109.53, 103.66, 102.84, 100.06, 60.75, 43.93, 23.70.

### 8-(2-(2,5-dihydro-1H-pyrrol-1-yl)ethyl)-6H-[1,3]dioxolo[4,5-e]indole

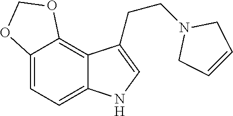 [SMILES: C1=CCN(CCc2c[nH]c3ccc4c(c23)OCO4)C1]

Synthesized according to general procedure C Method I. 8-(2-bromoethyl)-6H-[1,3]dioxolo[4,5-e]indole (50 mg, 0.18 mmol, 1 equiv.) was dissolved in 0.2 mL of MeOH and 3-pyrolline (1.8 mmol, 10 equiv.). The product was isolated on preparative TLC with 9:1 EtOAc:MeOH and 2% TEA as the modifier to yield 39 mg of the product, 82%. ¹H NMR (500 MHz, MeOD) δ 7.00 (s, 1H), 6.82 (d, J=8.5 Hz, 1H), 6.73 (d, J=8.5 Hz, 1H), 5.93 (s, 2H), 5.81 (s, 2H), 3.57-3.53 (m, 4H), 2.96 (m, 4H). ¹³C NMR (126 MHz, MeOD) δ 139.08, 137.97, 135.38, 126.86, 123.35, 113.38, 109.52, 103.62, 102.81, 100.07, 59.19, 57.78, 25.31.

### In Vitro Pharmacokinetic Studies

Microsomal stability characterizations of 4-F, 5-MeO-Tryptamines

### Equipment

Gradient HPLC system API 4000 QTRAP mass spectrometer with Turbo V ion source (AB Sciex, Framingham, MA, USA) Nitrogen generator N2-04-L1466, nitrogen purity 99%+(Whatman) Incubator/Shaker Innova® 4080 (New Brunswick Scientific, Edison, NJ, USA) Water purification sys. Millipore Milli-Q® Gradient A10 (Millipore, France) Multichannel pipettors 1-30 μL, 2-125 μL, 30-850 μL (Thermo Scientific, Waltham, MA, USA).

### Analytical System

All measurements performed using Shimadzu HPLC system including vacuum degasser, gradient pumps, reverse phase HPLC column, column oven, and autosampler (Shimadzu, Columbia, MD, USA). Mass spectrometric analysis performed using API 4000 QTRAP mass spectrometer with Turbo V ion source (AB Sciex, Framingham, MA, USA). The TurboIonSpray ion source was used in both positive and negative ion modes. The data acquisition and system control was performed using Analyst® 1.6.3 software (AB Sciex, Framingham, MA, USA.

### Methods

Microsomal incubations were carried out in 96-well plates in 5 aliquots of 30 μL each (one for each time point). Liver microsomal incubation medium comprised of phosphate buffer (100 mM, pH 7.4), MgCl₂ (3.3 mM), NADPH (3 mM), glucose-6-phosphate (5.3 mM), glucose-6 phosphate dehydrogenase (0.67 units/ml) with 0.42 mg of liver microsomal protein per ml. In the control reactions the NADPH-cofactor system was substituted with phosphate buffer. Test compounds (2 μM, final solvent concentration 1.6%) were incubated with microsomes at 37° C., shaking at 100 rpm. Each reaction was performed in duplicates. Five time points over 90 minutes were analyzed. The reactions were stopped by adding 5 volumes of methanol containing internal standard to incubation aliquots, followed by protein sedimentation by centrifuging at 5500 rpm for 4 minutes. Supernatants were analyzed using the HPLC system coupled with tandem mass spectrometer. The elimination constant (kₑₗ), half-life (t_{1/2}) and intrinsic clearance (Clint) were determined in plot of ln(AUC) versus time, using linear regression analysis.

5-HT1A-Mediated cAMP Reduction Assay

Reduction in cellular cAMP levels mediated by 5-HT1A was determined in HEK293-T cells using a cAMP biosensor (GloSensor, Promega, Madison, WI, USA). First, HEK293-T were cultured in Dulbecco's Modified Eagle Medium (DMEM) supplemented with 10% v/v fetal bovine serum (FBS) and penicillin-streptomycin (Invitrogen). For transfection, cells were plated in 10 cm plates and media was exchanged to DMEM supplemented with 1% v/v dialyzed FBS (dFBS). Once cells reached 6-7 million in a 10 cm plate, transfection was done forming particles in 500 μl Opti-MEM™ (Invitrogen, Waltham, MA, USA) using 4 μg of human 5-HT1A with an N-terminal HA signal sequence and FLAG tag cloned into a pcDNA3 vector, 4 μg of GloSensor DNA, and 16 μl of Polyethyleneimine (PEI, Alfa Aesar, Haverhill, MA, USA). The following day, media was aspirated, and cells were trypsinized and plated into each well of a poly-L-lysine-coated clear-bottom 384-well plate (Greiner Bio One) in DMEM supplemented with 1% v/v dFBS. 40 μl of cells at a density of 500,000/ml were plated per well. The next day, media was aspirated and D-luciferin (GoldBio, St. Louis, MO, USA) was loaded into cells by adding 30 μl of a 1.2 mM solution in Hank's balanced salt solution (HBSS) supplemented with 20 mM Hepes pH 7.4, 0.1% BSA w/v, and 0.01 ascorbic acid w/v. Loading was performed at 37° C. for hour, followed by addition of 15 μl of 3× drug solutions for 15 min at room temperature. To measure agonist activity for 5-HT1A, we added 15 μl of Isoproterenol in HBSS+20 mM Hepes pH 7.4 at a final concentration of 400 nM (to activate Gs via endogenous adrenergic receptors). Luminescence intensity was determined 15 min later in a MicroBeta® TriLux liquid scintillation counter (Perkin Elmer, Waltham, MA, USA). Data was plotted and analyzed in GraphPad Prism® 8.0 (GraphPad Software, San Diego, CA, USA).

5-HT7A-Mediated cAMP Stimulation Assay

HEK293-T cells were cultured and transfected as described above with 4 μg of human 5-HT7A with an N-terminal HA signal sequence and FLAG tag cloned into a pcDNA3 vector and 4 μg of GloSensor DNA (Wacker et al. and Wang et al. 2013). Transfected cells were split, plated, and loaded with luciferin in the same manner as above. Cells were stimulated with 15 μl of 3× drug for 30 min at room temperature. Assay plates were ready immediately after following incubation in a MicroBeta TriLux liquid scintillation counter (Perkin Elmer). Data was plotted and analyzed in GraphPad Prism 8.0.

### Bioluminescence Resonance Energy Transfer (BRET) Signaling Assays

We determined both 5-HT1A-mediated activation of heterotrimeric Gi1 (Gαi1/Gβ3/Gy9) and 5-HT2A-mediated activation of heterotrimeric Gq (Gq/Gβ3/Gy9) using previously published TRUPATH reporter constructs¹⁹. Assays were essentially performed as previously described. In brief, receptor, as well as RLuc-Gα, Gβ, and eGFP-Gy constructs were transfected with Polyethyleneimine (Alfa Aesar) into HEK293-T cells using a ratio of 1:1:1:1 (5-HT1A:Gαi1:Gβ3:Gy9) and 2:5:5:5 (5-HT2A:Gq:Gβ3:Gy9). Typically, we transfected 6-7 million cells in DMEM supplemented with 1% v/v dFBS in a 10 cm plate with a total of 8 μg of DNA. The next day, 20,000 cells were plated into each well of a poly-L-lysine-coated white-bottom 384-well plate (Greiner Bio One) in DMEM supplemented with 1% v/v dFBS. The following day, the day of the experiment, media was aspirated and the cells were washed with Hank's balanced salt solution (HBSS) supplemented with 20 mM Hepes pH 7.4. 15 μl of 3× drugs dilutions were then added in 30 μl HBSS supplemented with 20 mM Hepes pH 7.4, 0.1% w/v bovine serum albumin, and 0.01% w/v ascorbic acid. Cells were incubated for 30 min at 37° C. followed by addition of 15 μl of freshly prepared 30 μM coelenterazine 400a (Gold Biotechnology). Plates were then immediately read in a Victor NIVO plate reader (Perkin Elmer) with 395 nm (RLuc8-coelenterazine 400a) and 510 nm (GFP2) emission filters, at integration times of 1 s per well. BRET2 ratios were computed as the ratio of the GFP2 emission to RLuc8 emission. Data was plotted as a function of drug concentration, normalized to %5-HT stimulation, and analyzed using “log(agonist) vs. response” in GraphPad Prism 8.0.

### Arrestin-Recruitment Assay

To determine off-target effects we performed PRESTO-TANGO arrestin-recruitment assays for all serotonin receptors essentially as previously described (Kroeze et al. 2015). The receptor contructs were obtained from Addgene and contain the TEV cleavage site and the tetracycline transactivator (tTA) fused to the C terminus of the receptor. For the assay, constructs were transfected into HTLA cells (kindly provided by Dr. Bryan Roth, University of North Carolina), which express TEV fused-β-Arrestin2 and a tetracycline transactivator-driven luciferase and are necessary for this assay. A day before the assay cells were cultured in DMEM supplemented with 1% dFBS and transfected with 8 μg of DNA per 7 million cells in a 10 cm plate using PEI transfection. The next day, 20,000 cells in 40 μl were then plated in poly-L-lysine-coated clear-bottom 384-well plates in the same media and incubated for at least 5 hr before stimulation. 20 μl drug solutions in HBSS supplemented with 20 mM HEPES pH 7.4, 0.1% w/v BSA, and w/v 0.01% ascorbic were then added for overnight incubation. After 16-24 hr overnight incubation, media and drug solutions were aspirated and 20 μl per well of BrightGlo reagent (purchased from Promega, after 1:20 dilution) was added per well. The plate was incubated for 20 min at room temperature in the dark before being counted using a MicroBeta TriLux liquid scintillation counter. Relative luminescence units were then plotted as a function of drug concentration and compared to 5-HT stimulation.

### Neurotransmitter Transport Assays

### Serotonin Transporter (SERT) Uptake Inhibition

For determining inhibition of SERT, HEK293-T were cultured in Dulbecco's Modified Eagle Serum (DMEM) supplemented with 10% v/v fetal bovine serum (FBS) and penicillin-streptomycin (Invitrogen). For transfection, cells were plated in 10 cm plates and media was exchanged to DMEM supplemented with 1% v/v dialyzed FBS (dFBS). Once cells reached 6-7 million in a 10 cm plate, a transfection mixture composed of 8 μg of SERT cloned into a pcDNA3 vector, 500 μl of OptiMEM, and 16 μl of PEI was added dropwise to the cells. The following day, transfected cells were trypsinized and plated at a density of 25,000 cells per well of a poly-L-lysine coated clear-bottom 384-well plate. The uptake inhibition assay was performed the following day. Media was aspirated and cells were washed with HBSS supplemented with 20 mM HEPES pH 7.4. To each well, 30 μl of HBSS containing 20 mM HEPES, 0.1% BSA, and 0.01% ascorbic acid and 15 μl of 3× drug dilutions were added. Cells were then incubated at 37° C. for 45 min. A final concentration of 10 μM FFN246 (Aobious) (Henke et al. 2018) was added to each well, and cells were incubated for an additional hour at 37° C. The contents of each well were then aspirated, and each well washed twice with HBSS supplemented with 20 mM HEPES pH 7.4. 50 μl of HBSS supplemented with 20 mM HEPES pH 7.4 was added to each well, and the assay plate was read using a Victor NIVO plate reader using 355 nm excitation and 435 nm emission filters, and measuring over 0.1 s. Data was plotted as a function of drug concentration, normalized to % citalopram inhibition, and analyzed using “log(inhibitor) vs. response” in GraphPad Prism 8.0.

### OCT1, OCT2, and PMAT Uptake Inhibition

Inducible cell lines stably expressing OCT1, OCT2, or PMAT were generated using the Flp-In T-Rex cell line (ThermoFisher) according to manufacturer recommendations. Cells were maintained in DMEM supplemented with 10% FBS, 100 μg/ml hygromycin B (GoldBio), and 10 μg/ml blasticidin (GoldBio). To measure uptake inhibition, cells were seeded into poly-L-lysine coated clear-bottom 384-well plates at a density of 25,000 cells per well in DMEM containing 10% FBS and 2 μg/ml of tetracycline (Sigma) to induce transporter expression. 24 hours following induction, media was aspirated and cells were washed with HBSS supplemented with 20 mM HEPES pH 7.4. To each well, 30 μl of HBSS with 20 mM HEPES pH 7.4, 0.1% BSA w/v, and 0.01% ascorbic acid was added followed by 15 μl of 3× drug dilutions. Cells were incubated with drug for 30 min at 37° C. Fluorescent substrate was then added to the cells. To measure inhibition of OCT1 and PMAT, a final concentration of 12.5 μM of IDT307 (Sigma) was used, while for OCT2, a final concentration of 5 μM of the fluorescent substrate ASP+(ThermoFisher) and 250 μM trypan blue was added. Cells were incubated with respective substrates for an hour at 37° C. OCT2 cells were read directly in a Victor NIVO plate reader using 495 nm excitation and 580 emission filter for 0.1 s. OCT1 and PMAT cells were washed in HBSS supplemented with 20 mM HEPES pH 7.4 before reading in a Victor NIVO plate reader using 435 nm excitation and 480 nm emission filter for 0.1 s.

### Bioluminescence Resonance Energy Transfer (BRET) Signaling Assays

We determined both 5-HT1A-mediated activation of heterotrimeric Gi1 (Gαi1/Gβ3/Gy9) and 5-HT2A-mediated activation of heterotrimeric Gq (Gq/Gβ3/Gy9) using previously published TRUPATH reporter constructs (Halberstat et al. 2011). Assays were essentially performed as previously described. In brief, receptor, as well as RLuc-Gα, Gβ, and eGFP-Gy constructs were transfected with Polyethyleneimine (Alfa Aesar) into HEK293-T cells using a ratio of 1:1:1:1 (5-HT1A:Gαi1:Gβ3:Gy9) and 2:5:5:5 (5-HT2A:Gq:Gβ3:Gy9). Typically, we transfected 6-7 million cells in DMEM supplemented with 1% v/v dFBS in a 10 cm plate with a total of 8 μg of DNA. The next day, 20,000 cells were plated into each well of a poly-L-lysine-coated white-bottom 384-well plate (Greiner Bio One) in DMEM supplemented with 1% v/v dFBS. The following day, the day of the experiment, media was aspirated and the cells were washed with Hank's balanced salt solution (HBSS) supplemented with 20 mM Hepes pH 7.4. 15 μl of 3× drugs dilutions were then added in 30 μl HBSS supplemented with 20 mM Hepes pH 7.4, 0.1% w/v bovine serum albumin, and 0.01% w/v ascorbic acid. Cells were incubated for 30 min at 37° C. followed by addition of 15 μl of freshly prepared 30 μM coelenterazine 400a (Gold Biotechnology). Plates were then immediately read in a Victor NIVO plate reader (Perkin Elmer) with 395 nm (RLuc8-coelenterazine 400a) and 510 nm (GFP2) emission filters, at integration times of 1 s per well. BRET2 ratios were computed as the ratio of the GFP2 emission to RLuc8 emission. Data was plotted as a function of drug concentration, normalized to %5-HT stimulation, and analyzed using “log(agonist) vs. response” in GraphPad Prism 8.0 (San Diego, CA).

### In Vivo Pharmacokinetics (PK) Characterization of Tryptamine Analogs

Test System: Healthy male C57BL/6 mice (8-12 weeks old) weighing between 20 to 35 g were procured from Global (India). Three mice were housed in each cage. Temperature and humidity were maintained at 22±3° C. and 30-70%, respectively and illumination was controlled to give a sequence of 12 hr light and 12 hr dark cycle. Temperature and humidity were recorded by auto-controlled data logger system. All animals were provided laboratory rodent diet (Envigo Research Private Ltd, Hyderabad, India). Reverse osmosis water treated with UV light was provided ad libitum.

Study Design: Total twenty-seven male mice were used in this study with 3 mice/time point design. Animals were administered subcutaneously (SB) with solution formulation of compound at the requited dose.

Formulation Preparation: Accurately weighed of the desired compound was taken in a labeled bottle. To this the appropriate amount of normal saline was added, vortexed and followed by addition of 2 mol. equiv. of glacial acetic acid. The above formulation was again vortexed and sonicated for about 1 h with intermittent vortexing to obtain clear solution. The final dose formulation was filtered through 0.45 micron filter. No adverse clinical signs in any animals during study period.

Sample Collection: Blood samples (approximately 60 μL) were collected under light isoflurane anesthesia (Surgivet®) from retro orbital plexus from a set of three mice at 0.083, 0.25, 0.5, 1, 2, 4, 8, and 12 hr. Immediately after blood collection, plasma was harvested by centrifugation at 4000 rpm, 10 min at 40 C.° and samples were stored at −70±10° C. until bioanalysis. Following blood collection, immediately animals were sacrificed followed by abdominal vena-cava was cut open and whole body was perfused from heart using 10 mL of normal saline. Brain samples were collected from set of three mice at 0.083, 0.25, 0.5, 1, 2, 4, 8, and 12 hr. After isolation, brain samples were rinsed three times in ice cold normal saline (for 5-10 seconds/rinse using ˜5-10 mL normal saline in disposable petri dish for each rinse) and dried on blotting paper. Brain samples were homogenized using ice-cold phosphate buffer saline (pH-7.4). Total homogenate volume was three times the tissue weight. All homogenates were stored below −70±10° C. until bioanalysis.

### Bioanalysis

Concentrations of the compound in mouse plasma and brain samples were determined by fit for purpose LC-MS/MS method. The sample processing and extraction procedure, chromatographic and mass spectrometric conditions as follows:

Parameters

| Mobile Phase | A: 0.1% Formic acid in Acetonitrile | |
|---|---|---|
| | B: 0.1% Formic acid in Water | |
| Column | Acquity Premier BEH C18 50 × 2.1 mm, 1.7 | |
| | μm | |
| Injection Volume | 1 | μL |
| Column Oven Temperature | 45° | C. |

### Data Analysis

Non-Compartmental-Analysis tool of Phoenix WinNonlin® Version 8.0 (Certara, Princeton, NJ, USA) was used to assess the pharmacokinetic parameters. Peak plasma concentration (Cₘₐₓ) and time for the peak plasma concentration (Tₘₐₓ) were the observed values. The areas under the concentration time curve (AUCₗₐₛₜ and AUC_{inf}) were calculated by linear trapezoidal rule. The terminal elimination rate constant, ke was determined by regression analysis of the linear terminal portion of the log plasma concentration-time curve. Tissue-Kps were calculated using Microsoft Excel® (Microsoft Corporation, Seattle, WA, USA).

### Behavioral Assays of 5-MeO-DMT Analogs

### General Mouse Use

All experimental procedures involving animals were approved by the Columbia University Institutional Animal Care and Use Committee (IACUC) and adhered to principles described in the National Institutes of Health Guide for the Care and Use of Laboratory Animals. The studies were conducted at AAALAC accredited facilities. Animals received regular veterinary care (weekly by institutional veterinarians) including daily health monitoring (by experimenters) of the animals (observing home cage behaviors, nesting, and body weight). All procedures were designed to minimize any stress/distress. Healthy adult male mice C57BL/6J (10-15 weeks old) were purchased from the Jackson Lab. (Bar Harbor, ME, USA) and housed 5 mice per cage with food and water available ad libitum. Mice were maintained on a 12-h light/dark cycle (lights on 7:00-19:00) and all testing was done in the light cycle. Temperature was kept constant at 22±2° C., and relative humidity was maintained at 50±5%.

### Drug Preparation and Administration for Animal Studies

All samples are prepared the same day testing is performed. Solids were weighed into small vials and dissolved in USP grade 0.85% saline with addition of 2 molar equivalents of glacial acetic acid. Sonication and gentle heating were applied until complete dissolution. The compounds were subsequently filtered through 0.45 μm filters into a new glass vial. All compounds were administered at a selected subcutaneous dose.

### Animals

Mice arrived at 7 weeks old and were allowed a one-week habituation period before any experimental manipulation was done. Mice were housed in clear polypropylene cages containing wood shavings and had unrestricted access to food and water throughout the entire experiment. Adult male CD-1 retired breeders were used as aggressors in the social defeat stress paradigm. The housing environment was maintained at a constant temperature and the mice were on a 12-hour light/dark cycle.

### Head Twitch Response (HTR) Protocol

The head-twitch response evaluation was performed by trained observers who were blinded to both drug and dose. Mice were habituated to the testing room 30 min. The body weight of each mouse was recorded. The HTR was defined as a rapid rotational movement of the head around the longitudinal axis of the animals' body. Mice were administered with vehicle or WAY100,635 (1 mg/kg or 2 mg/kg, sc). Fifteen minutes after the pretreatment injection mice were administered either 4F, 5-MeO-MET (0.1, 0.3, 1 or 3 mg/kg, sc) or 5-MeO-MET (0.3, 1, 3, and 10 mg/kg, sc) and placed in a novel observation cage. An overhead camera (GoPro HERO9 at a 120 Hz, San Mateo, CA, US) recorded their movements. Observers scored HTR from recorded footage for the first 10-min period.

### Social Defeat Stress Paradigm

The current set of experiments employed the chronic social defeat stress (CSDS) paradigm to evaluate the antidepressant and anxiolytic efficacy of the compounds of interest. The translational validity of this assay has been amply validated by tricyclic antidepressants, SSRIs, ketamine. (Donahue et al. 2014). Briefly, male c57Bl/6J mice were randomly assigned to either the control or stress condition. Mice in the stress condition were subjected to 10 consecutive days of subordination stress by being placed into the territorialized cage of a larger, aggressive CD-1 mouse. Stressed-exposed mice are housed overnight in the compartment adjacent to the recently encountered CD-1 mouse and are introduced to a novel aggressor each day for a 10-minute defeat bout. Control (CON) mice were housed in pairs, one on each side of a perforated Plexiglas partition, and handled daily.

### Social Interaction Teat

Twenty-four hours after the last defeat bout, mice were tested in the social interaction test to evaluate stress-induced social avoidance. For this experiment, one hour after the last defeat bout on day 10, mice were injected with 4-F, 5-MeO-PyrT (1.0 mg/kg), and 4-F, 5-MeO-MET (10.0 and 3.0 mg/kg, respectively) or vehicle. All compounds were prepared on the same day of administration. Solids were weighed and dissolved in 0.85% saline with the addition of 2 molar equivalents of glacial acetic acid. The compounds were then filtered through 0.45 μm filters into a new glass vial. All compounds were administered via a subcutaneous (SC) injection at a volume of 10 mL/kg of body weight.

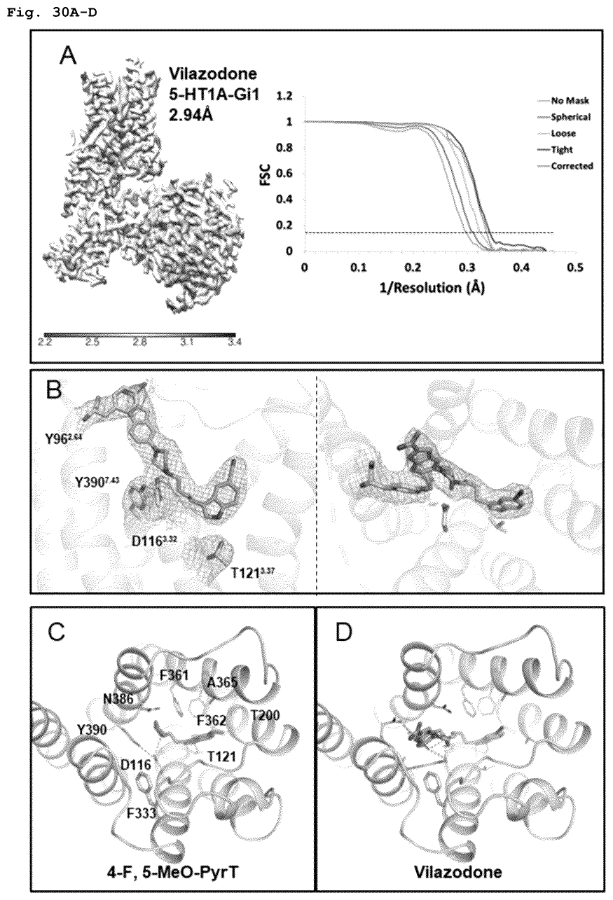

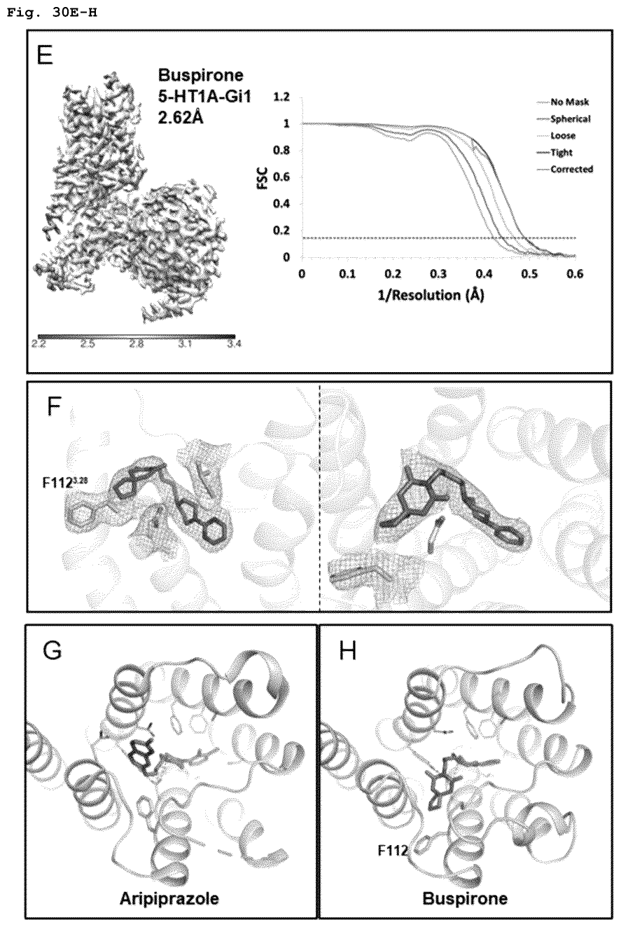

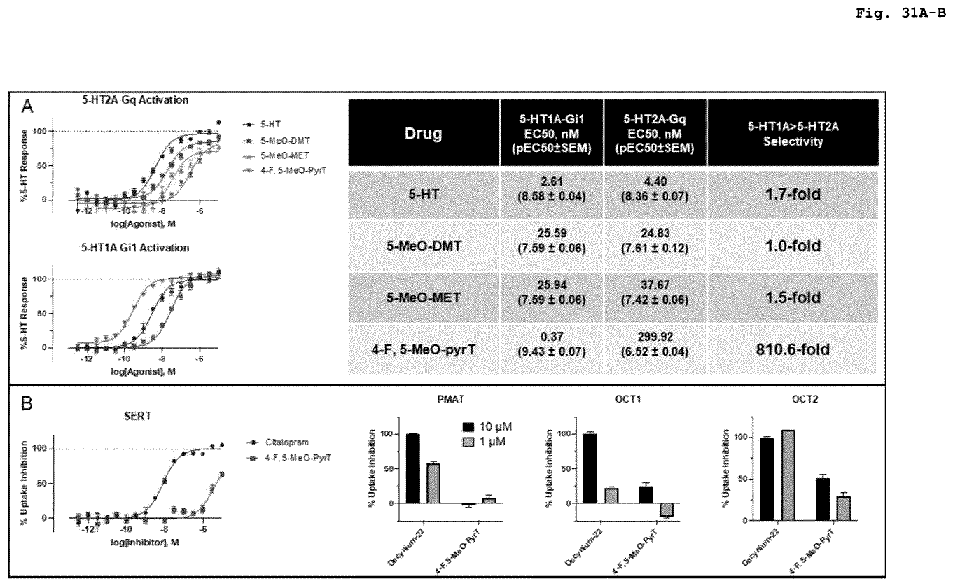

The social interaction (SI) test is a two-session test consisting of a “target” and “no target” session. In the “no target” session, the experimental mouse is allowed to explore an open field arena for 2.5 min. The mouse is then removed, and a novel CD-1 male mouse is placed into a wire mesh cage, which is situated along one side of the arena. This area is the “interaction zone”. For the “target” session, the test mouse is placed back into the arena for another 2.5 min and the amount of time spent in the interaction zone is measured. Socially defeated mice explore the interaction zone significantly less in the presence of the CD-1 mouse. Data is represented as ratio of time spent in interaction zone between the “target” and “no-target” sessions (interaction ratio, IR). Locomotion data is taken from the “no target” session. Data were averaged from three independent experiments. A total of 46 mice were administered vehicle treatment (17 control, 219 stress), 53 received 4-F, 5-MeO-PyrT (17 control, 36 stress), 15 received WAY-100635 (5 control, 10 stress), and 29 received 4-F, 5-MeO-PyrT+WAY-100635 (10 control, 19 stress). More animals were used for the stressed groups due to the inherent resilience of mice to social defeat stress. Analysis of different treatment conditions of control and stressed animals was done using two-way ANOVA. The interaction of all treatments with control and stressed animals shows a p=0.1778, with a row (treatment) factor of p=0.0741 and a column (control/stress) factor of p=0.0001. Comparison of individual treatments as shown in FIG. 30D was done via multiple comparison post hoc analysis using Fisher's LSD test. We additionally analyzed our data taking into account that a proportion of mice are resilient in this paradigm. Accordingly, we used a Fisher's exact test to analyze and compare the number of mice that are resilient (SI ratio >1) and susceptible (SI ratio <1) in the vehicle- and 4-F, 5-MeO-PyrT-treated stressed cohorts.

### Sucrose Preference Test

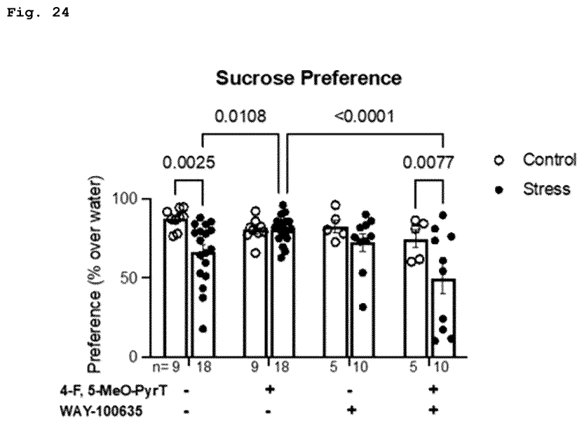

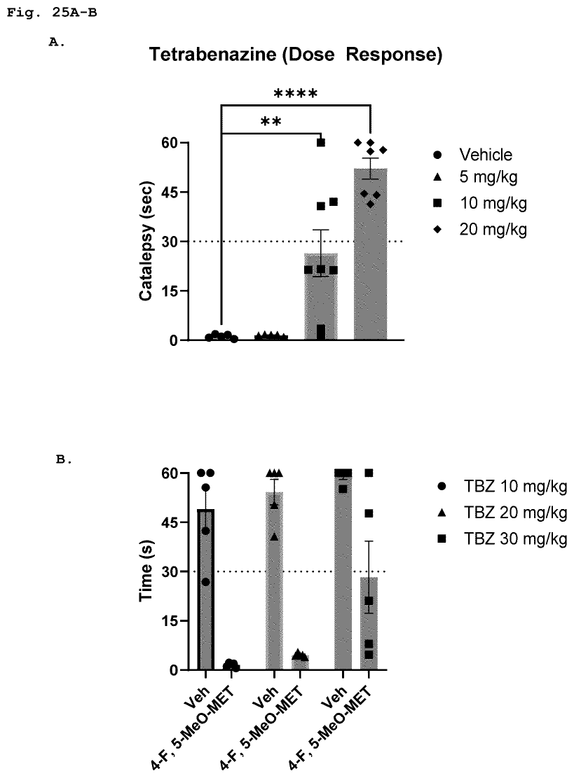

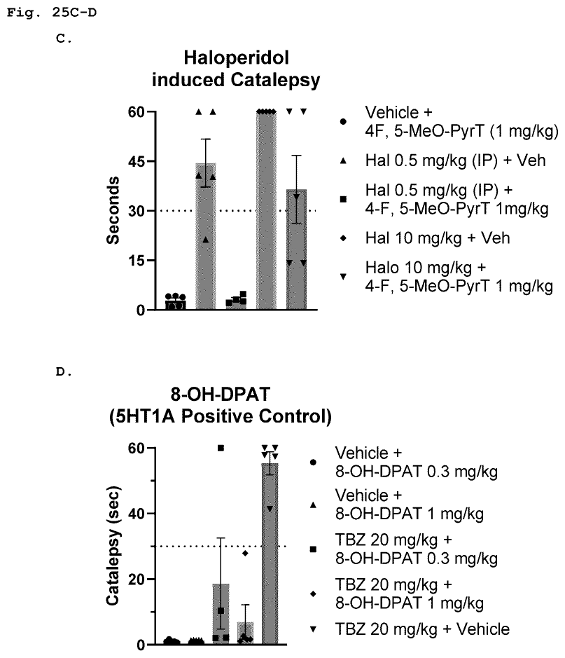

The sucrose preference test is a volitional two-bottle choice procedure in which mice are given the choice between consuming water and a 1% sucrose solution. This paradigm has been used extensively to assess the effects of stress-induced anhedonia. On day 10 of CSDS mice were individually housed and habituated to two bottles containing just water. Twenty-four hours later, following the SI test, one bottle was replaced with a 1% sucrose solution (w/v) made with water from the animal vivarium. Mice had ad libitum overnight access to both bottles and preference for sucrose over water was calculated as follows: [sucrose/(sucrose+water)*100]. Data were averaged from two independent experiments. A total of 27 mice were administered vehicle (9 control, 18 stress), 27 received 4-F, 5-MeO-PyrT (9 control, 18 stress), 15 received WAY-100635 (5 control, 10 stress), and 15 received 4-F, 5-MeO-PyrT+WAY-100635 (5 control, 10 stress). More animals were used for the stressed groups due to the inherent resilience of mice to social defeat stress (Krishnan et al. 2007). Analysis of different treatment conditions of control and stressed animals was done in GraphPad Prism using two-way ANOVA. The interaction of all treatments with control and stressed animals shows a p=0.0773, with a row (treatment) factor of p=0.0159 and a column (control/stress) factor of p=0.0008. Comparison of individual treatments (FIG. 24) was done via multiple comparison post hoc analysis using Fisher's LSD test.

### Catalepsy Bar Test

Catalepsy, characterized by muscular rigidity and fixity of posture irrespective of external stimuli (Mann et al. 2015, Karuppagounder et al 2012) is a recognized model for Parkinson's disease (PD) in rodents, induced by dopamine (DA) antagonists such as haloperidol, and DA depleting agents like tetrabenazine (TBZ) (Jastrzębska-Więsek et al. 2022, Bhangale et al. 2016). TBZ, a reversible inhibitor of the type-2 vesicular monoamine transporter, primarily affects dopamine neurons by blocking vesicular storage and depleting monoamines (Jastrzębska-Więsek et al. 2022). In contrast, Haloperidol, a nonselective D2 dopamine antagonist, interferes with intracellular catecholamine storage, leading to dopamine depletion in nerve endings (Bhangale et al. 2016). C57BL6J mice are administered TBZ/Haloperidol at t=0 min and 4-F, 5-MeO-PyrT at t=30 min via subcutaneous injection, with Haloperidol (0.5 mg/kg) given intraperitoneal injection. Mice are tested individually at t=60 min, and catalepsy is defined by the absence of movement for 30-60 seconds with a cutoff time set to 60 second. The well-established 5HT1A agonist, 8-OH-DPAT, is utilized as a positive control (Ohno et al. 2008). The automated Maze Engineers Catalepsy Bar test, featuring sensors to detect forelimb removal from the bar, facilitates automatic recording of forelimb movement duration from the bar to the ground. These findings indicate that 4F,5-MeO-PyrT has potential therapeutic effect in alleviating the motor deficits associated with PD.

### Results

### Bioluminescence Resonance Energy Transfer (BRET) Signaling Assays

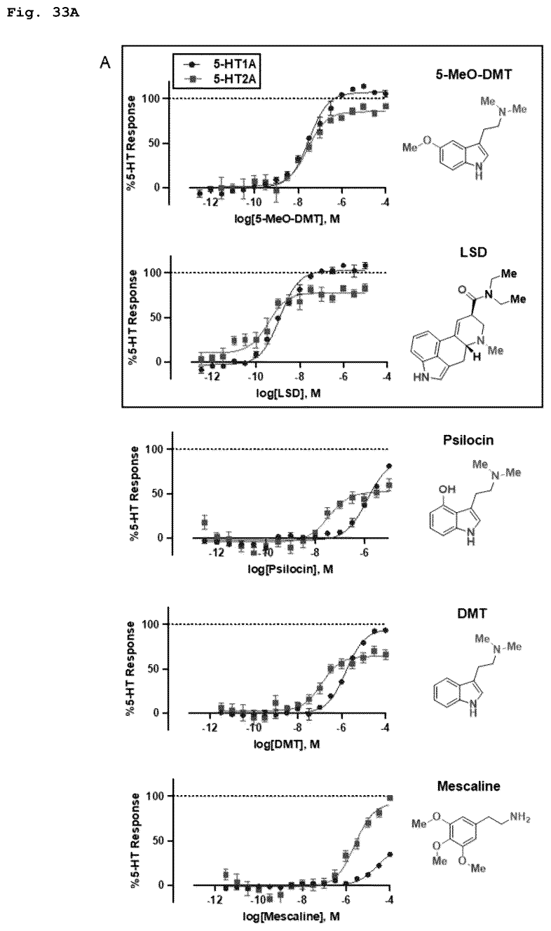

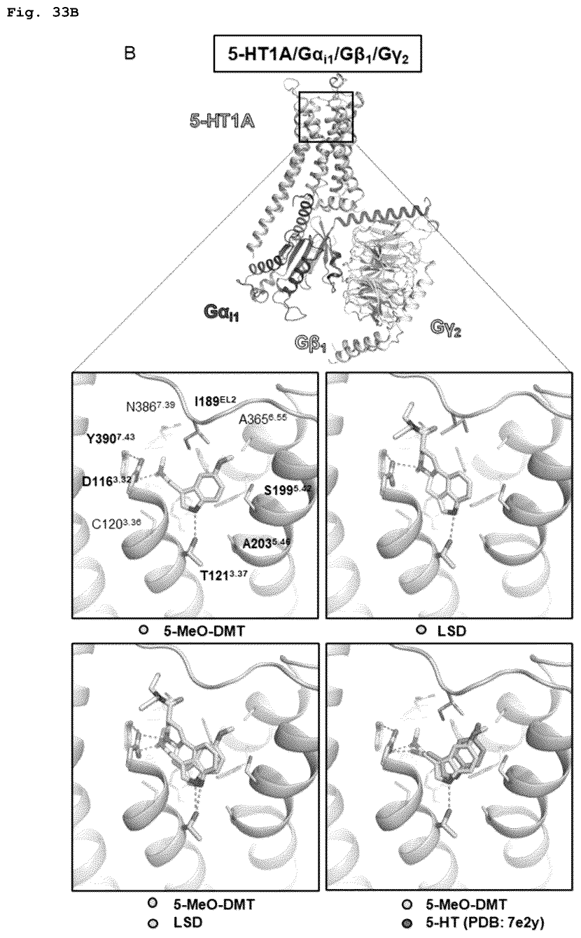

We found that all tested classic psychedelics activated both 5-HT2A and 5-HT1A in our assays, albeit with distinct potencies, efficacies, and receptor-preferences. In line with previous reports (Nichols et al. 2018 and Nichols et al. 2018), most psychedelic drugs tested at 5-HT2A showed partial agonism, ranging from efficacies near 50% of that of serotonin for psilocin (active metabolite of psilocybin) to nearly 100% as seen for mescaline (main psychoactive alkaloid in peyote). We observed that DMT (psychedelic compound in ayahuasca), psilocin, and mescaline are more potent at 5-HT2A, while LSD and 5-MeO-DMT are effectively equipotent at 5-HT1A and 5-HT2A (FIG. 33A). Despite variety in selectivity and potency, the tryptamine compounds are full or near-full agonists of 5-HT1A-mediated G protein signaling in our assays as compared to 5-HT (FIG. 33A, Table 4). These findings validate the potency and efficacy of both LSD and 5-MeO-DMT at 5-HT1A, complementing prior studies that demonstrated the importance of 5-HT1A in the in vivo pharmacology of both drugs (Krebs-Thomson et al. 2006 and Halberstadt et al. 2011).

### Cryoelectron Microscopy Studies of Psychedelic-Bound 5-HT1A Signaling Complexes

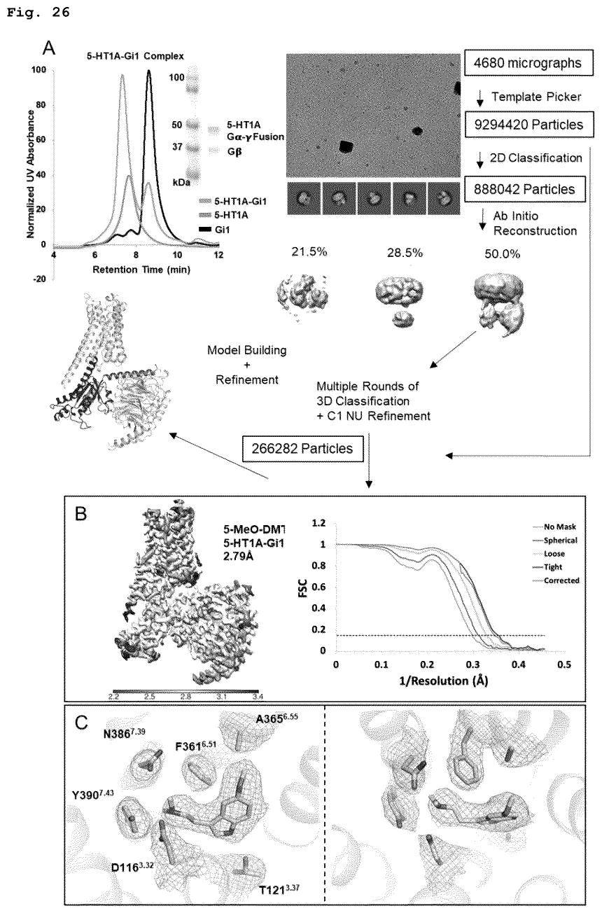

To elucidate the structural basis of how 5-MeO-DMT and LSD activate 5-HT1A, we determined cryoelectron microscopy (cryoEM) structures of drug-bound 5-HT1A Gi signaling complexes (FIG. 33B, FIG. 26A). We used reported receptor and G protein constructs that we (Kim et al. 2020) and others (Xu et al. 2021) previously used to determine 5-HT2A and 5-HT1A structures (see methods for detail). Briefly, for 5-HT1A we replaced the first 24 residues with BRIL to facilitate expression and introduced a stabilizing L125^{3.41}W mutation (Roth et al. 2008). For the heterotrimeric G protein, we co-expressed G01 with a Gy2-Gail fusion (Kim et al. 2020) containing stabilizing mutations (Xu et al. 2021).

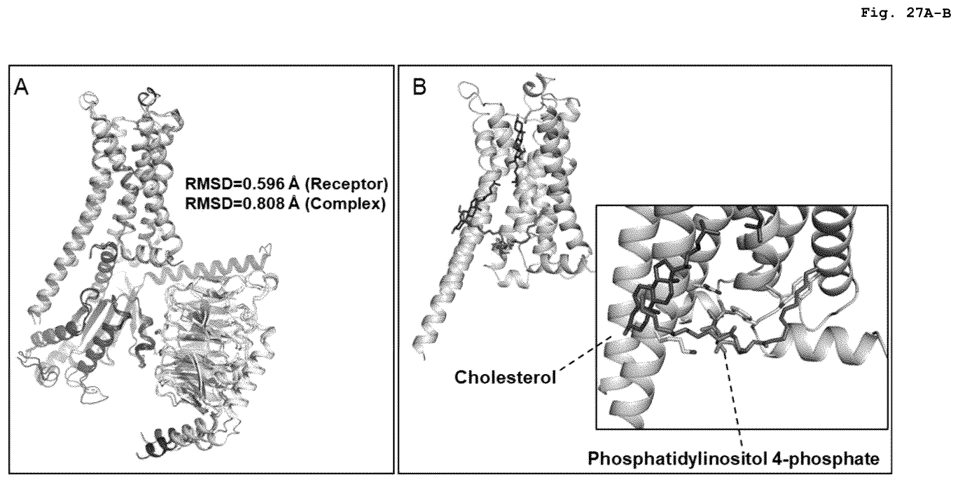

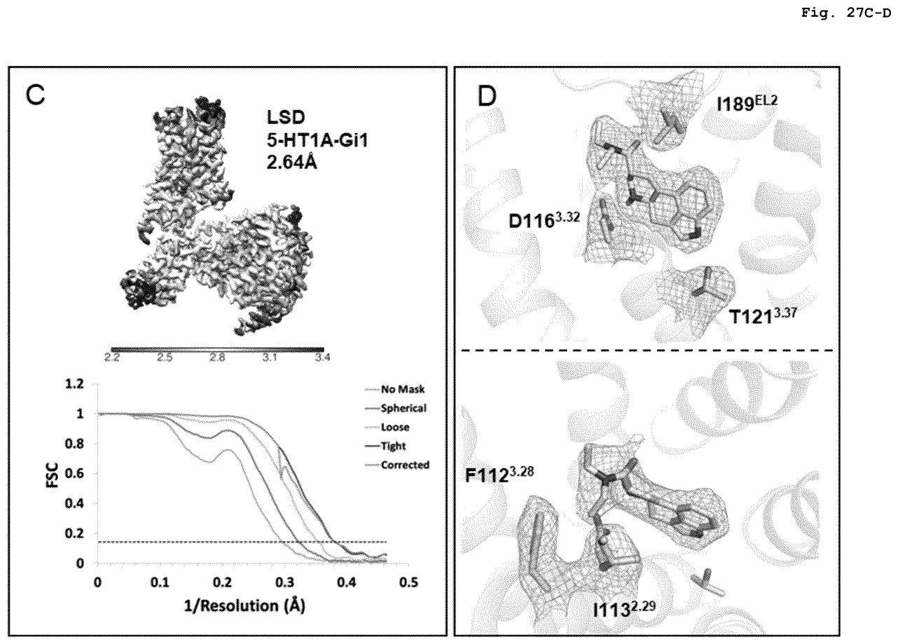

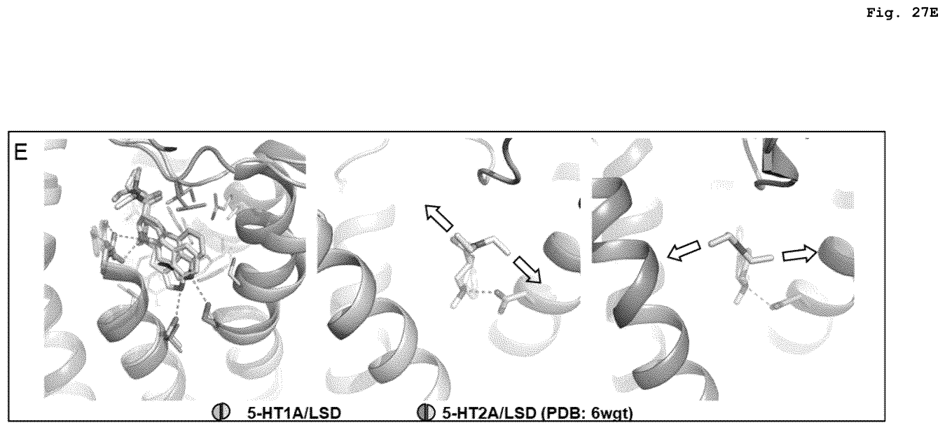

The cryoEM structures of 5-MeO-DMT and LSD-bound 5-HT1A-Gi signaling complexes were obtained at global nominal resolutions of 2.79 Å and 2.64 Å, respectively (FIG. 33B, FIG. 26, Table 9). Our structures allowed us to unambiguously resolve backbones and sidechains, elucidate waters, sterols and lipids, and characterize drug-receptor interactions in molecular detail (FIG. 33B, FIG. 27B). The structures display conserved features of active state GPCRs, such as an outward-rotated receptor TM6 and the C-terminal α5 helix of the Gail protein bound to the cytoplasmic transducer site of 5-HT1A (Xu et al. 2021 and Rasmussen et al. 2011). We observe a similar overall receptor conformation compared to previous 5-HT1A structures with an RMSD of 0.547 Å (FIG. 27A) and were able to elucidate additional 5-HT1A residues such as a complete extracellular loop 2 (EL2). Perhaps most strikingly, our structures uncover distinct drug-receptor interactions in the orthosteric binding pockets (OBP), which may drive differences in drug pharmacology. Primarily anchored by a conserved ionic interaction with D116^{3.32} (Superscripts denote Ballesteros-Weinstein numbering (Ballesteros et al. 1995)), 5-MeO-DMT and LSD bind 0.8 Å deeper in the pocket (measured from the indole nitrogen to the oxygen of T121^{3.37}) compared to the structurally similar serotonin (FIG. 33B) (Xu et al. 2021). In addition, we observe a hydrogen bond between the drugs' indole nitrogen and T121^{3.37} at the “bottom” of the 5-HT1A binding pocket, which is not observed for serotonin. This leads to a slight 8.5° rotation around an axis formed by the amine-D116^{3.32} bond for 5-MeO-DMT, an apparent feature of serotonergic ligands we previously observed for ergoline compounds such as LSD at other 5-HT receptors (Wacker et al. 2017 and Wacker et al. 2013). And indeed, LSD displays a 15.4° rotation towards the receptor core compared to the poses observed in our previous LSD-bound 5-HT2B and 5-HT2A structures. In this position it forms a similar interaction with T121^{3.37} as observed for 5-MeO-DMT (FIG. 33B, FIG. 27E). Starting from the conserved interaction with D116^{3.32}, LSD's diethylamide substituent extends towards the extracellular site forming hydrophobic interactions with I189^{EL2} in EL2—a similar interaction with L229^{EL2} of 5-HT2A was suggested to be critical for LSD's slow binding kinetics and distinct pharmacological profile (Kim et al. 2020 and Wacker et al. 2017). Overall, we observe that the diethylamide substituent of LSD is accommodated 1 Å closer to TM3 (measured as the distance to the Cα of I^{3.29}) with one of LSD's ethyl groups sandwiched between the sidechains of F112^{3.28} and I113^{3.29} (FIG. 27E). This configuration is distinct from that observed in LSD-bound 5-HT2A, which is intriguing as the precise configuration of LSD's ethyl groups appears pivotal for the drug's pharmacology and psychedelic effects in vivo (Wacker et al. 2017, Nichols et al. 2002, Nichols et al. 1996).

Our structural data reveals that LSD adopts distinct binding modes at 5-HT1A and 5-HT2A. We also show that 5-MeO-DMT assumes a different binding pose from related serotonin at 5-HT1A, however it is unclear whether these differences are driven by the methylation of the amine, the methylation of the hydroxyl group, or both.

### Structure-Activity-Relationship Studies of 5-Methoxytryptamines at 5-HT1A

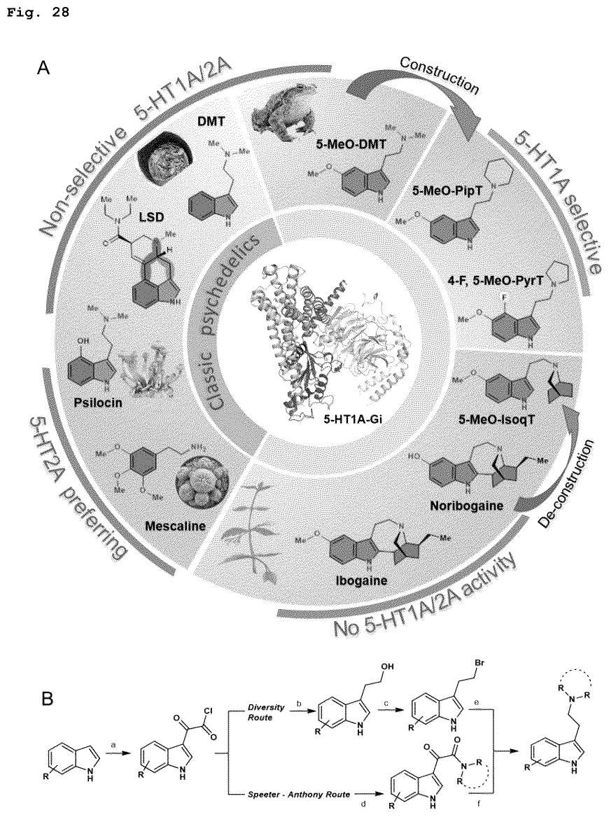

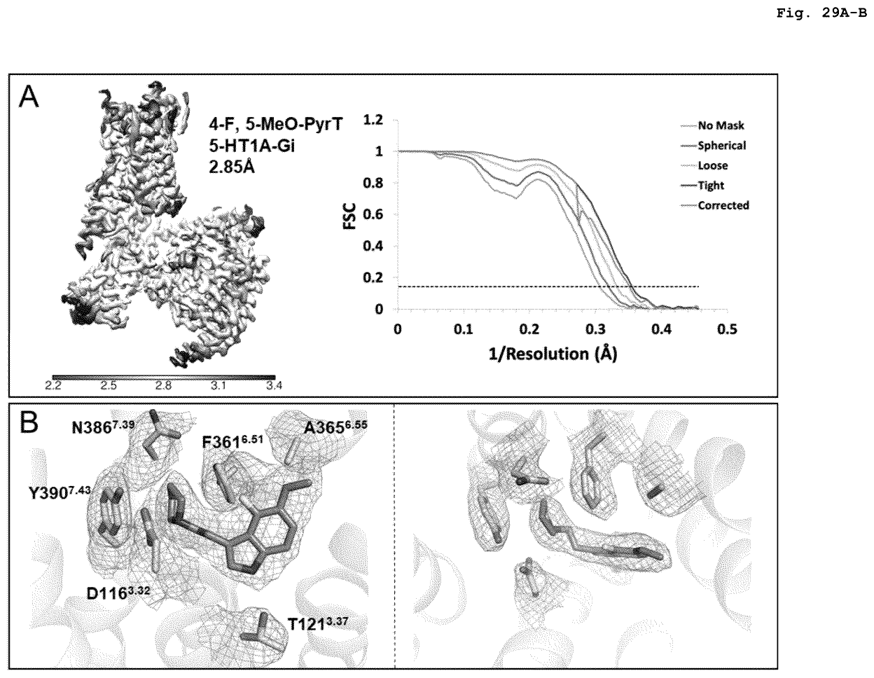

Using 5-MeO-DMT as the starting point, we set out to systematically examine the SAR of 5-MeO-tryptamines and uncover determinants of potency and selectivity at 5-HT1A and 5-HT2A receptors. The compounds were synthesized using indoles with desired substitution and the oxalylation-amidation-reduction sequence (Speeter et al. 1954) to prepare the corresponding tryptamine analogs (FIG. 28).

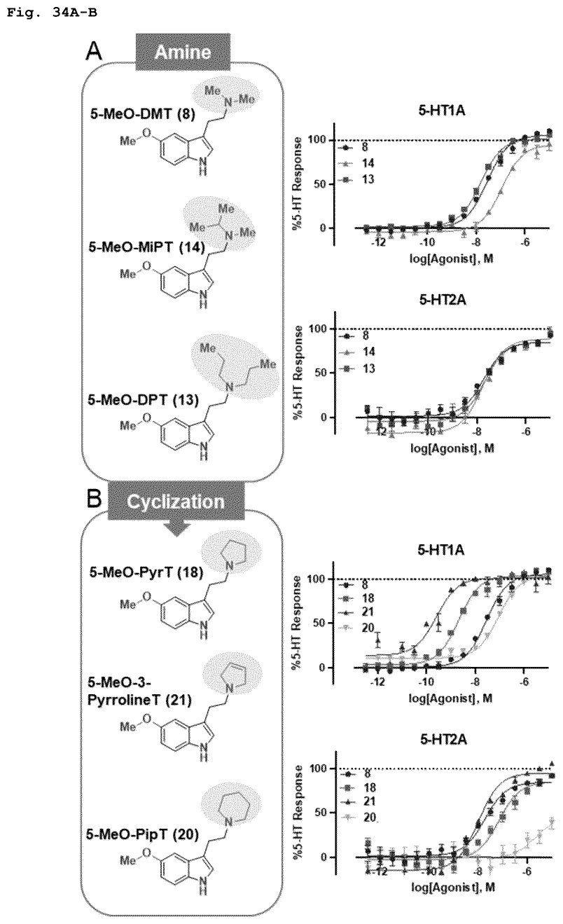

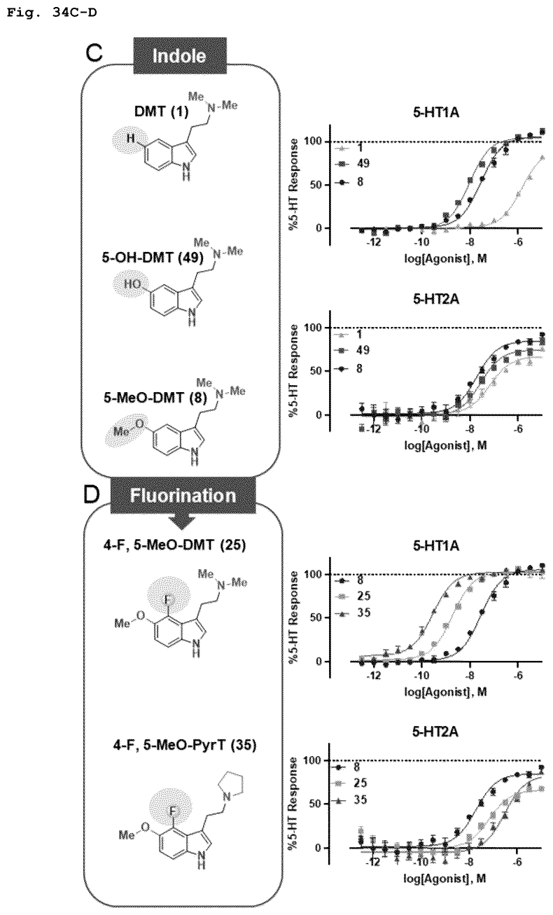

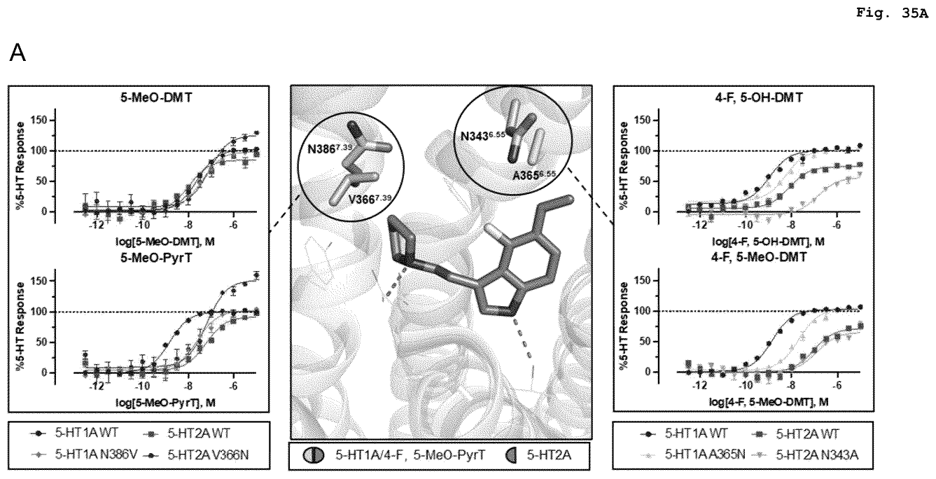

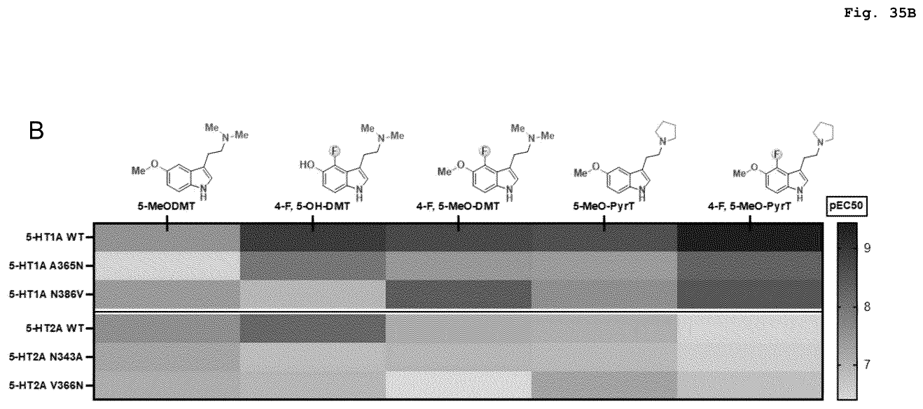

As the first structural variable, we investigated the effect of different amine modifications including the comparison of acyclic and cyclic amines on 5-HT1A and 5-HT2A signaling (FIG. 34, Table 4).

Several of these compounds are known as “designer tryptamines” with anecdotal reports of psychedelic activity in humans (Table 7), including 5-methoxy-N,N-dipropyl-tryptamine (5-MeO-DPT), 5-methoxy-N-methyl,N-isopropyl-tryptamine (5-MeO-MiPT), and 5-methoxy-N,N-pyrrolidinyl-tryptamine (5-MeO-PyrT)^{32,33}. Extension of 5-MeO-DMT's methyl groups (Gi BRET EC₅₀=25.6 nM) to one (5-MeO-MET, Gi BRET EC₅₀=25.9 nM) or two ethyl groups (5-MeO-DET, Gi BRET EC₅₀=37.1 nM) only marginally affected 5-HT1A potency while retaining full efficacy, with similarly small effects at 5-HT2A receptors (FIG. 34, Table 4). In contrast, the cyclic pyrrolidine substituent increases potency at 5-HT1A by ˜12-fold (5-MeO-PyrT, Gi BRET EC₅₀=2.1 nM), and decreases 5-HT2A potency by ˜3-fold relative to 5-MeO-DMT. Thus, cyclization of the amine moiety results in a ˜38-fold increase in 5-HT1A>5-HT2A selectivity. Next, we modestly decreased steric demand of the pyrrolidine via removal of two C—H bonds and installation of a pi-bond and observed a further ˜8-fold increase in potency at 5-HT1A (5-MeO-3-PyrrolineT, Gi BRET EC₅₀=0.3 nM). By contrast, when we further increased the ring size to a six-membered piperidine (5-MeO-PipT, Gi BRET EC₅₀=88.5 nM), we observed an approximate 42-fold loss of potency relative to 5-MeO-PyrT, indicating sensitivity to steric bulk at 5-HT1A (FIG. 34B). Similarly, further elaboration to isoquinuclidine containing tryptamines related to ibogaine leads to a complete loss of 5-HT1A activity (Table 4, FIG. 28).

As the second structural variable, we tested different modifications of the indole nucleus. We mapped the SAR of several positions of the indole to investigate the specificity of the 5-MeO-DMT core for potent 5-HT1A activity (FIG. 34, Table 4). Consistent with previous studies showing that indole fluorination strongly affects 5-HT1 and 5-HT2 binding affinities (Blair et al. 2023 and Laban et al. 2001), we prepared 4-F, 5-MeO-DMT and found a 14-fold increase in signaling potency at 5-HT1A and a 3-fold decrease in potency at 5-HT2A. This trend is consistent across different analogs, where 4-fluorination causes a ˜10-fold increase in potency at 5-HT1A and a ˜5-fold potency decrease at 5-HT2A (except for 5-MeO-3-PyrrolineT, see Table 4). Consequently, combining amine cyclization and 4-fluorination generates the highly potent 5-HT1A compounds 4-F, 5-MeO-PyrT (Gi BRET EC₅₀=370 pM) and 4-F, 5-MeO-3-PyrrolineT (Gi BRET EC₅₀=220 pM). Among those, 4-F, 5-MeO-PyrT is the more selective compound with a greater than 800-fold selectivity for 5-HT1A>5-HT2A, which is driven by both increased potency at 5-HT1A and decreased potency at 5-HT2A (FIG. 34).

### Determinants of 5-HT1A Potency and Selectivity as a Function of Amine Alkylation

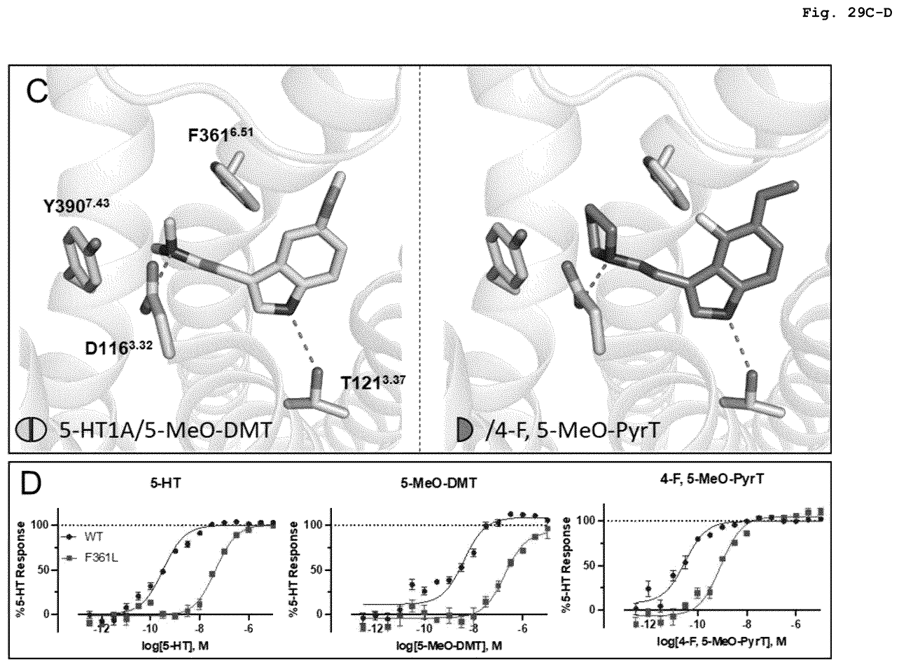

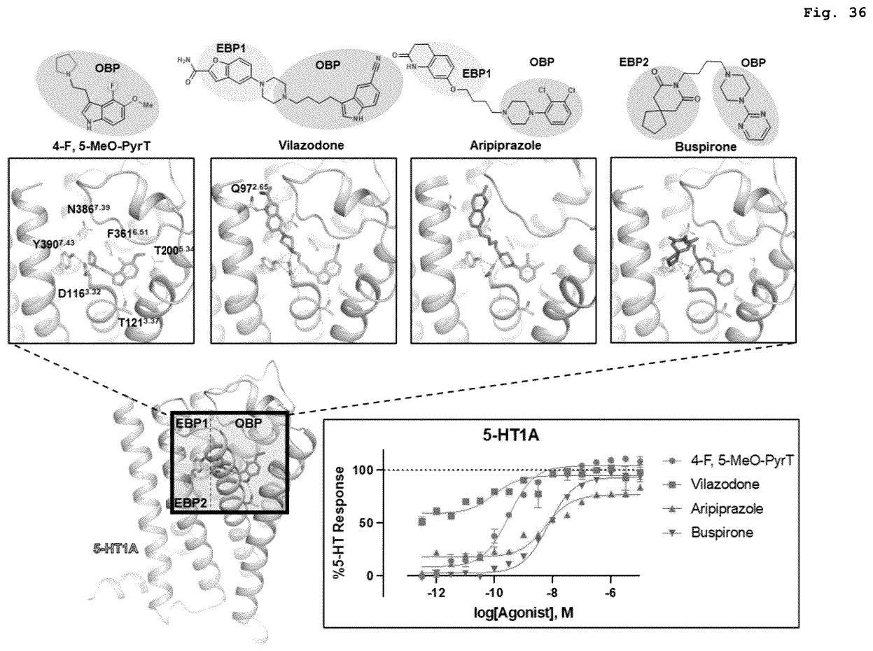

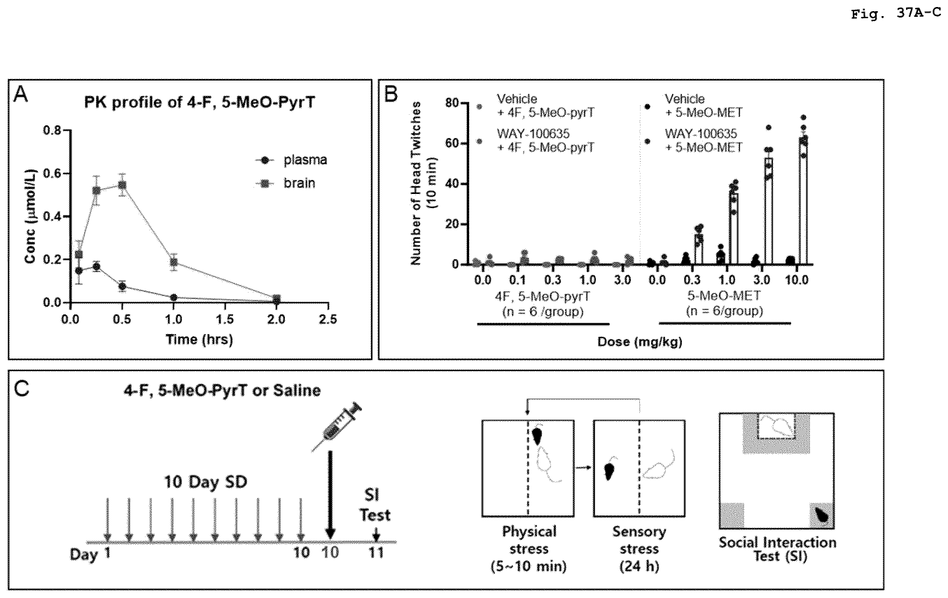

To investigate the structural determinants of the observed SAR effects, we next determined a cryoEM structure of the 5-HT1A signaling complex bound to 4-F, 5-MeO-PyrT at a global resolution of 2.85 Å (FIG. 35, FIG. 29, Table 9). Our structure elucidated that the ligand modifications did not alter the binding pose, and we observe virtually indistinguishable interactions with D116^{3.3} and T121^{3.37} compared to 5-MeO-DMT but distinct from 5-HT (FIG. 33, FIG. 36, FIG. 29). Reminiscent of 5-MeO-DMT's methyl groups, 4-F, 5-MeO-PyrT's pyrrolidyl substituent is wedged between F361^{6.51}, Y390^{7.43}, and N386^{7.39} (FIG. 35, FIG. 27C).

We first probed this interaction by mutation of the conserved F361^{6.51} to a leucine and successively smaller hydrophobic sidechains and observed that tryptamines with smaller or no amine substituents, such as 5-MeO-DMT and serotonin, showed greater 5-HT1A potency loss than 4-F, 5-MeO-PyrT (FIG. 29D, Table 8). These results indicate that F361^{6.51} not only stabilizes the overall tryptamine scaffold, as shown by previous studies (Wang et al. 2013), but plays a key role in the potencies of different amine substitutions. We next investigated the interaction with N386^{7.39} by mutation to the corresponding valine found at this position in 5-HT2A and observed a strong reduction of 5-HT1A potency for 5-MeO-PyrT but not 5-MeO-DMT. Conversely, the V366N^{7.39} mutation in 5-HT2A resulted in a small increase in potency only with cyclic amines. Interestingly, this mutation also leads to an increase in signaling efficacy at 5-HT2A across N-substituted tryptamines, including LSD. These findings illuminate the importance of interactions with residues in position 7.39 for the activities of tryptamine ligands (FIG. 35). Our results show that the residues F361^{6.51}, Y390^{7.43}, and N386^{7.39} create a milieu that accommodates small amine rings that in turn leads to greater signaling potencies of these compounds.

### Determinants of 5-HT1A Potency and Selectivity as a Function of Indole Substitution

Our SAR confirms that introduction of a fluorine in the 4-position or different groups in the 5-position of the indole nucleus greatly increases the signaling potency at 5-HT1A and affects 5-HT1A>5-HT2A selectivity (FIG. 34, Table 4). To investigate the mechanistic basis of these differences we focused on A365^{6.55}, as this alanine is unique to 5-HT1A among 5-HT receptors and is situated in proximity to the 4- and 5-indole substituents. We therefore mutated A365^{6.55} to the corresponding asparagine found at this position in 5-HT2A. We observed a reduction in potency of all examined tryptamines, including fluorinated analogs, with the largest decrease of ˜18-fold for 5-OH-DMT (bufotenine) (Table 8). 5-HT1A's A365^{6.55} is thus important for the high potency of 5-substituted tryptamines. We propose that this is likely due to providing space for substituents and accommodating groups with a variety of chemical properties. On the other hand, specific interactions with A365^{6.55} are not responsible for potency increases of fluorinated tryptamines, which are likely driven by the changes in electron density distribution in the fluorinated compounds and the resulting interactions with larger surfaces of the receptor.

Conversely, the N343^{6.55} mutation in 5-HT2A to 5-HT1A's alanine had little effect on potency of 4-F, 5-MeO-tryptamines, but decreased the potency of serotonin and 4-F, 5-OH-DMT by ˜133-fold and ˜19-fold, respectively. Since we only observed minor effects on DMT, we propose that 5-HT2A's N343^{6.55} forms a hydrogen bond with the 5-hydroxyl group during activation. We thus further suggest that the decreased 5-HT2A potency of 4-fluorinated tryptamines is due to an already diminished ability of 5-MeO-DMT to form hydrogen bonds with N343^{6.55}, as addition of a fluorine further changes the electronic character of the 5-position oxygen.

Structural Studies of Clinically used 5-HT1A Anxiolytics and Antidepressants

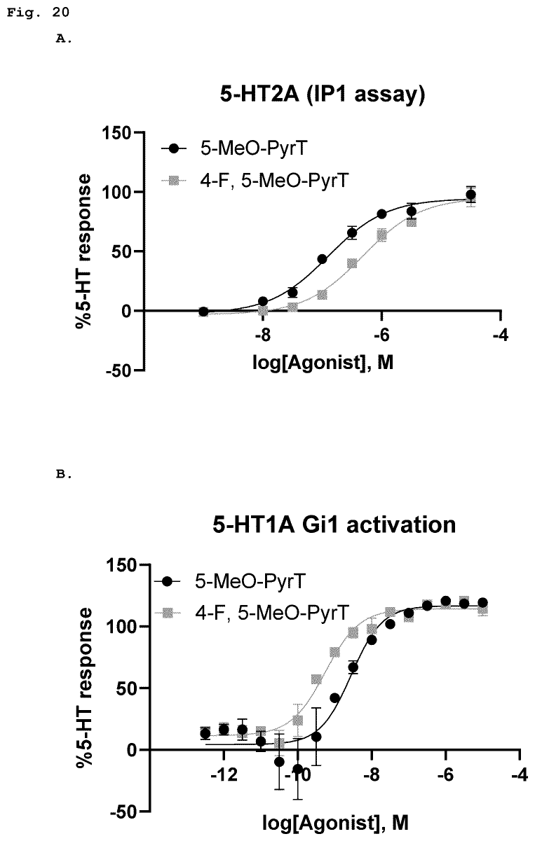

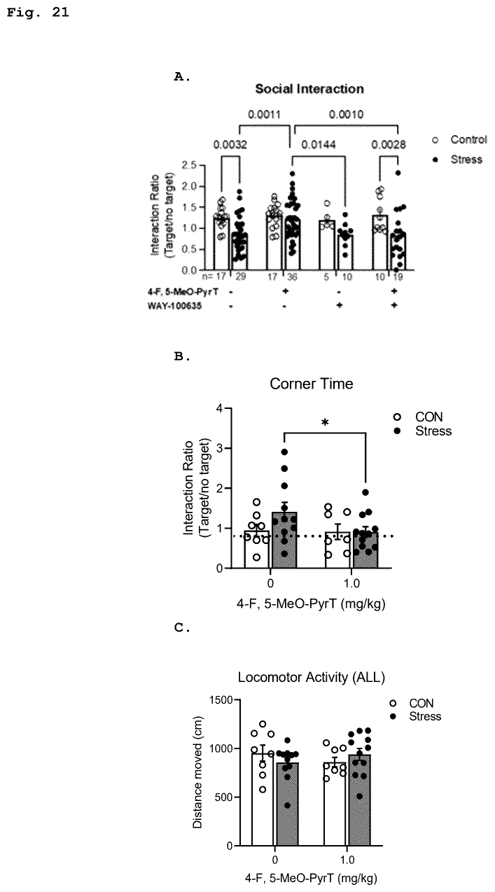

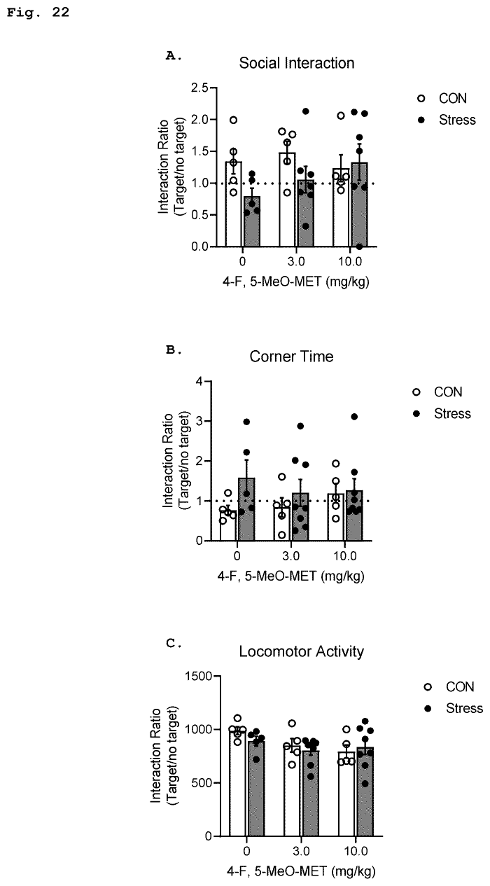

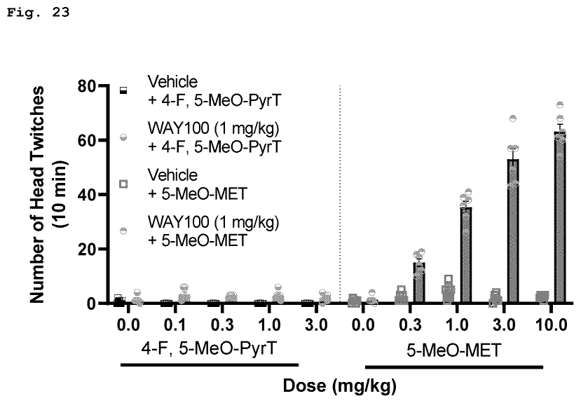

While psychedelics are useful tools to study serotonin receptor structure and function, a renewed interest in their actions has been sparked by their therapeutic potential in the treatment of psychiatric disorders such as treatment resistant depression and anxiety disorders (Carhart-Harris et al. 2018, Gasser et al. 2015, Goodwin et al. 2022, and Von Rotz et al. 2023). Although the contribution of 5-HT1A activity to the clinical efficacy of tryptamines and ergolines remains unclear, the 5-HT1A receptor is a bona fide therapeutic target for the clinically used anxiolytic buspirone (e.g. Buspar®), the antidepressant vilazodone (Viibryd®), as well as other compounds such as befiradol that are currently under clinical investigation (Iderberg et al. 2015). To better understand 5-MeO-DMT molecular pharmacology in the context of prescribed medications, we thus performed a structural and functional characterization of buspirone and vilazodone, and compared it to that of 4-F, 5-MeO-PyrT, a 5-HT1A-selective analog of the psychedelic 5-MeO-DMT (FIG. 36). Since it is unknown how buspirone and vilazodone bind to 5-HT1A, we determined cryoEM structures of drug-bound 5-HT1A-Gi complexes at nominal resolutions of 2.62 Å and 2.94 Å, respectively (FIG. 36, FIG. 20, Table 9). The binding pockets of all 5-HT1A-drug complexes analyzed here show only subtle differences with essentially all relevant side chains assuming similar states, except for an observed rotamer switch in the Buspirone-bound receptor described below. Vilazodone's indole core is bound to the orthosteric binding pocket forming a similar H-bond with T121^{3.37} as observed for 5-MeO-DMT and 4-F, 5-MeO-PyrT, while its benzofuran-carboxamide group extends towards the extracellular site forming an H-bond with Q97^{2.65} in TM2. Although this overall binding pose is reminiscent of aripiprazole's (Abilify®) (Xu et al. 2021), an antipsychotic with add-on use in the treatment of depression, vilazodone's piperazine moiety is surprisingly located further towards the extracellular space than observed in any of the other compounds (FIG. 36, FIG. 30). This is likely due to the opposite binding configurations of vilazodone and aripiprazole. Specifically, aripiprazole's N-aryl substituent and vilazodone's N-alkyl substituent bind to the orthosteric pocket, and D116^{3.32} and Y390^{7.43} thus interact with the same piperazine amine in both compounds. Buspirone, on the other hand, assumes an unusual overall binding mode, where its azaspirodecane-7,9-dione group does not extend towards the extracellular space as observed for the piperazine substituents of aripiprazole or vilazodone. Instead, buspirone adopts a kinked conformation in which the azaspirodecane-7,9-dione group bends into a crevice between TM2 and TM3. There it appears to displace the sidechain of F112^{3.28}, which switches its rotamer state to face the membrane (FIG. 30). In the orthosteric binding pocket, buspirone's piperazine interacts with D116^{3.32} in a similar fashion as observed for aripiprazole, and its pyrimidine group is located near TM3 and TM5, forming mostly hydrophobic interactions. Overall, we note that despite their diverse chemical scaffolds, vilazodone, aripiprazole, and buspirone assume similar poses in the core orthosteric pocket with their aromatic piperazine substituents primarily being stabilized by phenylalanines in TM6 (F361^{6.51} and F362^{6.52}). 5-MeO-DMT and the 5-HT1A-selective analog 4-F, 5-MeO-PyrT thus exhibit similar binding modes as antidepressant medications in the orthosteric binding pocket, which implies related pharmacological activities.

To test this hypothesis, we performed signaling assays to characterize and contrast the in vitro pharmacological activities of the different prescription drugs to those of 4-F, 5-MeO-PyrT (FIG. 36). In BRET assays determining 5-HT1A-mediated activation of Gi1, we found that buspirone (Eₘₐₓ=93.4% of 5-HT), vilazodone (Eₘₐₓ=97.4%), and 4-F, 5-MeO-PyrT (Eₘₐₓ=102.8%) are all high efficacy agonists, while aripiprazole (Eₘₐₓ=77.1%) showed modestly reduced efficacy. We note that vilazodone shows activity at concentrations as low as 30 pM, which we suspect is likely due to slow binding kinetics of the drug. Vilazodone (Gi BRET EC₅₀=480 pM) shows the highest potency of medications tested, which could be in part owed to its atypical binding mode described above. In addition to a similar binding mode in the orthosteric pocket as prescribed antidepressants, the 5-HT1A-selective indoleamine 4-F, 5-MeO-PyrT (Gi BRET EC₅₀=370 pM) shows comparable potency and efficacy to vilazodone. However, while these findings underscore that 4-F, 5-MeO-PyrT exhibits 5-HT1A activity comparable to that of antidepressant medications, it also reveals subtle differences in their efficacies. This is in line with our observation that vilazodone, buspirone, and aripiprazole also bind to extended binding pockets, and thus likely stabilize conformational ensembles distinct from that of 5-MeO-DMT and 4-F, 5-MeO-PyrT. Thus, 4-F, 5-MeO-PyrT as a 5-HT1A/5-HT2A selective tryptamine could enable the exploration of 5-HT1A-mediated behavioral aspects of the 5-MeO-tryptamine class of psychedelics such as 5-MeO-DMT.

### In Vivo Pharmacology and Activity of 5-HT1A-selective 5-MeO-DKT Derivatives

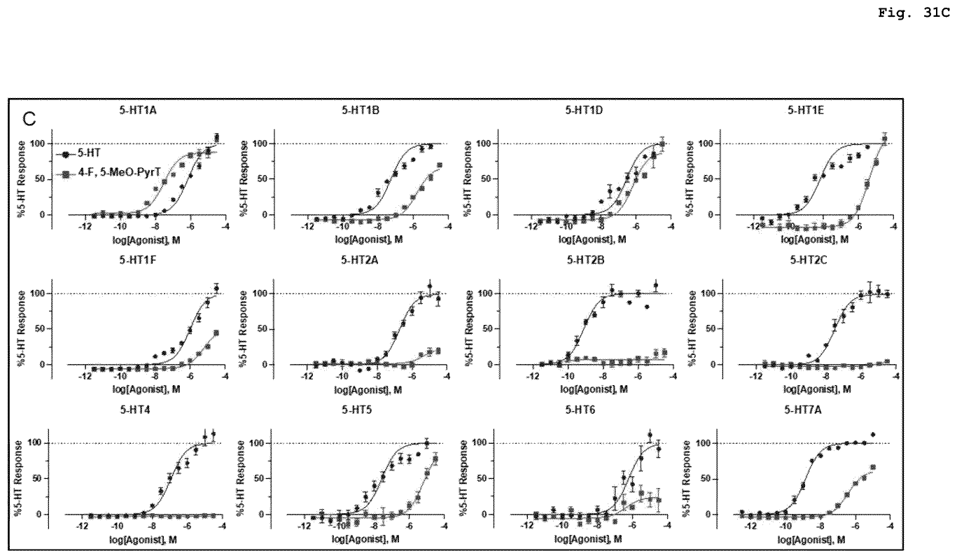

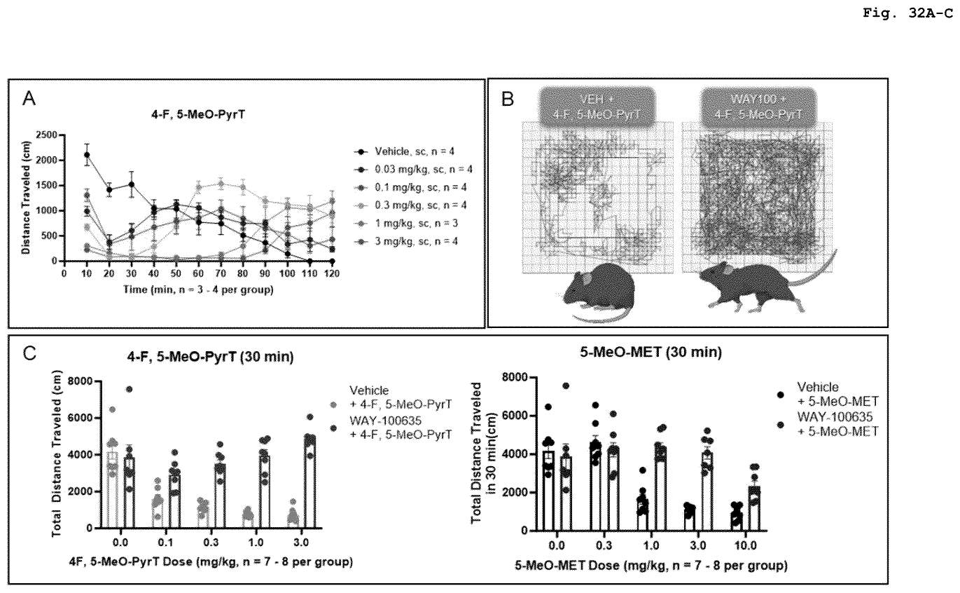

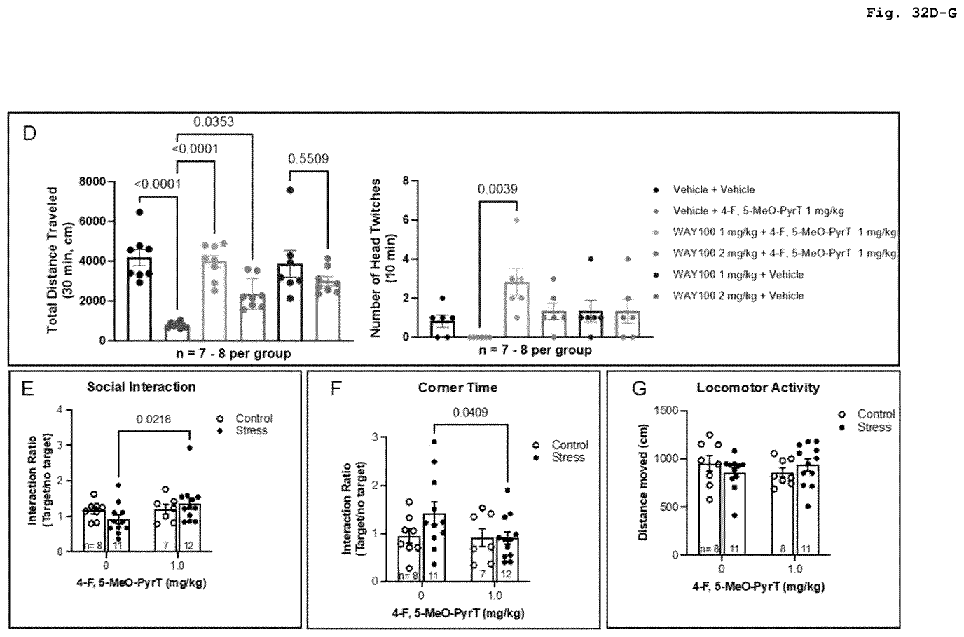

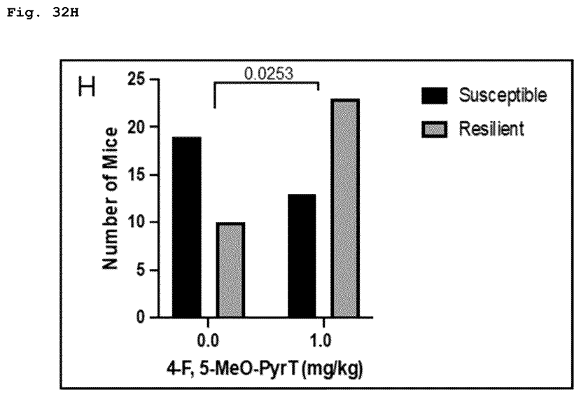

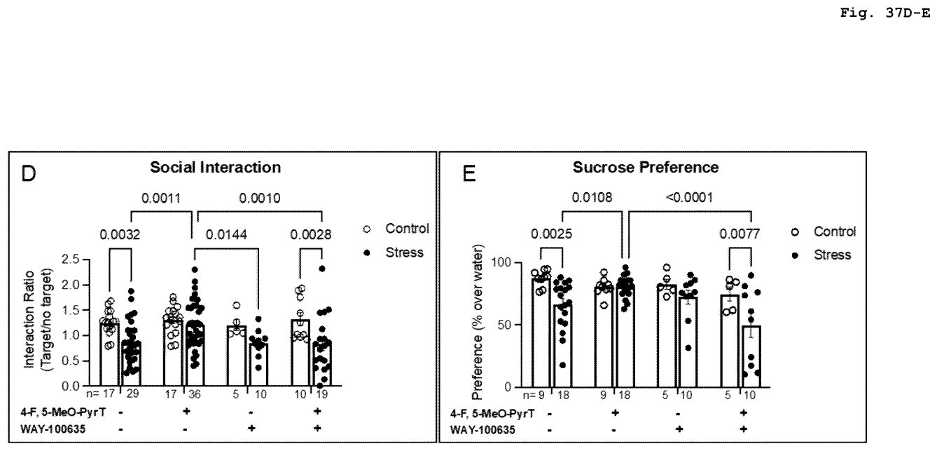

We next tested the usefulness of 4-F, 5-MeO-PyrT as a target-selective in vivo probe to interrogate 5-HT1A's role in both the hallucinogenic and the potential therapeutic effects of the 5-MeO-tryptamine structural class of psychedelics. Using in vitro studies we not only determined that 4-F, 5-MeO-PyrT is more than 800-fold selective for 5-HT1A over 5-HT2A (FIG. 31, Table 4), but also found that the compound has substantially lower activity at all other G protein-coupled serotonin receptors, the serotonin transporter, and other neurotransmitter transporters (FIG. 31). In vivo, 4-F, 5-MeO-PyrT shows good brain penetration that peaks 30 minutes following drug administration (total brain:plasma ratio of 3.3, unbound brain:plasma ratio of 0.91, C_{max/brain}=143 ng/mL after 1 mg/kg subcutaneous (s.c.) administration), and the compound is largely cleared within 2 hr (FIG. 37A, Table 11). At 82.5% brain tissue binding, the estimated free drug concentration in the brain at Tₘₐₓ is ˜100 nM following a dose of 1 mg/kg s.c., which is expected to exert high 5-HT1A engagement in vivo (Gi BRET EC₅₀=370 pM), with substantially lower engagement of 5-HT2A (Gq BRET EC₅₀=300 nM) (Table 11, Table 4). To test these estimates in functional assays in vivo, we examined acute locomotor activity suppression (sedation) as a measure of 5-HT1A activation and the head-twitch response (HTR) as a measure of 5-HT2A activation (Halberstadt et al. 2011). We observed a dose-dependent locomotor suppression in the open field (OF) test for both 4-F-5-MeO-PyrT and 5-MeO-MET (FIG. 32), a non-scheduled, balanced 5-HTA/5-HT2A agonist with near-identical in vitro activity to 5-MeO-DMT (FIG. 29A). In HTR, 5-MeO-MET showed robust dose-dependent activity following co-administration of the 5-HTA-selective antagonist WAY-100635, indicating that HTR was suppressed by 5-HT1A activation in our experimental design, which is a well-documented effect of 5-HT1A agonism (FIG. 37B) (Darmani et al. 1990 and Arnt et al. 1989). In contrast, 4-F, 5-MeO-PyrT did not produce notable HTR at doses up to 3 mg/kg in the presence or absence of WAY-100635 (FIG. 37B, FIG. 32D). 4-F, 5-MeO-PyrT thus does not display apparent 5-HT2A activity in vivo following comparatively high doses (up to 100-fold higher than the lowest dose producing suppression of locomotion, 0.03 mg/kg), with or without 5-HT1A antagonist. Together, these studies suggest that in vivo 4-F, 5-MeO-PyrT is highly potent and selective for 5-HT1A, readily enters the brain, and does not functionally engage 5-HT2A receptors at the tested doses.

### Behavioral Characterizations of Tryptamines

As 5-MeO-DMT is reported to have anxiolytic and antidepressant activity in human subjects (Ermakova et al. 2022, David et al. 2019, and Uthaug et al. 2019), we next investigated 5-HT1A-mediated behaviors in preclinical models using 4-F, 5-MeO-PyrT. We used a chronic social defeat stress (CSDS) model to induce a depressive-like phenotype in mice, which has been amply validated with SSRIs (chronic dosing) and ketamine (single administration) Bagot et al. 2017, Berton et al. 2006, and Donahue et al. 2014).

Briefly, male C57BL/6J mice were introduced to a novel CD-1 aggressor for 10 consecutive days of 10-minute defeat bouts. Twenty-four hours after the last defeat bout, mice were tested in a two-part social interaction (SI) test (FIG. 37C) where experimental mice are permitted to explore an open field (no target). In the second phase, a novel CD-1 aggressor is placed into the enclosure (target). The interaction ratio (IR) is then determined by calculating the time the experimental mouse spends in the interaction zone during the “no target” and “target” phases. Given the social nature of rodents, control mice tend to spend more time with the novel target mouse than with the empty enclosure, however, stress susceptible mice exhibit a generalized avoidance to conspecifics and spend less time interacting with the novel mouse (Golden et al. 2011 and Krishnan et al. 2007).

To test the effect of 4-F, 5-MeO-PyrT in this paradigm, we injected 1 mg/kg of drug or vehicle s.c. 1 hr after the last SD session on Day 10, followed by the SI test 24 hr post drug administration on Day 11. This delayed readout alleviates the confounding acute sedative effects of 4-F, 5-MeO-PyrT and acts as a test of lasting therapeutic-like effects beyond initial drug exposure.

Strikingly, this phenotype was rescued by 4-F, 5-MeO-PyrT treatment, evidenced by an increased IR in the stress-exposed mice, with one administration of 4-F, 5-MeO-PyrT (1.0 mg/kg), or 4-F, 5-MeO-MET (10.0 mg/kg) rescuing the social interaction deficit induced by CSDS and was able to shift the resulting phenotype of the stressed mice such that 60-70% showed a stress-resilient phenotype.

Control mice receiving either vehicle or drug did not show any distinguishable differences in IR, indicating that the drug has measurable effects in a stress-experienced population. Mice were also co-administered with the selective 5-HT1A antagonist WAY-100635 to validate that the effects of 4-F, 5-MeO-PyrT in this paradigm were 5-HT1A-mediated. Indeed, we observe that WAY-100635 co-administration blocked 4-F, 5-MeO-PyrT's ability to ameliorate SI deficits in this model. Given the high affinity and selectivity of 4-F, 5-MeO-PyrT, and the inhibition of its behavioral effect by the established 5-HT1A antagonist WAY-100635, these results suggest that the observed effects of 4-F, 5-MeO-PyrT are 5-HT1A mediated.

We also recorded locomotor activity on a cohort of animals following SD and treatment with vehicle or 4-F, 5-MeO-PyrT to eliminate the possibility of confounding factors due to decreased locomotion (FIG. 32F). The amount of time each animal spent in the corners was also quantified. There were no apparent alterations in locomotor activity of treated mice at the time of the social interaction test, confirming that any observed behavioral differences were not due to any motion deficits. We found that vehicle-treated defeated mice spent more time in the corners, confirming previous observations that defeated mice not only show reduced social interaction but also engage in more vigilant, antisocial behaviors (FIG. 32E-G). As expected due to the increased SI, this behavior was no longer observed in drug-treated mice. Lastly, it has been reported that approximately one third of mice appear to be stress resilient in this paradigm as defined by an SI ratio of greater than 1 (Krishnan et al. 2018). This likely leads to an underestimation of the effect of 4-F, 5-MeO-PyrT in our cohorts. We, therefore, wanted to account for this resilience, and determine whether 4-F, 5-MeO-PyrT treatment could increase the proportion of resilient mice. And indeed, comparison of the vehicle- and drug-treated stressed animals shows that 4-F, 5-MeO-PyrT markedly increases the number of resilient mice (FIG. 32H). Together, these findings further support the conclusion that 4-F, 5-MeO-PyrT treatment ameliorates stress-related social deficits.

To evaluate additional depressive-like behaviors induced by SD, we next investigated anhedonia via a sucrose preference test immediately following SI (Krishnan et al. 2007) (FIG. 37E). Briefly, mice were allowed ad libitum access to two bottles, one containing water and the other 1% sucrose. Stressed mice treated with vehicle had a significantly reduced preference for sucrose compared to non-stressed mice, further validating the depressive-like phenotype induced by SD. However, stressed mice treated with 4-F, 5-MeO-PyrT had significantly increased sucrose preference relative to vehicle-treated animals and were essentially indistinguishable from non-stressed mice receiving either vehicle or drug. As observed for the social interaction experiment, co-administration of WAY-100635 reversed the effects of 4-F, 5-MeO-PyrT in stressed animals, suggesting that the anti-anhedonic effect of 4-F, 5-MeO-PyrT is 5-HTA-mediated. While WAY-100635 treatment alone appears to increase sucrose preference in stressed mice, this effect is not statistically significant, and a similar effect was not observed in the SI experiment.

Overall, our studies thus demonstrate that 4-F, 5-MeO-PyrT can ameliorate social deficits and anhedonia in a social defeat mouse model in a similar fashion as has been shown for ketamine and SSRIs (Bagot et al., Gottschalk et al. 2018, Willner et al. 1987, and Li et al. 2011). suggesting that 5-HT1A might play a key role in the observed therapeutic effects of 5-MeO-DMT and related tryptamines.

TABLE 1
Names and Structures of Tested Compounds

| Compound # | Name | Structure |
|---|---|---|
| 1 | 2-(4-fluoro-5-methoxy-1H- indol-3-yl)-N,N-dimethylethan- 1-amine | 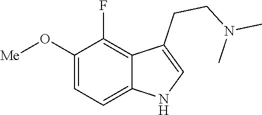 [SMILES: COc1ccc2[nH]cc(CCN(C)C)c2c1F] |
| 2 | N-ethyl-2-(4-fluoro-5-methoxy- 1H-indol-3-yl)-N-methylethan- 1-amine | 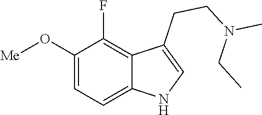 [SMILES: CCN(C)CCc1c[nH]c2ccc(OC)c(F)c12] |
| 3 | N,N-diethyl-2-(4-fluoro-5- methoxy-1H-indol-3-yl)ethan-1- amine | 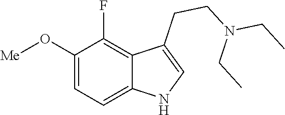 [SMILES: CCN(CC)CCc1c[nH]c2ccc(OC)c(F)c12] |
| 4 | N-(2-(4-fluoro-5-methoxy-1H- indol-3-yl)ethyl)-N- methylpropan-1-amine | 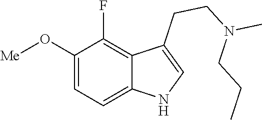 [SMILES: CCCN(C)CCc1c[nH]c2ccc(OC)c(F)c12] |
| 5 | N-ethyl-(2-(4-fluoro-5- methoxy-1H-indol-3- yl)ethyl)propan-1-amine | 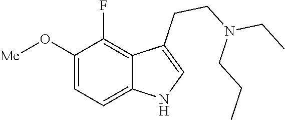 [SMILES: CCCN(CC)CCc1c[nH]c2ccc(OC)c(F)c12] |
| 6 | N-(2-(4-fluoro-5-methoxy-1H- indol-3-yl)ethyl)-N- propylpropan-1-amine | 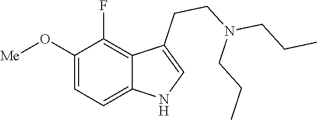 [SMILES: CCCN(CCC)CCc1c[nH]c2ccc(OC)c(F)c12] |
| 7 | N-(2-(4-fluoro-5-methoxy-1H- indol-3-yl)ethyl)-N- methylpropan-2-amine | 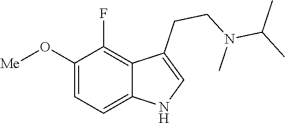 [SMILES: COc1ccc2[nH]cc(CCN(C)C(C)C)c2c1F] |
| 8 | N-(2-(4-fluoro-5-methoxy-1H- indol-3-yl)ethyl)-N- isopropylpropan-1-amine | 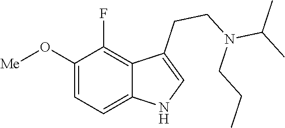 [SMILES: CCCN(CCc1c[nH]c2ccc(OC)c(F)c12)C(C)C] |
| 9 | N-(2-(4-fluoro-5-methoxy-1H- indol-3-yl)ethyl)-N- isopropylpropan-2-amine | 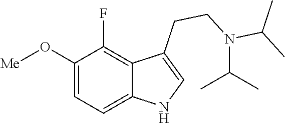 [SMILES: COc1ccc2[nH]cc(CCN(C(C)C)C(C)C)c2c1F] |
| 10 | N-(2-(4-fluoro-5-methoxy-1H- indol-3-yl)ethyl)-N- methylprop-2-en-1-amine | 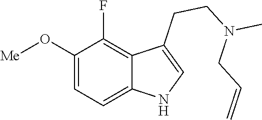 [SMILES: C=CCN(C)CCc1c[nH]c2ccc(OC)c(F)c12] |
| 11 | 4-fluoro-5-methoxy-1-methyl-3- (2-(pyrrolidin-1-yl)ethyl)-1H- indole | 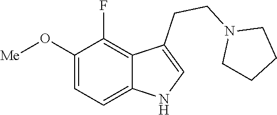 [SMILES: COc1ccc2[nH]cc(CCN3CCCC3)c2c1F] |
| 12 | 4-fluoro-5-methoxy-3-(2- (piperidin-1-yl)ethyl)-1H- indole | 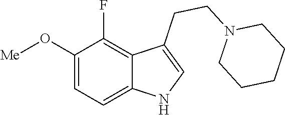 [SMILES: COc1ccc2[nH]cc(CCN3CCCCC3)c2c1F] |
| 13 | 3-(2-(2,5-dihydro-1H-pyrrol-1- yl)ethyl)-4-fluoro-5-methoxy- 1H-indole | 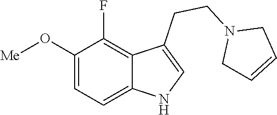 [SMILES: COc1ccc2[nH]cc(CCN3CC=CC3)c2c1F] |
| 14 | (1S,4R)-2-(2-(4-fluoro-5- methoxy-1H-indol-3-yl)ethyl)- 2-azabicyclo[2.2.2]oct-5-ene | 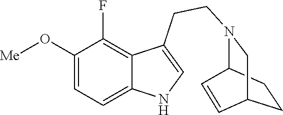 [SMILES: COc1ccc2[nH]cc(CCN3CC4C=CC3CC4)c2c1F] |
| 15 | (1S,4R,7S)-7-ethyl-2-(2-(4- fluoro-5-methoxy-1H-indol-3- yl)ethyl)-2- azabicyclo[2.2.2]oct-5-ene | 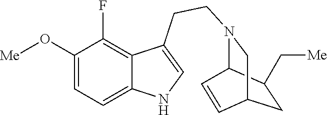 [SMILES: CCC1CC2C=CC1N(CCc1c[nH]c3ccc(OC)c(F)c13)C2] |
| 16 | 3-(2-(dimethylamino)ethyl)-4- fluoro-1H-indol-5-ol | 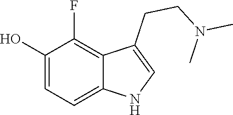 [SMILES: CN(C)CCc1c[nH]c2ccc(O)c(F)c12] |
| 17 | 3-(2-(diethylamino)ethyl)-4- fluoro-1H-indol-5-ol | 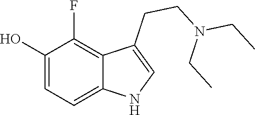 [SMILES: CCN(CC)CCc1c[nH]c2ccc(O)c(F)c12] |
| 18 | 4-fluoro-3-(2-(pyrrolidin-1- yl)ethyl)-1H-indol-5-ol | 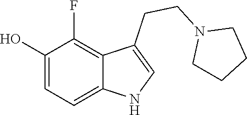 [SMILES: Oc1ccc2[nH]cc(CCN3CCCC3)c2c1F] |
| 19 | 4-fluoro-5-methoxy-1-methyl-3- (2-(pyrrolidin-1-yl)ethyl)-1H- indole | 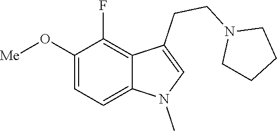 [SMILES: COc1ccc2c(c(CCN3CCCC3)cn2C)c1F] |
| 20 | 3-(2-(2,5-dihydro-1H-pyrrol-1- yl)ethyl)-4-fluoro-5-methoxy- 1-methyl-1H-indole | 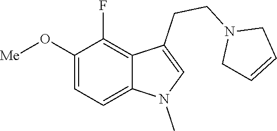 [SMILES: COc1ccc2c(c(CCN3CC=CC3)cn2C)c1F] |
| 5-HT | 5-Hydroxytryptamine (serotonin) | 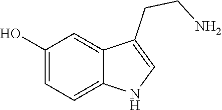 [SMILES: NCCc1c[nH]c2ccc(O)cc12] |

TABLE 2
pEC50 and Emax summary of selected 4-F, 5-MeO-tryptamines

| | EC₅₀ (nM) Mean | | | Normalized (%5HT) Top Mean | | |
|---|---|---|---|---|---|---|
| | Gio(cAMP)-1A | BRET | BRET | Gio(cAMP) | BRET | BRET |
| Compound | (pEC₅₀, SEM) | Gi1-1A | Gq-2A | 1A (SEM) | Gi1-1A | Gq-2A |
| 1 | 0.19 | 1.77 | 86.50 | 110.2 | 103.5 | 68.8 |
| | (9.73, | (8.75, | (7.06, | (2.08) | (1.59) | (2.08) |
| | 0.12) | 0.04) | 0.08) | | | |
| 2 | 0.32 | 1.77 | 99.31 | 108.5 | 101.5 | 70.0 |
| | (9.50, | (8.75, | (7.00, | (1.50) | (0.72) | (4.27) |
| | 0.39) | 0.10) | 0.21) | | | |
| 3 | 0.21 | 3.18 | 672.98 | 110.5 | 104.0 | 69.0 |
| | (9.69, | (8.50, | (6.17, | (1.70) | (1.68) | (12.80) |
| | 0.10) | 0.04) | 0.18) | | | |
| 4 | 0.42 | 3.06 | 114.55 | 112.5 | 103.9 | 79.0 |
| | (9.38, | (8.51, | (6.94, | (2.80) | (0.92) | (0.60) |
| | 0.27) | 0.03) | 0.01) | | | |
| 5 | 0.15 | 0.95 | 154.53 | 112.9 | 99.7 | 90.1 |
| | (9.82, | (9.02, | (6.81, | (3.72) | (4.61) | (1.42) |
| | 0.28) | 0.10) | 0.03) | | | |
| 6 | 1.31 | 3.70 | 142.23 | 106.2 | 98.2 | 95.3 |
| | (8.88, | (8.43, | (6.85, | (0.99) | (1.89) | (1.09) |
| | 0.24) | 0.11) | 0.05) | | | |
| 7 | 2.98 | 14.16 | 151.71 | 109.0 | 103.5 | 83.4 |
| | (8.53, | (7.85, | (6.82, | (3.99) | (0.47) | (7.06) |
| | 0.14) | 0.08) | 0.003) | | | |
| 8 | 5.96 | 16.79 | 249.46 | 104.4 | 100.6 | 86.3 |
| | (8.23, | (7.78, | (6.60, | (0.49) | (0.72) | (5.33) |
| | 0.12) | 0.09) | 0.31) | | | |
| 9 | 6.00 | 35.81 | 338.06 | 98.5 | 100.1 | 96.9 |
| | (8.22, | (7.45, | (6.47, | (1.30) | (1.89) | (5.69) |
| | 0.07) | 0.004) | 0.19) | | | |
| 10 | 2.57 | 8.41 | 156.31 | 104.7 | 100.0 | 66.8 |
| | (8.59, | (8.08, | (6.81, | (1.90) | (2.84) | (9.87) |
| | 0.22) | 0.05) | 0.27) | | | |
| 11 | 0.03 | 0.37 | 299.92 | 105.9 | 102.8 | 83.6 |
| | (10.55, | (9.43, | (6.52, | (1.07) | (1.61) | (2.45) |
| | 0.08) | 0.07) | 0.04) | | | |
| 12 | 2.42 | 8.97 | ND* | 110.8 | 105.3 | ND* |
| | (8.62, | (8.05, | | (2.41) | (1.46) | |
| | 0.19) | 0.003) | | | | |
| 13 | 0.0023 | 0.22 | 120.22 | 111.1 | 105.9 | 95.6 |
| | (11.64, | (9.66, | (6.92, | (2.94) | (0.62) | (3.72) |
| | 0.70) | 0.32) | 0.10) | | | |
| 14 | 173.38 | — | — | 106.8 | — | — |
| | (6.76, | | | (10.56) | | |
| | 0.29) | | | | | |
| 15 | 47.32 | — | — | 96.8 | — | — |
| | (7.33, | | | (2.29) | | |
| | 0.11) | | | | | |
| 16 | 0.25 | 1.13 | 7.46 | 105.1 | 101.0 | 71.0 |
| | (9.60, | (8.95, | (8.13, | (2.11) | (2.74) | (4.01) |
| | 0.82) | 0.10) | 0.08) | | | |
| 17 | 0.76 | — | — | 106.4 | — | |
| | (9.12, | | | (3.58) | | |
| | 0.14) | | | | | |
| 18 | 0.040 | — | — | 110.3 | — | — |
| | (10.38, | | | (2.53) | | |
| | 0.11) | | | | | |
| 19 | 100.00 | — | — | 110.9 | — | — |
| | (7.00, | | | (6.23) | | |
| | 0.10) | | | | | |
| 20 | 2.74 | — | — | 107.5 | — | — |
| | (8.56, | | | (0.92) | | |
| | 0.15) | | | | | |
| 5-HT | 0.21 | 2.61 | 4.40 | 100.0 | 100.0 | 100.0 |
| | (9.68, | (8.58, | (8.36, | | | |
| | 0.04) | 0.04) | 0.07) | | | |

TABLE 3
Summary of EC₅₀ and Eₘₐₓ of IP1 functional assay 5-HT2A/B

| | | | 5-HT2A | 5-HT2B | |
|---|---|---|---|---|---|
| | | 5-HT2A | (IP1) | (IP1) | |
| | Compound Structure | (IP1) | Eₘₐₓ | EC₅₀ | 5-HT2B (IP1) |
| Entry | and Code | EC₅₀ (nM) | (%5-HT) | (nM) | Eₘₐₓ (%5-HT) |
| 1 | 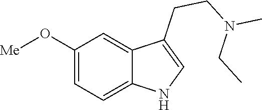 [SMILES: CCN(C)CCc1c[nH]c2ccc(OC)cc12] | 96 | 107 | 130 | 62 |
| | 5-MeO-MET | | | | |
| 2 | 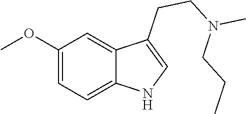 | 79 | 89 | 100 | 61 |
| | 5-MeO-MPT | | | | |
| 3 | 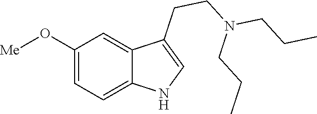 [SMILES: CCCN(CCC)CCc1c[nH]c2ccc(OC)cc12] | 82 | 94 | 76 | 108 |
| | 5-MeO-DPT | | | | |
| 4 | 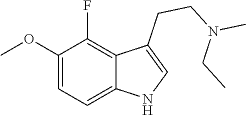 | 230 | 90 | 66 | 39 |
| | 4-F, 5-MeO-MET | | | | |
| 5 | 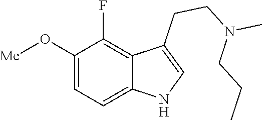 [SMILES: CCCN(C)CCc1c[nH]c2ccc(OC)c(F)c12] | 250 | 106 | 72 | 47 |
| | 4-F, 5-MeO-MPT | | | | |
| 6 | 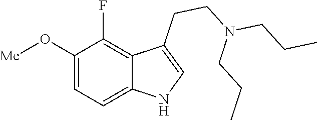 [SMILES: CCCN(CCC)CCc1c[nH]c2ccc(OC)c(F)c12] | 180 | 100 | 99 | 86 |
| | 4-F, 5-MeO-DPT | | | | |
| 7 | 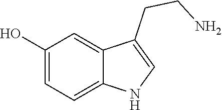 [SMILES: NCCc1c[nH]c2ccc(O)cc12] | 31 | 3.5 | 100 | 100 |
| | 5-HT | | | | |

TABLE 4
EC₅₀ and Efficacy Summary of Tryptamine Analogs

| | | EC₅₀ (nM) | | | Efficacy as %5HT | | |
|---|---|---|---|---|---|---|---|
| | | (pEC₅₀, SEM) | | | (SEM) | | |
| | | | 5-HT1A | 5-HT2A | | 5-HT1A | 5-HT2A |
| | | 5-HT1A | BRET | BRET | 5-HT1A | BRET | BRET |
| # | Structure | cAMP | Gi1 | Gq | cAMP | Gi1 | Gq |
| 1 | 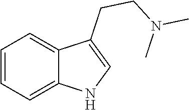 | 651.63 (6.19, 0.14) | 1584.89 (5.80, 0.02) | 94.41 (7.03, 0.16) | 98.3 (5.94) | 95.1 (1.01) | 67.2 (2.51) |
| | DMT | | | | | | |
| 2 | 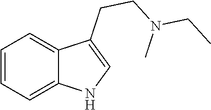 | 302.00 (6.52, 0.08) | — | — | 90.0 (4.09) | — | — |
| | MET | | | | | | |
| 3 | 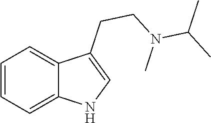 [SMILES: CC(C)N(C)CCc1c[nH]c2ccccc12] | 1976.97 (5.70, 0.04) | — | — | 73.5 (13.60) | — | — |
| | MiPT | | | | | | |
| 4 | 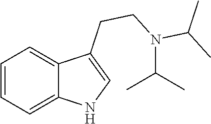 [SMILES: CC(C)N(CCc1c[nH]c2ccccc12)C(C)C] | 1327.39 (5.88, 0.05) | — | — | 59.8 (1.14) | — | — |
| | DiPT | | | | | | |
| 5 | 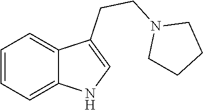 [SMILES: c1ccc2c(CCN3CCCC3)c[nH]c2c1] | 19.10 (7.72, 0.20) | — | — | 99.0 (6.13) | — | — |
| | PyrT | | | | | | |
| 6 | 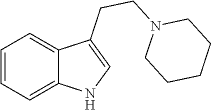 [SMILES: c1ccc2c(CCN3CCCCC3)c[nH]c2c1] | 485.29 (6.31, 0.27) | — | — | 88.2 (18.97) | — | — |
| | PipT | | | | | | |
| 7 | 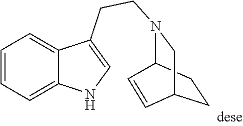 [SMILES: C1=CC2CCC1CN2CCc1c[nH]c2ccccc12] | 15454.5 (4.81, 0.18) | — | — | 75.0 (21.47) | — | — |
| | desethyl | | | | | | |
| | isoquinuclidineT | | | | | | |
| 8 | 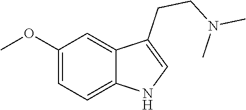 | 3.67 (8.44, 0.11) | 25.59 (7.59, 0.06) | 24.83 (7.61, 0.12) | 104.2 (4.78) | 105.2 (1.52) | 85.46 (0.93) |
| | 5-MeO-DMT | | | | | | |
| 9 | 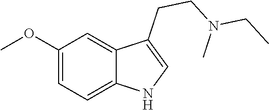 [SMILES: CCN(C)CCc1c[nH]c2ccc(OC)cc12] | 4.44 (8.35, 0.06) | 25.94 (7.59, 0.06) | 37.67 (7.42, 0.06) | 95.6 (1.93) | 104.1 (3.95) | 74.8 (3.24) |
| | 5-MeO-MET | | | | | | |
| 10 | 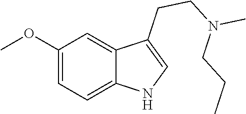 | 18.58 (7.73, 0.14) | 60.26 (7.22, 0.07) | 28.31 (7.55, 0.04) | 105.5 (4.16) | 96.4 (10.97) | 80.1 (2.60) |
| | 5-MeO-MPT | | | | | | |
| 11 | 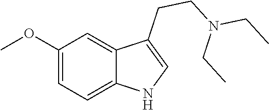 [SMILES: CCN(CC)CCc1c[nH]c2ccc(OC)cc12] | 11.04 (7.96, 0.15) | 37.07 (7.43, 0.01) | 171.79 (6.77, 0.18) | 118.8 (2.44) | 101.9 (2.65) | 75.0 (6.13) |
| | 5-MeO-DET | | | | | | |
| 12 | 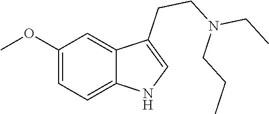 [SMILES: CCCN(CC)CCc1c[nH]c2ccc(OC)cc12] | 2.47 (8.61, 0.03) | 8.36 (8.08, 0.03) | 22.08 (7.66, 0.06) | 105.2 (4.09) | 100.4 (5.01) | 81.8 (5.70) |
| | 5-MeO-EPT | | | | | | |
| 13 | 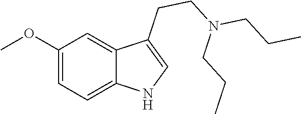 [SMILES: CCCN(CCC)CCc1c[nH]c2ccc(OC)cc12] | 3.57 (8.36, 0.10) | 15.42 (7.81, 0.01) | 20.18 (7.70, 0.06) | 111.6 (6.8) | 101.5 (2.28) | 84.9 (3.63) |
| | 5-MeO-DPT | | | | | | |
| 14 | 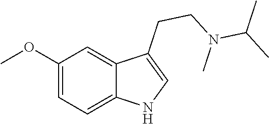 [SMILES: COc1ccc2[nH]cc(CCN(C)C(C)C)c2c1] | 51.76 (7.29, 0.04) | 112.46 (6.95, 0.04) | 17.34 (7.76, 0.01) | 100.1 (2.11) | 94.8 (7.94) | 88.3 (3.07) |
| | 5-MeO-MiPT | | | | | | |
| 15 | 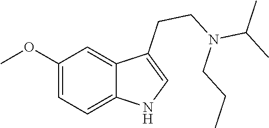 [SMILES: CCCN(CCc1c[nH]c2ccc(OC)cc12)C(C)C] | 26.67 (7.57, 0.12) | 101.16 (7.00, 0.01) | 51.17 (7.29, 0.02) | 105.4 (5.96) | 100.5 (0.87) | 88.9 (2.67) |
| | 5-MeO-PriPT | | | | | | |
| 16 | 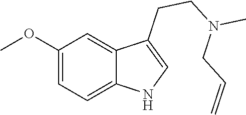 | 9.64 (8.02, 0.20) | 42.66 (7.37, 0.07) | 24.47 (7.59, 0.14) | 107.2 (5.91) | 99.7 (6.52) | 85.3 (0.36) |
| | 5-MeO-MALT | | | | | | |
| 17 | 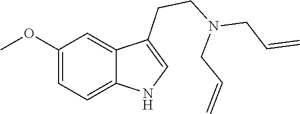 [SMILES: C=CCN(CC=C)CCc1c[nH]c2ccc(OC)cc12] | 4.66 (8.33, 0.06) | — | — | 94.6 (3.64) | — | — |
| | 5-MeO-DALT | | | | | | |
| 18 | 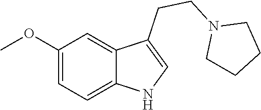 [SMILES: COc1ccc2[nH]cc(CCN3CCCC3)c2c1] | 0.16 (9.78, 0.09) | 2.13 (8.67, 0.05) | 81.47 (7.09, 0.10) | 102.8 (3.13) | 103.7 (1.51) | 87.9 (1.75) |
| | 5-MeO-PyrT | | | | | | |
| 19 | 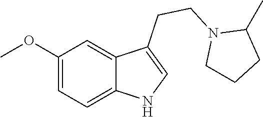 | 2.40 (8.62, 0.24) | — | — | 108.3 (5.34) | — | — |
| | 5-MeO-α-Me-PyrT | | | | | | |
| 20 | 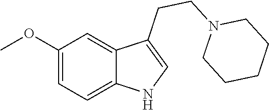 [SMILES: COc1ccc2[nH]cc(CCN3CCCCC3)c2c1] | 22.23 (7.65, 0.17) | 88.51 (7.05, 0.01) | ND* | 106.3 (7.13) | 103.8 (2.07) | ND* |
| | 5-MeO-PipT | | | | | | |
| 21 | 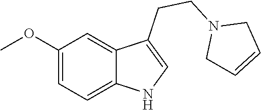 [SMILES: COc1ccc2[nH]cc(CCN3CC=CC3)c2c1] | 0.35 (10.59, 0.20) | 0.26 (9.59, 0.02) | 9.91 (8.00, 0.12) | 101.3 (2.70) | 101.3 (2.18) | 95.0, 3.50 |
| | 5-MeO-3-PyrrolineT | | | | | | |
| 22 | 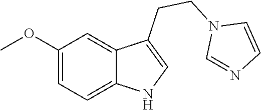 [SMILES: COc1ccc2[nH]cc(CCn3ccnc3)c2c1] | 292.42 (6.53, 0.17) | — | — | 69.6 (9.33) | — | — |
| | 5-MeO-ImidT | | | | | | |
| 23 | 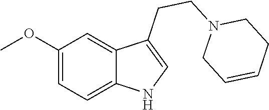 [SMILES: COc1ccc2[nH]cc(CCN3CC=CCC3)c2c1] | 8.77 (8.06, 0.14) | — | — | 105.1 (2.15) | — | — |
| | 5-MeO-dehydro-PipT | | | | | | |
| 24 | 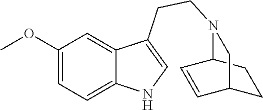 [SMILES: COc1ccc2[nH]cc(CCN3CC4C=CC3CC4)c2c1] | 628.06 (6.20, 0.12) | — | — | 93.6 (3.98) | — | — |
| | 5-MeO-desethyl | | | | | | |
| | isoquinuclidineT | | | | | | |
| 25 | 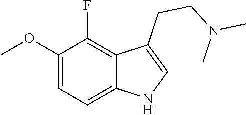 | 0.19 (9.73, 0.12) | 1.77 (8.75, 0.04) | 86.50 (7.06, 0.08) | 110.2 (2.08) | 103.5 1.59) | 68.8 (2.08) |
| | 4-F, 5-MeO-DMT | | | | | | |
| 26 | 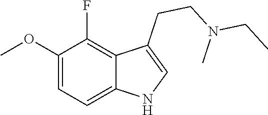 [SMILES: CCN(C)CCc1c[nH]c2ccc(OC)c(F)c12] | 0.32 (9.50, 0.39) | 1.77 (8.75, 0.10) | 99.31 (7.00, 0.21) | 108.5 (1.50) | 101.5 (0.72) | 70.0 (4.27) |
| | 4-F, 5-MeO-MET | | | | | | |
| 27 | 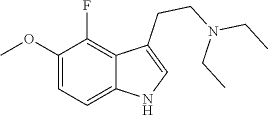 [SMILES: CCN(CC)CCc1c[nH]c2ccc(OC)c(F)c12] | 0.21 (9.69, 0.10) | 3.18 (8.50, 0.04) | 672.98 (6.17, 0.18) | 110.5 (1.70) | 104.0 (1.68) | 69.0 (12.80) |
| | 4-F, 5-MeO-DET | | | | | | |
| 28 | 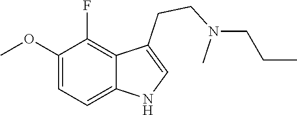 [SMILES: CCCN(C)CCc1c[nH]c2ccc(OC)c(F)c12] | 0.42 (9.38, 0.27) | 3.06 (8.51, 0.03) | 114.55 (6.94, 0.01) | 112.5 (2.80) | 103.9 (0.92) | 79.0 (0.60) |
| | 4-F, 5-MeO-MPT | | | | | | |
| 29 | 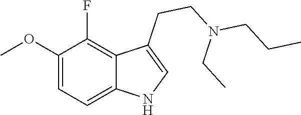 [SMILES: CCCN(CC)CCc1c[nH]c2ccc(OC)c(F)c12] | 0.15 (9.82, 0.28) | 0.95 (9.02, 0.10) | 154.53 (6.81, 0.03) | 112.9 (3.72) | 99.7 (4.61) | 90.1 (1.42) |
| | 4-F, 5-MeO-EPT | | | | | | |
| 30 | 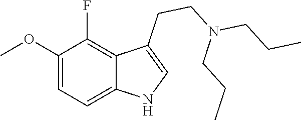 [SMILES: CCCN(CCC)CCc1c[nH]c2ccc(OC)c(F)c12] | 1.31 (8.88, 0.24) | 3.70 (8.43, 0.11) | 142.23 (6.85, 0.05) | 106.2 (0.99) | 98.2 (1.89) | 95.3 (1.09) |
| | 4-F, 5-MeO-DPT | | | | | | |
| 31 | 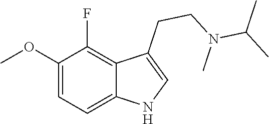 [SMILES: COc1ccc2[nH]cc(CCN(C)C(C)C)c2c1F] | 2.98 (8.53, 0.14) | 14.16 (7.85, 0.08) | 151.71 (6.82, 0.003) | 109.0 (3.99) | 103.5 (0.47) | 83.4 (7.06) |
| | 4-F, 5-MeO-MiPT | | | | | | |
| 32 | 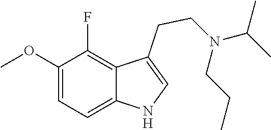 [SMILES: CCCN(CCc1c[nH]c2ccc(OC)c(F)c12)C(C)C] | 5.96 (8.23, 0.12) | 16.79 (7.78, 0.09) | 249.46 (6.60, 0.31) | 104.4 (0.49) | 100.6 (0.72) | 86.3 (5.33) |
| | 4-F, 5-MeO-PriPT | | | | | | |
| 33 | 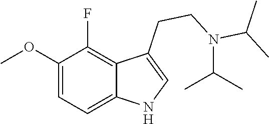 [SMILES: COc1ccc2[nH]cc(CCN(C(C)C)C(C)C)c2c1F] | 6.00 (8.22, 0.07) | 35.81 (7.45, 0.004) | 338.06 (6.47, 0.19) | 98.5 (1.30) | 100.1 (1.89) | 96.9 (5.69) |
| | 4-F, 5-MeO-DiPT | | | | | | |
| 34 | 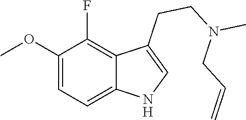 | 2.57 (8.59, 0.22) | 8.41 (8.08, 0.05) | 156.31 (6.81, 0.27) | 104.7 (1.90) | 100.0 (2.84) | 66.8 (9.87) |
| | 4-F, 5-MeO-MALT | | | | | | |
| 35 | 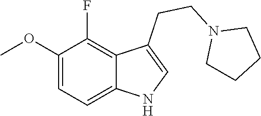 [SMILES: COc1ccc2[nH]cc(CCN3CCCC3)c2c1F] | 0.03 (10.55, 0.08) | 0.37 (9.43, 0.07) | 299.92 (6.52, 0.04) | 105.9 (1.07) | 102.8 (1.62) | 83.6 (2.45) |
| | 4-F, 5-MeO-PyrT | | | | | | |
| 36 | 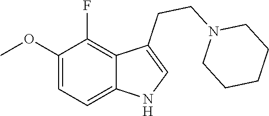 [SMILES: COc1ccc2[nH]cc(CCN3CCCCC3)c2c1F] | 2.42 (8.62. 0.19) | 8.97 (8.05, 0.003) | ND* | 110.8 (2.41) | 105.3 (1.46) | ND* |
| | 4-F, 5-MeO-PipT | | | | | | |
| 37 | 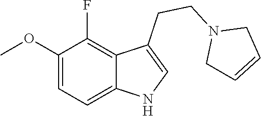 [SMILES: COc1ccc2[nH]cc(CCN3CC=CC3)c2c1F] | 0.0023 (11.64, 0.70) | 0.22 (9.66, 0.32) | 120.22 (6.92, 0.10) | 111.1 (2.94) | 105.9 (0.62) | 95.6 (3.72) |
| | 4-F, 5-MeO-3- | | | | | | |
| | pyrrolineT | | | | | | |
| 38 | 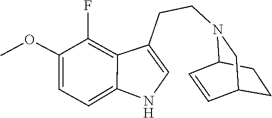 [SMILES: COc1ccc2[nH]cc(CCN3CC4C=CC3CC4)c2c1F] | 173.38 (6.76, 0.29) | — | — | 106.8 (10.56) | — | — |
| | 4-F, 5-MeO-desethyl | | | | | | |
| | isoquinuclidine | | | | | | |
| 39 | 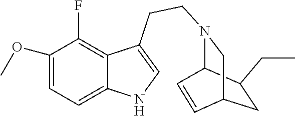 [SMILES: CCC1CC2C=CC1N(CCc1c[nH]c3ccc(OC)c(F)c13)C2] | 47.32 (7.33, 0.11) | — | — | 96.8 (2.29) | — | — |
| | 4-F, 5-MeO-ethyl | | | | | | |
| | isoquinuclidine | | | | | | |
| 40 | 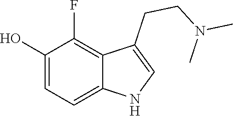 [SMILES: CN(C)CCc1c[nH]c2ccc(O)c(F)c12] | 0.25 (9.60, 0.82) | 1.13 (8.95, 0.10) | 7.46 (8.13, 0.08) | 105.1 (2.11) | 101.0 (2.74) | 71.0 (4.01) |
| | 4-F, 5-OH-DMT | | | | | | |
| 41 | 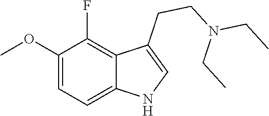 [SMILES: CCN(CC)CCc1c[nH]c2ccc(OC)c(F)c12] | 0.76 (9.12, 0.14) | — | — | 106.4 (3.58) | — | |
| | 4-F, 5-OH-DET | | | | | | |
| 42 | 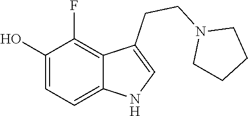 [SMILES: Oc1ccc2[nH]cc(CCN3CCCC3)c2c1F] | 0.040 (10.38, 0.11) | — | — | 110.3 (2.53) | — | — |
| | 4-F, 5-OH-PyrT | | | | | | |
| 43 | 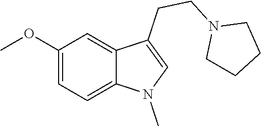 [SMILES: COc1ccc2c(c1)c(CCN1CCCC1)cn2C] | 89.13 (7.05, 0.20) | — | — | 104.9 (8.52) | — | — |
| | 1-Me-indole-5-MeO- | | | | | | |
| | pyrT | | | | | | |
| 44 | 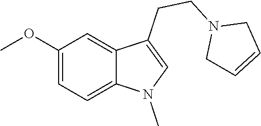 [SMILES: COc1ccc2c(c1)c(CCN1CC=CC1)cn2C] | 209.41 (6.68, 0.21) | — | — | 99.4 (4.26) | — | — |
| | 1-Me-indole-5-MeO- | | | | | | |
| | 3-pyrrolineT | | | | | | |
| 45 | 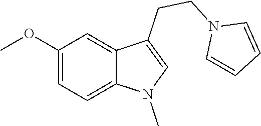 [SMILES: COc1ccc2c(c1)c(CCn1cccc1)cn2C] | 6576.58 (5.18, 0.0004) | — | — | 94.4 (5.25) | — | — |
| | 1-Me-indole-5-MeO- | | | | | | |
| | PyrroleT | | | | | | |
| 46 | 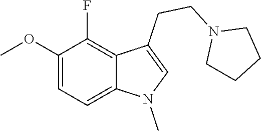 [SMILES: COc1ccc2c(c(CCN3CCCC3)cn2C)c1F] | 100.00 (7.00, 0.10) | — | — | 110.9 (6.23) | — | — |
| | 1-Me-indole-4-F, 5- | | | | | | |
| | MeO-PyrT | | | | | | |
| 47 | 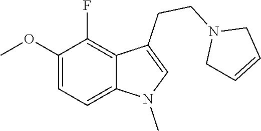 [SMILES: COc1ccc2c(c(CCN3CC=CC3)cn2C)c1F] | 2.74 (8.56, 0.15) | — | — | 107.5 (0.92) | — | — |
| | 1-Me-indole-4-F, 5- | | | | | | |
| | MeO-3-pyrrolineT | | | | | | |
| 48 | 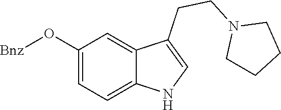 | 33.04 (7.48, 0.05) | — | — | 109.6 (4.47) | — | — |
| | 5-Bnz-PyrT | | | | | | |
| 49 | 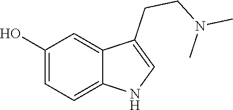 | — | 9.29 (8.03, 0.01) | 27.86 (7.56, 0.04) | — | 104.5 (0.65) | 74.6 (4.30) |
| | Bufotenine | | | | | | |
| | (5-OH-DMT) | | | | | | |
| 50 | 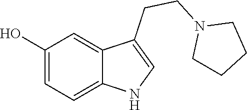 [SMILES: Oc1ccc2[nH]cc(CCN3CCCC3)c2c1] | 0.10 (10.010.34) | — | — | 102.8 (2.16) | — | — |
| | 5-OH-PyrT | | | | | | |
| 51 | 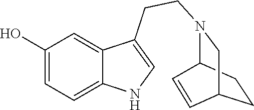 [SMILES: Oc1ccc2[nH]cc(CCN3CC4C=CC3CC4)c2c1] | 149.28 (6.83, 0.20) | — | — | 106.6 (5.46) | — | — |
| | 5-OH-desethyl | | | | | | |
| | isoquinuclidineT | | | | | | |
| 52 | 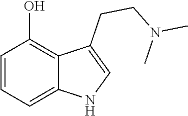 | 511.68 (6.29, 0.11) | 1083.93 (5.97, 0.13) | 35.89 (7.45, 0.05) | 102.0 (4.48) | 88.7 (4.78) | 52.6 (7.70) |
| | Psilocin | | | | | | |
| 53 | 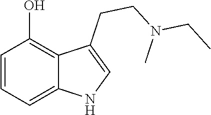 [SMILES: CCN(C)CCc1c[nH]c2cccc(O)c12] | 769.19 (6.11, 0.06) | — | — | 85.1 (6.93) | — | — |
| | 4-OH-MET | | | | | | |
| 54 | 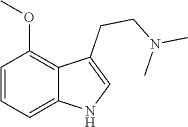 [SMILES: COc1cccc2[nH]cc(CCN(C)C)c12] | 558.47 (6.25, 0.23) | — | — | 100.2 (11.45) | — | — |
| | 4-MeO-DMT | | | | | | |
| 55 | 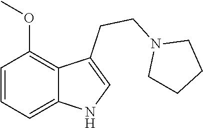 [SMILES: COc1cccc2[nH]cc(CCN3CCCC3)c12] | 163.68 (6.79, 0.30) | — | — | 107.0 (3.58) | — | — |
| | 4-MeO-PyrT | | | | | | |
| 56 | 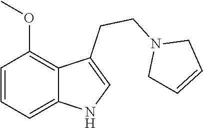 [SMILES: COc1cccc2[nH]cc(CCN3CC=CC3)c12] | 0.22 (8.47, 0.16) | — | — | 106.2 (2.17) | — | — |
| | 4-MeO-3-pyrrolineT | | | | | | |
| 57 | 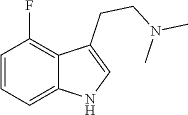 | 87.70 (7.06, 0.18) | — | — | 101.8 (2.94) | — | — |
| | 4-F-DMT | | | | | | |
| 58 | 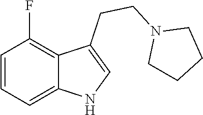 [SMILES: Fc1cccc2[nH]cc(CCN3CCCC3)c12] | 16.75 (7.78, 0.08) | — | — | 109.1 (3.46) | — | — |
| | 4-F-PyrT | | | | | | |
| 59 | 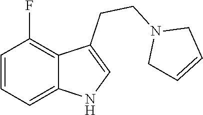 [SMILES: Fc1cccc2[nH]cc(CCN3CC=CC3)c12] | 0.88 (9.06, 0.06) | | — | 106.7 (2.34) | — | — |
| | 4-F, 3-pyrrolineT | | | | | | |
| 60 | 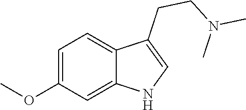 | 3235.94 (5.49, 0.11) | — | — | 102.4 (6.71) | — | — |
| | 6-MeO-DMT | | | | | | |
| 61 | 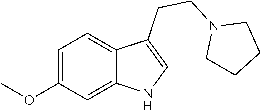 [SMILES: COc1ccc2c(CCN3CCCC3)c[nH]c2c1] | 492.04 (6.31, 0.09) | — | — | 111.0 (7.06) | — | — |
| | 6-MeO-PyrT | | | | | | |
| 62 | 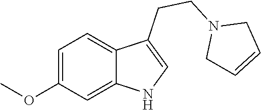 [SMILES: COc1ccc2c(CCN3CC=CC3)c[nH]c2c1] | 30.90 (7.51, 0.03) | — | — | 100.5 (3.95) | — | — |
| | 6-MeO-3-pyrolineT | | | | | | |
| 63 | 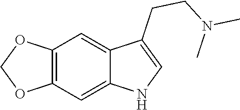 [SMILES: CN(C)CCc1c[nH]c2cc3c(cc12)OCO3] | 64.42 (7.19.0.01) | — | — | 105.0 (9.93) | — | — |
| | 5,6-methylenedioxy- | | | | | | |
| | DMT | | | | | | |
| 64 | 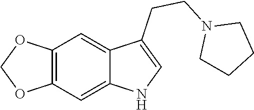 [SMILES: c1[nH]c2cc3c(cc2c1CCN1CCCC1)OCO3] | 3.50 (8.46, 0.09) | — | — | 105.3 (5.78) | — | — |
| | 5,6-methylenedioxy- | | | | | | |
| | PyrT | | | | | | |
| 65 | 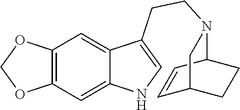 [SMILES: C1=CC2CCC1CN2CCc1c[nH]c2cc3c(cc12)OCO3] | 9885.53 (5.01, 0.10) | — | — | 115.8 (2.48) | — | — |
| | 5,6-methylenedioxy- | | | | | | |
| | desethyl- | | | | | | |
| | isoquinuclidine | | | | | | |
| 66 | 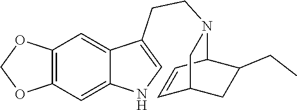 [SMILES: CCC1CC2C=CC1N(CCc1c[nH]c3cc4c(cc13)OCO4)C2] | 1690.44 (5.77, 0.25) | — | — | 88.7 (23.74) | — | — |
| | 5,6-methylenedioxy- | | | | | | |
| | isoquinuclidine | | | | | | |
| 67 | 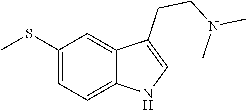 | 2.01 (8.70, 0.08) | 11.69 (7.93, 0.07) | 30.06 (7.52, 0.08) | 102.0 (2.63) | 106.6 (0.99) | 73.13 (0.87) |
| | 5-MeS-DMT | | | | | | |
| 68 | 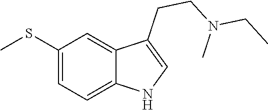 [SMILES: CCN(C)CCc1c[nH]c2ccc(SC)cc12] | 3.35 (8.48, 0.08) | — | — | 101.9 (1.93) | — | — |
| | 5-MeS-MET | | | | | | |
| 69 | 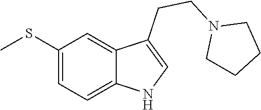 [SMILES: CSc1ccc2[nH]cc(CCN3CCCC3)c2c1] | 0.51 (9.30, 0.12) | 1.05 (8.98, 0.05) | 174.58 (6.76, 0.03) | 103.7 (0.64) | 99.5 (2.02) | 78.9 (2.97) |
| | 5-MeS-PyrT | | | | | | |
| 70 | 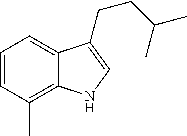 | 1207.81 (5.92, 0.05) | — | — | 91.5 (15.22) | — | — |
| | 7-Me-indole-DMT | | | | | | |
| 71 | 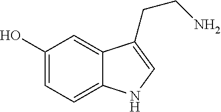 [SMILES: NCCc1c[nH]c2ccc(O)cc12] | 0.21 (9.68, 0.04) | 2.61 (8.58, 0.04) | 4.40 (8.36, 0.07) | 100.0 | 100.0 | 100.0 |
| | 5-HT | | | | | | |

*not detected-out of assay range

TABLE 5
EC₅₀ and Efficacy Summary of Azaindolyl and Benzofuranyl
Tryptamine Analogs

| | | Activity (5-HT1A cAMP) | |
|---|---|---|---|
| # | Structure | EC₅₀ (nM) (pEC50, SEM) | Efficacy (%5HT) (SEM) |
| 72 | 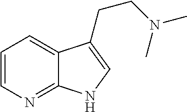 | 1905.46 (5.72, 0.25) | 88.3 (20.41) |
| | 7-azaindole-DMT | | |
| 73 | 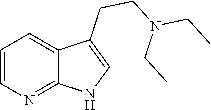 [SMILES: CCN(CC)CCc1c[nH]c2ncccc12] | 7798.30 (5.11, 0.22) | 108.8 (5.02) |
| | 7-azaindole-DET | | |
| 74 | 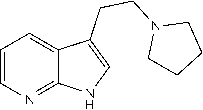 [SMILES: c1cnc2[nH]cc(CCN3CCCC3)c2c1] | 3243.40 (5.49, 0.14) | 95.6 (2.11) |
| | 7-azaindole-PyrT | | |
| 75 | 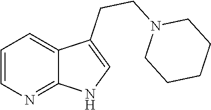 [SMILES: c1cnc2[nH]cc(CCN3CCCCC3)c2c1] | 18492.69 (4.73, 0.01) | 101.5 (0.02) |
| | 7-azaindole-PipT | | |
| 76 | 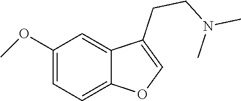 | 178.24 (6.75, 0.18) | 105.4 (1.13) |
| | 5-MeO-benzofuran- | | |
| | DMT | | |
| 77 | 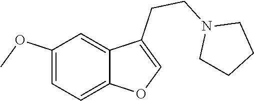 [SMILES: COc1ccc2occ(CCN3CCCC3)c2c1] | 7.19 (8.14, 0.23) | 112.8 (2.95) |
| | 5-MeO-benzofuran- | | |
| | PyrT | | |
| 78 | 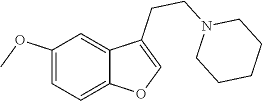 [SMILES: COc1ccc2occ(CCN3CCCCC3)c2c1] | 415.91 (6.38, 0.26) | 114.1 (8.86) |
| | 5-MeO-benzofuran- | | |
| | PipT | | |
| 79 | 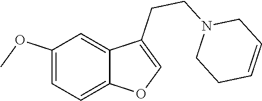 [SMILES: COc1ccc2occ(CCN3CC=CCC3)c2c1] | 7128.53 (5.15, 0.18) | 125.9 (9.71) |
| | 5-MeO-benzofuran-dihydro-PipT | | |
| 71 | 5-HT | 0.21 | 100.0 |
| | | (9.68, 0.04) | |

TABLE 6
EC₅₀ and Efficacy Summary of Commercial Ligands

| | | EC₅₀ (nM) (pEC₅₀, SEM) | | | Efficacy as %5HT (SEM) | | |
|---|---|---|---|---|---|---|---|
| | | | 5-HT1A | 5-HT2A | | 5-HT1A | 5-HT2A |
| | | 5-HT1A | BRET | BRET | 5-HT1A | BRET | BRET |
| # | Compound | cAMP | Gi1 | Gq | cAMP | Gi1 | Gq |
| 80 | LSD | 0.26 | 1.20 | 0.25 | 119.8 | 100.9 | 82.4 |
| | | (9.08, | (8.92, | (9.60, | (7.02) | (1.73) | (3.23) |
| | | 0.26) | 0.03) | 0.19) | | | |
| 81 | Mescaline | — | 19588.45 | 2404.36 | — | 42.0 | 92.6 |
| | | | (4.71, | (5.62, | | (6.38) | (1.36) |
| | | | 0.33) | 0.15) | | | |
| 82 | 8-OH-DPAT | 0.46 | — | — | 108.5 | — | — |
| | | (10.41, | | | (12.09) | | |
| | | 0.46) | | | | | |
| 83 | SEP | 1081.43 | — | — | 70.4 | — | — |
| | | (5.97, | | | (6.33) | | |
| | | 0.32) | | | | | |
| 84 | N-(1- | 50.35 | — | — | 137.0 | — | — |
| | naphthyl) | (7.30, | | | (14.09) | | |
| | piperazine | 0.29) | | | | | |
| 85 | Vilazodone | 0.03 | 0.48 | | 110.5 | 97.4 | — |
| | | (10.60, | (9.32, | | (3.72) | (0.46) | |
| | | 0.20) | 0.547) | | | | |
| 86 | Tandospirone | 1.51 | — | — | 96.3 | — | — |
| | | (8.82, | | | 4.50) | | |
| | | 0.21) | | | | | |
| 87 | Gepiron | 16.18 | — | — | 95.4 | — | — |
| | | (7.79, | | | (4.29) | | |
| | | 0.10) | | | | | |
| 88 | Buspirone | 1.44 | 9.68 | — | 96.0 | 93.4 | — |
| | | (8.84, | (8.01, | | (0.62) | (0.66) | |
| | | 1.44) | 0.16) | | | | |
| 89 | Aripiprazole | — | 5.83 | — | — | 77.1 | — |
| | | | (8.24, | | | (2.72) | |
| | | | 0.002) | | | | |
| 71 | 5-HT | 0.21 | 2.61 | 4.40 | 100.0 | 100.0 | 100.0 |
| | | (9.68, | (8.58, | (8.36, | | | |
| | | 0.04) | 0.04) | 0.07) | | | |

TABLE 7
Anecdotal Reports of “Designer” Tryptamine-Mediated
Effects in Humans

| Name | Structure | References |
|---|---|---|
| 5-MeO-DMT | 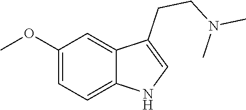 | https://erowid.org/experiences/subs/exp_5MeODMT.shtml https://isomerdesign.com/PiHKAL/read.php?domain=tk&id=38 |
| 5-MeO-MET | 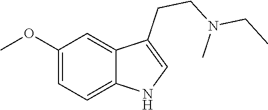 [SMILES: CCN(C)CCc1c[nH]c2ccc(OC)cc12] | https://erowid.org/experiences/exp.cgi?S1=910&S2=−1&C1=−1&Str https://drugs-forum.com/threads/5-meo-met.40412/ |
| 5-MeO-DPT | 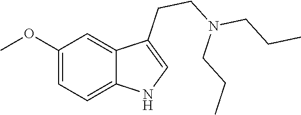 [SMILES: CCCN(CCC)CCc1c[nH]c2ccc(OC)cc12] | https://erowid.org/experiences/exp.cgi?S1=391&OldSort=RA_PDD&NewSort= SA&Start=0&ShowViews=0&Cellar=0 https://drugs-forum.com/threads/5-meo-dpt-trip-reports.26997/ https://isomerdesign.com/PiHKAL/read.php?domain=tk&id=36 |
| 5-MeO-DET | 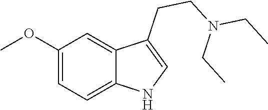 [SMILES: CCN(CC)CCc1c[nH]c2ccc(OC)cc12] | https://erowid.org/experiences/exp.php?ID=43242 https://erowid.org/library/book_online/tihkal/tihkal36.shtml |
| 5-MeO-MiPT | 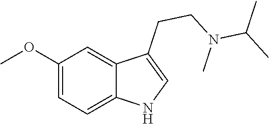 [SMILES: COc1ccc2[nH]cc(CCN(C)C(C)C)c2c1] | https://erowid.org/experiences/subs/exp_5MeOMIPT.shtml https://isomerdesign.com/PiHKAL/read.php?domain=tk&id=40 |
| 5-MeO-EiPT | 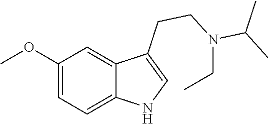 [SMILES: CCN(CCc1c[nH]c2ccc(OC)cc12)C(C)C] | https://erowid.org/experiences/exp.cgi?S1=727&S2=−1&C1=−1&Str |
| 5-MeO-DiPT | 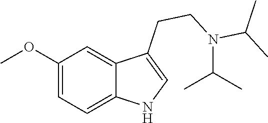 [SMILES: COc1ccc2[nH]cc(CCN(C(C)C)C(C)C)c2c1] | https://erowid.org/experiences/subs/exp_5MeODiPT.shtml http://isomerdesign.com/PiHKAL/read.php?id=37&domain=tk |
| 5-MeO-MALT | 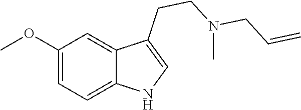 [SMILES: C=CCN(C)CCc1c[nH]c2ccc(OC)cc12] | https://erowid.org/experiences/exp.cgi?S1=883&S2=−1&C1=−1Str |
| 5-MeO-DALT | 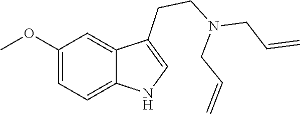 [SMILES: C=CCN(CC=C)CCc1c[nH]c2ccc(OC)cc12] | https://erowid.org/experiences/subs/exp_5MeODALT.shtml http://isomerdesign.com/PiHKAL/read.php?domain=tk&code=5-MEO-DALt |
| 5-MeO-PyrT | 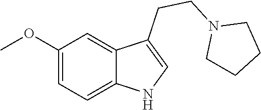 [SMILES: COc1ccc2[nH]cc(CCN3CCCC3)c2c1] | https://isomerdesign.com/PiHKAL/read.php?domain=tk&id=43 https://www.youtube.com/watch?v=x9WmEVRxebE&ab_channel= HamiltonMorris |
| 5-MeO-PipT | 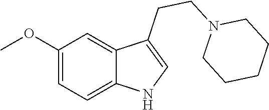 [SMILES: COc1ccc2[nH]cc(CCN3CCCCC3)c2c1] | https://erowid.org/experiences/exp.php?ID=115247 https://www.reddit.com/r/researchchemicals/comments/ltyf38/5meopipt_trial_ run_and_dosage_estimation/ https://www.reddit.com/r/Psychedelics/comments/s8rg40/oral_low_to_ moderate_dose_5meopipt_compared_with/ |

TABLE 8
Compound Potency and Efficacy at Mutant 5-HT1A and 5-HT2A Receptors

| | Gi1 BRET | | | | | |
|---|---|---|---|---|---|---|
| | 5-HT1A WT | | 5-HT1A A365N^{6.55} | | 5-HT1A N386V^{7.39} | |
| | EC₅₀ (nM) | Efficacy | EC₅₀ (nM) | Efficacy | EC₅₀ (nM) | Efficacy |
| | (pEC₅₀, | %5-HT | (pEC₅₀, | %5-HT | (pEC₅₀, | %5-HT |
| Compound | SEM) | (SEM) | SEM) | (SEM) | SEM) | (SEM) |
| 5-HT | 2.61 | 100.0 | 13.46 | 100.0 | 26.24 | 100.0 |
| | (8.58, | | (7.87, | | (7.58, | |
| | 0.04) | | 0.1) | | 0.04) | |
| DMT | 1584.89 | 95.1 | 5382.7 | 86.2 | — | — |
| | (5.8, | (1.01) | (5.27, | (3.62) | | |
| | 0.02) | | 0.09) | | | |
| 5-MeO-DMT | 25.59 | 105.2 | 196.79 | 100 | 38.19 | 99.4 |
| | (7.59, | (1.52) | (6.71, | (1.78) | (7.42, | (1.33) |
| | 0.06) | | 0.11) | | 0.06) | |
| 4-F, 5-OH- | 1.13 | 101 | 5.68 | 102.7 | — | — |
| DMT | (8.95, | (2.74) | (8.25, | (0.98) | | |
| | 0.1) | | 0.14) | | | |
| 4-F, 5- | 1.77 | 103.5 | 20.14 | 103.7 | 3.71 | 102.4 |
| MeO-DMT | (8.75, | (1.59) | (7.7, | (1.16) | (8.43, | (0.9) |
| | 0.04) | | 0.14) | | 0.09) | |
| 5-MeO-PyrT | 2.13 | 103.7 | 28.91 | 104.8 | 24.89 | 101.7 |
| | (8.67, | (1.51) | (7.54, | (0.85) | (7.6, | (0.47) |
| | 0.05) | | 0.09) | | 0.05) | |
| 4-F, 5- | 0.37 | 102.8 | 5.08 | 101.9 | 3 | 101.2 |
| MeO-PyrT | (9.43, | (1.62) | (8.29, | (0.02) | (8.52, | (2.08) |
| | 0.07) | | 0.05) | | 0.07) | |
| LSD | 1.2 | 100.9 | 1.73 | 101.1 | 0.37 | 92.3 |
| | (8.92, | (1.73) | (8.76, | (1.2) | (9.44, | (0.68) |
| | 0.03) | | 0) | | 0.04) | |
| Bufotenine | 9.29 | 104.5 | 171 | 103.8 | — | — |
| (5-OH-DMT) | (8.03, | (0.65) | (6.77, | (1.38) | | |
| | 0.01) | | 0.01) | | | |
| 5-MeS-DMT | 11.69 | 106.6 | 30.55 | 105.1 | — | — |
| | (7.93, | (0.99) | (7.52, | (1.19) | | |
| | 0.07) | | 0.06) | | | |
| 5-MeS-PyrT | 1.05 | 99.5 | 5.08 | 101.1 | — | — |
| | (8.98, | (2.03) | (8.29, | (1.02) | | |
| | 0.05) | | 0.03) | | | |
| | Gq BRET | | | | | |
| | 5-HT2A WT | | 5-HT2A N343A^{6.55} | | 5-HT2A V366N^{7.39} | |
| | EC₅₀ (nM) | Efficacy | EC₅₀ (nM) | Efficacy | EC₅₀ (nM) | Efficacy |
| | (pEC50, | %5-HT | (pEC50, | %5-HT | (pEC50, | %5-HT |
| Compound | SEM) | (SEM) | SEM) | (SEM) | SEM) | (SEM) |
| 5-HT | 4.4 | 100.0 | 583.45 | 100.0 | 15.38 | 100.0 |
| | (8.36, | | (6.23, | | (7.81, | |
| | 0.07) | | 0.09) | | 0.14) | |
| DMT | 94.41 | 67.2 | 479.73 | 34.8 | — | — |
| | (7.03, | (2.51) | (6.32, | (11.09) | | |
| | 0.16) | | 0.13) | | | |
| 5-MeO-DMT | 24.83 | 85.5 | 68.55 | 55.6 | 77.27 | 126.6 |
| | (7.61, | (0.93) | (7.16, | (6.58) | (7.11, | (1.94) |
| | 0.12) | | 0.08) | | 0.4) | |
| 4-F, 5-OH- | 7.46 | 71 | 140.93 | 57 | — | — |
| DMT | (8.13, | (4.01) | (6.85, | (6.08) | | |
| | 0.08) | | 0.07) | | | |
| 4-F, 5- | 86.5 | 68.8 | 140.6 | 67.3 | 391.74 | 101.6 |
| MeO-DMT | (7.06, | (2.08) | (6.85, | (6.04) | (6.41, | (2.43) |
| | 0.08) | | 0.25) | | 0.11) | |
| 5-MeO-PyrT | 81.47 | 87.9 | 111.94 | 68.7 | 55.59 | 150.3 |
| | (7.09, | (1.75) | (6.95, | (5.2) | (7.26, | (0.52) |
| | 0.1) | | 0.1) | | 0.28) | |
| 4-F, 5- | 299.92 | 83.6 | 257.63 | 77.8 | 157.04 | 129.4 |
| MeO-PyrT | (6.52, | (2.45) | (6.59, | (16.65) | (6.8, | (5.89) |
| | 0.04) | | 0.22) | | 0.15) | |
| LSD | 0.25 | 82.4 | 0.07 | 77.1 | 0.06 | 111.4 |
| | (9.6, | (3.23) | (10.17, | (6.05) | (10.2, | (0.74) |
| | 0.19) | | 0.1) | | 0.06) | |
| Bufotenine | 27.86 | 74.6 | 279.25 | 56.3 | — | — |
| (5-OH-DMT) | (7.56, | (4.3) | (6.55, | (3.36) | | |
| | 0.04) | | 0.36) | | | |
| 5-MeS-DMT | 30.06 | 73.1 | 26.49 | 51.2 | — | — |
| | (7.52, | (0.87) | (7.58, | (1.63) | | |
| | 0.08) | | 0.18) | | | |
| 5-MeS-PyrT | 174.58 | 78.9 | 111.17 | 64 | — | — |
| | (6.76, | (2.97) | (6.95, | (5.7) | | |
| | 0.03) | | 0.09) | | | |

| | cAMP | | | | |
|---|---|---|---|---|---|
| | 5-HT1A WT | | 5-HT1A F361L^{6.51} | | |
| | EC₅₀ (nM) | Efficacy | EC₅₀ (nM) | Efficacy | Fold-Change |
| | (pEC₅₀, | (%5-HT) | (pEC₅₀, | (%5-HT) | (F361L EC₅₀/ |
| Compound | SEM) | (SEM) | SEM) | (SEM) | WT EC₅₀) |
| 5-HT | 0.31 | 100.0 | 42.36 | 100.0 | 138 |
| | (−9.51, | | (−7.37, | | |
| | 0.09) | | 0.06) | | |
| 5-MeO- | 3.49 | 107.5 | 181.97 | 92.4 | 52 |
| DMT | (−8.46, | (6.04) | (−6.74, | (2.71) | |
| | 0.19) | | 0.02) | | |
| 4-F, 5- | 0.03 | 99.5 | 0.84 | 103 | 29 |
| MeO-PyrT | (−10.54, | (0.99) | (−9.08, | (1) | |
| | 0.07) | | 0.04) | | |

TABLE 9
Cryo-EM Data Collection, Model Refinement and Validation
Data Collection and Processing

| | | | 5-HT1A | | |
|---|---|---|---|---|---|
| | 5-HT1A | | Gi1 | | |
| | Gi1 DNG | 5-HT1A | 4-F, 5- | 5-HT1A | 5-HT1A |
| | 5-MeO- | Gi1 | MeO- | Gi1 | Gi1 DNG |
| Parameter | DMT | LSD | PyrT | Vilazodone | Buspirone |
| Magnification | 64000 | 81000 | 64000 | 81000 | 105000 |
| Voltage (kV) | 300 | 300 | 300 | 300 | 300 |
| Electron Exposure | 52.13 | 53.61 | 52.23 | 54.3 | 50 |
| (e−/A2) | | | | | |
| Defocus Range (μm) | −0.5 to −1.8 | −0.5 to −1.8 | −0.5 to −1.8 | −0.5 to 1.8 | −0.5 to 1.8 |
| Pixel Size | 1.076 | 1.058 | 1.076 | 1.069 | 0.825 |
| Symmetry imposed | C1 | C1 | C1 | C1 | C1 |
| Initial Particle | 9294420 | 12502189 | 4565297 | 18088270 | 5152166 |
| images | | | | | |
| Final particle | 266282 | 318050 | 265975 | 785536 | 586489 |
| images | | | | | |
| Map resolution | 2.79 | 2.64 | 2.85 | 2.94 | 2.62 |
| FSC threshold | 0.143 | 0.143 | 0.143 | 0.143 | 0.143 |
| Sharpening B- | 120.5 | 91.6 | 110.2 | 167.3 | 105.1 |
| factor | | | | | |

Refinement

| | | | | | |
|---|---|---|---|---|---|
| Model Composition | | | | | |
| Non-hydrogen atoms | 7297 | 7306 | 7365 | 7312 | 7367 |
| Protein resiudes | 914 | 914 | 914 | 914 | 914 |
| Ligands | 4 | 4 | 6 | 4 | 6 |
| Waters | 9 | 6 | 9 | 3 | 7 |
| RMS deviations | | | | | |
| bond lengths (Å) | 0.008 | 0.005 | 0.004 | 0.004 | 0.005 |
| Bond angles (°) | 1.151 | 0.659 | 0.584 | 0.55 | 0.995 |
| Clashscore | 5.78 | 6.17 | 6.32 | 5.28 | 7.07 |
| Rotamers outliers | 0.13 | 0 | 0 | 0 | 0 |
| (%) | | | | | |
| Ramachandran | | | | | |
| Plot (%) | | | | | |
| Favored | 97.45 | 97.78 | 97.67 | 98.12 | 97.45 |
| Allowed | 2.55 | 2.22 | 2.33 | 1.88 | 2.55 |
| Outliers | 0 | 0 | 0 | 0 | 0 |

Data Availability

| | | | 5-HT1A | | |
|---|---|---|---|---|---|
| | 5-HT1A | 5-HT1A | Gi1 | 5-HT1A | 5-HT1A |
| | Gi1 DNG | Gi1 | 4-F, 5-MeO- | Gi1 | Gi1 DNG |
| Source | 5-MeO-DMT | LSD | PyrT | Vilazodone | Buspirone |
| PDB | 8FY8 | 8FYT | 8FYE | 8FYL | 8FYX |
| EMDB | EMD-29560 | EMD-29597 | EMD-29571 | EMD-29585 | EMD-29599 |

TABLE 10
AUCₗₐₛₜ and Cₘₐₓ of 4-F, 5-MeO-PyrT
at 1 mg/kg SC administration

| Total | AUCₗₐₛₜ ((hr*ng)/mL) | Total | Cₘₐₓ (ng/mL) |
|---|---|---|---|
| Plasma | 26.40 | Plasma | 44.02 |
| Brain | 128.34 | Brain | 143.35 |

TABLE 11
Plasma and Brain Tissue Binding

| | | | | | | | | % |
|---|---|---|---|---|---|---|---|---|
| | | | | | | | | Compound |
| | % Bound | | | | % | | % | remaining |
| Compound | R1 | R2 | Mean | STDV | Unbound | fu | Recovery | at 4 h |

Mouse Brain Tissue Binding

| | | | | | | | | |
|---|---|---|---|---|---|---|---|---|
| Carbamazepine (1) | 89.0 | 87.4 | 88.2 | 1.1 | 11.8 | 0.118 | 72 | 96 |
| 4-F, 5-MeO- | 84.3 | 80.7 | 82.5 | 2.5 | 17.5 | 0.175 | 88 | 96 |
| PyrT (2) | | | | | | | | |

Mouse Plasma Protein Binding

| | | | | | | | | |
|---|---|---|---|---|---|---|---|---|
| Warfarin (1) | 93.9 | 93.3 | 93.6 | 0.4 | 6.4 | 0.064 | 90.9 | 103.3 |
| 4-F, 5-MeO- | 32.04 | 42.68 | 37.36 | 7.5 | 62.64 | 0.626 | 84.7 | 105.6 |
| PyrT (2) | | | | | | | | |

Human Plasma Protein Binding

| | | | | | | | | |
|---|---|---|---|---|---|---|---|---|
| Warfarin (1) | 98.0 | 98.5 | 98.3 | 0.3 | 1.7 | 0.017 | 108.1 | 108.8 |
| 4-F, 5-MeO- | 22.19 | 29.83 | 26.01 | 5.4 | 73.99 | 0.740 | 81.8 | 97.9 |
| PyrT (2) | | | | | | | | |

### Discussion

5-MeO-DMT is emerging as a promising transdiagnostic therapeutic as the recent preliminary clinical data and naturalistic surveys suggest the drug to be efficacious with rapid and lasting therapeutic effects. Our work here, as well as previous studies, show that 5-MeO-DMT has comparable signaling potency and efficacy at 5-HT1A and 5-HT2A receptors in vitro, and both receptors contribute to its in vivo pharmacology (Krebs-Thomson et al. 2006, Halberstadt et al. 2011, Halberstadt et al. 2011 and Riga et al. 2016). In light of previous work showcasing that 5-HT2A-selective agonists can alleviate anxiety-like and depression-like states in pre-clinical models, we wanted to investigate the role of 5-HT1A activity in shaping both psychedelic and therapeutic effects of 5-MeO-DMT and related 5-MeO-tryptamine psychedelics. While 5-HT1A is a validated therapeutic target for several approved medications (e.g. vilazodone and buspirone) (Celada et al. 2013), the importance of 5-HT1A agonism to the therapeutic effects of tryptamine psychedelics has not been conclusively addressed.

Since 5-HT2A receptors are responsible for the visual and other sensory disturbances elicited by classical psychedelics, it is typically assumed that these receptors also mediate therapeutic effects; however, there is currently no clinical evidence in support of this hypothesis, and the preclinical evidence to date is mixed. For example, the 5-HT2A antagonist ketanserin did not block psilocybin-mediated attenuation of anhedonia induced by chronic stress in mice (Hesselgrave et al. 2021), while in another study ketanserin blocked 5-MeO-DMT-mediated effects in a forced swim test (Cameron et al. 2023). In the latter study, psilocybin-mediated attenuation of anhedonia was abolished in 5-HT2A knock out (KO) mice (Cameron et al. 2023). Similarly, synaptogenesis readouts considered relevant for the therapeutic effects of psychedelics were not mediated by 5-HT2A in one study, while synaptic remodeling was 5-HT2A-dependent in another study (Shao et al. 2021 and de la Fuente et al. 2021).

Our findings that 4-F, 5-MeO-PyrT, a highly 5-HT1A-selective agonist, rescued social interaction deficits and anhedonia in mice induced by a social defeat model is thus of considerable interest. Although further systematic investigations will be required, our studies indicate that 5-HT1A may contribute to the reported therapeutic effects of 5-MeO-DMT. This hypothesis is also in line with the proposed complementary contributions of 5-HT1A and 5-HT2A to stress moderation (Carhart-Harris et al. 2017), the role of 5-HT1A in stress resilience (Bickle et al. 2023), as well as the reported antidepressant efficacy of clinical (Chauhan et al. 2022 and Robinson et al. 1990) and preclinical (Newman-Tancredi et al. 2018) 5-HT1A drugs. Furthermore, our results show that 5-HT1A-selective tryptamines lack the preclinical indications of classic psychedelic effects (e.g. HTR), suggesting that these compounds may not be hallucinogenic while retaining therapeutic effects.

With respect to acute psychedelic effects, studies further suggest that receptors other than 5-HT2A also modulate subjective experience. For example, LSD has recently been reported to acutely enhance emotional empathy and prosocial behavior in healthy volunteers, however the empathogenic effect was not blocked by ketanserin (Becker et al. 2022). Other studies specifically point to a major role of 5-HT1A, as the 5-HT1A antagonist pindolol increased the subjective psychedelic effects of DMT (Strassman et al. 1996). Moreover, the 5-HT1A agonist buspirone attenuates the visual hallucinogenic effects of psilocybin in healthy subjects Pokorny et al. 2016), further implying that unique subjective effects of 5-MeO-DMT's psychedelic experience could be shaped by 5-HT1A. In fact, examining the anecdotal reports on 5-MeO-DMT analogs studied herein reveals a wide spread of psychoactive effects (Shulgin et al. 1997) (Table 7). However, systematic human data is not available to enable meaningful correlation between the subjective experience of different 5-MeO-DMT derivatives and 5-HT1A potency, and/or 5-HT1A-5-HT2A relative potency. It should be noted though that 5-MeO-PyrT, an analog for which we report >38-fold selectivity for 5-HT1A over 5-HT2A, induces effects described as white-out and amnesia (Shulgin et al. 1997) (Table 7). While 5-MeO-DMT has some amnesic elements to its subjective experience (Reckweg et al. 2021), this effect appears greatly amplified in 5-MeO-PyrT (Shulgin et al. 1997), implying a possible role of 5-HT1A activation by 5-MeO-tryptamines in the mediation of these brain states and effects.

Despite 5-HT1A's importance in the effects of psychedelics, comparatively little is known about the structural pharmacology of different psychedelics at 5-HT1A. We addressed this important task in the present work by integrating cryoEM with systematic receptor mutagenesis and medicinal chemistry. Together, we provide both a global comparative map of receptor structural pharmacology for different drug classes, and detailed analyses of critical binding areas and specific amino acid residues that determine the signaling potency and efficacy at 5-HT1A as well as the selectivity for 5-HT1A over 5-HT2A. We uncover how receptor-specific sub-pockets determine both the potency and efficacy of tryptamine ligands at both receptors. Our findings provide a structure-guided framework that enables the development of tryptamine probes with finely tuned pharmacological activities and varying degrees of 5-HT1A/5-HT2A selectivity, including potent and highly 5-HT1A-selective compounds. Moreover, we elucidate how 5-MeO-DMT and a selective analog engage 5-HT1A in a virtually identical way, thereby showcasing the usefulness of this probe in studying 5-HT1A-mediated aspects of 5-MeO-DMT. These binding poses partially overlap with those of the 5-HT1A medications buspirone, vilazodone, and aripiprazole²⁴. However, these medications additionally occupy extended binding pockets, suggesting the stabilization of distinct conformational ensembles and thus the generation of signal outputs distinct from those of 5-MeO-DMT. These differences, as well as their engagement of other targets (Hughes et al. 2005, Le Foll et al. 2016, and Shapiro et al. 2003), likely play a role in the medications' distinct physiological effects compared to the psychedelic 5-MeO-DMT, but further experiments are required to investigate the precise correlates in detail. Nonetheless, we demonstrate that modification of the 5-MeO-DMT scaffold can produce highly selective probes that engage 5-HT1A in a structural and pharmacological manner that is similar to clinical drugs in vitro. But perhaps more importantly, the anxiolytic-like and antidepressant-like effects of these probes emphasize their utility in elucidating 5-HT1A-mediated effects of psychedelics in vivo, and potentially empower the exploration of therapeutic applications in future work.

The invention shows that a series of novel 5-MeO-DMT analogs having substitution in the 4th position of the indole with a fluorine atom exhibit a varying degree of selectivity between 5-HT1A and 5-HT2A receptors, including compounds with very high 5-HT1A selectivity over 5-HT2A receptors (1000-fold) and analogs with no practical level of activity at 5-HT2A receptors. We show that the featured compounds also exhibit high 5-HT1A functional selectivity versus 5-HT2B receptors, thus providing wide safety margins against the latter target which has been linked to adverse cardiac effects (Rothman et al. 2000). Namely, we demonstrate that 4-fluorination leads to a decrease in 5-HT2B receptor signaling efficacy across the featured tryptamine compound class, a novel finding with important safety consequences. We also demonstrate that the novel compounds are poor substrates for the monoamine oxidase enzymes A and B. As additional novel properties, we discovered that the novel compounds show species dependent metabolic stability (microsomal metabolism), including improved stability in human microsomes as compared to the corresponding DMT analogs. We show for the first time that 5-HT1A-selective tryptamines exhibit antidepressant and anxiolytic effects in a validated mouse model of depression and anxiety, with effects comparable to ketamine (Donahue et al. 2014), and thereby highlighting the therapeutic potential of novel 5-HT1A-preferring and selective tryptamines. We also demonstrate that 5-HT1A-selective tryptamines lack the preclinical signs of hallucinogenic effects. As such these compounds represent compelling therapeutic candidates.

### REFERENCES

Alper, R.; Lotsof, H. S.; Frenken, G. M. N; Luciano, D. J.; Jan Bastiaans, K. (1999) Treatment of acute opioid withdrawal with ibogaine. American Journal on Addictions, 8(3), 234-242.
Armstrong, S. B., Xin, Y., Sepeda, N. D., Polanco, M., Averill, L. A. & Davis, A. K. (2023) Prospective associations of psychedelic treatment for co-occurring alcohol misuse and posttraumatic stress symptoms among United States Special Operations Forces Veterans. Military Psychology, 1-8.
Arnt, J. & Hyttel, J. (1989) Facilitation of 8-Ohdpat-Induced Forepaw Treading of Rats by the 5-Ht2 Agonist Doi. European Journal of Pharmacology 161, 45-51.
Bagot, R. C. et al. (2017) Ketamine and Imipramine Reverse Transcriptional Signatures of Susceptibility and Induce Resilience-Specific Gene Expression Profiles. Biol Psychiatry 81, 285-295.
Ballesteros, J. A. & Weinstein, H. (1995) Integrated methods for the construction of three-dimensional models and computational probing of structure-function relations in G protein-coupled receptors. Methods in Neurosciences 25, 366-428.
Becker, A. M. et al. (2022) Ketanserin reverses the acute response to LSD in a randomized, double-blind, placebo-controlled, crossover study in healthy subjects. Int J Neuropsychopharmacol. https://doi.org:10.1093/ijnp/pyac075
Berton, O. et al. (2006) Essential role of BDNF in the mesolimbic dopamine pathway in social defeat stress. Science 311, 864-868.
Bickle, J. G. et al. (2023) 5HT1A receptors on dentate gyrus granule cells confer stress resilience. Biol Psychiatry https://doi.org:10.1016/j.biopsych.2023.10.007
Bhangale, J. O.; Acharya, S. R. (2016) Anti-Parkinson Activity of Petroleum Ether Extract of Ficus Religiosa (L.) Leaves. Advances in Pharmacological and Pharmaceutical Sciences 2016, e9436106. https://doi.org/10.1155/2016/9436106.
Blair, J. B., Kurrasch-Orbaugh, D., Marona-Lewicka, D., Cumbay, M. G., Watts, V. J., Barker, E. L., & Nichols, D. E. (2000) Effect of ring fluorination on the pharmacology of hallucinogenic tryptamines. Journal of medicinal chemistry, 43(24), 4701-4710.
Cameron, L. P. et al. 5-HT2ARs Mediate Therapeutic Behavioral Effects of Psychedelic Tryptamines. ACS Chem Neurosci. https://doi.org:10.1021/acschemneuro.2c00718
Cameron, L. P. et al. (2021) A non-hallucinogenic psychedelic analogue with therapeutic potential. Nature 589, 474-479. https://doi.org:10.1038/s41586-020-3008-z
Cao, D. et al. (2022) Structure-based discovery of nonhallucinogenic psychedelic analogs. Science 375, 403-411. https://doi.org:10.1126/science.abl8615
Carhart-Harris, R. L. & Nutt, D. J. (2017) Serotonin and brain function: a tale of two receptors. J Psychopharmacol 31, 1091-1120. https://doi.org:10.1177/0269881117725915
Carhart-Harris, R. L. et al. (2018) Psilocybin with psychological support for treatment-resistant depression: six-month follow-up. Psychopharmacology (Berl) 235, 399-408. https://doi.org:10.1007/s00213-017-4771-x
Celada, P., Bortolozzi, A. & Artigas, F. (2013) Serotonin 5-HT1A receptors as targets for agents to treat psychiatric disorders: rationale and current status of research. CNS Drugs 27, 703-716. https://doi.org:10.1007/s40263-013-0071-0
Chauhan, M., Parry, R. & Bobo, W. V. (2022) Vilazodone for Major Depression in Adults: Pharmacological Profile and an Updated Review for Clinical Practice. Neuropsychiatr Dis Treat 18, 1175-1193. https://doi.org:10.2147/ndt.S279342
Darmani, N. A., Martin, B. R., Pandey, U. & Glennon, R. A. Do Functional-Relationships Exist between 5-Ht1a and 5-Ht2 Receptors. (1990) Pharmacol Biochem Be 36, 901-906. https://doi.org:Doi 10.1016/0091-3057(90)90098-3
Davis, A. K., Averill, L. A., Sepeda, N. D., Barsuglia, J. P. & Amoroso, T. (2020) Psychedelic Treatment for Trauma-Related Psychological and Cognitive Impairment Among US Special Operations Forces Veterans. Chronic Stress (Thousand Oaks) 4, 2470547020939564. https://doi.org:10.1177/2470547020939564
Davis, A. K., So, S., Lancelotta, R., Barsuglia, J. P. & Griffiths, R. R. (2019) 5-methoxy-N,N-dimethyltryptamine (5-MeO-DMT) used in a naturalistic group setting is associated with unintended improvements in depression and anxiety. Am J Drug Alcohol Abuse 45, 161-169. https://doi.org:10.1080/00952990.2018.1545024
de la Fuente Revenga, M. et al. (2021) Prolonged epigenomic and synaptic plasticity alterations following single exposure to a psychedelic in mice. Cell Rep 37, 109836. https://doi.org:10.1016/j.celrep.2021.109836
Donahue, R. J.; Muschamp, J. W.; Russo, S. J.; Nestler, E. J.; Carlezon Jr, W. A. (2014). Effects of striatal ΔFosB overexpression and ketamine on social defeat stress-induced anhedonia in mice. Biological psychiatry, 76(7), 550-558.
Emsley, P., Lohkamp, B., Scott, W. G. & Cowtan, K. (2010) Features and development of Coot. Acta Crystallogr D Biol Crystallogr 66, 486-501. https://doi.org:10.1107/S0907444910007493
Ermakova, A. O.; Dunbar, F.; Rucker, J.; Johnson, M. W. (2022). A narrative synthesis of research with 5-MeO-DMT. Journal of Psychopharmacology, 36(3), 273-294.
Gasser, P., Kirchner, K. & Passie, T. (2015) LSD-assisted psychotherapy for anxiety associated with a life-threatening disease: a qualitative study of acute and sustained subjective effects. J Psychopharmacol 29, 57-68. https://doi.org:10.1177/0269881114555249
Glennon, R. A. et al. (2000) Binding of beta-carbolines and related agents at serotonin (5-HT(2) and 5-HT(1A)), dopamine (D(2)) and benzodiazepine receptors. Drug Alcohol Depend 60, 121-132. https://doi.org:10.1016/s0376-8716(99)00148-9
Golden, S. A., Covington, H. E., 3rd, Berton, O. & Russo, S. J. (2011) A standardized protocol for repeated social defeat stress in mice. Nat Protoc 6, 1183-1191. https://doi.org:10.1038/nprot.2011.361
Goodwin, G. M. et al. (2022) Single-Dose Psilocybin for a Treatment-Resistant Episode of Major Depression. New England Journal of Medicine 387, 1637-1648. https://doi.org:10.1056/NEJMoa2206443
Gottschalk, M. G., Mortas, P., Haman, M., Ozcan, S., Biemans, B. & Bahn, S. (2018) Fluoxetine, not donepezil, reverses anhedonia, cognitive dysfunctions and hippocampal proteome changes during repeated social defeat exposure. Eur. Neuropsychopharmacol 28, 195-210. https://doi.org:10.1016/j.euroneuro.2017.11.002
Feldman, P. and Rapoport (1986). Convenient synthesis of 6-methoxyindole and 6-methoxytryptophyl bromide. Synthesis, 9, 735-737.
Halberstadt, A. L. and Geyer, M. A. (2011). Multiple receptors contribute to the behavioral effects of indoleamine hallucinogens. Neuropharmacology, 61(3), 364-381.
Halberstadt, A. L., Koedood, L., Powell, S. B. & Geyer, M. A. (2011) Differential contributions of serotonin receptors to the behavioral effects of indoleamine hallucinogens in mice. J Psychopharmacol, 25, 1548-1561. https://doi.org:10.1177/0269881110388326
Henke, A. et al. (2018) Toward Serotonin Fluorescent False Neurotransmitters: Development of Fluorescent Dual Serotonin and Vesicular Monoamine Transporter Substrates for Visualizing Serotonin Neurons. ACS Chem Neurosci, 9, 925-934. https://doi.org:10.1021/acschemneuro.7b00320
Hesselgrave, N., Troppoli, T. A., Wulff, A. B., Cole, A. B. & Thompson, S. M. (2021) Harnessing psilocybin: antidepressant-like behavioral and synaptic actions of psilocybin are independent of 5-HT2R activation in mice. Proc Natl Acad Sci USA 118. https://doi.org:10.1073/pnas.2022489118
Hughes, Z. A. et al. (2005) Neurochemical evaluation of the novel 5-HT1A receptor partial agonist/serotonin reuptake inhibitor, vilazodone. Eur J Pharmacol 510, 49-57. https://doi.org:10.1016/j.ejphar.2005.01.018
Iderberg, H. et al. (2015) NLX-112, a novel 5-HT1A receptor agonist for the treatment of L-DOPA-induced dyskinesia: Behavioral and neurochemical profile in rat. Exp Neurol, 271, 335-350. https://doi.org:10.1016/j.expneurol.2015.05.021
Jastrzębska-Więsek, M.; Wesołowska, A.; Kołaczkowski, M.; Varney, M. A.; Newman-Tancredi, A.; Depoortere, R. (2022) The Selective 5-HT1A Receptor Agonist, NLX-112, Overcomes Tetrabenazine-Induced Catalepsy and Depression-like Behavior in the Rat. Behavioural Pharmacology, 33, 5, 333.
Kaplan, A. L. et al. (2022) Bespoke library docking for 5-HT(2A) receptor agonists with antidepressant activity. Nature, 610, 582-591. https://doi.org:10.1038/s41586-022-05258-z
Karuppagounder, S. S.; Ahuja, M.; Buabeid, M.; Parameshwaran, K.; Abdel-Rehman, E.; Suppiramaniam, V.; Dhanasekaran, M. (2012) Investigate the Chronic Neurotoxic Effects of Diquat. Neurochem Res, 37, 5, 1102-1111. https://doi.org/10.1007/s11064-012-0715-3.
Kim, K. et al. (2020) Structure of a Hallucinogen-Activated Gq-Coupled 5-HT2A Serotonin Receptor. Cell 182, 1574-1588 e1519. https://doi.org:10.1016/j.cell.2020.08.024
Kline, T. B., Benington, F., Morin, R. D., & Beaton, J. M. (1982). Structure-activity relationships in potentially hallucinogenic N, N-dialkyltryptamines substituted in the benzene moiety. Journal of Medicinal Chemistry, 25(8), 908-913.
Kozell, L. B. et al. (2023) Pharmacologic Activity of Substituted Tryptamines at 5-Hydroxytryptamine (5-HT) (2A) Receptor (5-HT(2A)R), 5-HT(2C)R, 5-HT(1A)R, and Serotonin Transporter. J Pharmacol Exp Ther 385, 62-75. https://doi.org:10.1124/jpet.122.001454
Krebs-Thomson, K., Ruiz, E. M., Masten, V., Buell, M. & Geyer, M. A. (2006) The roles of 5-HT1A and 5-HT2 receptors in the effects of 5-MeO-DMT on locomotor activity and prepulse inhibition in rats. Psychopharmacology (Berl) 189, 319-329. https://doi.org:10.1007/s00213-006-0566-1
Krishnan, V. et al. (2007) Molecular adaptations underlying susceptibility and resistance to social defeat in brain reward regions. Cell 131, 391-404. https://doi.org:10.1016/j.cell.2007.09.018
Kroeze, W. K. et al. (2015) PRESTO-Tango as an open-source resource for interrogation of the druggable human GPCRome. Nat Struct Mol Biol 22, 362-369. https://doi.org:10.1038/nsmb.3014
Kruegel, A. C.; Rakshit, S.; Li, X.; Sames, D. (2015). Constructing Iboga alkaloids via C—H bond functionalization: examination of the direct and catalytic union of heteroarenes and isoquinuclidine alkenes. The Journal of Organic Chemistry, 80(4), 2062-2071.
Laban, U.; Kurrasch-Orbaugh, D.; Marona-Lewicka, D.; & Nichols, D. E. (2001). A novel fluorinated tryptamine with highly potent serotonin 5-HT1A receptor agonist properties. Bioorganic & medicinal chemistry letters, 11(6), 793-795.
Le Foll, B. et al. (2016) Occupancy of Dopamine D3 and D2 Receptors by Buspirone: A [11C]-(+)-PHNO PET Study in Humans. Neuropsychopharmacology 41, 529-537. https://doi.org:10.1038/npp.2015.177
Li, N. et al. (2011) Glutamate N-methyl-D-aspartate receptor antagonists rapidly reverse behavioral and synaptic deficits caused by chronic stress exposure. Biol Psychiatry 69, 754-761. https://doi.org:10.1016/j.biopsych.2010.12.015
Liebschner, D. et al. (2019) Macromolecular structure determination using X-rays, neutrons and electrons: recent developments in Phenix. Acta Crystallogr D Struct Biol 75, 861-877. https://doi.org:10.1107/S2059798319011471
Loane, C.; Politis, M. (2012). Buspirone: what is it all about? Brain research, 1461, 111-118.
Mann, A.; Chesselet, M.-F. (2015) Chapter 8—Techniques for Motor Assessment in Rodents. In Movement Disorders (Second Edition); LeDoux, M. S., Ed.; Academic Press: Boston, 139-157. https://doi.org/10.1016/B978-0-12-405195-9.00008-1.
Mastinu, A.; Anyanwu, M.; Carone, M.; Abate, G.; Bonini, S. A.; Peron, G.; Memo, M. (2023). The Bright Side of Psychedelics: Latest Advances and Challenges in Neuropharmacology. International Journal of Molecular Sciences, 24(2), 1329.
Newman-Tancredi, A., Bardin, L., Auclair, A., Colpaert, F., Depoortere, R. & Varney, M. A. (2018) NLX-112, a highly selective 5-HT(1A) receptor agonist, mediates analgesia and antidepressant-like activity in rats via spinal cord and prefrontal cortex 5-HT(1A) receptors, respectively. Brain Res 1688, 1-7. https://doi.org:10.1016/j.brainres.2018.03.016
Nichols, D. E. (2018). Chemistry and structure-activity relationships of psychedelics. Behavioral Neurobiology of Psychedelic Drugs, 1-43.
Nichols, D. E., Frescas, S., Marona-Lewicka, D. & Kurrasch-Orbaugh, D. M. (2002) Lysergamides of isomeric 2,4-dimethylazetidines map the binding orientation of the diethylamide moiety in the potent hallucinogenic agent N,N-diethyllysergamide (LSD). J Med Chem 45, 4344-4349. https://doi.org:10.1021/jm020153s
Ohno, Y.; Shimizu, S.; Imaki, J.; Ishihara, S.; Sofue, N.; Sasa, M.; Kawai, Y. (2008) Anticataleptic 8-OH-DPAT Preferentially Counteracts with Haloperidol-Induced Fos Expression in the Dorsolateral Striatum and the Core Region of the Nucleus Accumbens. Neuropharmacology 55, 5, 717-723. https://doi.org/10.1016/j.neuropharm.2008.06.005
Olsen, R. H. J. et al. (2020). TRUPATH, an open-source biosensor platform for interrogating the GPCR transducerome. Nat Chem Biol, 16, 841-849. https://doi.org:10.1038/s41589-020-0535-8
Pokorny, T., Preller, K. H., Kraehenmann, R. & Vollenweider, F. X. (2016) Modulatory effect of the 5-HT1A agonist buspirone and the mixed non-hallucinogenic 5-HT1A/2A agonist ergotamine on psilocybin-induced psychedelic experience. Eur Neuropsychopharmacol, 26, 756-766. https://doi.org:10.1016/j.euroneuro.2016.01.005
Porter, R. H. P.; Benwell, K. R.; Lamb, H.; Malcolm, C. S.; Allen, N. H.; Revell, D. F.; Sheardown, M. J. (1999). Functional characterization of agonists at recombinant human 5-HT2A, 5-HT2B and 5-HT2C receptors in CHO-K1 cells. British journal of pharmacology, 128(1), 13.
Punjani, A., Rubinstein, J. L., Fleet, D. J. & Brubaker, M. A. (2017) cryoSPARC: algorithms for rapid unsupervised cryo-EM structure determination. Nat Methods 14, 290-296. https://doi.org:10.1038/nmeth.4169
Punjani, A., Zhang, H. & Fleet, D. J. (2020) Non-uniform refinement: adaptive regularization improves single-particle cryo-EM reconstruction. Nat Methods 17, 1214-1221. https://doi.org:10.1038/s41592-020-00990-8
Rasmussen, S. G. et al. (2011) Crystal structure of the beta2 adrenergic receptor-Gs protein complex. Nature 477, 549-555. https://doi.org:10.1038/nature10361
Reckweg, J. T.; Uthaug, M. V.; Szabo, A.; Davis, A. K.; Lancelotta, R.; Mason, N. L.; Ramaekers, J. G. (2022). The clinical pharmacology and potential therapeutic applications of 5-methoxy-N, N-dimethyltryptamine (5-MeO-DMT). Journal of Neurochemistry 162(1), 128-146.
Riga, M. S., Bortolozzi, A., Campa, L., Artigas, F. & Celada, P. (2016) The serotonergic hallucinogen 5-methoxy-N,N-dimethyltryptamine disrupts cortical activity in a regionally-selective manner via 5-HT1A and 5-HT2A receptors. Neuropharmacology 101, 370-378. https://doi.org:https://doi.org/10.1016/j.neuropharm.2015.10.016
Robinson, D. S. et al. (1990) Clinical effects of the 5-HT1A partial agonists in depression: a composite analysis of buspirone in the treatment of depression. J Clin Psychopharmacol 10, 67S-76S. https://doi.org:10.1097/00004714-199006001-00013
Roth, C. B., Hanson, M. A. & Stevens, R. C. (2008) Stabilization of the human beta2-adrenergic receptor TM4-TM3-TM5 helix interface by mutagenesis of Glu122(3.41), a critical residue in GPCR structure. J Mol Biol 376, 1305-1319. https://doi.org:10.1016/j.jmb.2007.12.028
Rothman, R. B.; Baumann, M. H.; Savage, J. E.; Rauser, L.; McBride, A.; Hufeisen, S. J.; Roth, B. L. (2000) Evidence for Possible Involvement of 5-HT2B Receptors in the Cardiac Valvulopathy Associated With Fenfluramine and Other Serotonergic Medications Circulation 102:2836-2841 https://doi.org/10.1161/01.CIR.102.23.2836
Sames, D.; Li, X.; Li, S.; Kruegel, A.; Karpowicz, R.; Carrera, I.; Rakshit, S. (2018). U.S. Pat. No. 9,988,377. Washington, DC: U.S. Patent and Trademark Office.
Schwelm, H. M.; Zimmermann, N.; Scholl, T.; Penner, J.; Autret, A.; Auwärter, V.; Neukamm, M. A. (2022). Qualitative and quantitative analysis of tryptamines in the poison of Incilius alvarius (Amphibia: bufonidae). Journal of Analytical Toxicology, 46(5), 540-548.
Shao, L. X. et al. (2021) Psilocybin induces rapid and persistent growth of dendritic spines in frontal cortex in vivo. Neuron 109, 2535-2544 e2534. https://doi.org:10.1016/j.neuron.2021.06.008
Shapiro, D. A. et al. (2003) Aripiprazole, a novel atypical antipsychotic drug with a unique and robust pharmacology. Neuropsychopharmacology 28, 1400-1411. https://doi.org:10.1038/sj.npp.1300203
Shulgin, A. T. & Shulgin, A. (1997) TIHKAL. The Continuation. Transform Press.
Speeter, M. E. & Anthony, W. C. (1954) The Action of Oxalyl Chloride on Indoles—a New Approach to Tryptamines. J Am Chem Soc 76, 6208-6210. https://doi.org:DOI 10.1021/ja01652a113
Strassman, R. J. (1996) Human psychopharmacology of N,N-dimethyltryptamine. Behav Brain Res 73, 121-124. https://doi.org:10.1016/0166-4328(96)00081-2
Takayama, H.; Maeda, M.; Ohbayashi, S.; Kitajima, M.; Sakai, S. I.; Aimi, N. (1995). The first total synthesis of (−)-mitragynine, an analgesic indole alkaloid in Mitragyna speciosa. Tetrahedron Letters, 36(51), 9337-9340.
The PyMOL Molecular Graphics System, Version 2.0 (Schrödinger, LLC).
Uthaug, M. V. et al. (2019) A single inhalation of vapor from dried toad secretion containing 5-methoxy-N,N-dimethyltryptamine (5-MeO-DMT) in a naturalistic setting is related to sustained enhancement of satisfaction with life, mindfulness-related capacities, and a decrement of psychopathological symptoms. Psychopharmacology (Berl) 236, 2653-2666. https://doi.org:10.1007/s00213-019-05236-w
von Rotz, R. et al. (2023) Single-dose psilocybin-assisted therapy in major depressive disorder: A placebo-controlled, double-blind, randomised clinical trial. EClinicalMedicine 56, 101809. https://doi.org:10.1016/j.eclinm.2022.101809
Wacker, D. et al. (2017) Crystal Structure of an LSD-Bound Human Serotonin Receptor. Cell 168, 377-389 e312. https://doi.org:10.1016/j.cell.2016.12.033
Wacker, D. et al. (2013) Structural features for functional selectivity at serotonin receptors. Science 340, 615-619. https://doi.org:10.1126/science.1232808
Wang, C. et al. (2013) Structural basis for molecular recognition at serotonin receptors. Science 340, 610-614. https://doi.org:10.1126/science.1232807
Weil, A. T. and Davis, W. (1994). Bufo alvarius: a potent hallucinogen of animal origin. Journal of ethnopharmacology, 41(1-2), 1-8.
Willner, P., Towell, A., Sampson, D., Sophokleous, S. & Muscat, R. (1987) Reduction of sucrose preference by chronic unpredictable mild stress, and its restoration by a tricyclic antidepressant. Psychopharmacology (Berl) 93, 358-364. https://doi.org:10.1007/BF00187257
Winter, J. C., Filipink, R. A., Timineri, D., Helsley, S. E. & Rabin, R. A. (2000) The paradox of 5-methoxy-N,N-dimethyltryptamine: an indoleamine hallucinogen that induces stimulus control via 5-HT1A receptors. Pharmacol Biochem Behav 65, 75-82. https://doi.org:10.1016/s0091-3057(99)00178-1
Xu, P. et al. (2021) Structural insights into the lipid and ligand regulation of serotonin receptors. Nature 592, 469-473. https://doi.org:10.1038/s41586-021-03376-8
Yamashita, K., Palmer, C. M., Burnley, T. & Murshudov, G. N. (2021) Cryo-EM single-particle structure refinement and map calculation using 1Servalcat. Acta Crystallogr D Struct Biol 77, 1282-1291. https://doi.org:10.1107/S2059798321009475
Zheng, S. Q., Palovcak, E., Armache, J. P., Verba, K. A., Cheng, Y. & Agard, D. A. (2017) MotionCor2: anisotropic correction of beam-induced motion for improved cryo-electron microscopy. Nat Methods 14, 331-332. https://doi.org:10.1038/nmeth.4193

1. A compound having the structure: 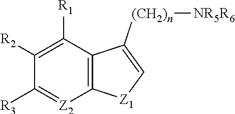
wherein
Z₁ is NR₄, O, or S;
Z₂ is CR₇ or N;
n is 1 to 5;
R₁ is —H, halogen, —O-alkyl, or together with R₂ forms —O—CH₂—O—;
R₂ is —H, —OH, —O-alkyl, —O-alkyl-aryl, —S-alkyl, —S-alkyl-aryl, or together with R₁ or R₃ forms —O—CH₂—O—;
R₃ is —H, —O-alkyl or together with R₂ forms —O—CH₂—O—;
R₄ is H or alkyl;
R₅ and R₆ are each independently H, alkyl, alkenyl, alkynyl or NR₅R₆ together form a substituted or unsubstituted heterocycle, wherein the heterocycle may be monocyclic or bicyclic,
R₇ is H or alkyl;
wherein R₁ is F, when R₂ is —O-(n-propyl) or when at least one of R₅ or R₆ is H, and
wherein the compound is other than a compound where
Z₁ is NR₄ and R₁, R₂, R₃, and R₄ are H;
n is 2, R₁ is halogen, R₂ is —OCH₃, R₃ is —H, R₄ is —H, and R₅ and R₆ are both methyl;
n is 2, R₁ is —F, R₂ is —OCH₃, R₃ is —H, R₄ is —H, and NR₅R₆ together are —N-pyrrolidine;
n is 2, R₁ and R₂ together forms —O—CH₂—O—, R₃ is —H, R₄ is —H, and R₅ and R₆ are both methyl; and
n is 2, R₁ and R₂ together forms —O—CH₂—O—, R₃ is —H, R₄ is —H; and R₅ and R₆ are both isopropyl,
or a pharmaceutically acceptable salt thereof.

2. The compound of claim 1 having the structure: 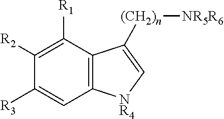
wherein
n is 1 to 5;
R₁ is a halogen or together with R₂ forms —O—CH₂—O—;
R₂ is —OH, —O-alkyl, —O-alkyl-aryl, —S-alkyl, —S-alkyl-aryl, or together with R₁ or R₃ forms —O—CH₂—O—;
R₃ is H or together with R₂ forms —O—CH₂—O—;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic,
wherein R₁ is F, when R₂ is —O-(n-propyl) or when at least one of R₅ or R₆ is H, and
wherein the compound is other than a compound where
n is 2, R₁ is halogen, R₂ is —OCH₃, R₃ is —H, R₄ is —H, and R₅ and R₆ are both methyl;
n is 2, R₁ is —F, R₂ is —OCH₃, R₃ is —H, R₄ is —H, and NR₅R₆ together are —N-pyrrolidine;
n is 2, R₁ and R₂ together forms —O—CH₂—O—, R₃ is —H, R₄ is —H, and R₅ and R₆ are both methyl; and
n is 2, R₁ and R₂ together forms —O—CH₂—O—, R₃ is —H, R₄ is —H; and R₅ and R₆ are both isopropyl,
or a pharmaceutically acceptable salt thereof.

3. The compound of claim 2, wherein
(a) R₁ is —F, —Cl or —Br,
(b)R₁ is —F or —Cl;
(c) R₁ is —F or —Br;
(d) R₁ is —F;
(e) n is 2;
(f) R₃ is —H;
(g) R₄ is H or C₁-C₂ alkyl;
(h) R₂ is —OH, —O-alkyl, —O-alkyl-aryl, —S-alkyl or —S-alkyl-aryl; and/or
(i) R₅ and R₆ are each independently methyl, ethyl, propyl, isopropyl, butyl, tert-butyl, or allyl; or wherein NR₅R₆ together form —N-azetidine, —N-pyrrolidine, —N-(3-methyl-pyrrolidine), —N-piperidine, —N-(3-pyrolline), —N-(1,2,3,6-tetrahydropyridine), —N-2-azabicyclo[2.2.2]oct-5-ene, or —N-7-ethyl-2-azabicyclo[2.2.2]oct-5-ene.

4. The compound of claim 2 having the structure: 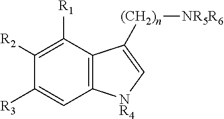
wherein
n is 1 to 5;
R₁ is —F or together with R₂ forms —O—CH₂—O—;
R₂ is —OH, —O-alkyl, —O-alkyl-aryl, —S-alkyl, —S-alkyl-aryl, or together with R₁ or R₃ forms —O—CH₂—O—;
R₃ is H or together with R₂ forms —O—CH₂—O—;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic, and
wherein the compound is other than a compound where
n is 2, R₁ is —F, R₂ is —OCH₃, R₃ is —H, R₄ is —H, and R₅ and R₆ are both methyl;
n is 2, R₁ is —F, R₂ is —OCH₃, R₃ is —H, R₄ is —H, and NR₅R₆ together are —N-pyrrolidine;
n is 2, R₁ and R₂ together forms —O—CH₂—O—, R₃ is —H, R₄ is —H, and R₅ and R₆ are both methyl; and
n is 2, R₁ and R₂ together forms —O—CH₂—O—, R₃ is —H, R₄ is —H; and R₅ and R₆ are both isopropyl,
or a pharmaceutically acceptable salt thereof.

5-6. (canceled)

7. The compound of claim 2, wherein
R₂ is —OH, —O—CH₃, —O—CH₂-Ph, —S—CH₃, or —S—CH₂-Ph; or
R₅ and R₆ are each independently methyl, ethyl, propyl, isopropyl, butyl, tert-butyl, or allyl; or wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

8-9. (canceled)

10. The compound of claim 1 wherein —NR₅R₆ is:

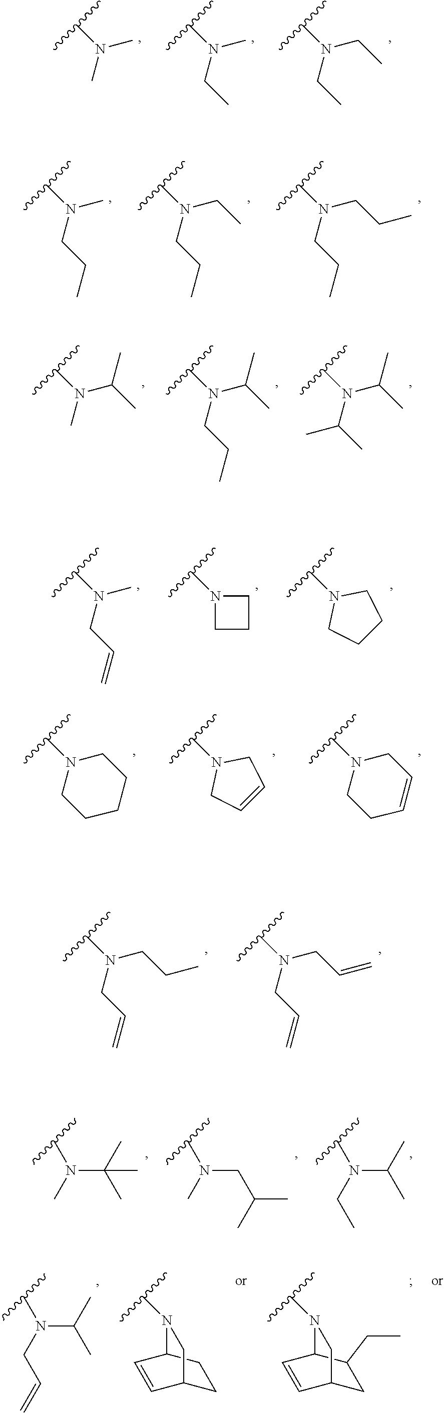

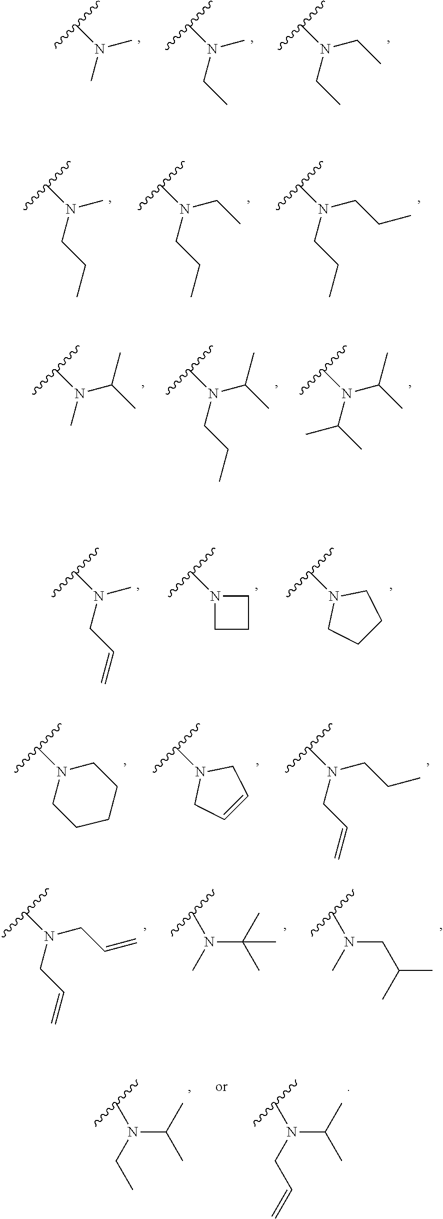

11. (canceled)

12. The compound of claim 2 having the structure: 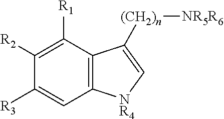
wherein n is 2;
R₂ is —OH, —O—CH₃, —S—CH₃, —O—CH₂-Ph, or —S—CH₂-Ph;
R₃ is —H; R₄ is H or —CH₃, and
R₅ and R₆ are each independently methyl, ethyl, propyl, isopropyl, tert-butyl, or allyl; or —NR₅R₆ together form —N-azetidine, —N-pyrrolidine, —N-(3-methyl-pyrrolidine), —N-piperidine, —N-(3-pyrolline), or —N-(1, 2, 3, 6-tetrahydropyridine); or 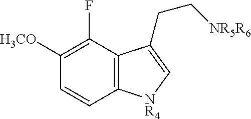
wherein R₄ is —H or —CH₃; and
R₅ and R₆ are each independently methyl, ethyl, propyl, isopropyl, tert-butyl, or allyl; or wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

13. (canceled)

14. The compound of claim 12 having the structure: 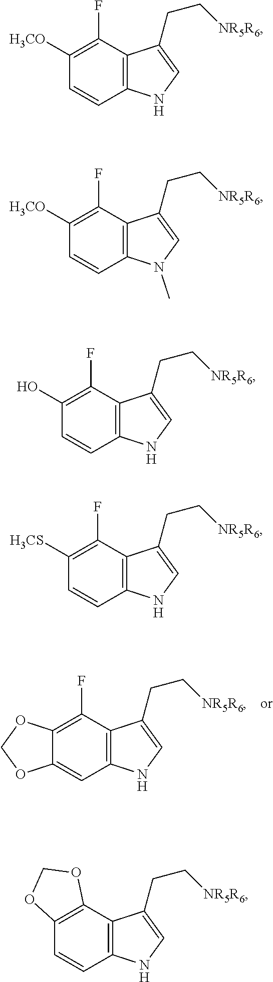
wherein R₅ and R₆ are each independently methyl, ethyl, propyl, isopropyl, tert-butyl, or allyl; or
wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine); or 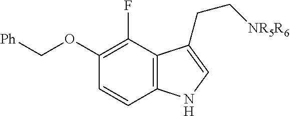
wherein NR₅R₆ together form —N-azetidine, —N-pyrrolidine, —N-(3-methyl-pyrrolidine), —N-piperidine, —N-(3-pyrolline), or —N-(1,2,3,6-tetrahydropyridine).

15-20. (canceled)

21. The compound of claim 2 having the structure:

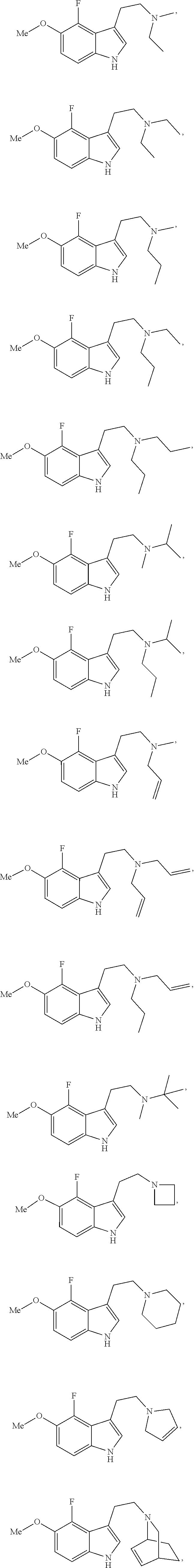

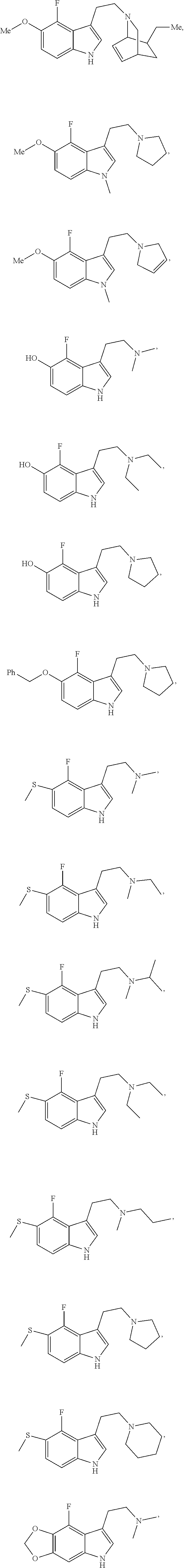

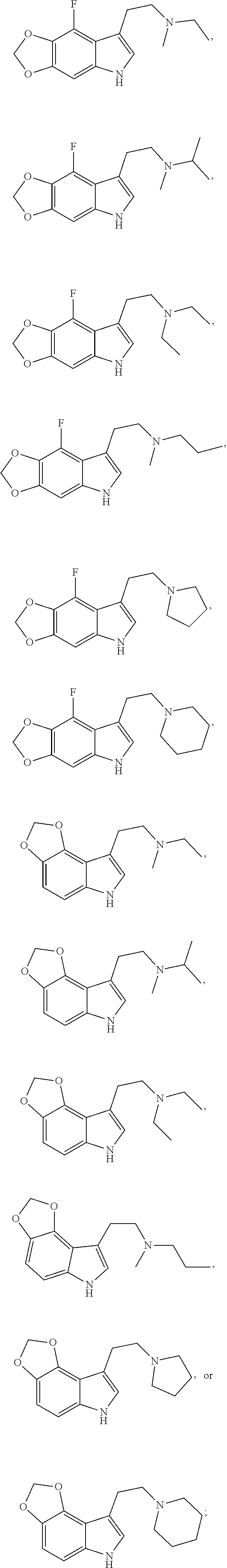

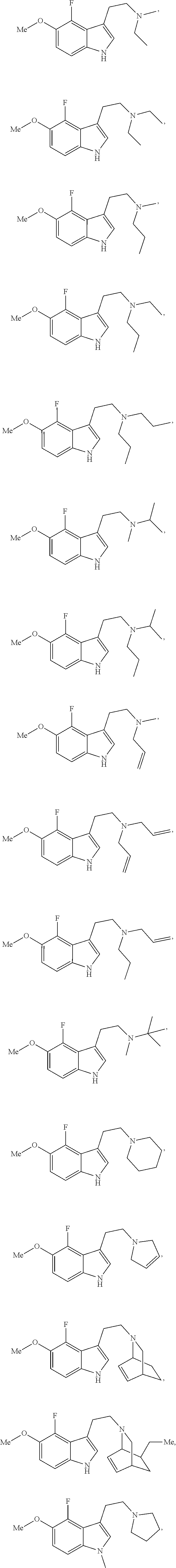

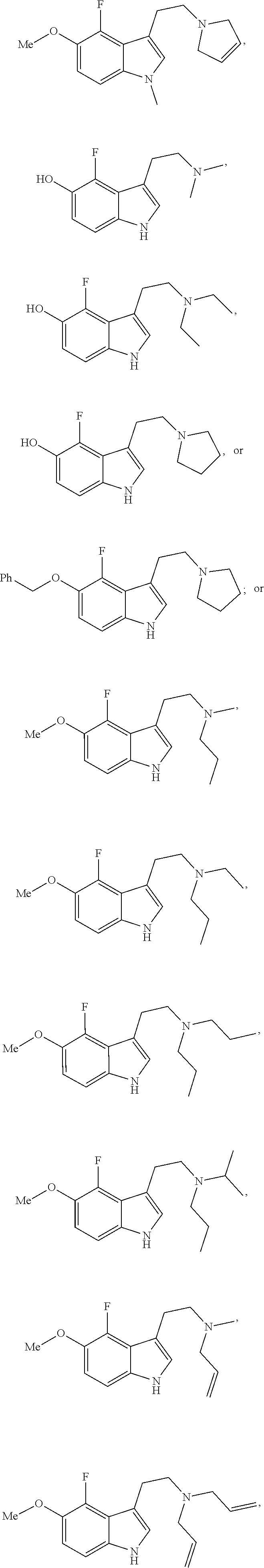

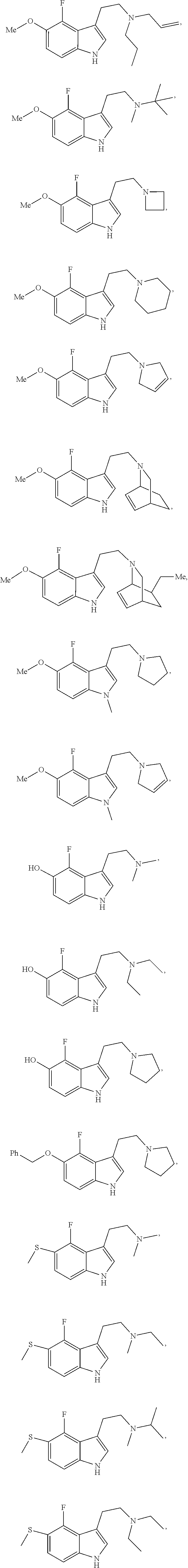

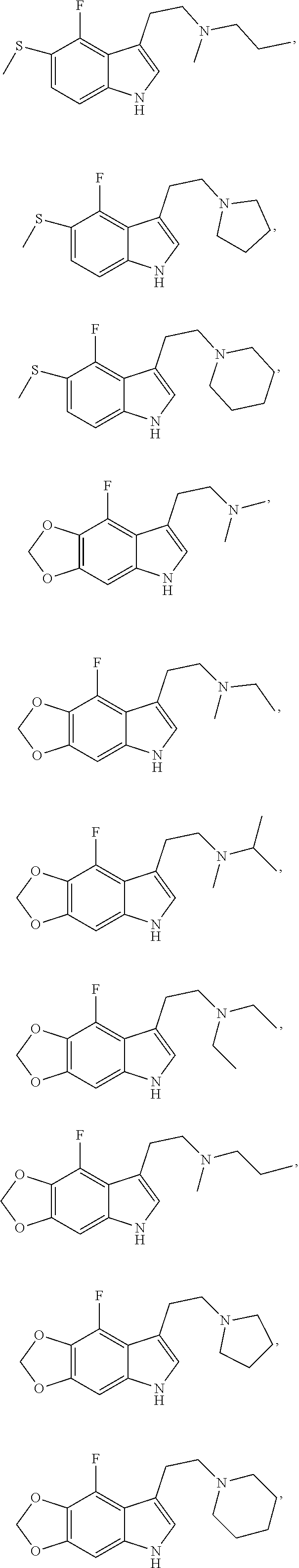

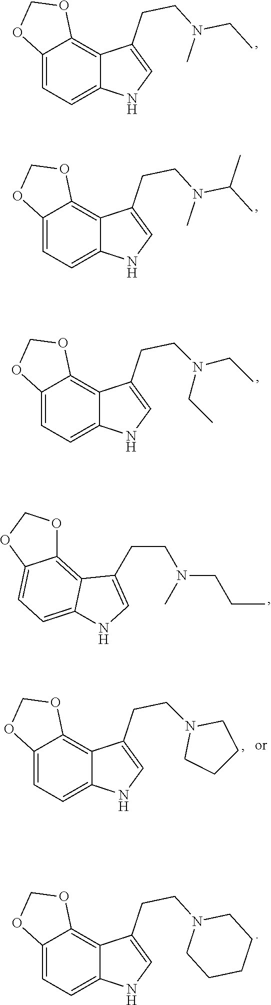 [SMILES: CCN(C)CCc1c[nH]c2ccc3c(c12)OCO3 ; CC(C)N(C)CCc1c[nH]c2ccc3c(c12)OCO3 ; CCN(CC)CCc1c[nH]c2ccc3c(c12)OCO3 ; CCCN(C)CCc1c[nH]c2ccc3c(c12)OCO3 ; c1cc2[nH]cc(CCN3CCCC3)c2c2c1OCO2 ; c1cc2[nH]cc(CCN3CCCCC3)c2c2c1OCO2]

22. (canceled)

23. The compound of claim 1 having the structure: 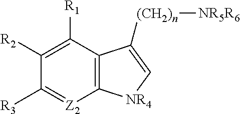
wherein
Z₂ is CR₇ or N;
n is 1 to 5;
R₁ is —H, halogen, —O-alkyl, or together with R₂ forms —O—CH₂—O—;
R₂ is —H, —OH, —O-alkyl, —O-alkyl-aryl, —S-alkyl, —S-alkyl-aryl, or together with R₁ or R₃ forms —O—CH₂—O—;
R₃ is —H, —O-alkyl or together with R₂ forms —O—CH₂—O—;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl; or. 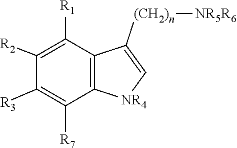
wherein n is 1 to 5;
R₁ is —H, a halogen, —O-alkyl, or together with R₂ forms —O—CH₂—O—;
R₂ is —H, —OH, —O-alkyl, —O-alkyl-aryl, —S-alkyl, —S-alkyl-aryl, or together with R₁ or R₃ forms —O—CH₂—O—;
R₃ is —H, —O-alkyl or together with R₂ forms —O—CH₂—O—;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl.

24. (canceled)

25. The compound of claim 23 having the structure: 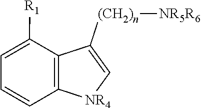
wherein
n is 2;
R₁ is a halogen or O-alkyl;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; or 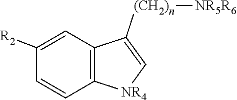
wherein
n is 1 to 5;
R₂ is —OH, —O-alkyl, —O-alkyl-aryl, —S-alkyl, or —S-alkyl-aryl;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; or 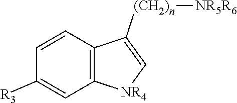
wherein
n is 1 to 5;
R₃ is —H or —O-alkyl;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; or 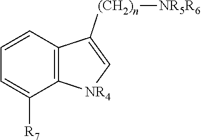
wherein
n is 2;
R₄ is H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is alkyl; or 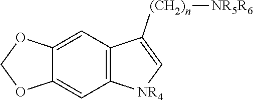
wherein
n is 1 to 5;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl; or 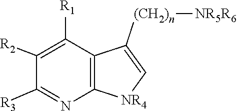
wherein
n is 1 to 5;
R₁ is —H, halogen, —O-alkyl, or together with R₂ forms —O—CH₂—O—;
R₂ is —H, —OH, —O-alkyl, —O-alkyl-aryl, —S-alkyl, —S-alkyl-aryl, or together with R₁ or R₃ forms —O—CH₂—O—;
R₃ is —H, —O-alkyl or together with R₂ forms —O—CH₂—O—;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic.
preferably wherein the compound has the structure: 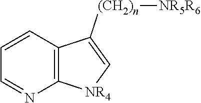
wherein
n is 1 to 5;
R₄ is —H or alkyl; and
R₅ and R₆ are each independently H, alkyl, alkenyl, alkynyl or NR₅R₆ together form a substituted or unsubstituted heterocycle, wherein the heterocycle may be monocyclic or bicyclic.

26-27. (canceled)

28. The compound of claim 1 having the structure: 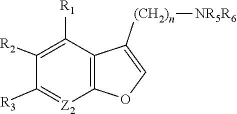
wherein
Z₂ is CR₇ or N;
n is 1 to 5;
R₁ is a halogen or together with R₂ forms —O—CH₂—O—;
R₂ is —OH, —O-alkyl, —O-alkyl-aryl, —S-alkyl, —S-alkyl-aryl, or together with R₁ or R₃ forms —O—CH₂—O—;
R₃ is H or together with R₂ forms —O—CH₂—O—; and
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl;
preferably wherein the compound has structure: 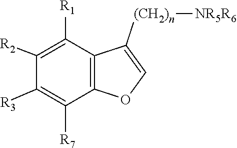
wherein n is 1 to 5;
R₁ is —F or together with R₂ forms —O—CH₂—O—;
R₂ is —OH, —O-alkyl, —O-alkyl-aryl, —S-alkyl, —S— alkyl-aryl, or together with R₁ or R₃ forms —O—CH₂—O—;
R₃ is H or together with R₂ forms —O—CH₂—O—; and
R₅ and R₆ are each independently H, alkyl, alkenyl, alkynyl or NR₅R₆ together form a substituted or unsubstituted heterocycle, wherein the heterocycle may be monocyclic or bicyclic
further preferably wherein the compound has the structure: 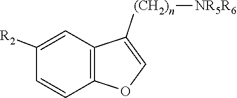
wherein n is 1 to 5;
R₂ is —OH, —O-alkyl, —O-alkyl-aryl, —S— alkyl, —S-alkyl-aryl, or together with R₁ or R₃ forms —O—CH₂—O—;
R₅ and R₆ are each independently H, alkyl, alkenyl, alkynyl or NR₅R₆ together form a substituted or unsubstituted heterocycle, wherein the heterocycle may be monocyclic or bicyclic.

29. The compound of claim 1 having the structure: 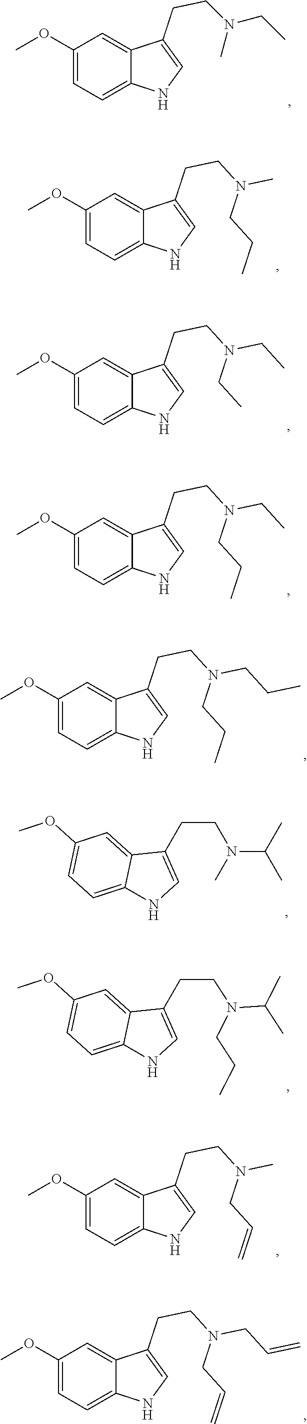 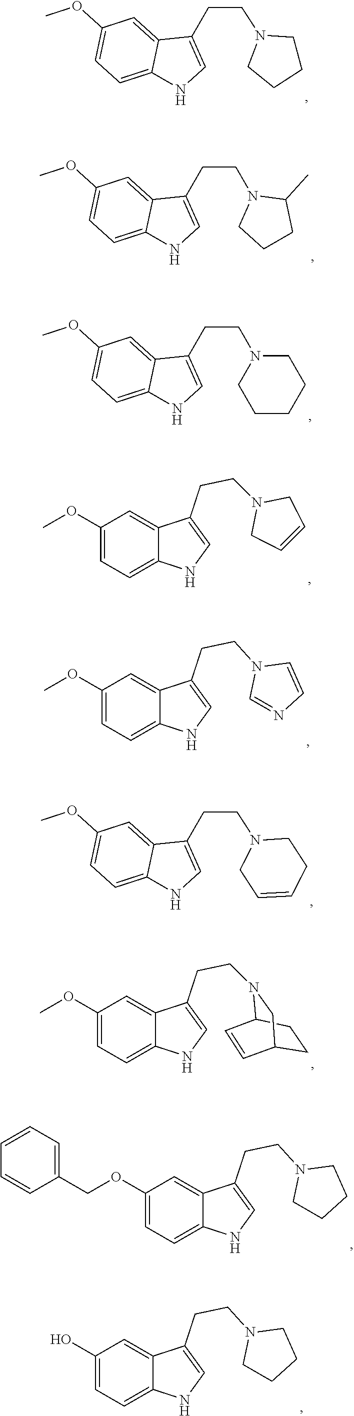 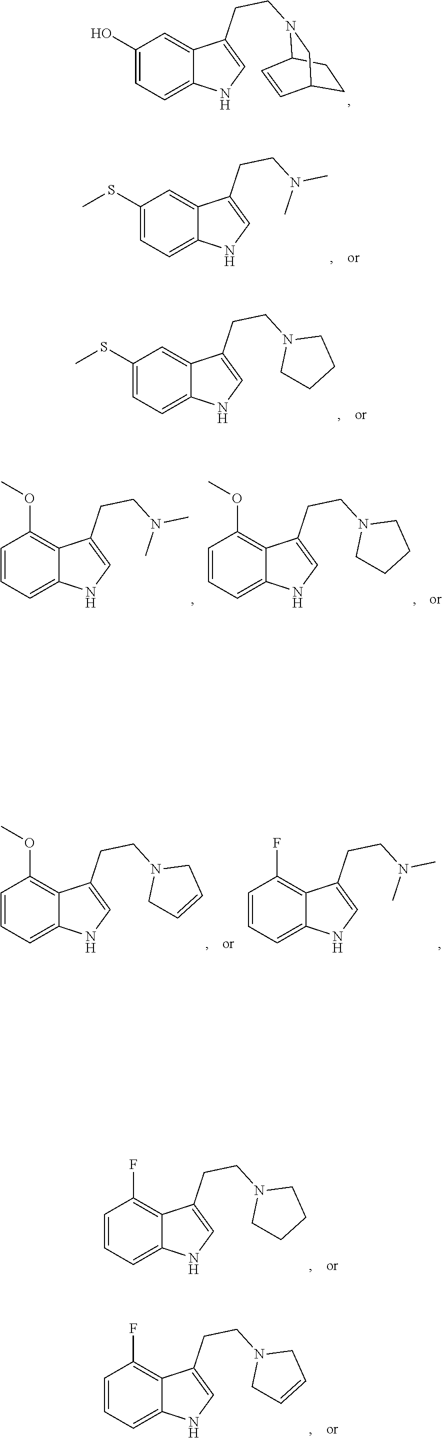 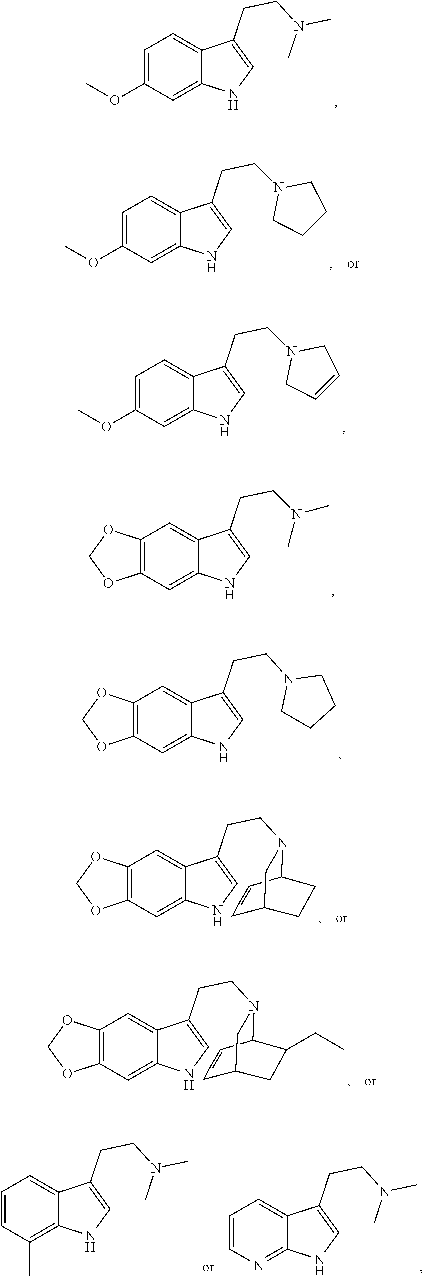 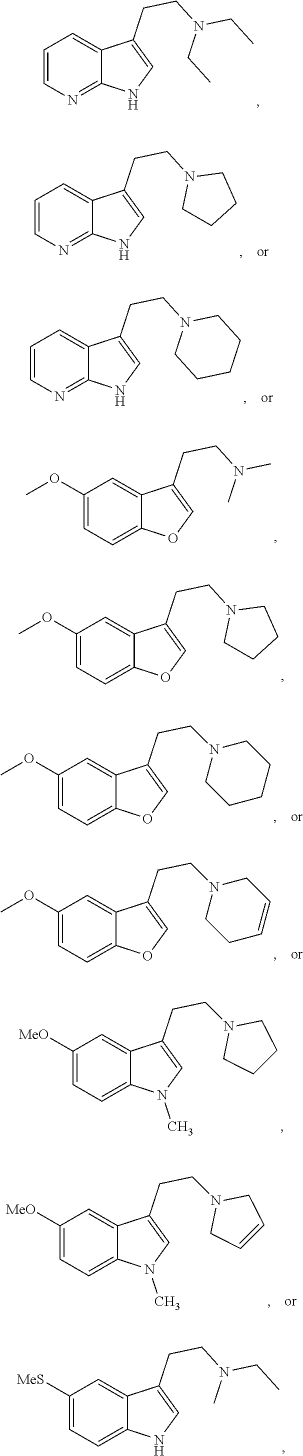
or a pharmaceutically acceptable salt thereof.

30. A composition comprising the compound of claim 1 and a carrier; preferably wherein the carrier is a pharmaceutically acceptable carrier.

31. A method of
(a) activating or selectively activating the 5HT1A receptor, or of simultaneously activating the 5HT1A and 5HT2A receptors, in a subject comprising administering to the subject an amount of the compound of claim 1 effective to activate or selectively activate the 5HT1A receptor, or simultaneously activate the 5HT1A and 5HT2A receptors;
(b) treating a subject afflicted with a neurological disease, psychiatric disorder, or substance use disorder, comprising administering to the subject an amount of the compound of claim 1;
(c) treating a subject afflicted with of a neurological disease, psychiatric disorder, or substance abuse disorder comprising administering to the subject a compound of claim 1 in combination with a compound having the structure: 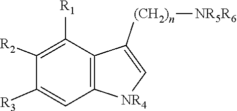
wherein n is 1 to 5;
R₁ is a halogen or together with R₂ forms —O—CH₂—O—;
R₂ is OH, O-alkyl, O-alkyl-aryl, S-alkyl, S-alkyl-aryl, or together with R₁ or R₃ forms —O—CH₂—O—;
R₃ is H or together with R₂ forms —O—CH₂—O—;
R₄ is H or alkyl; and
R₅ and R₆ are each independently H, alkyl, alkenyl, alkynyl or NR₅R₆ together form a substituted or unsubstituted heterocycle, and wherein the heterocycle may be mono or bicyclic;
together in an amount effective to treat the subject; or
(d) activating or selectively activating the 5HT1A receptor, or of simultaneously activating the 5HT1A and 5HT2A receptors, in a subject comprising administering to the subject an amount of a compound having the structure: 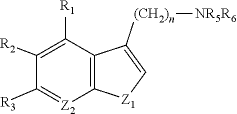
wherein
Z₁ is NR₄, O, or S;
Z₂ is CR₇ or N;
n is 1 to 5;
R₁ is —H, halogen, —O-alkyl, or together with R₂ forms —O—CH₂—O—;
R₂ is —H, —OH, —O-alkyl, —O-alkyl-aryl, —S-alkyl, —S-alkyl-aryl, or together with R₁ or R₃ forms —O—CH₂—O—;
R₃ is —H, —O-alkyl or together with R₂ forms —O—CH₂—O—;
R₄ is H or alkyl;
R₅ and R₆ are each independently H, alkyl, alkenyl, alkynyl or NR₅R₆ together form a substituted or unsubstituted heterocycle, wherein the heterocycle may be monocyclic or bicyclic; and
R₇ is H or alkyl;
or pharmaceutically acceptable salt thereof.

32. The method of claim 31, wherein
(a) the neurological disease is Parkinson's disease, dystonia, Huntington's disease, essential tremor, ataxia, chorea, myoclonus, ballismus, dysmetria, postural disorders, spasticity, blepharospasm, multiple sclerosis or cerebral palsy;
(b) psychiatric disorder, wherein the psychiatric disorder is depression, anxiety disorders, a mood disorder or anorexia;
(c) the substance use disorder is opioid use disorder, alcohol use disorder or stimulant use disorder including nicotine use disorder;
(d) the subject is a mammal; or
(e) the effective amount is from 25 mg to 500 mg of the compound.

33. (canceled)

34. The method of claim 32, wherein
(a) the mammal is a human;
(b) the effective amount is from 100 mg to 300 mg of the compound per kilogram of body weight;
(c) wherein the effective amount is 0.1-20 mg of compound per kilogram of body weight;
(d) further comprising administering a pharmaceutically acceptable carrier.

35-36. (canceled)

37. A process for
(a) the preparation of the compound of claim 1 comprising a step of dissolving a compound having the structure: 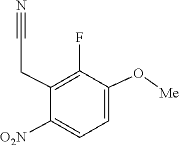 [SMILES: COc1ccc([N+](=O)[O-])c(CC#N)c1F]
in a suitable solvent followed by treatment with a suitable acid in the presence of a palladium catalyst at elevated pressure in an atmosphere of hydrogen; or
(b) the preparation of the compound of claim 1 comprising a step of dissolving a compound having the structure: 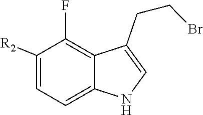
in a suitable solvent followed by treatment with a suitable amine.

38. The process of claim 37, wherein
(a) the solvent is methanol,
(b) the amine is a symmetric or unsymmetric alkyl amine; or the amine is a substituted or unsubstituted heterocyclic amine;
(c) the solvent is ethanol;
(d) the acid is acetic acid; or
(e) the elevated pressure is 60 psi.

39.-40. (canceled)

41. The compound of claim 1, wherein
a) R₁ is H, —O-alkyl, or together with R₂ forms —O—CH₂—O—;
b) R₂ is H, —OH, —O-alkyl-aryl, —S-alkyl, —S-alkyl-aryl, or R₂ together with R₁ or R₃ forms —O—CH₂—O—;
c) R₅ is H, methyl, n-propyl, alkenyl, alkynyl or NR₅R₆ together form a substituted or unsubstituted heterocycle, wherein the heterocycle is monocyclic or bicyclic, and
R₆ is H, methyl, n-propyl, tert-butyl, alkenyl, alkynyl or NR₅R₆ together form a substituted or unsubstituted heterocycle, wherein the heterocycle is monocyclic or bicyclic;
d) R₅ is H, ethyl, alkenyl, alkynyl or NR₅R₆ together form a substituted or unsubstituted heterocycle, wherein the heterocycle is monocyclic or bicyclic, and
R₆ is H, n-propyl, alkenyl, alkynyl or NR₅R₆ together form a substituted or unsubstituted heterocycle, wherein the heterocycle is monocyclic or bicyclic; or
e) R₅ is H, n-propyl, alkenyl, alkynyl or NR₅R₆ together form a substituted or unsubstituted heterocycle, wherein the heterocycle is monocyclic or bicyclic, and
R₆ is H, isopropyl, alkenyl, alkynyl or NR₅R₆ together form a substituted or unsubstituted heterocycle, wherein the heterocycle is monocyclic or bicyclic,
or a pharmaceutically acceptable salt thereof.
